(19) United States
(12) Patent Application Publication
(10) Pub. No.: US 2005/0239804 A1
(43) Pub. Date: Oct. 27, 2005

(54) Method for improved chemical synthesis of guanidinium alkaloids

(75) Inventors: Larry E. Overman, Corona del Mar, CA (US); Frank Stappenbeck, San Francisco, CA (US); Andrew I. McDonald, Irvine, CA (US)

Correspondence Address: STOUT, UXA, BUYAN & MULLINS LLP, 4 VENTURE, SUITE 300, IRVINE, CA, 92618, US

(73) Assignee: The Regents of the University of California

(21) Appl. No.: 10/815,023

(22) Filed: Mar. 30, 2004

Related U.S. Application Data
(63) Continuation of application No. 10/018,630, filed on Dec. 14, 2001, now abandoned.
(60) Provisional application No. 60/142,027, filed on Jun. 30, 1999.
(60) Provisional application No. 60/142,028, filed on Jun. 30, 1999.

(51) Int. Cl. A61K 31/519; C07D 491/14

(52) U.S. Cl. 514/257; 544/231

(57) ABSTRACT

Improved methods for convergent, total enantioselective synthesis of guanidinium alkaloid compounds including ones having cis- or -trans-1-oxo-and 1-iminohexahydropyrrolo [1,2c]pyrimidine units including, 13,14,15-isocrambescidin 800, crambescidin 800 and ptilomycalin A, for use as therapeutic agents having antifungal and/or antiviral and/or antitumor activity are provided. Methods for preparing novel pentacyclic intermediates for the preparation of the crambescidin/ptilomycalin family of guanidinium alkaloids and congeners are also disclosed.

This invention was made with Government Support under Grant No. NIH NHLBIS (HL-25854), awarded by the National Institutes of Health. The Government may have certain rights in this invention.

Throughout this application various publications are referenced. The disclosures of these publications in their entireties are hereby incorporated by reference into this application in order to more fully describe the state of the art to which this invention pertains.

### FIELD OF THE INVENTION

The present invention relates to methods for improved synthesis of guanidinium alkaloids, and more particularly to the total, convergent synthesis of the Crambescidin/Ptilomycalin family of guanidinium alkaloids.

### BACKGROUND OF THE INVENTION

Crambe crambe, a bright red encrusting sponge commonly found at shallow depths along the rocky coast of the Mediterranean is a rich source of structurally novel, bioactive alkaloids (FIG. 1). Among the most remarkable marine guanidine natural products are the family of alkaloids depicted in FIG. 1 that have a rigid pentacyclic guanidine carboxylic acid core linked to an ω-hydroxycarboxylic acid, ester or polyamine amide. This family, exemplified by ptilomycalin A (compound 1), the crambescidins (compounds 2-6), celeromycalin and fromiamycalin (compound 10) are characterized by a structurally unique pentacyclic guanidinium core that has a spermidine or hydroxyspermidine residue tethered by a long chain ω-hydroxycarboxylic acid spacer.

The alkaloid, ptilomycalin A, was reported by Kashman, Kakisawa and co-workers from sponges collected in the Caribbean and Red Sea (Kashman et al., J. Am. Chem. Soc., 1989, 111:8925). Ptilomycalin A exhibits cytotoxicity against P388 (IC₅₀ 0.1 μg/mL), L1210 (IC₅₀ 0.4 μg/mL) and KB (IC₅₀ 1.3 μl/mL), antifungal activity against Candida albicans (MIC 0.8 μg/mL) as well as considerable antiviral activity against Herpes simplex virus, type 1 (HSV-1) at a concentration of 0.2 μg/mL (Overman, L. E.; et al. supra). Recently, ptilomycalin A has been shown to inhibit the brain Na⁺, K⁺-ATPase and Ca²⁺-ATPase from skeletal sarcoplasmic reticulum with IC₅₀ values of 2 μM and 10 μM, respectively (Ohtani, I.; et al. Euro. J. Pharm. 1996, 310, 95).

In addition to Ptilomycalin A, numerous other complex marine alkaloids having a hydropyrrolo[1,2-c]pyrimidine-4-carboxylate part structure have been isolated including 13,14,15-isocrambescidin 800, crambescidin 800 and crambescidin 816 from Crambe crambe (Jares-Erijman et al., J. Org. Chem.,1991, 56:5712-5715; Jares-Erigman etal., J. Org. Chem. 1993, 58:4805-4808; Tavares et al., Biochem. Syst. Ecol., 1994, 22:645-646; Berlinck et al., J. Nat. Prod. 1993, 56, 1007-10015.)

Ptilomycalin A and several of the crambescidins show substantial antitumor, antiviral and antifungal activities. Crambescidin alkaloids have been described for use in inhibition of calcium channels (Jares-Erijman, et al., J. Org. Chem. 1993, 58:4805); inhibition of Na⁺, K⁺ and Ca²⁺-ATPases (Ohizumi et al., Eur. J. Pharmacol., 1996, 310:95). Batzelladine alkaloids, exemplified by batzelladines B and D (FIG. 1, Patil et al., J. Org. Chem., 1995, 60:1182; Patil et al., J. Org. Chem. , 1997, 62:1814; and Patil et al., J. Nat. Prod., 1997, 60:704), are reported to modulate protein-protein interactions that are important for immunological responses (Patil et al., 1995 and J. Org. Chem., 1997, supra).

As a result of its low abundance, 13,14,15-isocrambescidin 800 has not been extensively screened, although it is reported to be less cytotoxic to L-1210 cells than other crambescidins. (Jares-Erijman et al., J. Org. Chem., 1993, 58:4805-4868, supra).

The defining structural feature of the crambescidin alkaloids is a pentacyclic guanidine unit linked by a straight chain ω-hydroxycarboxylic acid tether to a spermidine or hydroxyspermidine unit. Extensive NMR studies demonstrated that the relative stereochemistry of the pentacyclic cores of crambescidin 800, crambescidin 816 and ptilomycalin A is identical (Jares-Erijman et. al., supra and Tavares et al., supra, while 13,14,15-isocrambescidin 800 isepimeric at C13, C14 and C15 relative to other members of the crambescidin family (Jares-Erijman et al., J. Org. Chem., 1993, supra, and Berlinck et al., J. Nat. Prod., supra). The absolute configuration of the guanidine moieties of 13,14,15-isocramescidin 800 and crambescidin 816 was established by oxidative degradation of the oxepene rings of these alkaloids to yield (S)-2-hydroxybutanoic acid (Jares-Erijman et al., J. Org. Chem., 1993, supra), while the absolute configuration of the hydroxyspermidine unit of crambescidin 816 was assigned using Mosher's method (Berlinck et al., supra, and Dale et al., J. Am. Chem. Soc., 1973, 95:512-519). Since ¹H NMR and ¹³C NMR chemical shifts in the hydroxyspermidine fragments of 13,14,15-isocrambescidin 800 are nearly identical to those of 2 and 3, it had been assumed that the stereochemistry at C43 is the same for all Crambescidins (Berlinck et al., supra

Apparent in the alkaloid compounds described (FIG. 1), is the occurrence of the hydropyrrolo[1,2c]pyrimidine unit with either the syn or anti relationship of the hydrogens flanking the pyrrolidine nitrogen.

In 1893, Biginelli reported the synthesis of dihydropyrimidines from the condensation of ethyl acetoacetate, aromatic aldehydes and urea. (Biginelli, P., Gazz. Chem. Ital., 1893, 23:360 (1893). Since Biginelli's disclosure, variations in all three components have led to the synthesis of an array of functionalized dihydropyrimidines and analogues. (Kappe, C. O., Tetrahedron, 49:6937 (1993). In 1993, we reported on the viability of “tethered Biginelli” condensations and verified that the cis orientation of the methine hydrogens was preferentially realized when the dehydrative condensation was promoted under Knoevenagel conditions to form cisi-1-oxohexahydropyrollo[1,2-c]pyrimidine products. (Overman et al., J. Org. Chem., 1993, 58:3235-3237). These reactions represented the first use of the Biginelli reactions in stereocontrolled organic synthesis. Tethered Biginelli condensations have already proved to be powerful reactions for the construction of Crambescidin (Overman et al., J. Am. Chem. Soc., 1995, 117:265) and batzelladine alkaloids (Franklin et al., J. Org. Chem, 1999, 62:6379). Recently it was reported to use acetals in place of alkenes to generate the aldehyde component of a Biginelli cyclization (Cohen et al., Organic Letters, 1999, V1 N13:2169-2172).

In 1995, an enantioselective total synthesis of (−)-Ptilomycalin A (Overman et al., J. Am. Chem. Soc., 117:2657 (1995)) was reported, which was the first total synthesis of a member of the Crambescidin alkaloid family.

There remains a need for improved methods of total, convergent synthesis of alkaloid compounds having biological activity, such as antifimgal, antiviral and/or anti-tumor activity.

### SUMMARY OF THE INVENTION

Accordingly, the present invention provides improved methods for convergent, total enantioselective synthesis of guanidinium alkaloid compounds including compounds having cis- or -trans-1-oxo-and 1-iminohexahydropyrrolo [1,2-c]pyrimidine units such as, 13,14,15-Isocrambescidin 800, Crambescidin 800 and Ptilomycalin A, for use as therapeutic agents having antifungal, antiviral and/or antitumor activity.

The compounds of the invention may be represented by the formulae:

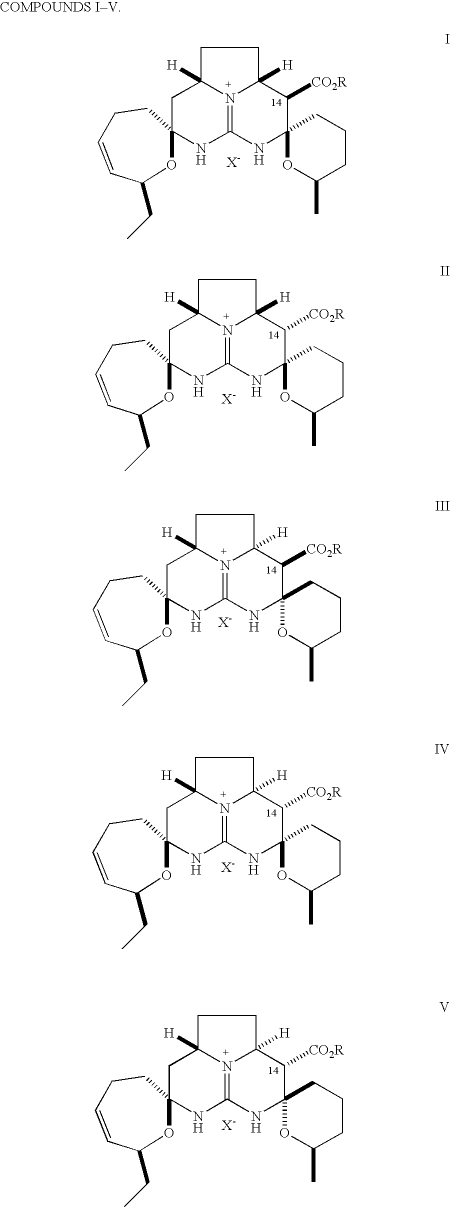

In which R═H, a carboxylic acid protecting group, an ω-alkoxycarboxylic acid or an ω-alkoxycarboxylic acid ester, and X=any pharrnaceutically acceptable counterion.

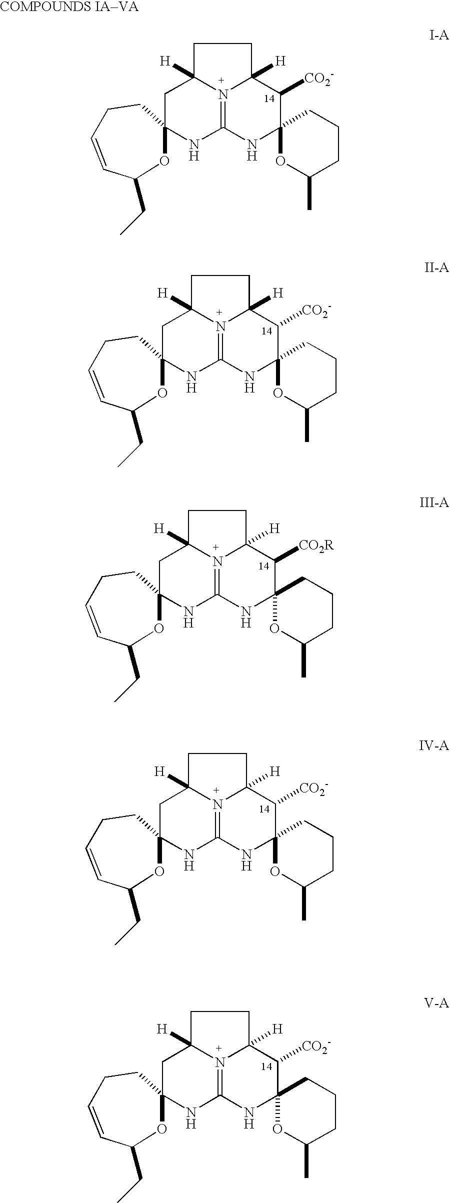

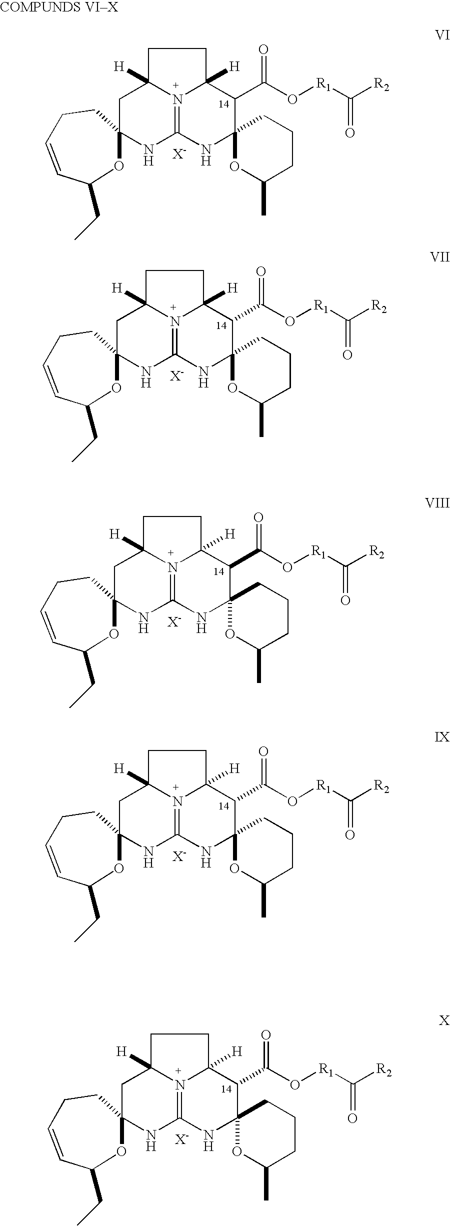

In which, R₁=any alkyl, aryl or substituted alkyl group, R₂═O⁻, OH, OG₁, a spermidine moiety or a substituted spermidine moiety, where G₁=a carboxylic acid protecting group and X=any pharmaceutically acceptable counterion.

The methods of the invention employ a convergent strategy for obtaining the compounds of the invention.

### BRIEF DESCRIPTION OF THE DRAWINGS

FIG. 1 depicts pentacyclic marine guanidine alkaloids obtained from marine organisms.

FIG. 2 depicts a molecular mechanics model of the Ptilomycalin A/Crambescidin core.

FIG. 3 depicts a hexahydropyrrolopyrimidine (compound B) having trans stereochemistry prepared by the methods of the invention.

FIG. 4 illustrates Biginelli condensations of tethered ureido aldehydes using the methods of the invention, as described in Example I, infra.

FIG. 5 is a synthetic scheme for making compounds 23-24, as described in Example I, infra.

FIG. 6 is a synthetic scheme for making compounds 25-28, as described in Example I, infra.

FIG. 7 presents two hypotheses for tethered Bignelli condensations under Knoevenagel conditions (Y═OH or NR₂).

FIG. 8 illustrates syntheses for compounds 37-43 as described in Example II, infra.

FIG. 9 depicts reactions for synthesis of Ptilomycalin A (compounds 46 and 47) as described in Example II, infra.

FIG. 10 depicts syntheses of compounds 49 to 53 as described in Example II, infra.

FIG. 11 depicts syntheses of compounds 54 to 56, as described in Example II, infra.

FIG. 12 illustrates syntheses of compounds 58 and 54 from compounds 57 and 59, as described in Example II, infra.

FIG. 13 illustrates the syntheses of compounds 61-68 and Ptilomycalin A, as described in Example II, infra.

FIG. 14 is a model showing expected preference for axial addition in forming the oxepene ring, as described in Example II, infra.

FIG. 15 depicts the syntheses of Crambescidin 800 (compound 2) and compounds 71-75, as described in Example III, infra.

FIG. 16 depicts the syntheses of compounds 76 to 80, as described in Example III, infra.

FIG. 17 depicts the syntheses of compounds 81 to 84, as described in Example III, infra.

FIG. 18 depicts the syntheses of compounds 85 to 88, as described in Example III, infra.

FIG. 19 depicts the syntheses of compounds 89 to 93, and compound 2 (Crambescidin 800), as described in Example III, infra.

FIG. 20 is a molecular mechanics model of the 13, 14, 15-Isocrambescidin 800 core and the Ptilomycalin A/Crambescidin core, as described in Example IV, infra.

FIG. 21 is a retrosynthetic analysis of the Isocrambescidin core, as described in Example IV, infra.

FIG. 22 depicts syntheses of compounds 99-103 as described in Example IV, infra.

FIG. 23 depicts syntheses of compounds 105a-106 as described in Example IV, infra.

FIG. 24 shows the pentacyclic intermediate in the (−)-Ptilomycalin A synthesis as described in Example IV I, infra.

FIG. 25 shows the synthesis of compounds 105a and 105b through 108a and 108b, as described in Example IV, infra.

FIG. 26 shows the synthesis of Isocrambescidin 800 (compound 2) as described in Example IV, infra.

FIG. 27 depicts the creation of compounds 114-116 as described in Example IV, infra.

FIG. 28 depicts the synthesis of compound 117, as described in Example IV, infra.

FIG. 29 shows data for Mosher's derivatives of compounds 10 and 117 as described in Example IV, infra.

FIG. 30 is a scheme showing a Biginelli condensation between a tethered guanyl aldehyde and a ω-ketoester afforded 1-iminohexahydropyrrolo[1,2-c]pyrimidine intermediates with the trans relationship about the pyrrolidine ring thus providing a strategy for constructing the Isocrambescidin core.

FIG. 31 is three-dimensional models of methyl ester analogs of four pentacyclic guanidine units as described in Example V, infra. Models depicting only heavy atoms are oriented identical to the line drawings; models also showing hydrogen atoms are oriented with the guanidine unit projecting back.

FIG. 32 is a scheme showing a Biginelli condensation between a guanyl aldehyde (or aminal) and a ω-ketoester afforded 1-iminohexahydropyrrolo[1,2-c]pyrimidine intermediates with the trans relationship about the pyrrolidine ring thus providing a strategy for constructing the Isocrambescidin core.

FIG. 33 shows the retrosynthesis of the pentacyclic core of Isocrambescidin 800 (compound 10) as described in Example V, infra.

FIG. 34 depicts the synthesis of compound 134, as described in Example V, infra.

FIG. 35 depicts the formation of Pentacycle 135, as described in Example V, infra.

FIG. 36 depicts the formation of Pentacycle 135 using pyridinium p-toluenesulfonate, as described in Example V, infra.

FIG. 37 depicts the formation of Pentacycle 135 using HCl, as described in Example V, infra.

FIG. 38 depicts models of the methyl ester analog of 139 showing the two chair conformations of the hydropyran ring. In conformation A, the methyl group is axial and in conformation B it is equatorial.

FIG. 39 depicts the formation of Penacycle 135b using DCl, as described in Example V, infra.

FIG. 40 depicts.the formation of compounds 141-143, as described in Example V, infra.

FIG. 41 depicts the formation of compounds 141-143 from compound 138, as described in Example V, infra.

FIG. 42 depicts the formation of compounds 145-146, as described in Example V, infra.

FIG. 43 depicts the formation of compounds 141-147, as described in Example V, infra.

FIG. 44 depicts the F-19 NMR data for Mosher derivatives of compounds 10 and 147, as described in Example V, infra.

FIG. 45 depicts the relative energy of pentacyclic guanidine isomers, as described in Example V, infra.

FIG. 46 is a schematic diagram for a modified enantioselective total synthetic approach.

FIG. 47 is a schematic diagram of the tethered Biginelli condensation.

FIG. 48 is an improved synthetic approach of compound 152.

FIG. 49 is a schematic diagram for making enantiopure Iodide compound 166, as described in Example VI, infra.

FIG. 50 is a schematic diagram for coupling of the C(1)-C(7) fragment with the tricyclic intermediate, as described in Example VI, infra.

FIG. 51 is a schematic diagram for making a pentacyclic acid as described in Example VI, infra.

FIG. 52 is a schematic diagram for an improved method for making pentacyclic acids, as described in Example VII, infra.

FIG. 53 is a diagram showing the synthesis of compounds 180 to 183 as described in Example VII, infra.

FIG. 54 is a depiction of the synthesis of compounds 185-189 as described in Example VII, infra.

FIG. 55 is a depiction of the synthesis of compound 194 as described in Example VII, infra.

FIG. 56 is a mean graph response of Ptilomycalin A as described in Example VIII, infra.

FIG. 57 is a mean graph response of Isocrambescidin 800 trihydrochloride as described in Example VIII, infra.

FIG. 58 is a mean graph response of Triacetylcrambescidin 800 chloride as described in Example VIII, infra.

FIG. 59 is a mean graph response of Crambescidin 657 hydrochloride as described in Example VIII, infra.

FIG. 60 is a mean graph response of Crambescidin 800 trihydrochloride as described in Example VIII, infra.

FIG. 61 is a mean graph response of Triacetylisocrambescidin 800 chloride as described in Example VIII, infra.

FIG. 62 is a mean graph response of 13-Epiptilomycalin A as described in Example VIII, infra.

### DETAILED DESCRIPTION OF THE INVENTION

The present invention provides methods for enantioselective total synthesis of guanidinium alkaloids and congeners in convergent fashion using tethered Biginelli reactions. This invention allows all of the heavy atoms of the pentacyclic core of the Crambescidin/Ptilomycalin A and Isocrambescidin to be assembled in one key step. The compounds produced may be used for pharmacological screening using known methods, to identify compounds having desired biological therapeutic activity, for example as antiviral, antifungal and/or antitumor agents.

For synthesizing the compounds of the invention, a method was developed for controlling stereoselection in tethered Biginelli condensations to synthesize either the cis or trans stereoisomer of 1-oxo and 1-iminohexahydropyrrolo[1,2-c]pyrimidines.

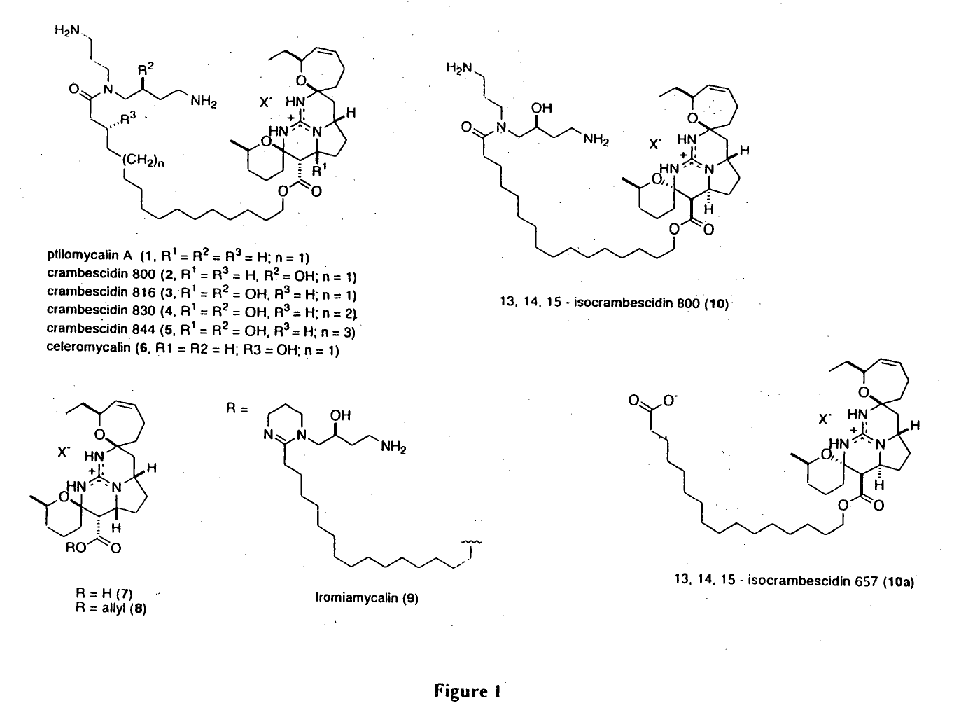

The invention also provides methods for the preparation ofpentacyclic acid (e.g., compound 7 of FIG. 1) and precursor allyl ester (e.g., compound 8 of FIG. 1) intermediates, that allows analogs to be prepared that are not available by degradation of the sponge extracts (Kashunan, Y.; et al. J. Am. Chem. Soc. 1989, 111, 8925; Ohtani, I.; et al. J. Am. Chem. Soc. 1992, 114, 8472; Jares-Erijman, E. A.; et al. J. Org. Chem. 1991, 56, 5712). It is expected that analogs will show improved pharmacological properties.

The present invention relates to compounds of the general formulae:

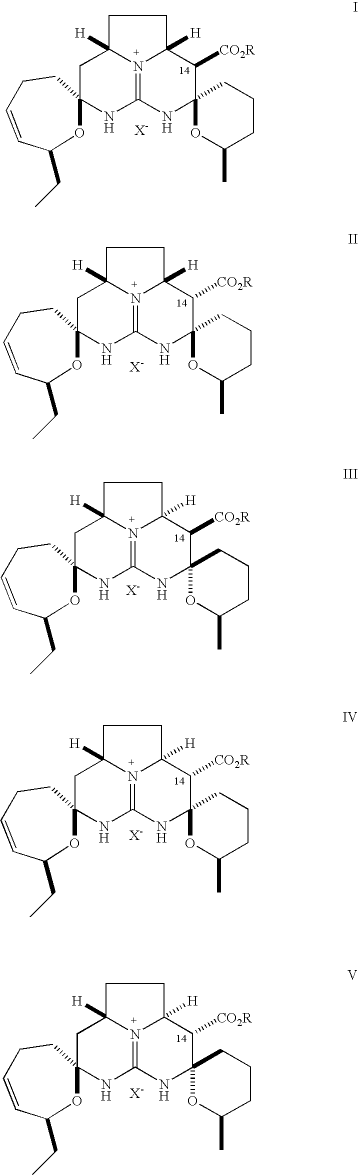

In which R═H, a carboxylic acid protecting group, an ω-allkoxycarboxylic acid or an ω-alkoxycarboxylic acid ester, and X=any pharmaceutically acceptable counterion.

In one embodiment R═H and X═Cl⁻.

In another embodiment R=allyl and X═Cl⁻.

In another embodiment R═(CH₂)₁₅CO₂H and X═Cl⁻,

The invention includes methods for preparing the compounds. In a method for preparing compound I having the formula: 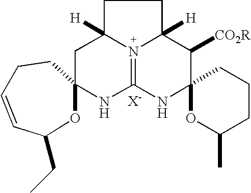
in which R=H, a carboxylic acid protecting group, an ω-alkoxycarboxylic acid or an ω-alkoxycarboxylic acid ester, and X=any pharmaceutically acceptable counterion
a compound having the formula: 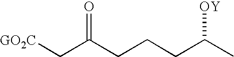
in which G=a carboxylic acid protecting group, an ω-alkoxycarboxylic acid or and ω-alkoxycarboxylic acid ester, and Y=alcohol protecting group, is reacted with a compound of the formula:

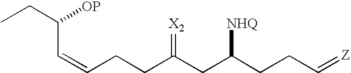

In which X₂=O or a ketone protecting group, Z=alkene or carbonyl protecting group, P=alcohol protecting group, and Q=amino carbonyl group, to produce a compound of the formula: 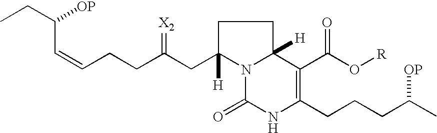
in which X₂=O or ketone protecting group, P=alcohol protecting group, and R=carboxylic acid protecting group, ω-alkoxycarboxylic acid or ω-alkoxycarboxylic acid ester which is subsequently converted to the pentacyclic compound by deprotection, incorporation of ammonia, and cyclization.

Another embodiment is a method for preparing compound II:

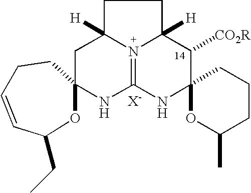

In which, R=H, a carboxylic acid protecting group, an ω-alkoxycarboxylic acid or an ω-alkoxycarboxylic acid ester, and X=any pharmaceutically acceptable counterion, by epimerizing the stereocenter at carbon-14 of the compound I.

In another embodiment, a method for preparing compounds IV and V: 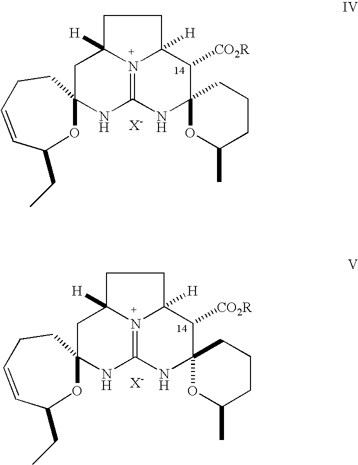
in which R=H, a carboxylic acid protecting group, an ω-alkoxycarboxylic acid or an ω-alkoxycarboxylic acid ester, and X=any pharmaceutically acceptable counterion by reacting compound 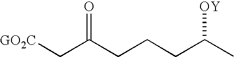
in which G=carboxylic acid protecting group, an ω-alkoxycarboxylic acid or an ω alkoxycarboxylic acid ester, and Y=alcohol protecting group, with compound

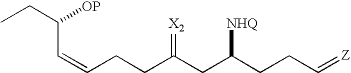

In which X₂=O or ketone protecting group, Z=alkene or carbonyl protecting group P=alcohol protecting group, and Q=amidinyl group to produce a compound of the formula

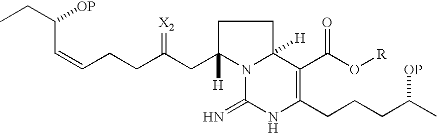

In which X₂=O or ketone protecting group, P=alcohol protecting group, R=carboxylic acid protecting group, an ω-alkoxycarboxylic acid or an ω-alkoxycarboxylic acid ester which is subsequently converted IV and V by deprotection and cyclization.

Another embodiment is a method for preparing compound III:

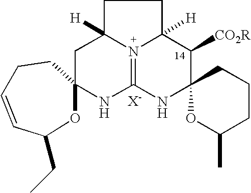

In which R═H, a carboxylic acid protecting group, an ω-alkoxycarboxylic acid or an ω-alkoxycarboxylic acid ester, and X=any pharmaceutically acceptable counterion by epimerizing the stereocenter at carbon-14 and carbon 15 of the compound IV.

Protecting groups and strategies for synthesis of organic compounds are well known in the art (Protective Groups in Organic Synthesis, 2^{nd} Ed. T. W. Greene, P.G.M. Wuts, J. Wiley and Sons, Inc. New York, 1991).

Carboxylic acid protecting groups may be chosen from the following groups including, but not limited to, esters and amides.

Alcohol protecting groups may be chosen from the following groups including, but not limited to, ether groups, silyl protecting groups, such as TIPS, TBDMS, SEM, THP, TES, TMS, or ester groups, such as acetates, benzoates, and mesitoates.

Carbonyl protecting groups may be chosen from the following groups including, but not limited to, ethers, cyclic or acyclic acetals, ketals, thioketals or thioacetals.

Amine protecting groups may be chosen from the following groups including, but not limited to, N-alkyl, such as benzyl, methyl, N-Silyl groups, N-acyl groups, N-carbamates.

The invention also provides compounds of the general formulas:

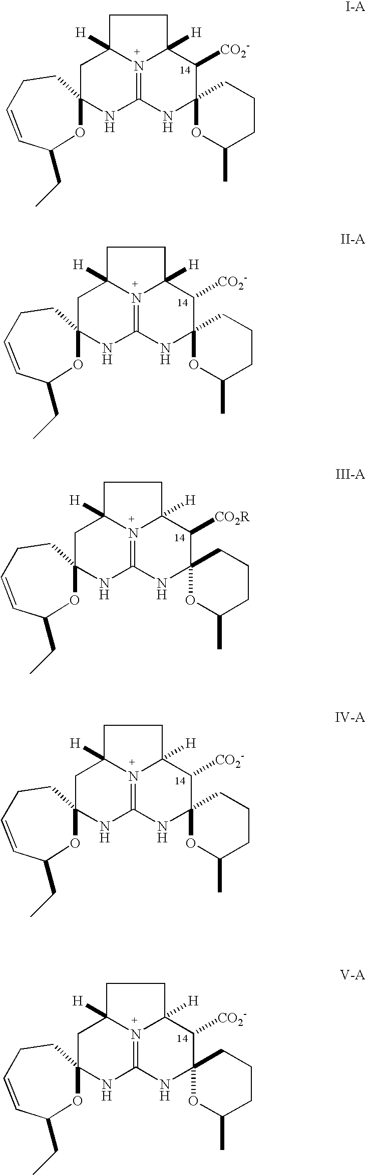

Another embodiment is a method for preparing compounds I-A to V-A, which can be prepared by following the method of preparing compounds I to V, respectively, and including an additional step of removing the carboxylic acid protecting group (R) or deprotecting the carboxylic acid.

Further, the invention provides compounds having the formula:

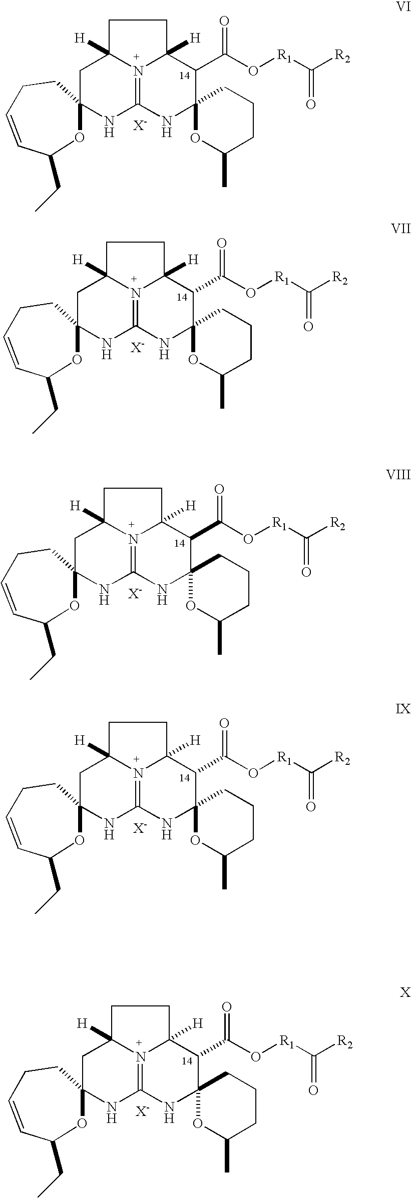

In which, R₁=any alkyl, aryl or substituted alkyl group, R₂=O⁻, OH, OG₁, spermidine moiety or substituted spermidine moiety, in which G₁=carboxylic acid protecting group and X=any pharmaceutically acceptable counterion

Methods for preparing compounds VI-X:

Compound VI is prepared as described for compound I in which R is an ω-alkoxycarboxylic acid as depicted in the figure below:

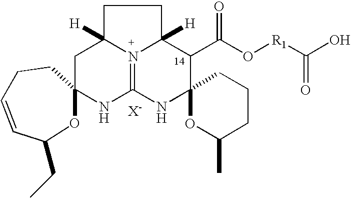

In which R₁=any alkyl, aryl or substituted alkyl group and including an additional step of reacting the pentacyclic compound of the formula above with a protected spermidine or a protected substituted sperimidine and subsequently deprotecting to produce VI.

Compound VII is prepared as described for compound II in which R is an ω-alkoxycarboxylic acid acid as depicted in the figure below:

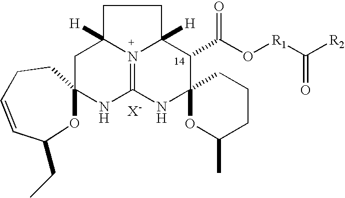

In which R₁=any alky, aryl or substituted alkyl group and including an additional step of reacting the pentacyclic compound of the formula above with a protected spermidine or a protected substituted sperimidine and subsequently deprotecting to produce VII.

Compound VIII is prepared as described for compound III in which R is an ω-alkoxycarboxylic acid acid as depicted in the figure below:

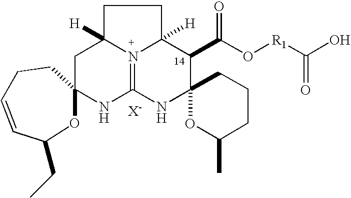

In which R₁=any alkyl, aryl or substituted alkyl group and including an additional step of reacting the pentacyclic compound of the formula above with a protected spermidine or a protected substituted sperimidine and subsequently deprotecting to produce VIII.
Compound IX is prepared as described for compound IV in which R is an ω-alkoxycarboxylic acid acid as depicted in the figure below:

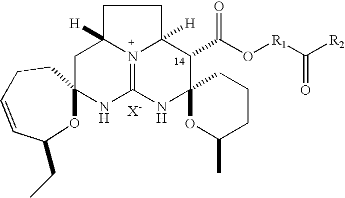

In which R₁=any alkyl, aryl or substituted alkyl group and including an additional step of reacting the pentacyclic compound of the formula above with a protected spermidine or a protected substituted sperimidine and subsequently deprotecting to produce IX.

Compound X is prepared as described for compound V in which R is an ω-alkoxycarboxylic acid acid as depicted in the figure below:

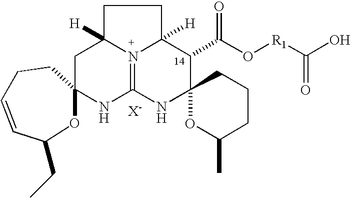

In which R₁=any alkyl, aryl or substituted alkyl group and including an additional step of reacting the pentacyclic compound of the formula above with a protected spermidine or a protected substituted sperimidine and subsequently deprotecting to produce X.

The compounds of the invention include where applicable, a geometric or optical isomer of the compound or racemic mixture thereof.

Pharmaceutically acceptable counterions may be chosen from the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate.

The compounds of the invention may be used in therapy as antiviral, antifungal and/or as itumor agents. For such uses, the compounds are administered intravenously, intramuscularly, topically, transdermally by means of skin patches, bucally, suppositorally or orally to man or other animals. The compositions can be presented for administration to humans and animals in a variety of dosage forms which include, but are not limited to, liquid solutions or suspensions, tablets, pills, powders, suppositories, polymeric microcapsules or microvesicles, liposomes, and injectable or infusible solutions, granules, sterile parenteral solutions or suspensions, oral solutions or suspensions, oil in water and water in oil emulsions containing suitable quantities of the compound, suppositories and in fluid suspensions or solutions. The preferred form depends upon the mode of administration and the therapeutic application.

For oral administration, either solid or fluid unit dosage forms can be prepared. For preparing solid compositions such as tablets, the compound can be mixed with conventional ingredients e.g. talc, magnesium stearate, dicalcium phosphate, magnesium aluminum silicate, calcium sulfate, starch, lactose, acacia, methylcellulose, and ftmctionally similar materials as pharmaceutical diluents or carriers. Capsules are prepared by mixing the compound with an inert pharmaceutical diluent and filling the mixture into a hard gelatin capsule of suitable size. Soft gelatin capsules are prepared by machine encapsulation of a slurry of the compound with vegetable oil, light liquid petrolatum or other inert oil.

Dosage forms for oral administration include syrups, elixirs, and suspensions. The forms can be dissolved in an aqueous vehicle along with sugar, aromatic flavoring agents and preservatives to form a syrup. Suspensions can be prepared with an aqueous vehicle with the aid of a suspending agent for example acacia, tragacanth, methylcellulose and the like.

For parenteral administration, fluid unit dosage forms can be prepared utilizing the compound and a sterile vehicle. In preparing solutions the compound can be dissolved in the vehicle for injection and filter sterilized before filling into a suitable vial or ampoule and sealing. Adjuvants such as a local anesthetic, preservative and buffering agents can be dissolved in the vehicle. The composition can be frozen after filling into a vial and the water removed under vacuurn. The dry lyophilized powder can then be sealed in the vial and reconstituted prior to use.

The most effective mode of administration and dosage regimen for the molecules of the present invention depends upon the severity and course of the disease, the subject's health and response to treatment and the judgment of the treating physician. Accordingly, the dosages of the molecules should be titrated to the individual subject.

Adjustments in the dosage regimens and/or modes of administration may be made to optimize the antiviral, antifumgal or antitumor efficacy of the compounds of the invention.

Efficacy of the compounds of the invention in therapy may be assessed using known methods. For example, efficacy of the compounds as anti-tumor agents may be assessed by tumor biopsy or non-invasive procedures to determine tumor growth inhibition. Similarly, efficacy of the compounds as anti-viral or anti-fungal agents may be determined using standard protocols such as as assays to detect decreases in numbers of viral particles or fungal cells, or in the numbers of virally or fungally infected cells.

The following examples are presented to illustrate the present invention and to assist one of ordinary skill in making and using the same. The examples are not intended in any way to otherwise limit the scope of the invention.

### EXAMPLE I

Synthesis of cis- or trans-1-Oxo-and 1-Iminohexahydropoyrrolo[1,2-c]pyrimidines

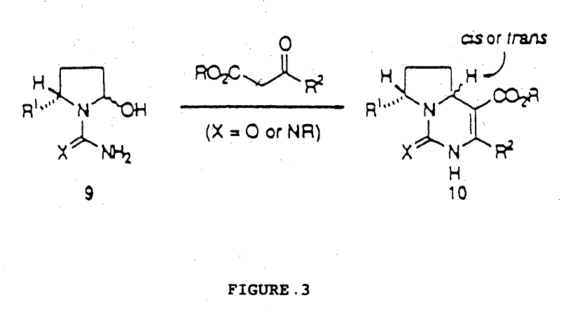

This Example describes a method for controlling the stereoselectivity of tethered Biginelli condensations. Modification of the electrophilic reaction component permits access to hexahydropyrrolopyrimidines (Compound 10 in FIG. 3) having either the cis or trans stereochemistry.

Materials and Methods

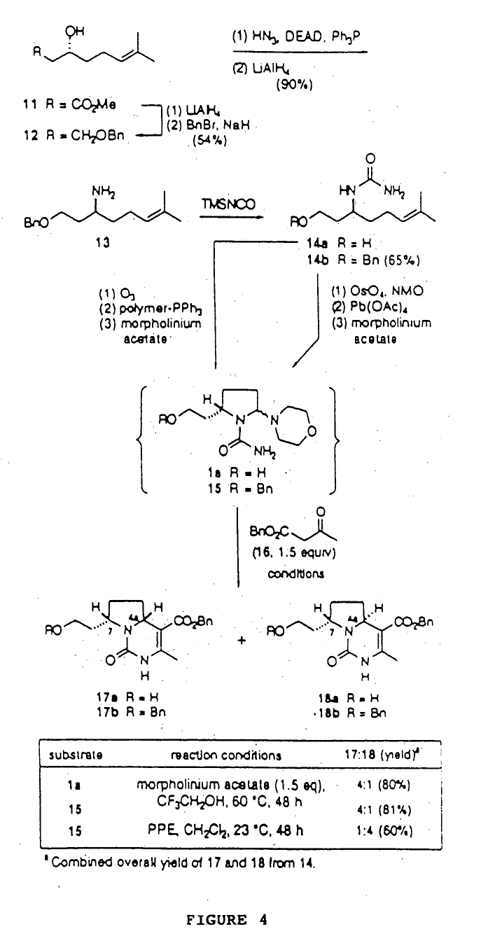

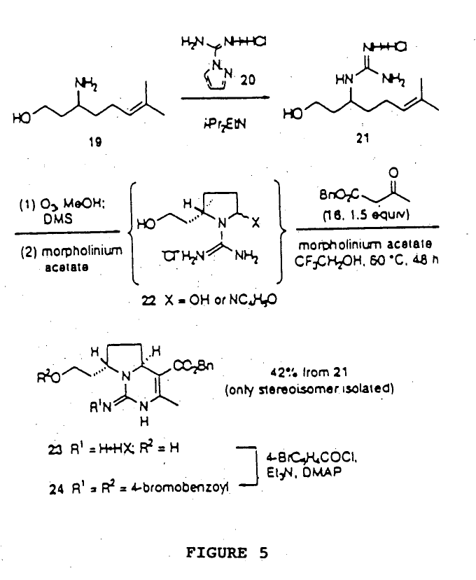

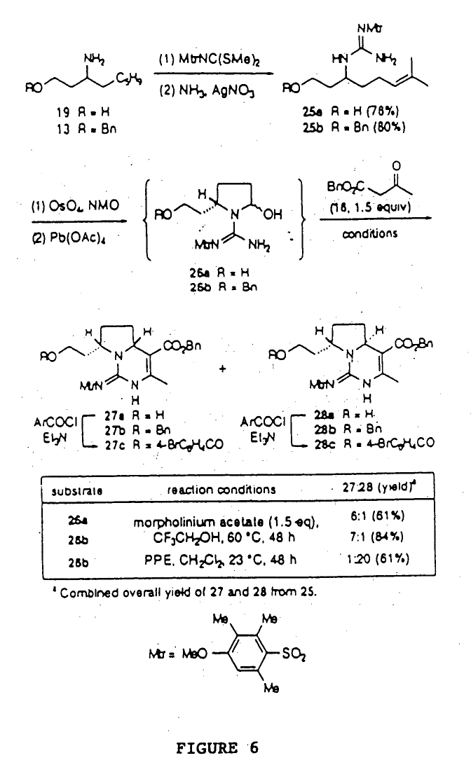

The strategy for synthesis of compounds 12-18 is depicted in FIG. 4, for compounds 21-24 in FIG. 5 and for compounds 25-28 in FIG. 6. Methods of synthesis were used as previously disclosed and known in the art, e.g. Minor and Overman, J. Org. Chem., 1997, 62:6379, incorporated by reference herein.

2D Synthesis of (R)-Benzyloxy-7-methyloct-6-en-3-ol (Compound 12).

A solution of (R)-methyl-3-hydroxy-7-methyl-6-octenoate (Kitamuram et al., Org. Synth., 1992 71:1) (21.5 g, 0.115 mol) and Et₂O (100 mL) was added dropwise to a 0° C. suspension of LiAlH₄ (6.8 g, 0.18 mol) and Et₂O (0.5 L). After 1 h, H₂O (6.8 mL), 3 M NaOH (6.8 mL), and H₂O (20.4 mL) were added sequentially. The resulting mixture was filtered through a pad of Celite, the filtrate was concentrated, and the resulting oil was purified on silica gel (1:1 hexanes-EtOAc) to provide 13.8 g (76%) of (R)-7-methyloct-6-ene-1,3-diol as a colorless oil: ¹H NMR (500 MHz, CDCl₃) δ 5.04-5.08 (m, 1H) 3.82 (s, 2H) 3.68-3.79 (m, 3H) 1.97-2.05 (m, 2H) 1.59-1.67 (m, 4H) 1.54-1.60 (m, 4H) 1.40-1.48 (m, 2H); ¹³C NMR (125 MHz, CDCl₃) 131.8, 123.8, 70.8, 60.7, 38.3, 37.5, 25.5, 24.1, 17.5 ppm; IR (film) 3356 cm⁻¹; [α]²³_{D}+3.5, [α]²³₅₇₇+4.5, [α]²³₅₄₆+4.7, [α]²³₄₂₅+7.3, [α]²³₄₀₅+8.1, (c 1.2, CHCl₃). Anal. Calcd for C₉H₁₈O₂: C, 68.31; H, 11.47. Found: C, 68.09; H, 11.54.

A solution of (R)-7-methyloct-6-ene-1,3-diol (7.00 g, 44.3 mmol) and DMF (80 mL) was added dropwise to a −40° C. suspension of NaH (3.20 g, 133 mmol, prewashed with hexanes 3×50 mL) and DMF (130 mL). After 15 min, benzyl bromide (5.30 mL, 44.3 mmol) was added, and the reaction was warmed to −10° C. over 1 h. The reaction was quenched by pouring into saturated aqueous NH₄Cl (300 mL), and the resulting mixture was extracted with Et₂O (4×150 mL). The combined organic layers were washed with brine (50 mL), dried (MgSO₄), and filtered, and the filtrate was concentrated. The crude oil was purified on silica gel (9:1 hexanes-EtOAc to 4:1 hexanes-EtOAc) to provide 7.74 g (71%) of 12 as a colorless oil: ¹H NMR (500 MHz, CDCl₃) δ 7.32-7.36 (m, 4H) 7.26-7.31 (m, 1H) 5.14-5.17 (m, 1H) 4.51 (s, 2H) 3.78-3.83 (m, 1H) 3.66-3.73.(m, 1H) 3.62-3.65 (m, 1H) 3.04 (s, 1H) 2.05-2.16 (m, 2H) 1.73-1.77 (m, 2H) 1.71 (s, 3H) 1.63 (s, 3H) 1.44-1.57 (m, 2H); ¹³C NMR(125 MHz, CDCl₃) 137.8, 131.5, 128.2, 127.5, 127.4, 124.0, 73.0, 70.4, 68.8, 37.3, 36.3, 25.5, 24.0, 17.5 ppm, IR(film)3443 cm⁻¹; [α]²³_{D}+13.0, [α]²³₅₇₇+13.9, [α]²³₅₄₆+15.6, [α]²³₄₃₅+26.5, [α]²³₄₀₅+31.3 (c 1.4, CHCl₃). Anal. Calcd for C₁₆H₂₄O₂: C, 77.38; H, 9.74. Found: C, 77.25; H, 9.74.

Synthesis of (S)-3-Amino-1-benzyloxy-7-methyl-6-octene (compound 13).

Diethyl azodicarboxylate (4.12 g, 23.7 mrnol) was added dropwise to a solution of 13 (5.05 g, 20.3 mmol), Ph₃P (6.22 g, 23.7 mmol), HN₃ (12 mL, 2.0 M in toluene), and toluene (75 mL) at 0° C. After 15 min, hexanes (0.2 L) was added, the resulting mixture was filtered through a plug of silica gel (the plug was washed with 30 mL of hexanes), and the eluent was concentrated to yield the crude azide as a slightly yellow oil that was used without further purification.

A solution of this crude azide and Et₂O (20 mL) was added dropwise to a stirred 0° C. suspension of LiAlH₄ (0.91 g, 24.0 mmol) and Et₂O (100 mL), and after 15 min the reaction was warmed to room temperature. After 1 h, the reaction was cooled to 0° C., and H₂O (1 mL), 3 M NaOH (1 mL), and H₂O (3 mL) were added sequentially. The resulting mixture was filtered through a pad of Celite, and the filtrate was concentrated to provide 4.53 g (90%) of amine 13 as a colorless oil that was used without further purification: ¹HMR (400 MHz, CDCl₃) δ 7.35-7.38 (m, 4H) 7.27-7.32 (m, 1H) 5.11-5.14 (m, 1H) 4.52 (s, 2H) 3.56-3.65 (m, 2H) 2.88-2.95 (m, 1H) 2.00-2.12 (m, 2H) 1.74-1.82 (m, 1H) 1.70 (s, 3H) 1.62 (s, 3H) 1.42-1.60 (m, 2H) 1.21-1.37 (m, 3H); ¹³C NMR (100 MHz, CDCl₃) 138.4, 131.5, 128.3, 127.5, 127.4, 124.1, 72.9, 68.1, 48.8, 38.4, 37.6,25.6, 24.6, 17.6 ppm; IR (film) 3366 cm⁻¹; [α]²³_{D}−3.3, [α]²³₅₇₇−2.7, [α]²³₅₄₆−3.2, [α]²³₄₃₅−4.9, [α]²³₄₀₅−6.3 (c 1.0, CHCl₃). Anal. Calcd for C₁₆H₂₅NO—HCl: C, 67.71; H, 9.23; N, 4.93. Found: C, 67.68; H, 9.27; N, 5.00.

Synthesis of(S)-1-Benzyloxy-7-methyl-3-ureido-6-octene (Compound 14b).

Trimethylsilyl isocyanate (0.90 mL, 6.7 mmol) was added to a solution of crude 13 (1.15 g, 4.65 mmol) and i-PrOH (7 mL) at room temperature. After 4 h, the reaction was concentrated, and the resulting oil was purified on silica gel (3:1 hexanes-EtOAc to EtOAc) to provide 873 mg (65%) of 14b as a colorless solid: mp 79-81° C.; ¹H NMR (500 MHz, CDCl₃) δ 7.27-7.36 (m, 5H) 5.45 (s, 1H) 5.08-5.11 (m, 1H) 5.93 (s, 2H) 4.94 (s, 2H) 3.53-3.63 (m, 3H) 2.05 (m, 2H) 1.83-1.90 (m, 1H) 1.69 (s, 3H) 1.60 (m, 4H) 1.42-1.54 (m, 2H); ¹³C NMR(125 MHz,CDCl₃) 159.4, 138.0, 131.8, 128.3, 127.6, 127.5, 123.6, 72.9, 67.3, 47.9, 35.7, 35.3, 25.6, 24.4, 17.6 ppm; IR (film) 3340, 1653, 1602 cm⁻¹; [α]²³_{D}+16.0, [α]²³₅₇₇+17.3, [α]²³₅₄₆+19.6, [α]²³₄₃₅+34.5, [α]²³₄₀₅+42.6 (c 1.0, CHCl₃). Anal. Calcd for C₁₇H₂₆N₂O₂: C, 70.31; H, 9.02; N, 9.65. Found: C, 70.39; H; 9.09; N, 9.55.

Conversion of Compound 14a to Intermediate la with Ozone.

Ozone was bubbled through a solution of urea 14a (120 mg, 0.60 mmol), CH₂Cl₂ (5 mL), and MeOH (1 mL) at −78° C. until the solution was saturated (blue color appeared and persisted for 10 min). Nitrogen was then bubbled through the solution to remove excess ozone, Ph₃P-polystyrene (550 mg, 3 mmol P/g resin) was added, and the reaction was allowed to warm to room temperature. After 2 h, the reaction mixture was filtered, morpholinium acetate (140 mg, 0.90 mmol) was added to the filtrate, and the resulting solution was concentrated to give a colorless oil that was used without further purification.

Representative Procedure for Biginelli Condensation under Knoevenagel Conditions. Conversion of Compound la to 17 and 18a.

A solution of crude aminal 1a (0.60 mmol), benzyl acetoacetate (0.16 mL, 0.90 mmol), morpholiniurn acetate (140 mg, 0.90 mmol), and 2,2,2-trifluoroethanol (0.6 mL) was maintained at 60° C. for 2 d. After being cooled to room temperature, the reaction was partitioned between Et₂O (20 mL) and 50% aqueous NH₄Cl (5 mL). The layers were separated, the organic layer was dried (MgSO₄) and filtered, and the filtrate was concentrated. The resulting oil was purified on silica gel (2:1 hexanes-EtOAc to 1:1 hexanes-EtOAc) to give 126 mg (64%) of 17a and 32 mg (16%) of 18a.

(4aR,7S)-7-(2-Hydroxyethyl)-3-methyl-1-oxo-1,2,4a,5,6,7-hexahydropyrrolo[1,2c]pyrimidine-4-carboxylic acid benzyl ester (17a):

¹H NMR (500 MHz, CDCl₃) δ 8.67 (s, 1H) 7.29-7.35 (m, 5H) 5.10-5.20 (m, 2H) 4.25 (dd, J=11.3, 4.7Hz, 1H) 4.11 (dd, J=13.8, 8.2 Hz, 1H)3.84 (s, 1H) 3.56 (m, 2H)2.43-2.48 (m, 1H) 2.22 (s, 3H) 2.02-2.08 (m, 1H) 1.81-1.87 (m, 1H) 1.65-1.74 (m, 3H); ¹³C NMR (125 MHz, CDCl₃) 165.6, 154.9, 149.3, 135.9, 128.5, 128.3, 128.1, 102.2, 65.9, 59.0, 58.4, 52.2, 39.3, 30.6, 29.8, 18.0 ppm; IR(film) 3356, 1707, 1673, 1627 cm⁻¹; [α]²³_{D}−26.5, [α]²³₅₇₇−26.8, [α]²³₅₄₆−37.1, [α]²³₄₃₅−119, [α]²³₄₀₅−184 (c 1.00, CHCl₃); HRMS (CI) m/z 331.1657 (MH, 331.1658 calcd for C₁₈H₂₃N₂O₄).

(4aS,7S)-7-(2-Hydroxyethyl)-3-methyl-1-oxo-1,2,4a,5,6,7-hexahydropyrrolo[1,2-c]pyrimidine-4-carboxylic acid benzyl ester (18a):

¹H NMR (500 MHz, CDCl₃) δ 8.40 (s, 1H) 7.30-7.38 (m, 5H) 5.12-5.22 (m, 2H) 4.42 (m, 1H) 4.35 (dd, J=10.2, 4.5 Hz, 1H) 4.33-4.44 (br s, 1H) 3.60 (m, 2H) 2.40-2.45(m, 1H) 2.45 (s, 3H) 2.06-2.10 (m, 1H) 1.76-1.84 (m, 1H) 1.39-1.55 (m, 3H); ¹³C NMR (125 MHz, CDCl₃) 165.8, 153.0, 146.0, 136.1, 128.6, 128.6, 128.1, 99.1, 65.9, 58.9, 57.3, 53.6, 38.3, 34.9, 28.2, 18.3 ppm; IR (film) 3377, 3232, 1713, 1682, 1633 cm⁻¹; [α]²³_{D}−29.2, [α]²³₅₇₇−29.0, [α]²³₅₄₆−31.0, [α]²³₄₃₅−30.2 (c 1.05, CHCl₃); HRMS (CI) m/z 331.1629 (MH, 331.1658 calcd for for C₁₈H₂₃N₂O₄). Anal. Calcd for C₁₈H₂₂N₂₂O₄: C, 65.44; H, 6.71; N, 8.48.

Represenative Procedure for Generating Tethered Biginelli Precursors by Dihydroxylation and 1,2-Diol Cleavage. Conversion of 14b to 15.

Osmium tetroxide (0.4 mL, 0.1 M in t-BuOH) was added to a solution of 14b (120 mg, 0.41 mmol), N-methylmorpholine N-oxide (230 mg, 1.96 mmol), pyridine (30 mL, 0.4 mmol), and 10:1 TIF-H₂O (8 mL). After 30 min, Florisil (1 g), NaHSO₃ (1 g), and EtOAc (20 mL) were added, and the resulting mixture was stirred. After 30 min, the reaction mixture was filtered, and the filtrate was concentrated to provide the corresponding 1,2-diol as a colorless oil that was used without further purification.

A solution of this crude diol, Pb(OAc)₄ (0.21 g, 0.48 mmol), and CH₂Cl₂ (8 mL) was maintained for 30 min at room temperature. The reaction mixture was then filtered through a plug of Celite, morpholinium acetate (92 mg, 0.62 mmol) was added to the filtrate, and this solution was concentrated to provide crude aminal 15 as a slightly yellow oil (Garigipati et al., J. Am. Chem. Soc., 1985, 107:7790).

Conversion of Compound 15 to 17b and 18b under Knoevenagel Biginelli Conditions.

Following the representative procedure for Biginelli condensation under Knoevenagel conditions, crude aminal 15 (0.41 mmol) was condensed with 16, and the crude product was purified on silica gel (2:1 hexanes-EtOAc to 1:1 hexanes-EtOAc) to provide 140 mg (81%) of a 4:1 mixture of 17b and 18b. The isomers were separated by medium-pressure liquid chromatography (MPLC) on silica gel (2:1 hexanes-EtOAc to 1 hexanes-EtOAc).

(4aR,7S)-7-(2-Benzyloxyethyl)-3-methyl-l-oxo-1,2,4a,5,6,7-hexahydropyrrolo[1,2c]pyrimidine-4-carboxylic acid benzyl ester (17b):

¹H NMR (500 MHz, CDCl₃) δ 8.21 (s, 1H) 7.25-7.38 (m, 10H) 5.11-5.21 (m, 2H) 4.43-4.53 (m, 2H) 4.28-4.31 (m, 1H) 3.98-4.02 (m, 1H) 3.51-3.55 (m, 2H) 2.43-2.48 (m, 1H) 2.22-2.28 (m, 1H) 2.20 (s, 3H) 1.86-1.95 (m, 2H) 1.74-1.78(m, 1H) 1.61-1.66(m, 1H); ¹³C NMR (125 MHz, CDCl₃) 165.9, 152.7, 148.9, 138.4, 136.1, 128.5, 128.3, 128.3, 128.1, 127.5, 127.4, 101.4, 72.6, 67.8, 65.8, 58.0, 54.4, 33.4, 30.6, 28.9, 18.2 ppm; IR (film) 1682, 1633 cm⁻¹; [α]²³_{D}−18.7, [α]²³₅₇₇−20.3, [α]²³₅₄₆-25.0, [α]²³₄₃₅−71.7, [α]²³₄₀₅−108 (c 1.4, CHCl₃). Anal. Calcd for C₂₅H₂₈N₂O₄: C, 71.41; H, 6.71; N, 6.66. Found: C, 71.31; H, 6.80; N, 6.69.

(4aS,7S)-7-(2-Benzyloxyethyl)-3-methyl-l-oxo-1,2,4a,5,6,7-hexahydropyrriolo[1,2-c]pyrimidine-4-carboxylic acid benzyl ester (18b):

¹H NMR (500 Mz, CDCl₃) δ 8.94 (s, 1H) 7.33-7.40 (m, 9H) 7.26-7.32 (m, 1H) 5.14-5.24 (m, 2H) 4.47-4.56 (m, 2H) 4.33-4.41 (m, 2H) 3.60-3.62 (m, 2H) 2.42-2.47 (m, 1H) 2.26 (s, 3H) 2.00-2.12 (m, 2H) 1.73-1.79 (m, 1H) 1.44-1.55 (m, 2H); ¹³C NMR (125 MHz, CDCl₃) 166.0, 151.8, 147.1, 138.4, 136.3, 128.4, 128.2, 128.0, 127.9, 127.5, 127.4, 98.2, 72.8, 67.7, 65.5, 57.2, 54.6, 35.2, 34.8, 28.1, 18.2 ppm; IR (film) 1681, 1640 cm⁻¹; [α]²³_{D}−37.5, [α]²³₅₇₇−37.0, [α]²³₅₄₆−39.7, [α]²³₄₃₅−34.5, [α]²³₄₀₅−14.1 (c 1.0, CHCl₃). Anal. Calcd forC₂₅H₂₈N₂O₄: C,71.41; H, 6.71; N, 6.66. Found: C, 71.30; H, 6.73; N, 6.59.

Representative Procedure for Biginelli Condensation in the Presence of PPE. Conversion of Compound 14b to 17b and 18h.

Urea 14b (115 mg, 0.400 mmol) was converted to 15 following the general olefin dihydroxylation and 1,2-diol cleavage procedure. A solution of the resulting crude aminal 15, benzyl acetoacetate (110 mg, 0.59 mmol), polyphosphate ester (0.2 mL), and CH₂Cl₂ (0.2 mL) was maintained at room temperature for 2 d. The reaction was then quenched by adding Et₂O (20 mL) and 50% aqueous NaHCO₃ (5 mL). The layers were separated, the organic layer was dried (MgSO₄) and filtered, and the filtrate was concentrated. The resulting oil was purified on silica gel (2:1 hexanes-EtOAc to 1:1 hexanes-EtOAc) to provide a 101 mg (60%) of a 4:1 mixture of 18b and 17b.

(4aS,7S)-7-(2-Hydroxyethyl)-l-imino-3-methyl-1,2,4a,5,6,7-hexahydropyrrolo[1,2-c]pyrimidine-4-carboxylic acid benzyl ester hydroformate (Compound 23).

Following the general procedure of Bernatowicz, (Bematowicz et al., J. Org. Chem. 1992, 57:2497), a solution of (S)-3-amino-7-methyl-6-octenol (Overman et al., J. Am. Chem. 1995, 117:2657) (0.95 g, 6.0 mmol), 1H-pyrazole-1-carboxamidine hydrochloride (0.95 g, 6.1 mmol), i-Pr₂EtN (1.1 mL, 6.3 mmol), and DMF (2.7 mL) was heated at 60° C. After 4 h, the reaction mixture was concentrated, and the resulting crude 21, a colorless oil, was used without further purification.

Ozone was bubbled through a solution of this sample of crude 21 and MeOH (25 mL) at −78° C. until the solution was saturated. Nitrogen was then bubbled through the solution to remove excess ozone, Me₂S (1 mL) was added, and the reaction was allowed to warm to room temperature. After 1 h, the reaction mixture was dried (MgSO₄) and filtered, and the filtrate was concentrated to give 22 as a yellow oil that was used without further purification.

Following the representative procedure for Biginelli condensation under Knoevenagel conditions, aminal 22 was condensed with compound 16 and the crude product was purified on silica gel (100% CHCl₃ to 10:1 CHCl₃-i-PrOH to 10:1:0.1 CHCl₃-i-PrOH-HCO₂A) to yield 0.95 g (42%) of trans-Biginelli product 23 as a colorless oil: ¹H NMR (500 MHz, CDCl₃) δ 10.03 (br s, 2H) 8.29 (s, 2H) 7.27-7.35 (m, 5H) 5.19 (d, J=12.3 Hz, 1H) 5.12 (d, J=12.3 Hz, 1H) 4.28-4.38 (m, 2H) 3.76-3.78 (m, 1H) 3.49-3.53 (m, 1H) 2.45-2.50 (m, 1H) 2.28 (s, 3H) 2.11-2.17 (m, 1H) 1.81-1.87 (m, 1H) 1.58-1.67 (m, 2H) 1.47-1.54 (m, 1H), the OH signal was too broad to observe; ¹³C NMR (125 MHz, CDCl₃) 166.6, 164.9, 150.7, 143.8, 135.5, 128.5, 128.2, 128.1, 101.1, 66.2, 57.1, 56.1, 56.0, 36.0, 34.1, 28.0, 17.2 ppm; IR (film) 3180, 1684, 1572 cm⁻¹; [α]²³_{D}−30.7, [α]²³₅₇₇−32.2, [α]²³₅₄₆−35.7 (c 3.1, CDCl₃); HRMS (FAB) m/z 330.1820 (MH, 330.1818 calcd for C₁₈H₂₄O₃N₃).

(4aS,7S)-l-(4-Bromobenzoylimino)-7-[2-(4-bromobenzoyloxy)ethyl]-3-methyl-1,2,4a,5,6,7-hexahydropyrrolo[1,2-c]pyrimidine-4-carboxylicAcid Benzyl Ester (24).

4-Bromobenzoyl chloride (400 mg, 1.81 mmol) was added at 0° C. to a solution of 23 (220 mg, 0.60 mmol), Et₃N (0.50 mL, 3.6 mmol), CH₂Cl₂ (10 mL), and a crystal of 4-(dimethylamino)pyridine. After 1 h, the reaction was partitioned between Et₂O (50 mL) and saturated aqueous NH₄Cl (10 mL). The layers were separated, the organic layer was washed with brine (10 mL), dried (MgSO₄), and filtered, and the filtrate was concentrated. The residue was purified on silica gel (4:1 hexanes-EtOAc) to provide 150 mg (36%) of 24 as a colorless solid: mp 175-176° C.: ¹H NMR (500 MHz, CDCl₃) δ 7.98 (d, J=7.8 Hz, 2H) 7.88 (d, J=7.8 Hz, 2H) 7.56 (d, J=7.8 Hz, 2H) 7.37-7.40 (m, 5H) 7.31 (d, J=7.8Hz, 2H) 5.15-5.25 (m, 2H) 4.79-4.82 (m, 1H) 4.52-4.53 (m, 2H) 4.41-4.45 (m, 1H) 2.56-2.61 (m, 1H) 2.48-2.53 (m, 1H) 2.31 (s, 3H) 2.13-2.19 (m, 1H) 1.92-1.96 (m, 1H) 1.56-1.73 (m, 2H), the NH signal was too broad to observe; ¹³C NMR (125 MHz, CDCl₃) 176.9, 165.7, 165.4, 152.7, 143.7, 136.8, 135.8, 131.8, 131.0, 131.0, 130.6, 128.9, 128.6, 128.3, 128.3, 128.2, 126.4, 101.0, 66.1, 62.3, 56.0, 55.9, 34.7, 33.7, 27.4, 18.9 ppm; IR (film) 1716, 1608 cm⁻¹; [α]²³_{D}−3.3, [α]²³₅₇₇−2.8, [α]²³₅₄₆−1.0, [α]²³₄₃₅+32.5, [α]²³₄₀₅+68.5, (c 1.75, CHCl₃). Anal. Calcd for C₃₂H₂₉Br₂N₃O₅: C, 55.27; H, 4.20; N, 6.04. Found.: C, 55.20; H, 4.16; N, 6.04.

(S)-N-[(Aminomethylene)-4-methoxy-2,3,6-trimethylbenzenesulfonamide]-3-amino-7-methyl-6-octenol (25a).

A solution of (S)-3-amino-7-methyl-6-octenol (Overman et al., J. M. Chem. Soc. 1995. 117:2657) (19, 1.00 g, 6.36 mmol), S,S,-dimethyl N-(4-methoxy-2,3,6-trimethylbenzenesulfonyl)carbonimidodithioate (1.78 g, 5.34 mmol), and benzene (6 mL) was maintained at reflux for 2 h. The reaction was quenched by adding Et₂O (50 mL) and 0.1 M HCl (5 mL). The layers were separated, the organic layer was dried (MgSO₄) and filtered, and the filtrate was concentrated. The resulting crude oil was purified by MPLC (1:1 hexanes-EtOAc) to provide 1.81 g (77%) of the corresponding pseudothiourea as a colorless oil: ¹H NMR (500 MHz, CDCl₃) δ 7.86 (d, J=9.8 Hz, 1H) 6.40 (s, 1H) 5.04-5.06 (m, 1H) 3.85 (s, 3H) 3.77-3.84 (m, 1H) 3.66-3.73 (m, 2H) 2.72 (s, 3H) 2.64 (s, 3H) 2.36 (s, 3H) 2.15 (s, 3H) 1.96-2.02 (m, 2H) 1.84-1.92 (m, 2H) 1.69 (m, 3H) 1.60-1.68 (m, 2H) 1.56 (m, 3H), the OH signal was too broad to observe; ¹³C NMR (125 MHz, CDCl₃) 167.4, 158.8, 138.8, 137.0, 132.8, 132.4, 124.9, 122.7, 111.6, 58.7, 55.4, 52.2, 37.7, 35.4, 25.7, 24.1, 24.0, 18.4, 17.6, 14.2, 11.8 ppm; IR (film) 3480, 3290 cm⁻¹; [α]²³_{D}−15.3, [α]²³₅₇₇-14.7, [α]²³₅₄₆−17.9, [α]²³₄₃₅−31.8, [α]²³₄₀₅−39.2 (c 1.9, CHCl₃). Anal. Calcd for C₂₁H₃₄N₂O₄S₂: C, 56.98; H, 7.74; N, 6.33. Found: C, 56.90; H, 7.69; N, 6.34.

Silver nitrate (26 mL, 0.2 M in MeCN) was added dropwise to a 0° C. solution of a 1.59 g (3.60 mmol) portion of this pseudothiourea and MeCN (75 mL) that had been saturated with NH₃. (Burgess et al., J. Org Chem. 1994, 59:2179). The reaction mixture was allowed to warm to room temperature, and after 18 h, EtOAc (100 mL) was added and the resulting mixture was filtered through a plug of Celite. The eluent was concentrated to provide 1.46 g (99%) of 25a as a colorless solid: mp 107-109° C.: ¹H NMR (500 MHz, CDCl₃) δ 6.51 (s, 2H) 6.15 (s, 1H) 4.90 (app s, 1H) 4.36 (s, 1H) 3.80 (app s, 4H) 3.53-3.66 (m, 3H) 2.64 (s, 3H) 2.56(s, 3H) 2.10(s, 3H) 1.85-1.86(m, 2H) 1.71 (m, 1H) 1.56(m, 3H) 1.39-1.32(m,6H); ¹³C NMR (125 MHz, CDCl₃) 158.5, 156.8, 138.3, 136.6, 132.9, 131.9, 124.8, 123.2, 111.7, 58.1, 55.3, 47.5, 38.4, 35.3, 25.5, 24.5, 24.1, 18.2, 17.4, 11.9ppm; IR (film)3442, 3354, 1621 cm⁻¹; [α]²³_{D}−20.0, [α]²³₅₇₇−20.7, [α]²³₅₄₆−23.0, [α]²³₄₃₅−33.2, [α]²³₄₀₅−35.7 (c 2.4, CHCl₃). Anal. Calcd for C₂₀H₃₃N₃O₄S: C, 58.37; H, 8.08; N, 10.21. Found: C, 58.31; H, 8.05; N, 10.21.

(S)-N-[(Aminomethylene)-4-methoxy-2,3,6-trimethylbenzenesuyfonamide]-3-amino-l-benzyloxy-7-methyl-6-octene (25b).

Following the procedure described for preparing 25a, 13 (0.807 g, 3.262 mmol) was converted in 80% overall yield to 25b a colorless oil; ¹H NMR 500 MHz, DMSO, 80° C.) δ 7.25-7.32 (m, 5H) 6.65 (s, 1H) 6.45 (s, 1H) 6.42 (s, 1H) 5.01 (m, 1H) 4.35 (s, 2H) 3.77 (s, 3H) 3.73 (m, 1H) 3.38-3.41 (m, 2H) 3.09 (s, 3H) 2.63 (s, 3H) 2.56 (s, 3H) 1.88 (m, 2H) 1.69 (m, 1H) 1.60 (m, 4H) 1.49 (s, 3H) 1.36-1.42 (m, 2H); ¹³C NMR (125 MHz, DMSO, 80° C.) 157.3, 155.6, 138.2, 137.2, 135.3, 134.8, 130.5, 127.7, 126.9, 126.8, 123.4, 123.2, 111.6, 71.6, 66.5, 55.1, 47.5, 34.3, 34.2, 24.8, 23.5, 22.8, 17.4, 16.9, 11.2 ppm; IR (film) 3445, 3336, 1622, 1538 cm⁻¹; [α]²³_{D}+14.6, [α]²³₅₇₄+15.3, [α]²³₅₄₆+18.2, [α]²³₄₃₅+37.4, [α]²³₄₀₅+48.9 (c 1.80, CHCl₃). Anal. Calcd for C₂₇H₃₉N₃O₄S: C, 64.64; H, 7.84; N, 8.38. Found: C, 64.77; H, 7.88; N, 8.32.

Conversion of 25a to 27c and 28c under Knoevenagel Biginelli Conditions.

Following the represenative olefin dihydroxylation and 1,2-diol cleavage procedure, 25a (100 mg, 0.24 mmol) was converted to 26a. Aminal 26a was then condensed with 16 following the representative procedure for Biginelli condensation under Knoevenagel conditions with the exception that the concentration of 26a in 2,2,2-trifluoroethanol was 0.5 M. Purification of the crude product on silica gel (1:1 hexanes-EtOAc) provided 80 mg (61%) of a 6:1 mixture of 27a and 28a.

A 120 mg (0.22 mmol) sample of a comparable product was esterified with 4-bromobenzoyl chloride (160 mg, 0.72 mmol) following the procedure described for the preparation of 24 to provide a crude residue that was purified on silica gel (3:1 hexanes-EtOAc) to provide 160 mg (100%) of a 6:1 mixture 27c and 28c. These isomers were separated by HPLC.(6:1 hexanes-EtOAc; 20 mL/min, 300×22 mm 10 μm silica Alltech column) to give pure samples of 27c (t_{R}=62 min) and 28c (t_{R}=53 min).

(4aR,7S)-7-[2-(4-Bromobenzoyloxy)ethyl]-l-(4-methoxy-2,3,6-trimethylbenzenesulfonylimino)-3-methyl-1,2,4a,5,6,7-hexahydropyrrolo[1,2-c]pyrimidine-4-carboxylic acid benzyl ester(27c):

¹H NMR (500 MHz, CDCl₃) δ 9.33 (s, 1H) 7.76 (d, J=8.4 Hz, 2H) 7.51 (d, J=8.4 Hz, 2H) 7.32-7.39 (m, 5H) 6.48 (s, 1H) 5.12-5.21 (m, 2H) 4.20-4.29 (m, 2H) 4.13-4.18 (m, 1H) 4.05-4.09 (m, 1H) 3.78 (s, 3H) 2.66 (s, 3H) 2.59 (s, 3H) 2.46-2.55 (m, 1H) 2.34 (s, 3H) 2.13-2.19 (m, 1H) 2.06 (s, 3H) 1.93-2.00 (m, 1H) 1.75-1.87 (m, 2H) 1.64-1.71 (m, 1H); ¹³C NMR (125 MHz, CDCl₃) 165.5, 164.9, 158.5, 148.1, 145.6, 138.4, 136.5, 135.6, 132.9, 131.6, 131.0, 128.7, 128.6, 128.4, 128.3, 128.0, .124.7, 111.6, 103.8, 66.3, 62.4, 57.0, 55.3, 54.9, 32.5, 30.0, 28.5, 24.1, 18.5, 18.3, 11.8 ppm; IR (film) 3292, 1716, 1614 cm⁻¹; [α]²³_{D}+55.5, [α]²³₅₇₇+57.7, [α]²³₅₄₆+66.5, [α]²³₄₃₅+121, [α]²³₄₀₅+150 (c 2.1, CHCl₃). Anal. Calcd for C₃₅H₃₈BrN₃O₇S: C, 58.01; H, 5.29; N, 5.80. Found: C, 57.98; H, 5.42; N, 5.52.

(4aS,7S)-7-[2-(4-Bromobenzoyloxy)ethyl]-l-(4-methoxy-2,3,6-trimethylbenzenesulfonylimino)-3-methyl-1,2,4a,5,6,7-hexahydropyrrolo[l,2-c]pyrimidine-4-carboxylic acid benzyl ester (28c):

¹H NMR (500 MHz, CDCl₃) δ 9.20 (s, 1H) 7.76 (d, J=8.4 Hz, 2H) 7.51 (d, J=8.4 Hz, 2H) 7.33-7.54 (m, 5H) 6.44 (s, 1H) 5.12-5.23 (m, 2H) 4.36-4.44 (m, 2H) 4.27-4.29 (m, 2H) 3.80 (s, 3H) 2.65 (s, 3H) 2.56 (s, 3H) 2.46-2.51 (m, 1H) 2.29 (s, 3H) 2.02-2.10 (m, 4H) 1.75-1.82 (m, 1H) 1.48-1.62 (m, 3H); ¹³C NMR (125 MHz, CDCl₃) 165.6, 165.3, 158.5, 146.7, 143.0, 138.4, 136.9, 135.8, 133.0, 131.6, 131.0, 128.7, 128.6, 128.3, 128.2, 128.0, 124.8, 111.6, 100.6, 66.2, 62.0, 56.2, 56.1, 55.4, 34.5, 33.2, 27.7, 24.0, 18.9, 18.3, 11.8 ppm; IR (film) 3298, 1716, 1614 cm⁻¹; [α]²³_{D}−17.7, [α]²³₅₇₇−16.1, [α]²³₅₄₆−18.3, [α]²³₄₃₅−19.4, [α]²³₄₀₅−13.3 (c 0.75, CHCl₃). Anal. Calcd for C₃₅H₃₈BrN₃O₇S: C, 58.01; H, 5.29; N, 5.80. Found: C, 58.06; H, 5.41; N, 5.55.

Conversion of Compound 25b to 27b and 28b under Knoevenagel Biginelli Conditions.

Following the represenative olefin dihydroxylation and 1,2-diol cleavage procedure, 25b (100 mg, 0.20 mmol) was converted to 26b, and this crude material was condensed with 16 following the representative procedure for Biginelli condensation under Knoevenagel conditions with the exception that the concentration of 26b in 2,2,2-trifluoroethanol was 0.5 M. Purification of the crude product on silica gel (4:1 hexanes-EtOAc to 2:1 hexanes-EtOAc) provided 106 mg (84%) of a 7:1 mixture of 27b and 28b. Characterization data for the major product (4aR,7S)-7-(2-Benzyloxyethyl)-1-(4-methoxy-2,3,6-trimethylbenzenesulfonylimino 3-methyl-1,2,4a,5,6,7-hexahydropyrrolo[1,2-c]pyrimidine-4-carboxylic acid benzyl ester (27b) as determined from this mixture: ¹H NMR (500 MHz, CDCl₃) δ 9.42 (s, 1H) 7.23-7.42 (m, 10H) 6.52 (s, 1H) 5.15-5.25 (m, 2H) 4.28-4.36 (m, 2H) 4.23 (d, J=11.1, 4.0 Hz, 1H) 4.03-4.07 (m, 1H) 3.82 (m, 3H) 3.40-3.42 (m, 2H) 2.70 (s, 3H) 2.62 (s, 3H) 2.48-2.50 (m, 1H) 2.31 (s, 3H) 2.13 (s, 3H) 2.00-2.05 (m, 1H) 1.93-1.95 (m, 2H) 1.79-1.83 (m, 1H) 1.47-1.53 (m, 1H); ¹³C NMR(125 MHz, CDCl₃) 165.1, 158.5, 148.1, 145.6, 138.5, 138.2, 136.4, 135.7, 133.2, 128.6, 128.4, 128.2, 128.2, 127.5, 127.4, 124.7, 111.7, 103.9, 72.5, 67.7, 66.3, 57.0, 55.9, 55.3, 33.4, 30.0, 28.8, 24.0, 18.5, 18.3, 11.9 ppm; IR(film)3289, 1704, 1614cm⁻¹. Anal. Calcd for C₃₅H₄₁N₃O₆S: C, 66.54; H, 6.54; N, 6.65. Found: C, 66.66; H, 6.57; N, 6.66.

Conversion of Compound 25b to (4aS,7S)-7-(2-Benzyloxyethyl)-1-(4-methoxy-2,3,6-trimethylbenzenesulfonylimino)-3-methyl-1,2,4a,5,6,7-hexahydropyrrolo[1,2-c]pyrimidine-4-carboxylic Acid Benzyl Ester (28b) by Biginelli Condensation in the Presence of PPE.

Following the representative procedure for olefin dihydroxylation and 1,2-diol cleavage, 25b (100 mg, 0.20 mmol) was converted to 26b. Crude aminal 26b was then condensed with 16 following the representative procedure for Biginelli condensation in the presence of PPE to give, after purification on silica gel (2:1 hexanes-EtOAc to 1:1 hexanes-EtOAc), 77 mg (61%) of 28b, which was contaminated with a trace of 27b (3%). 28b: ¹H NMR (500 MHz, CDCl₃) δ 9.23 (s, 1H) 7.22-7.42 (m, 10H) 6.54 (s, 1H) 5.16-5.26 (m, 2H) 4.36-4.40 (m, 2H) 4.26-4.35 (m, 2H) 3.84 (m, 3H) 3.45-3.48 (m, 2H) 2.72 (s, 3H) 2.65 (s, 3H) 2.45-2.50 (m, 1H) 2.32 (s, 3H) 2.15-2.20 (m, 1H) 2.14 (s, 3H) 2.00-2.05 (m, 1H) 1.62-1.72 (m, 1H) 1.51-1.60(m, 2H); ¹³C NMR(125 MHz, CDCl₃) δ 165.4, 158.5, 146.4, 142.9, 138.6, 136.4, 135.8, 133.4, 128.6, 128.3, 128.2, 128.2, 127.5, 127.4, 127.4, 124.7, 111.6, 100.5, 72.5, 67.2, 66.1, 56.4, 56.0, 55.3, 34.5, 34.1, 27.7, 24.0, 18.8, 18.3, 11.9 ppm; IR (film) 3290, 1712, 1614 cm⁻¹; [α]²³_{D}−65.8, [α]²³₅₇₇−67.5, [α]²³₅₄₆−76.7, [α]²³₄₃₅−117, [α]²³₄₀₅−128 (c 1.1, CHCl₃). Anal. Calcd for C₃₅H₄₁N₃O₆S: C, 66.54; H, 6.54; N, 6.65. Found: C, 66.49; H, 6.51; N, 6.56.

Conversion of Compound 28c to Compound 24.

A solution of 28c (15 mg, 20 mmol) and TFA (2 mL) was maintained for 1 h at room temperature. The reaction was concentrated, and the resulting crude oil was used without purification. 4-Bromobenzoyl chloride (22 mg, 0.10 mmol) was added to a 0° C. solution of this crude guanidine, Et₃N (0.15 mL, 1.08 mmol), CH₂Cl₂ (2 mL) and a crystal of 4-(dimethlyamino)-pyridine. After 1 h, the reaction was quenched to Et₂O (10 mL) and saturated aqueous NH₄Cl (2 mL). The layers were separated, the organic layer was dried (MgSO₄) and filtered, and the filtrate was concentrated. The residue was purified on silica gel (4:1 hexanes-EtOAc) to provide 4 mg (29%) of 24 as a colorless solid.

S,S-Dimethyl N-(4-Methoxy-2,3,6-trimethylbenzenesulfonyl)carbonimidodithioate guanylating agent (FIG. 6).

Ammonia was bubbled through a solution of 4-methoxy-2,3,6-trimethylbenzenesulfonyl chloride (Fujino et al., Chem. Pharm. Bull., 1981, 29:2825) (10.3 g, 43.6 mmol) and CH₂Cl₂ (100 mL) at 0° C. After 30 min, acetone (0.5 L) was added, and the reaction mixture was filtered through a plug of silica gel and concentrated. The resulting solid was trituated with Et₂O to provide 9.18 g (92%) of 4-methoxy-2,3,6-trimethylbenzenesulfonamide as a colorless solid: mp 175-176° C.; ¹H NMR (400 MHz, acetone-d₆) δ 6.75 (s, 1H) 6.36 (s, 2H) 3.86 (s, 3H) 2.63 (s, 3H) 2.58 (s, 3H) 2.05 (s, 3H); ¹³C NMR (100 MHz, acetone-d⁶) 159.7, 139.0, 138.0, 134.6, 125.3, 113.0, 56.2, 24.4, 18.5, 12.3 ppm; IR (KBr) 3385, 3279, 2983, 2942, 1582, 1560, 1486, 1309, 1148, 1113 cm⁻¹. Anal. Calcd. for C₁₀H₁₅NO₃S: C, 52.38; H, 6.59; N, 6.11. Found: C, 52.46; H, 6.55; N, 6.05. A solution of 4-methoxy-2,3,6-trimethylbenzenesulfonamide (9.15 g, 39.9 mmol) and DMF (50 mL) was added to a mixture of NaH (4.11 g, 98.6 mmol, washed 3×with hexanes) and DMF (20 mL) at 0° C. The reaction was allowed to warm to room temperature and was stirred vigorously for 10 min before CS₂ (6.9 mL, 11 mmol) was added. After another 10 min, MeI (7.85 mL, 126 mmol) was added. After another 15 min, the reaction was poured into saturated aqueous NH₄Cl (200 mL) and extracted with CHCl₃ (3×0.5 L). The combined organic layers were dried (MgSO₄), filtered through a plug of silica gel and concentrated. The crude solid was trituated with MeOH to provide 11.1 g (84%) of S,S-dimethyl N-(4-5 methoxy-2,3,6-trimethylbenzenesulfonyl)carbonimidodithioate as a colorless solid: mp 175-176° C.; ¹H NMR (400 MHz, CDCl₃) δ 6.56 (s, 1H) 3.84 (s, 3H) 2.71 (s, 3H) 2.57 (s, 3H) 2.52 (s, 6H) 2.13 (s, 3H); ¹³C NMR (100 MHz, CDCl₃) 182.3, 159.3, 139.2, 138.5, 130.3, 125.0, 111.7, 55.4, 23.9, 18.5, 16.3, 11.9 ppm; IR(film)2969, 2930, 1552, 1476, 1386, 1307, 1146, 997, 925, 804 cm⁻¹. Anal. Calcd. for C₁₃H₁₉NO₃S₃: C, 46.82; H, 5.74; N, 4.20. Found: C, 46.82; H, 5.73; N, 4.22.

Results

Biginelli Condensations of Tethered Ureido Aldehydes

To pursue whether the free hydroxyl group in intermediate compound 1 might be influencing stereoselection, Biginelli condensations of this intermediate and benzyl ether derivative 15 were examined (FIG. 4). Like compound 1, the benzyl ether congener was accessed from (R)-methyl-3-hydroxy-7-methyl-6-octenoate (11) (Kitamuram et al., Org. Synth., 1992, 71:1). Reduction of compound 11 with LiAlH₄ and selective monobenzylation of the resulting diol by reaction with excess NaH and benzyl bromide in DMF at −40 to −10° C. furnished compound 12. Mitsunobu inversion of alcohol 12 with HN₃, (Loibner et al., Helv. Chim. Acta, 1976, 59:2100) followed by reduction of the resulting azide and reaction of the resulting primary amine with trimethylsilyl isocyanate, provided urea compound 14b in 32% overall yield from compound 11.

In prior studies, the double bond of compound 14a had been cleaved with ozone, using a dimethyl sulfide workup, to generate compound 1 (Overman et al., supra). A more reproducible procedure was to add 1.5 equiv of morpholinium acetate to the crude reaction mixture after reductive workup of the ozonide, but prior to concentration. Replacing dimethyl sulfide with polymer-bound triphenylphosphine eliminated contamination with DMSO. Mass spectral data of the product compound 1a generated in this fashion indicated incorporation of morpholine (with loss of H₂O) and showed the virtual absence of higher molecular weight oligomers.

Alternatively, compound 15 was generated by dihydroxylation of the corresponding alkene precursor, followed by cleavage of the derived 1,2-diol with Pb-(Oac)₄ (Zelle et al., J. Org. Chem., 1986, 51:5032). Aminals 1a and 15 were never subjected to an aqueous workup or purification, but rather were used directly following removal of either the phosphine polymer or lead salts by filtration and concentration of the filtrate after adding morpholinium acetate. These intermediates are not simply a mixture of stereoisomers, but at least three components as judged by ¹H and ¹³C NMR data; multiple signals are observed for many carbon atoms in the ¹³C NMR spectra, while broad peaks are seen in the ¹H NMR spectra and no aldehyde signal is apparent.

Biginelli condensations of crude compound 15 or 1a (generated from 1 equivalent of compound 14a or 14b) were carried out under identical conditions by reaction with 1.5 equivalents of β-ketoester 16 and 1.5 equivalents of morpholinium acetate at 60° C. in 2,2,2-trifluoroethanol. These conditions provided the cis- and trans-1-oxohexahydropyrrolo[1,2-c]pyrimidines 17a and 18a in a 4:1 ratio (80% yield) and the corresponding benzyl ether analogues 17b and 18b in an identical 4:1 ratio (81% yield). The β-oxygen substituent of the side chain clearly plays no significant role. Trifluoroethanol was employed as the reaction solvent since earlier studies with related intermediates had shown that cis stereoselection under Knoevenagel conditions was optimal in this highly polar solvent. For example, stereoselection in the condensation of 1 and 16 was 2:1 when ethanol was employed. Products 17a and 18a did not interconvert upon resubmission to reaction conditions. Stereochemical assignments for the hexahydropyrrolo[1,2-c]pyrimidine products followed from diagnostic ¹H NMR signals of the angular methine hydrogens H4a and H7: 17a (4.25 and 4.11 ppm) and 17b (4.29 and 4.00 ppm) (Overman and Rabinowitz., J. Org. Chem., 1993, 58:3235).

In a recent investigation, Kappe reported (J. Org. Chem., 1997, 62:7201) that the mild dehydrating agent polyphosphate ester (PPE) (Cava et al., J. Org. Chem., 1969, 34:2665) was an excellent promoter of the classical three-component Biginelli condensation. Condensation of 15 with β-ketoester 16 at room temperature in a 1:1 mixture of PPE and CH₂Cl₂ provided Biginelli products 17b and 18b in 60% yield, with the trans isomer 18b now predominating to the extent of 4:1. Identical to what was observed under Knoevenagel conditions, compounds 17b and 18b were recovered unchanged when resubmitted to the PPE reaction conditions for 48 h.

Biginelli Condensations of Tethered Guanyl Aldehydes

Although three-component condensations of guanidines, aldehydes, and β-ketoesters are known, this modification of the Biginelli condensation has not been widely explored. (Kappe, Tetrahedron, 1993, 49:6937). To examine the tethered variant, unsaturated guanidinium alcohol 21 was prepared from (S)-amino alcohol 19 (Overman et al., J. Am.Chem. Soc., 1995, 117:2657) by condensation with 1H-pyrazole-1-carboxamidine hydrochloride (20) (FIG. 5, Bernatowicz et al., J. Org. Chem., 1992, 57:2497). Ozonolysis of 21 followed by workup with dimethyl sulfide and concentration provided 22, which like its urea counterpart was a mixture of several components. When 22 was concentrated with 1.5 equiv of morpholinium acetate, FAB mass spectral data indicated incorporation of morpholine with loss of H₂O; higher molecular weight oligomers were not observed for either 22 (X═OH) or its morpholine adduct. Both intermediates performed identically in Biginelli condensations. Without purification, 22 was condensed with β-ketoester 16, using Knoevenagel conditions identical to those employed in the urea series, to afford a single Biginelli adduct 23 in 42% overall yield from 19. This product had the trans stereochemistry as rigorously established by single-crystal X-ray analysis of dibenzoyl derivative 24 (Coordinates for compound 24 have been deposited with Cambridge Crystallographic Data Centre, 12 Union Road, Cambridge CB2 1EZ, U.K.).

To pursue the origin of the stereochemical reversal in the urea and guanidine series, Biginelli condensations of tethered N-sulfonylguanidine aldehydes 26 were investigated as depicted in FIG. 6. Since the pKₐ of N-sulfonylguanidinium salts is typically˜1, the sulfonylguanidine substituent electronically resembles more closely a urea than a guanidine (Tatlor et al., J. Chem. Soc. Perkin Trans. 2, 1986, 1765; Yamamoto et al., in Synthesis and Chemistry of Guanidine Derivatives, Yamamoto and Kojima, Ed., Wiley, N.Y., 1991 (Vol. 2, pp 485-526). The statistically corrected pKₐ of a monosubstituted guanidinium salt bearing an SO₂NH₂ substituent has been determined to be 1.83 in water. Using the linear free energy correlation developed by Tatlor et al., surra, the value for the corresponding SO₂Me-substituted guanidinium salt would be 0.2. Treatment of amino alcohol 19, or the corresponding amino ether 13, with S,S-dimethyl N-(4-methoxy-2,3,6-trimethylbenzenesulfonyl)-carbonimidodithioate, followed by aminolysis with NH₃ and AgNO₃, afforded the Mtr-protected guanidines 25 in good yield (Burgess et al., J. Org. Chem., 1994, 59:2179). Dihydroxylation of these intermediates, followed by diol cleavage, provided 26a and 26b. These intermediates were again not simple mixtures of stereoisomers; multiple signals were observed for many carbon atoms in the ¹³C spectra, while ¹H spectra exhibited broad peaks and showed no apparent aldehyde signal.

Biginelli condensation of crude 26b with β-keto ester 16 under Knoevenagel conditions identical to those employed with the other substrates proceeded in 84% yield to give the cis- and trans-1-iminohexahydropyrrolopyrimidines 27b and 28b in a 7:1 ratio. Nearly identical stereoselectivity. was realized in the hydroxyethyl series. In dramatic contrast, when the condensation of 26b and 16 was carried out with PPE, the trans-1-iminohexahydropyrorolopyrimidine 27b predominated to the extent of 20:1. Sulfonylguanidine products 27b and 28b were recovered unchanged when resubmitted for 48h to either the Knoevenagel or PPE reaction conditions.

Stereochemical assignments were made by chemical correlation of 28a with 24. Acylation of the crude product mixture produced from Biginelli condensation of 26a and 16 under Knoevenagel conditions with 4-bromobenzoyl chloride followed by separating the isomers by HPLC provided pure samples of 27c and 28c. Exposure of the minor product 28c to TFA at room temperature removed the Mtr group, and acylation of the resulting free guanidine with 4-bromobenzoyl chloride provided 24.

These results demonstrate that stereoselection in tethered Biginelli condensations to form 1-oxo- and 1-iminohexahydrophyrrolo[1,2-c]pyrimidines varies substantially depending on reaction conditions and the nature ofthe group X (FIG. 3). With substrates having urea and N-sulfonylguanidine functionality, cis stereoselection (4-7:1) is observed when the condensation is accomplished under Knoevenagel conditions, while trans stereoselection (4-20:1) is observed when the condensation is carried out in the presence of polyphosphatester (PPE). Under both conditions, stereoselectivity was highest in the N-sulfonylguanidine series. With a substrate having a basic guanidine unit, the trans product is formed exclusively under Knoevenagel conditions. Since the Knoevenagel conditions are notably mild (morpholinium acetate in CF₃CH₂OH at 60° C.), this latter guanyl aldehyde route to trans-1-15 iminohexahydropyrrolo[1,2-c]pyrimidines will like be particularly useful for the synthesis of Crambescidin and Batzelladine alkaloids having the anti relationship of the hydrogens flanking the pyrrolidine nitrogen. In the Examples below, the first total synthesis of Isocrambescidin 800 using this approach is described.

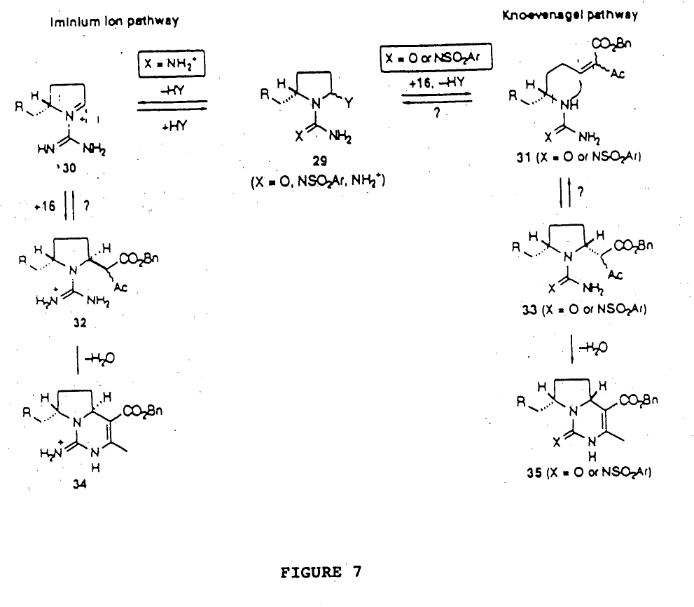

The origin of stereoselectivity is unknown. Without wishing to be bound by any theory, the following hypothesis is proposed (FIG. 7). Under Knoevenagel conditions, the stereochemistry-determining step in condensations of ureido or N-sulfonylimino aldehyde intermediates 29 could be cyclization of Knoevenagel adduct 31 to give 33. If this reaction has a late transition state, the cis-2,5-disubstituted pyrrolidine should be formed preferentially (molecular mechanics calculations on the model N-acylamino-2,5-disubstituted pyrrolidines 36 show that the cis isomer is 1.9 kcal/mol more stable than the trans isomer. Calculations were done using the MM2 force field and the Monte Carlo search routing of MacroModel V3.5X. In contrast, in the guanyl aldehyde series, loss of HY from 29 to form the corresponding iminium ion 30 should be particularly favorable, since the nitrogen substituent in 30 is a weakly electron-withdrawing amidine group. If addition of the enol (or enamine) derivative of 16 is controlled primarily by destabilizing interactions with the side chain, trans adduct 32 should be produced preferentially in what could be the stereochemistry-determining step. Alternatively, the stereochemistry-determining step could be [4+2]-cycloaddition of the enol (or enamine) or 30 from the face opposite the side chain, followed by loss of water (or morpholine). In accord with Kappe's recent investigations of the mechanism of the three component Biginelli reaction under classical acidic conditions (Kappe et al., supra), condensations of the ureido or N-sulfonylimino aldehyde intermediates 29 in the presence of polyphosphate ester (PPE) could also proceed by the iminium ion pathway to provide largely trans-1-oxo- and 1-iminohexahydropyrrolo[1,2-c]pyrimidines.

These results demonstrate that stereoselection in the tethered Biginelli condensations depicted in FIG. 3 can be tuned to give either the cis or trans product. Under optimum conditions, the trans isomer can be obtained in high stereoselectivity (>20:1) and the cis isomer in moderate selectivity (4−7:1). Tethered Biginelli condensations can be extended to include guanyl aldehyde substrates that produce Biginelli products, which should prove particularly useful for preparing complex guanidines.

### EXAMPLE II

Enantioselective Total Synthesis of Ptilomycalin A, Crambescidin 800 and Selected Congeners

Synthesis Plan.

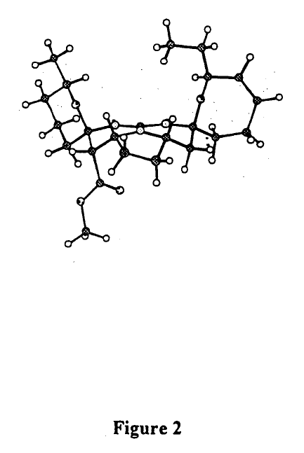

A molecular mechanics model of the methyl ester of the Ptilomycalin A/Crambescidin core is shown in FIG. 2. The triazacenaphthalene ring system of these alkaloids is nearly planar with the seven- and six-membered cyclic ethers being oriented on one face. Since the two C—O bonds are axial (cis to the C10 and C13 angular hydrogens), it was surmised that the C8 and C15 spirocenters might assemble with the required stereochemistry if the proper cis stereochemistry of the central triazacenaphthalene unit were in place. Setting the cis stereorelationship of the angular hydrogens at C10 and C13 and relating the chirality of this unit to the C3 and C19 stereogenic centers of the oxepene and hydropyran rings proved to be the critical elements in evolving a stereocontrolled strategy for preparing this class of guanidine alkaloids.

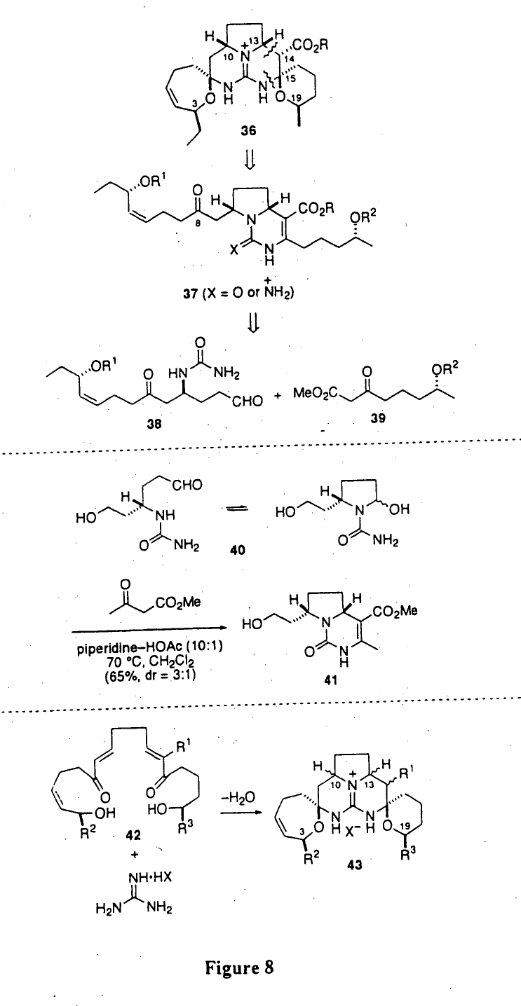

As illustrated in FIG. 8, disconnection of the C8 aminal and retrosynthetic cleavage of the C15-O bond of 36 leads to the 1-oxohexahydropyrrolo[1,2-c]pyrimidine (X═O) and 1-iminohexahydropyrrolo[1,2-c]pyrimidine intermediates. (X═NH₂) 37. The 4-alkoxycarbonyl-3,4-dihydropyrimidin-2(1H)-one part structure of 37 suggested that this essential bicyclic intermediate might be prepared by a novel modification of the three-component Biginelli condensation, in which the urea and aldehyde components would be linked as depicted in 38.

This analysis has the appeal of high convergence, since the left-hand three rings of 36 would derive from acyclic fragment 38, while the right two rings and the ester side chain would be incorporated as the simple β-ketoester unit 39.

Enantioselective Total Synthesis of Ptilomycalin A.

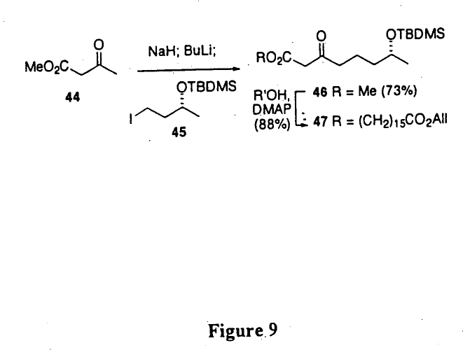

In light of the difficulty experienced during degradation studies in removing the ester side chain of 1, the 16-hydroxyhexadecanoic acid fragment was incorporated from the outset (FIG. 9). Alkylation of the dianion of methyl acetoacetate (44)(Huckin and Weiler, J. Am. Chem. Soc., 1974, 96:1082) with enantiopure (R)-siloxy iodide 45 provided 46 in 73% yield. Iodide 45 is conveniently available in high yield from methyl (R)-2-hydroxybutanoate (Kitamura et al., Org. Synth., 1992, 71: 1). Selective transesterification (Taber et al., J. Org. Chem., 1985 50:3618) of the β-ketoester functionality with allyl 16-hydroxyhexadecanoate using DMAP (4-dimethylaminopyridine) as catalyst gave 47 in 64% overall yield from 44.

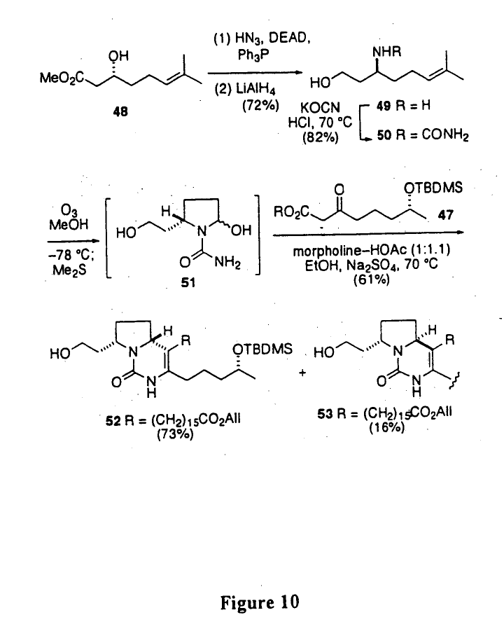

Since the tethered Biginelli condensation had just been verified, in this first generation approach this critical reaction was selected as early as possible in the synthetic sequence. For this reason, a less convergent strategy was pursued in which the electrophilic component of the Biginelli condensation was simplified by deletion of the C1-C7 fragment. The precursor of this intermediate, urea 50, was prepared in three steps from enantiopure methyl (R)-3-hydroxy-7-methyloct-6-enoate (48)(Kitamura et al., supra) as summarized in FIG. 10.

Mitsunobu displacement of 48 with hydrazoic acid followed by reduction of the crude β-azido ester with LiAlH₄ gave S amino alcohol 49 in 72% yield and in >98% ee. Entantiometric excess was determined by evaluation of the ¹⁹F NMR spectra of the corresponding (R)- and (S)-Mosher's amides. Use of other nitrogen nucleophiles such as phthalimide in the Mitsunobu reaction led to significant amounts of the corresponding a, β-unsaturated ester.

Condensation of 49 with potassium cyanate and HCl under standard conditions provided unsaturated urea 50 in 82% yield after recrystallization. Ozonolysis 50 in MeOH at −78° C., followed by reduction of the intermediate hydroperoxide with Me₂S and concentration furnished a viscous yellow oil. Further concentration of this product at 0.1 Torr for 5 days at 50° C. to remove residual Me₂SO lead to a nearly colorless amorphous powder. This intermediate is more complex than formulation 51 implies. Multiple signals were observed for many carbon atoms in the ¹³C NMR spectra and the ¹H NMR spectrum was broad; no aldehyde signal was apparent, and mass spectral data indicated an oligomeric consistency. All attempts to enhance the purity of 51 by chromatography were unsuccessful.

Biginelli condensation of crude 51 and 47 under the conditions developed during our previous model study (Overman et al., J. Org. Chem., 1993, 58:3235-3237), proceeded in low yield. A number of reaction parameters were surveyed and reaction efficiency was improved in polar solvents. The best result was achieved by heating a mixture of crude 51, 1.5 equivalent of β-ketoester 47, 1 equivalent of morpholinium acetate, a catalytic amount of acetic acid and excess Na₂SO₄ at 70° C. in EtOH. Purification of the resulting product on silica gel provided cis adduct 52 in 61% yield and trans adduct 53 in 8% yield. Stereochemical assignments for the hexahydropyrrolo[1,2-c]pyrimidine products followed from the similarity of their angular methine hydrogen signals (52: 4.25 and 4.11 ppm and 53: 4.44 and 4.09 ppm) with those of 41 and its trans epimer, the latter of which had earlier been analyzed by single-crystal X-ray analysis (Overman et al., J. Org. Chem., 1993, 58:3235-3237). In a recent detailed examination of stereoselection in related Biginelli condensations (McDonald and Overman, J. Org. Chem., 1999, 64: 1520-1528) a reproducible procedure for generating the electrophilic reaction component and carrying out the Biginelli condensation was developed; these conditions reliably provide cis adducts in yields of 60-65%.

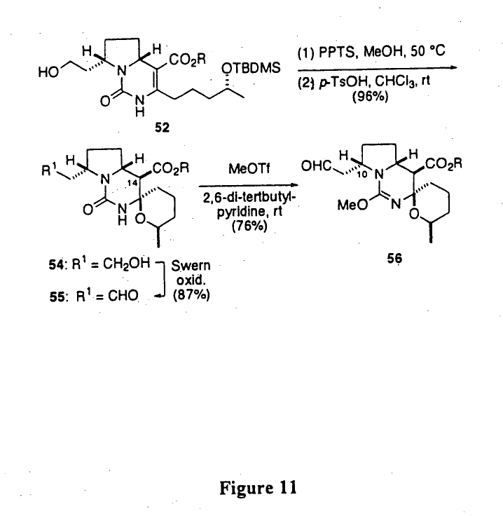

While 52 could be converted in one step to the spirotricyclic intermediate 54 by exposure to a slight excess of p-toluenesulfonic acid (p-TsOH), the reaction was more reproducible on a large scale if the TBDMS group was first cleaved with pyridinium p-toluenesulfonate (PPTS) in MeOH and the resulting alcohol cyclized at room temperature in CHCl₃ with a catalytic amount of p-TsOH (FIG. 11). This sequence provided a single tricyclic product 54 in near quantitative yield. That this compound was epimeric to Ptilomycalin A at C14 was signaled by the 11.5 Hz diaxial coupling constant of the C14 methine hydrogen. (The Crambescidin numbering system is employed here.)

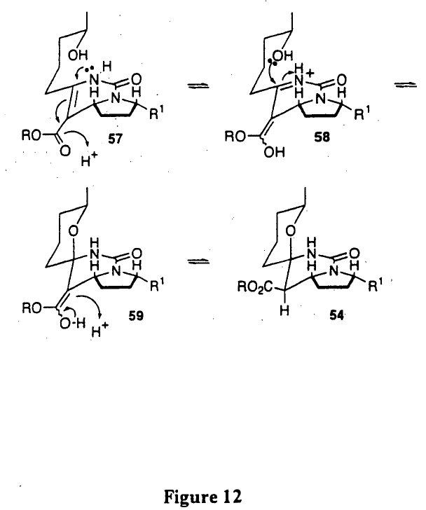

That high diastereoselectivity would be realized in forming the spirohydropyran was established in our earliest model study and can be rationalized as outlined in (FIG. 12). Protonation ofthe vinylogous carbamate 57 to generate 58 followed by spirocyclization from the convex β-face would produce the spiroaminal having the oxygen axially disposed. Axial protonation ofthe exocyclic ketene hemiacetal, either prior or subsequent to spirocyclization, would deliver 54.

Although epimerization of 55 to the axial ester might have been possible at this point, this adjustment was deferred to the final stage of the synthesis, hoping to benefit from a presumed thermodynamic preference for this group to be axial in the natural product. To prepare for the addition of the remaining carbons of the guanidine core, 54 was oxidized with the Swern reagent (Mancuso et al., J. Org. Chem. , 1978, 43:2480) to provide 55, whose urea moiety was protected and activated for subsequent guanidine formation by O-methylation. It was critical that this methylation be performed under carefully optimized mild conditions, and that pseudourea 56 be purified rapidly on Et ₃N-treated silica gel, or else significant epimerization at C10 resulted.

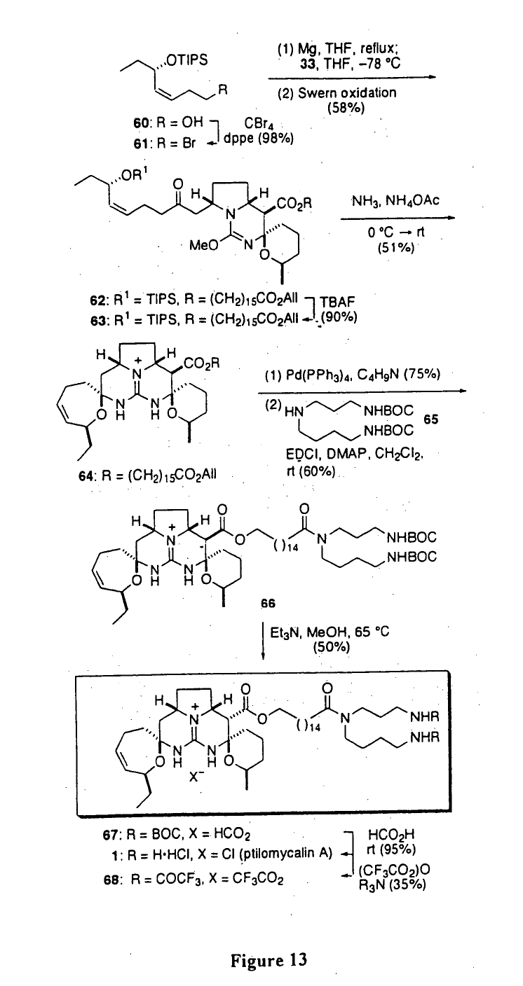

At this point the remaining C1-C7 carbons of the pentacyclic guanidine unit needed to be appended. This elaboration proved to be extremely challenging. In early studies, we were unsuccessful in efficiently coupling lithium, cerium, titanium, or zirconium reagents derived from bromide 61 (FIG. 13) to the benzyl ester congener of 56. A complicating issue was the rapid epimerization of 56 at C10 in the presence of Lewis acidic reagents. The Grignard reagent derived from 61 added in acceptable yield to 56 at −78° C. Quenching this reaction at low temperature with morpholinium acetate and immediate filtration to remove magnesium salts provided the corresponding adduct as a mixture of alcohol epimers. Direct oxidation of this intermediate under Swem conditions (Mancuso et al., J. Org. Chem., 1978, 43:2480) provided 62 in 58% yield from 56. Approximately 5% of a diastereomer, resulting from the minor enantiomer of 61, was removed at this point. Bromide 61 was originally prepared in 86% ee by an asymmetric reduction of an ynone precursor (Overman et al., J. Am. Chem. Soc., 1995, 117:2657-2658). This sequence was extremely sensitive and yields were markedly eroded if magnesium salts resulting from the Grignard step were not removed quickly and thoroughly.

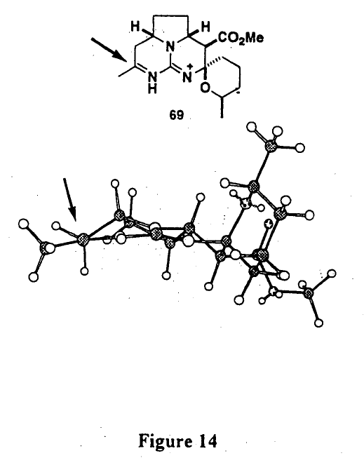

Cleavage of the silyl protecting group of 62 with TBAF furrished alcohol 63 which was then treated with ammonia and ammonium acetate, under conditions similar to those originally reported by Snider (Snider and Shi, J. Am. Chem. Soc., 1994, 116:549-557). After purification of the crude product on silica gel using an eluent containing formic acid, 64 was isolated in 60% as its formate salt (¹H NMR δ 8.23, ¹³C NMR δ 165.8). Only a single pentacyclic guanidine was detected, with formation of the spiroaminal again occurring preferentially by axial C—O bond formation. A model of a tetracyclic cation 69 that is the likely direct precursor of the pentacyclic guanidine is shown in FIG. 14; the C1-C7 side chain was replaced with a methyl group in generating this molecular mechanics model. A torsional preference for axial addition to the electron-deficient carbon is apparent in this figure and may be responsible for the high selectivity realized.

The total synthesis of Ptilomycalin A was readily completed from 64. The allyl ester of this intermediate was cleanly cleaved (Deziel, Tetrahedron Lett., 1987, 28:4371), usingpalladium (0) catalysis and the resulting acid was coupled with the bis-BOC-protected spermindine 65 (Cohen, et al., Chem. Soc. Chem. Commun., 1992, 298) to generate amide 66 (FIG. 13). The ester was then epimerized by heating in MeOH in the presence of excess Et₃N, however, equilibrium for this epimerization favored the β epimer to the extent of 2-3:1. As a result, three recycles were required to obtain α-ester 67 in 50% yield. The equatorial C14 methine hydrogen of 67 showed a diagnostic doublet (J=4.8 Hz) at δ 2.93. Finally, cleavage of the BOC protecting groups with HCO₂H, followed by concentration and washing with aqueous NaOH—NaCl provided (−)-Ptilomycalin A trihydrochloride (1) in high yield. Synthetic compound 1 showed ¹H and ¹³C NMR spectra consistent with those reported for (−)-Ptilomycalin A (Kashman et al., J. Am. Chem. Soc., 1989, 111:8925-8926; Ohtani et al., J. Am. Chem. Soc., 1992, 114:8472-8479) and was indistinguishable from an authentic sample by TLC comparisons on three adsorbents. Synthetic 1 was converted to derivative compound 68, which also exhibited ¹H and ¹³C NMR spectra indistinguishable from those reported (Ohtani et al., supra). Synthetic compound 68 showed [α]²³_{D}−15.9 (c 0.8, CHCl₃), nearly identical to the rotation, [α]²³_{D}−15.8 (c 0.7, CHCl₃), reported for this well-characterized derivative of the natural product (Ohtani et al., supra).

Second Generation Synthesis Plan.

A second generation synthesis of the Ptilomycalin A/Crambescidin alkaloids was undertaken with two specific goals in mind; (1) to achieve the high level of convergence originally illustrated in FIG. 8, where the entire carbon skeleton of pentacycle 36 derive from a Biginelli condensation between a fully elaborated electrophilic component (38) and β-keto ester unit 39; and (2) to gain access to either cis or trans 37 from a conrnon precursor, thereby providing a convenient route to both the Crambescidin and Isocrambescidin core from a common intermediate. Details of the total synthesis of 13,14,15-Isocrambescidin 800 (6) are described in the following Example. Critical to both these syntheses is the rapid and stereoselective construction of the commnon C1-C13 fragment (amine precursor to urea 38). This goal could be accomplished by combining the C1-C7 fragment 56 with the C8-C13 fragment 48 prior to the Biginelli condensation.

### EXAMPLE III

Synthesis of Crambescidin 800 (Compound 2).

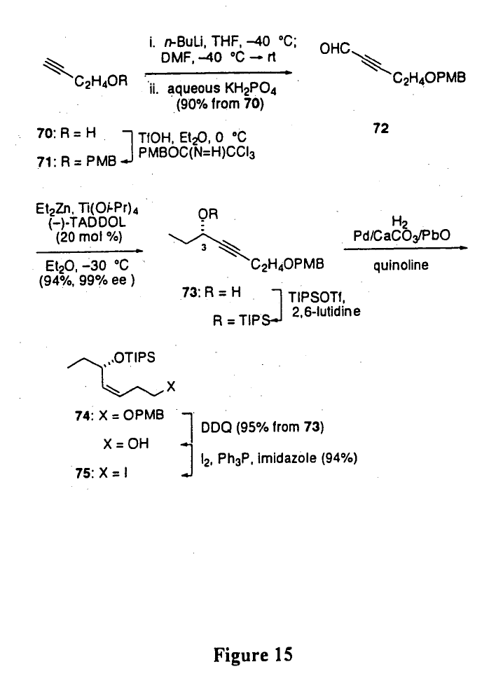

The synthesis of the C1-C13 fragment began with conversion of 3-butynol (compound 70) to the p-methoxybenzyl (PMB) ether 71 (FIG. 15). The alkyne of 71 was deprotonated with n-buthyllithium at −40° C. and the resulting acetylide treated with anhydrous DMF to provide ynal 72 in 90% yield, after quenching the intermediate α-aminoalkoxide into aqueous phosphate buffer (Journet et al., Tetrahedron Lett., 1988, 39:6427). The C3 stereocenter was introduced by the method of Weber and Seebach (Singh et al., J. Am. Chem. Soc., 987, 109:6187) through condensation of ynal 72 with Et₂Zn in the presence of (−)-TADDOL (20 mol %) and Ti(Oi-Pr)₄ to give (S)-73 in 94% yield and >98% ee. This asymmetric transformnation was reliably performed on a 45 g scale. Propargylic alcohol 73 was protected as the triisopropylsilyl (TIPS) ether and the alkyne partially hydrogenated with Lindlar's catalyst to provide cis alkene 74. The PMB protecting group was oxidatively removed with DDQ and the resulting alcohol converted to iodide 75 in an overall yield of 89% from 73.

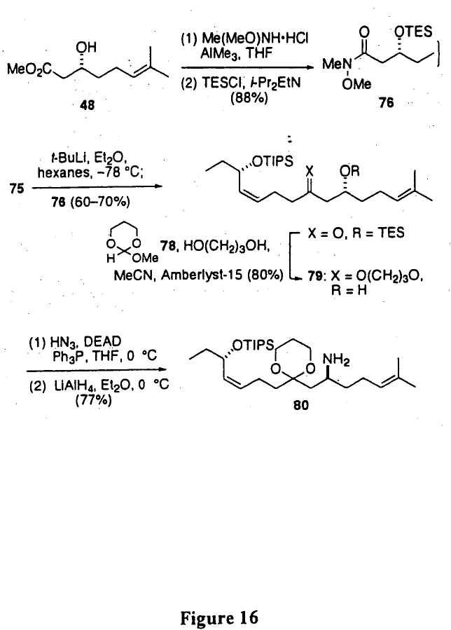

Enantiopure methyl (R)-3-hydroxy-7-methyloct-6-enoate (compound 48) (Kitamura et al., Org. Synth., 1992, 71:1) was converted to amide in 88% yield by reaction with N,O-dimethylhydroxylamine hydrochloride according to the procedure of Weinreb (Garigipati et al, J. Am. Chem. Soc., 1985, 107:7790) followed by protection of the secondary alcohol as the triethylsilyl (TES) ether (FIG. 16). Iodide 75 was converted to the corresponding lithium reagent and coupled with 76 to generate dienone 77 in 60-70% yield. Masking the C8 carbonyl of 77 as the ketal was necessary to prevent dehydration, which occurred under the Mitsunobu conditions employed to install the β-amino functionality. Ketalization was sluggish, however, when the β-hydroxy group was protected, so optimized reaction conditions were found which cleaved the TES group, did not promote β-hydroxy elimination of the intermediate β-hydroxy ketone and promoted ketalization. The novel ketalization conditions developed involved treatment of 77 with orthoester 78 (Roush and Gillis, J. Org. Chem., 1980, 45:4283-4287; Baganz and Domaschke, Chem. Ber., 1958, 91:650-653) and 1,3-propanediol in the presence of Amberlyst-15 to provide ketal 79 in 80% yield. Mitsunobu displacement of the secondary alcohol with azide followed by reduction to the amine provided compound 80 in 77% yield from compound 79. Amine 80, synthesized in 11 steps and in ˜30% overall yield from commercially available 3-butynol, was to serve as the common C1-C13 fragment for both the Crambescidin and Isocrambescidin syntheses (See FIG. 16).

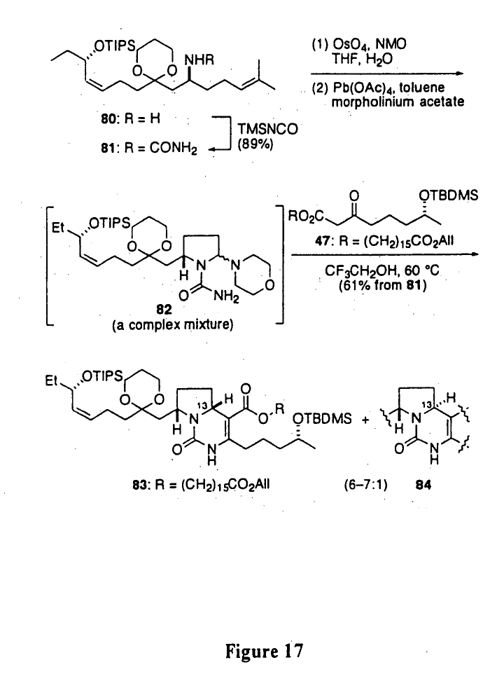

Condensation of amine 80 with TMSNCO yielded urea 81 in 89% yield (FIG. 17). Selective dihydroxylation of the trisubstituted double bond of 81 (Sharpless and Williams, Tetrahedron Lett., 1975, 3045-3046) followed by cleavage of the vicinal diol with Pb(OAc)₄ in toluene and addition of morpholinium acetate yielded intermediate 82, which was used without purification. Biginelli condensation of crude 82 with β-ketoester 47 under optimal Knoevenagel conditions (McDonald and Overman, J. Org. Chem., 1999, 64:1520-1528) provided an inseparable 6-7:1 mixture of desired cis adduct 83 and undesired trans adduct 84 in 61% overall yield from urea 81. Stereochemical assignments for the hexahydropyrrolo[1,2-c]pyrimidine products followed from the similarity of their H13 angular methine hydrogen signals (83: 4.22 ppm and 84: 4.44 ppm) with those of 41 and its trans epimer and 52 and 53 (FIG. 17).

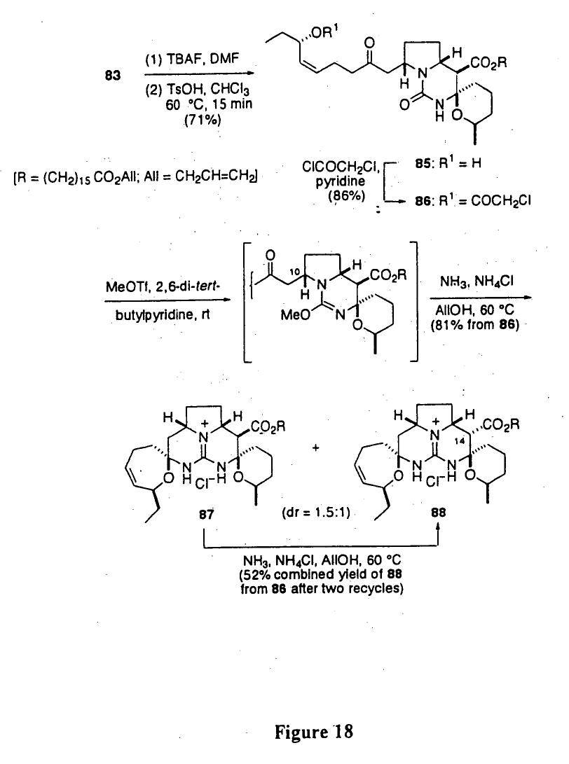

The silyl protecting groups of 83 were next discharged with TBAF to provide the corresponding urea diol (FIG. 18). Brief exposure of this crude diol to p-TsOH induced spirohydropyran formation and ketal deprotection, affording 85 in 71% for the two steps. After protection of the secondary alcohol of 85 as the chloroacetate the minor trans isomer (˜12%) was easily separated from the desired cis isomer 86, which was isolated in 86% yield. It was necessary to protect the C3 alcohol of 85 to prevent methyl ether formation during the methylation of the urea functionality. Exposure of urea 86 to excess MeOTf in the presence of a hindered pyridine base cleanly provided the corresponding methyl pseudourea, which was directly converted to the guanidine without intermediate silica gel purification. It was critical that the methyl pseudourea not be exposed to silica gel chromatography, as decomposition and epimerization at C10 resulted under typical purification conditions. The ability to transform the urea functionality to the guanidine without manipulations of the intermediate pseudourea represents one of the major advantages of the second generation synthesis over the first. After considerable experimentation, we found that optimal guanylation/cyclization conditions, saturated NH₃ in allyl alcohol buffered with NH₄Cl at 60° C. for 1 day, cleanly provided pentacycles 87 and 88 in 81% from 86 as a 1.5:1 diastereomeric mixture at C14. Subjection of pure compound 88 to the reaction conditions established this ratio as the thermodynamic equilibrium (FIG. 18).

These reaction conditions represent a significant improvement from those used in the first generation synthesis, as yield improved dramatically, and both deprotection of the C3 protecting group and the desired epimerization of C14 to its thermodynamic ratio occurred. The chloride counter ion was also obtained directly, eliminating detrimental aqueous washes. It should be noted that incomplete deprotection of the C3 alcohol occurred when a simple acetyl protecting group was employed, whereas the chloroacetyl protecting group was quantitatively removed under the guanylation/pentacyclization reaction conditions. Allyl alcohol was employed as solvent to avoid transesterification of the allyl ester that occurred when ethanol or methanol was employed. Furthermore, it was found necessary to saturate the reaction solution with NH₃ at 0° C. prior to heating in order to achieve thermodynamic equilibration of the C14 ester side chain. Unfortunately, the thermodynamic ratio of 1.5:1 favored the undesired β-epimer (H14: J=11.5 Hz). Pentacycles 87 and 88 were separated by medium pressure silica gel liquid chromatography, and the β-epimer twice recycled through the guanylation/cyclization conditions to provide the major α-ester pentacycle 88 in a 52% overall yield from tricyclic urea 86.

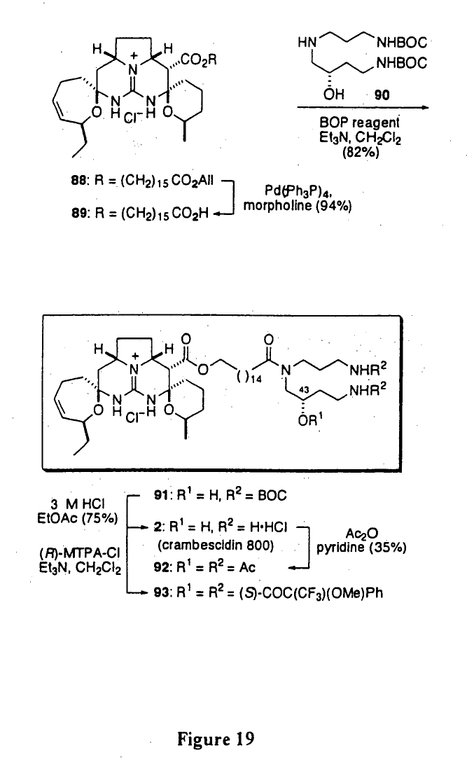

The synthesis of Crambescidin 800 (compound 2) was completed as follows (FIG. 19). After removal of the allyl protecting group of 88 with Pd(PPh₃)₄ and morpholine (Deziel, supra) acid 89 was coupled with (S)-7-hydroxyspermidine 90 using benzotriazol-1-yloxytris(dimethylamino)phosphonium hexafluorophosphate (BOP)(Castro et al. Tetrahedron Lett. 1975 1219-1222) to provide the corresponding amide 91 in 82% yield. Removal of the BOC groups with 3 M HCl in ethyl acetate (Stahl et al., J. Org. Chem., 1978 43:2285-2286) followed by purification of the crude product using reverse-phase HPLC provided the trihydrochloride salt of Crambescidin 800 (2) in 75% yield. The data for the trihydrochloride salt of synthetic 2 is in agreement with the ¹H and ¹³C NMR data reported for natural compound 2 (Jares-Erijman et al., J. Org. Chem., 1991,56:5712-5715; Berlinck et al., J. Nat. Prod., 1993, 56:1007-1015). Synthetic 2 was also converted to the triacetate derivative, 92. Data for synthetic 92 is also in agreement with ¹H and ¹³C NMR data reported for 92 prepared from natural 2 (Id.) The Mosher's derivatives of (43S)- and (43R)-crambescidin 800 (93) were made and compared to the corresponding Mosher's derivative prepared from ˜150 μg of natural compound 2. The ¹⁹F NMR data is identical for the Mosher's derivative prepared from natural 2 and synthetic 2, thereby for the first time unambiguously establishing that the C43 stereochemistry of Crambescidin 800 is S (FIG. 19).

Conclusion.

The first total synthesis of Crambescidin 800 (2) was accomplished in a convergent fashion with the longest linear sequence from commercially available starting material being 25 steps and in a 3.0% overall yield. This synthesis demonstrates for the first time that the tethered Biginelli condensation can be accomplished under suitable mild conditions that the aldehyde-urea fragment can contain all the atoms of the three left rings (C1-C13), thus allowing high convergence and efficiency in the synthesis of Crambescidin/Ptilomycalin A alkaloids. These investigations conftum the stereochemical assignment of 2 and rigorously establish that the absolute configuration of its hydroxyspermidine side chain is S.

### Experimental Section

General.

Dry THF, Et₂O, and CH₂Cl₂ from Aldrich were filtered through a column charged with Al₂O₃ (solvent purification system). Triethylamnine (Et₃N), pyridine, diisopropylethylamine (i-Pr₂NEt), diisopropylamine, and acetonitrile were distilled from CaH₂ at atmospheric pressure. Silica gel (0.040-0.063) by Merck was used for flash chromatography. The NMR spectra were recorded on the Bruker instruments (500 MHz and 400 MHz). The IR spectra were measured on Perkin-Elmer Series 1600 FTIR, and optical rotations were measured on Jasco DIP-360 polarimeter. Mass spectra were measured on a MicroMass Analytical 7070E (CI-isobutane) or a MicroMass AutoSpec E (FAB) spectrometer. Infrared spectra were recorded using a Perkin Elmer 1600 FTIR spectrometer. Microanalyses were performed by Atlantic Microlabs, Atlanta, GA. Other general experimental details have been described (Metais et al., J. Org. Chem. 1997, 62:9210, incorporated by reference herein).

Synthesis of ]-(4-Methyoxybenzyloxy)-3-butyne (71).

According to established procedures (Takaku et al., Tetrahedron lett., 1983, 24:5363; Nakajima et al., Tetrahedron Lett., 1988, 29:4139, both of which are incorporated by reference herein), TfOH (1.6 mL, 18 mmol) was added dropwise to a 0° C. solution of PMBOC(═NH)CCl₃ (169.3 g, 0.6 mol), 3-butyn-1-ol (70) (67 g, 0.66 mol) and dry Et₂O (600 mL). After 30 min the reaction mixture was quenched by the addition of saturated aqueous NaHCO₃ (100 mL). The phases were separated, and the aqueous phase was extracted with Et₂O (50 mL). The combined organic phases were washed with brine (50 mL), dried (MgSO₄), filtered and concentrated. The resulting residue was diluted with hexanes (300 mL), filtered through a plug of silica gel, concentrated and stirred under vacuum (0.1 mm Hg) at 50° C. for 12 h, yielding 114 g (˜100%) of 71, which was used without further purification: ¹H NMR (500 MHz, CDCl₃) δ 7.28 (d, J=8.4 Hz, 2 H), 6.89 (d, J=8.4 Hz, 2 H), 4.49 (s, 2 H), 3.80 (s, 3 H), 3.58 (t, J=7.0 Hz, 2 H), 2.49 (dt, J=7.0, 2.7 Hz, 2H), 2.00(t, J=2.6Hz, 1 H); ¹³C NMR (125 MHz,CDCl₃) δ 159.2, 130.0, 129.3, 113.7, 81.3, 72.5, 69.2, 67.8, 55.2, 19.8 ppm; IR (film) 3292, 3001, 2936, 2863, 1614, 1514, 823 cm⁻¹; Anal. Calcd for C₁₂H₁₄O₂: C, 75.76; H, 7.42. Found: C, 75.60; H, 7.49.

Synthesis of 5-(4-Methoxybenzyloxy)-2-pentynal (72). According to established procedures (Journet et al., sura) a hexane solution of n-BuLi (2.5 M, 32 mL) was added dropwise to a −40° C. solution of 71 (14.45 g, 76.22 mmol) in dry THF (0.2 L). The reaction temperature did not exceed −35° C. After 10 min anhydrous DMF (11.8 mL, 153 mmol) was added in one portion and the cold bath was removed. After 30 min the reaction mixture was quenched by pouring into a vigorously stirred and cooled (˜5° C.) solution of 10% aqueous KH₂PO₄ (0.4 L) and methyl tert-butyl ether (MTBE) (0.38 L). After 20 min the layers were separated and the organic layer was washed with H₂O (50 mL). The combined aqueous layers were back extracted with MTBE (100 mL), and the combined organic extracts were washed with brine (50 mL), dried (MgSO₄), filtered and the filtrate concentrated. Purification of the residue on silica gel (10:1 hexanes-EtOAc; 6:1 hexanes-EtOAc) provided 14.97 g (90%) of 72 as a slightly yellow oil: ¹H NMR (500 MHz, CDCl₃) δ 9.16 (s, 1 H), 7.26 (d, J=8.5 Hz, 2 H), 6.88 (d, J=8.6 Hz, 2 H), 4.48(s, 2H), 3.79 (s, 3 H), 3.61 (t, J =6.7 Hz, 2 H), 2.69 (t, J=6.7 Hz, 2 H); ¹³C NMR (125 MHz, CDCl₃) δ 177.0, 159.2, 129.6, 129.3, 113.8, 95.7, 81.9, 72.7, 66.5, 55.2, 20.6 ppm; IR (film) 3002, 2865, 2205, 1668, 1514, 824 cm⁻¹; Anal. Calcd for C₁₃H₁₄O₃: C, 71.54; H, 6.47. Found: C, 71.42; H, 6.54.

Synthesis of (5S)-Hydroxy-1-(4-methoxybenzyloxy)-3-heptyne (73).

According to the general procedure of Seebach (Webber and Seebach, Tetrahedron 1994, 50:7473-7484), incorporated by reference herein, Ti(Oi-Pr)₄ (12.2 mL, 41.0 mmol) was added to a 23° C. solution of (4R, 5R)-2,2-dimethyl-α,α,α′,α′-tetra(naphth-2-yl)-1,3-dioxolan-4,5-dimethanol (27.3 g, 41.0 mmol) and dry toluene (340 mL). After 3 h, solvent was removed under reduced pressure (0.1 mm). The resulting residue was dissolved in dry Et ₂O (560 mL) and the reaction vessel was cooled to −50° C., whereupon Ti(Oi-Pr)₄ (70 mL, 0.24 mmol) and 72 (44.7 g, 0.20 nimol) were added. Diethyl zinc (243 mL, 267 mmol, 1.1 M solution in toluene) was then added slowly over 1 h. The reaction vessel was then warmed to −27° C. After 18 h the reaction mixture was quenched with saturated aqueous NH₄Cl (100 mL). The organic phase was dried (MgSO₄), filtered through Celite® and concentrated. The resulting residue was purified on silica gel (20:1 hexanes-EtOAc; 5.6:1 hexanes-EtOAc; 1:1 hexanes-EtOAc) to provide 47.6 g (94%) of 73 as a coloriess oil: ¹H NMR (500 MHz, CDCl₃) δ 7.25 (d, J=8.4 Hz, 2 H), 6.86 (d, J=8.4 Hz, 2 H), 4.46 (s, 2 H), 4.26 (t, J=6.4 Hz, 1 H), 3.78 (s, 3 H), 3.53 (t, J=7.0Hz, 2 H), 2.58 (s, 1 H), 2.49 (dt, J=7.0, 1.5Hz, 2 H), 1.66 (m, 2 H), 0.97 (t, J=7.4 Hz, 3 H); ¹³C NMR (125 Mz, CDCl₃) δ 159.1, 129.9, 129.2, 113.7, 82.3, 81.7, 72.4, 67.9, 63.5, 55.1, 30.9, 19.9, 9.4 ppm; IR (film) 3418, 2965, 1613, 1514, 1249, 823, 733 cm⁻¹; Anal. Calcd. for C₁₅H₂₀O₃: C, 72.55; H, 8.12. Found: C, 72.26; H, 8.14, [α]²⁵_{D-}3.2, [α]²⁵₅₇₇−3.6, [α]²⁵₅₄₅−4.0, [α]²⁵₄₃₅−6.5, [α]²⁵₄₀₅−7.7, (c 2.35, CHCl₃).

Following the general procedure of Ward (Ward et al., Tetrahedron Lett., 1991, 32:7165-7166), incorporated by reference herein, 73 (23 mg) was treated with (R)-α-methoxy-α-(triflouromethyl)phenylacetic acid chloride [(R)-MTPACl] to give the corresponding (R)-MTPA ester. Capillary GC analysis [150° C. to 200° C./2.0° C. min⁻¹, t_{R} 73-(R)-MTA=3D21.13 min, t_{R} ent-50-(R)-MTPA=20.69 min] showed a ratio for 99.7:0.3 of 73-(R)-MTPA and ent-73-(R)-MTPA.

Synthesis of (S)-(Z)-1-(4-Methyloxybenzyloxy)-5-triisopropylsiloxy-3-hentene (74).

Triisopropylsilyl trifluoromethanelsulfonate (19.1 mL, 71.1 mmol) was added dropwise over 15 min to a 0° C. solution of 2,6-lutidine (10.3 mL, 88.4 mmol), 73 (14.6 g, 58.6 mmol) and dry CH₂Cl₂ (150 mL). After 1 h, the solution was poured into Et₂O (400 mL) and washed with 1N HCl (3×50 mL) and brine (20 mL). The organic phase was dried (MgSO₄), filtered and the filtrate concentrated. The crude oil was placed under vacuum (0.1 mm) overnight to provide 24.0 g (˜100%) of (S)-1-(4-methoxybenzyloxy)-5-triisopropylsiloxy-3-heptyne as a slightly yellow oil, which was used without futher purification: ¹H NMR (400 MHz, CDCl₃) δ (d, J=8.6 Hz, 2 H), 6.91 (d, J=8.6 Hz, 2 H), 4.50(s, 2 H), 4.24-4.45 (m, 1 H), 3.83 (s, 3 H),3.59(t, J=7.2Hz,2H),2.54(dt, J=7.2, 1.9Hz,2 H), 1.67-1.76(m,2 H), 1.01-1.19 (m, 21 H), 1.02 (t, J=7.4 Hz, 3 H); ¹³C NMR (100 MHz, CDCl₃) 159.1, 130.2, 129.2, 113.7, 82.9, 80.8, 72.5, 68.2, 64.3, 55.1, 32. 1, 20.1, 18.0, 12.2, 9.4 ppm; IR (film) 2942, 2866, 1614, 1514, 1464, 1249, 1100 cm⁻¹; Anal. Calcd. for C₂₄H₄₀O₃Si: C, 71.24; H, 9.86. Found: C, 71.18; H, 10.04; [α]²⁵_{D}−25.5, [α]²⁵₅₇₇−26.3, [α]²⁵⁵₄₆−30.5, [α]²⁵⁴₃₅−50.8, [α²⁵₄₀₅−60.8, (c 1.40,.CHCl₃).

A mixture of crude (S)-1-(4-methoxybenzyloxy)-5-triisopropylsiloxy-3-heptyne (24.0 g, 58.6 mmol), freshly distilled quinoline (0.14 mL, 1.18 mmol), Lindlar's catalyst (Pd/CaCO₃ poisoned with PbO, 1.51 g) and dry 3:1 hexanes-EtOAc (360 mL) was maintained at 23° C. under 1 atm H₂ for 17 h. This mixture was then filtered through a plug of Celite, and the eluent was concentrated to yield 24.0 g (˜100%) of 74, which was used without futher purification: ¹H NMR (400 MHz, CDCl₃) δ 7.30(d, J=8.6 Hz, 2 H), 6.91 (d, J=8.6 Hz, 2 H), 5.47-5.52 (m, 1 H), 5.37-5.43 (m, 1 H), 4.48-4.52 (m, 1 H), 4.48 (s, 2 H), 3.83 (s, 3 H), 3.46-3.50 (m, 2 H), 2.35-2.43 (m, 2 H), 1.59-1.68 (m, 1 H), 1.47-1.56 (m, 1 H), 1.09 (app s, 21 H), 0.89 (t, J=7.4 Hz, 3 H); ¹³C NMR (100 MHz, CDCl₃) 159.1, 135.8, 130.4, 129.2, 124.4, 113.7, 72.6, 69.9, 69.4, 55.2, 31.6, 28.7, 18.0, 12.3, 9.3 ppm; IR (film) 2942, 2866, 1613, 1514, 1248, 1097 cm⁻¹; Anal. Calcd. for C₂₄H₄₂O₃Si: C, 70.88; H, 10.41. Found: C, 71.06; H, 10.44; [α]²⁵_{D}18.5, [α]²⁵₅₇₇+19.7, [α]²⁵₅₄₆+22.6, [α]²⁵₄₃₅+41.9, [α]²⁵₄₀₅+52.0, (c 1.80, CHCl₃).

Synthesis of (S)-(Z)-1-Iodo-5-triisopropylsiloxy-3-heptene (75).

A solution of crude 74 (24.0 g, 58.6 mmol), DDQ (17.3 g, 76.2 mmol) and 20:1 CH₂Cl₂—H₂O (210 mL) was maintained at 23° C. for 1 h. The reaction mixture was quenched by pouring into Et₂O (600 mL) and washing with 1N NaOH (2×200 mL) and brine (200 mL). The organic phase was dried (MgSO₄), filtered and concentrated. Chromatagraphic separation of p-methoxybenzaldehyde was facilitated by reduction top-methoxybenzyl alchohol. Towards this end, a solution of the resulting residue, MeOH (200 mL) and NaBH₄ (2.9 g, 77 nmmol) was maintained at 23° C. for 1 h. The reaction mixture was quenched by pouring into Et₂O (300 mL) and washing with 1N HCl (50 mL) and brine (50 mL). The organic phase was dried (MgSO₄), filtered and the filtrate concentrated. The resulting oil was purified on silica gel (20:1 hexanes-EtOAc; 15:1 hexanes-EtOAc; 10:1 hexanes:EtOAc) to provide 16.0 g (95%) of (S)-(Z)-5-triisopropylsiloxy-3-heptenol as a colorless oil: ¹H NMR (500 MHz, CDCl₃) δ 5.55-5.51 (m, 1 H), 5.38-5.33 (m, 1 H), 4.47 (ddd, J=13.5, 6.5, 1.5 Hz, 1 H), 3.66 (m, 2 H), 2.35-2.30 (m, 2 H), 1.65-1.57 (m, 1 H), 1.53-1.46 (m, 1 H), 1.41 (br s, 1 H), 1.05 (br s, 21 H), 0.87 (t, J=7.5 Hz); ¹³C NMR (125 MHz, CDCl₃) 137.0, 124.1, 69.9, 62.3, 31.7, 31.6, 18.0, 12.3, 9.3; IR (film) 3313, 2970, 2867; 1485, 1085, 1052 cm⁻¹; Anal. Calcd for C₁₆H₃₄O₂Si: C, 67.07; H, 11.96. Found: C, 66.89; H, 11.89; [α]²⁵_{D}+23.2, [α]²⁵₅₇₇+25.1, [α]²⁵₅₄₆+29.2, [α]²⁵₄₃₅+52.9, [α]²⁵₄₀₅+66.1, (c 1.25, CHCl₃).

Following the general procedure of Corey (Singh et al., supra, incorporated by reference herein) iodine (5.03 g, 19.8 mmol) was added in portions over 15 min to a 0° C. solution of (S)-(Z)-5-triisopropylsiloxy-3-heptenol (5.17 g, 18.0 mmol), PPh₃ (5.19 g, 19.8 mmol), imidazole (1.35 g, 19.8 mmol) and Et₂O—MeCN (3:1, 135 mL) and then allowed to warm to 23° C. After 1 h the solution was partitioned between H₂O (150 mL) and Et₂O (150 mL). The aqueous phase was extracted with Et₂O (2×150 mL). The combined organic extracts were then washed with Na₂SO₃ (150 mL) and H₂O (150 mL), dried (MgSO₄) and filtered. Purification of the crude product by flash chromatography (95:5 hexanes-Et₂O) afforded 6.67 g (94%) of iodide 75 as a colorless oil: ¹H NMR (500 MHz, CDCl₃) δ 5.49-5.53 (m, 1 H), 5.28-5.32 (m, 1 H), 4.41 (dd, J=7.1, 5.9 Hz, 1 H), 3.10-3.14 (m, 2 H), 2.59-2.66 (m, 2 H), 1.58-1.62 (m, 1 H), 1.48-1.52 (m, 1 H), 1.05 (s, 21 H), 0.86 (t, J=7.4 Hz, 3 H); ¹³C NMR (125 MHz, CDCl₃) 136.2, 126.9, 70.0, 32.2, 31.6, 18.1, 12.3, 9.3, 4.4 ppm; IR (film) 3012, 2942, 1464, 1105, 883 cm⁻¹; Anal. Calcd for C₁₆H₃₃OSiI: C, 48.48; H, 8.39. Found: C, 48.63; H, 8.49; [α]²⁵_{D}+22.8, [α]²⁵₅₇₇+24.4, [α]²⁵₅₄₆+23.7, [α]²⁵₄₃₅+53.1, [α]²⁵₄₀₅+65.8, (c 1.2, CHCl₃).

Synthesis of (R)-Triethylsiloxy-N-methoxy-N-methyl-7-methyl-6-octenamide (Compound 76.

To a 0° C. solution of the known (Noyori, R. et. al. J. Am. Chem. Soc. 1987, 109, 5868) β-hydroxyester (10.0 g, 53.5 mmol) in dry THF (200 mL) was added N, O-dimethylhydroxylamine hydrochloride (14 g, 64.2 mmol, 1.2 eq) followed by a 2 M solution of trimethylaluminum in toluene (60 mL, 2.3 eq) (added dropwise via cannula). The mixture was allowed to warm to room temperature and maintained at this temperature for 3 h. Then the mixture was (carefully) poored into a cold (0° C.) 2 M solution of tartaric acid (500 mL). The resulting mixture was stirred for 5 h, after which the layers were separated and the aqueous layer was extracted with EtOAc (3×100 mL). The combined organic layers were dried (MgSO₄) and concentrated. Purification of the residue on silica gel yielded 10.2 g (88%) of Weinreb amide. The Weinreb amide (10.2 g, 47.5 mmol) was dissolved in CH₂Cl₂ (150 mL) and treated with Hünig's base (25 mL, 3eq). TESCl (8.6 g, 9.7 mL, 1 .2 eq) was then added dropwise to the mixture. The progress of the reaction was monitored by TLC (hexanes, EtOAc, 3:1), and, upon completion, the mixture was diluted with water, the layers separated and the aqueous layer extracted with Et₂O (3×100 mL). The combined organic layers were washed with 0.5 N HCl (2×100 mL) and water (2×100 mL), dried (MgSO₄), and concentrated. The residue was purified on silica gel (hexane-EtOAc, 3:1), to give 12.9 g (82%) of 76 as a pale yellow oil. ¹H NMR (CDCl₃, 300 MHz) δ 5.05 (m, 1 H), 4.20-4.10 (m, 1H), 3.65 (s, 3H), 3.14 (s, 3H), 2.8-2.6 (dd, J=17,2 Hz, 1H), 2.3-2.4 (dd, J=17, 3Hz, 1H), 2.1-1.9 (m, 2H), 1.65 (s, 3H), 1.55 (s, 3H), 1.55-1.4 (m, 2H), 1.0-0.9 (m, 9H), 0.6-0.4 (m, 6H); ¹³C NMR (CDCl₃, 75 MHz) d 172.38, 131.50, 124.10, 69.10, 61.17, 39.61, 37.96, 31.90, 25.55, 23.81, 17.56, 6.80, 5.24.

Synthesis of (6R, 11Z, 13S)-8-(1′,3′- 13S)-2-Methyl-6-triethylsilyloxy-13-trisopropylsiloxypentadeca-2,11-dien-8-one (77).

t-Buli (23.5mL, 40.0 mmol, 1.7 M) was added to a −78° C. solution of iodide 75 (6.67 g, 16.8 mmol) and Et₂O-hexanes (1:1, 100 mL). The solution was maintained at −78° C. for 30 min, then a solution amide 76 (6.10 g, 18. 5 mmol) and Et₂O-hexanes (1:1, 40 mL) was added. The resulting solution was maintained at −78° C. for 30 min then allowed to warm to 0° C. and maintained for 2 h. The solution was then added to saturated aqueous NH₄Cl (150 mL). The phases were separated, and the aqueous phase was extracted with Et₂O (2×150 mL). The combined organic extracts were dried (MgSO₄), filtered and concentrated. Purification of the crude product by flash chromatography (98:2 hexanes-Et₂O) afforded 5.93 g (65%) of 77 as a clear oil. The product was ca. 95% pure and was used without further purification. A small sample was further purified by flash chromatography (98:2 hexanes-Et₂O) to obtain the following data: ¹H NMR (400 MHz, CDCl₃) δ 5.41-5.36 (m, 1 H), 5.29-5.24 (m, 1 H), 5.08 (tt, J=7.1, 1.3 Hz, 1 H), 4.45 (app q, J=6.7 Hz, 1 H), 4.18 (quintet, J=6.0 Hz, 1 H), 2.60 (A of ABX, JAB=3D 15.3, JAX=3D 7.2 Hz, 1 H), 2.48-2.43 (m, 3 H), 2.30-2.24 (m, 2 H), 2.05-1.93 (m, 2 H), 1.68 (s, 3 H), 1.64-1.40(m, 4H), 1.59(s, 3 H), 1.04 (s, 21 H), 0.94 (t, J=7.9 Hz, 9 H), 0.85 (t, J=7.5 Hz, 3 H), 0.58 (q, J=7.9 Hz, 6 H); ¹³C NMR (100 MHz, CDCl₃) δ 208.9, 135.1, 131.8, 126.7, 123.8, 69.8, 68.7, 50.2, 44.1, 37.9, 31.6, 25.7, 23.8, 21.9, 18.1, 18.0, 17.6, 12.3, 9.3, 6.9, 4.9 ppm; (film) 2958, 2867, 1717, 1463, 1378, 1086, 1014 cm⁻¹; MS: HRMS (FAB) m/z 37.4141 (M−H), (537.4159 calcd for C₃₁H₆₁O₃Si₂); [α]²⁵_{D}+4.1, [α]²⁵₅₇₇+4.8, [α]²⁵₅₄₆+4.9, [α]²⁵₄₃₅+11.0, [α]²⁵₄₀₅+14.3 (c 1.6, CHCl₃).

Synthesis of (6R, 11Z, 13S)-8-(1′,3′-dioxan-2′-yl)-6-hydroxy-2-methyl-13-trisopropylsiloxypentadeca-2,11-diene (79).

A solution of ketone 77 (3.74 g, 6.94 mmol), orthoester 78 (4.10 g, 34.7 mmol), 1,3-propanediol (12.6 mL, 174 mmol), Amberlyst-15 resin (278 mg) and CH₃CN (70 mL) was maintained at rt for 7 h. The mixture was then filtered through Celite and the filtrate was partitioned between Et₂O (150 mL) and H₂O (50 mL). The phases were separated, and the organic phase was washed with H₂O (250 mL), dried (MgSO₄), filtered and concentrated. Purification of the crude product by flash chromatography (85:15 hexanes-Et₂O) afforded 2.68 g (80%) of ketal 79 as clear oil: ¹H NMR (500 MHz, CDCl₃) δ 5.42-5.29 (m, 2 H), 5.14 (broad t, J=7.1 Hz, 1 H), 4.45 (app q, J=7.5 Hz, 1 H), 4.11-4.08 (m, 1 H), 4.02-3.85 (m, 4 H), 3.80 (s, 1 H), 2.16-1.96 (m, 6 H), 1.84-1.76 (m, 202 H), 1.68 (s, 3 H), 1.65-1.36 (m, 6 H), 1.61 (s, 3 H), 1.05 (s, 21 H), 0.86 (t, J=7.4 Hz, 3 H); ¹³C NMR (125 MHz, CDCl₃) δ 134.6, 131.5, 127.5, 124.3, 101.1, 69.9, 67.0, 59.5, 59.5, 43.7, 37.5, 31.7, 31.3, 25.7, 25.2, 24.1, 22.3, 18.1, 18.0, 17.6, 12.4, 9.3 ppm; IR (film) 3532, 2960, 2866, 1464, 1381, 1246, 1109 cm⁻¹; MS: HRMS (FAB) m/z 505.3683 (M+Na), (505.3691 calcd for C₂₈H54O₄SiNa). Anal. Calcd for C₂₈H₅₄O₄Si: C, 69.65; H, 11.27. Found: C, 69.40; H, 11.28; [α]²⁵_{D}+13.3, [α]²⁵₅₇₇+14.2, [α]²⁵₅₄₆+16.8, [α]²⁵₄₃₅+30.1, [α]²⁵₄₀₅+37.4 (c 1.6, CHCl₃).

Synthesis of (6S, 11Z, 13S)-6-amino-8-(1′,3′-dioxan-2′-yl)-2-methyl-13-trisopropylsiloxypentadeca-2,11-diene (80).

Triphenylphosphine (2.89 g, 11.0 mmol) and hydrazoic acid (5.82 mL, 12.1 mmol, 2.08 M in toluene) were added to a 0° C. solution of alcohol 79 (2.65 g, 5.49 mmol) and THF (55 mL), then diethylazodicarboxylate (DEAD) (2.60 mL, 16.5 mmol) was added dropwise over a period of 15 min. The solution was maintained at 0° C. for 1.5 h, then approximately half of the solvent was removed in vacuo. The resulting solution was diluted with hexanes (30 mL) and filtered through a plug of silica gel using 97:3 hexanes-Et₂O as the eluant. The filtrate was concentrated, and the crude product was purified by flash chromatography (97:3 hexanes-Et2O) affording 2.45 g (88%) of the azide as a clear oil: ¹H NMR (500 MHz, CDCl₃) δ 5.41-5.29 (m, 2 H), 5.10 (broad t, J=7.1 Hz, 1 H), 4.47 (app q, J=7.4 Hz, 1 H), 3.96-3.86 (m, 4 H), 3.71-3.66 (m, 1 H), 2.12-2.07 (m, 3 H), 2.00-1.72 (m, 6 H), 1.70 (s, 3 H), 1.64 (s, 3 H), 1.63-1.42 (m, 5 H), 1.05 (s, 2 H), 0.87 (t, J=7.4 Hz, 3 H); ¹³C NMR (125 MHz, CDCl₃) δ 134.6, 132.4, 127.7, 123.3, 99.1, 69.9, 59.6, 59.6, 58.3, 42.2, 36.1, 32.2, 31.7, 25.7, 25.1, 24.7, 22.2, 18.1, 18.1, 17.6, 12.4, 9.4 ppm; IR(film) 2961, 2866, 2101, 1463, 1381, 1246, 1145, 1110 cm⁻¹; MS: HRMS(FAB)(M−H) m/z 506.3776, (506.3781 calcd for C₂₈H₅₂N₃O₃Si). Anal. Calcd for C₂₈H₅₃N₃O₃Si: C, 66.22; H, 10.52. Found: C, 66.27; H, 10.50. [α]²⁵_{D}+9.5, [α]²⁵₅₇₇+10.3, [α]²⁵₅₄₆+12.1, [α]²⁵₄₃₅+24.1, [α]²⁵₄₀₅+31.2 (c 1.6, CHCl₃).

A solution of the above azide (2.45 g, 4.82 mmol) and Et₂O (18 mL) was added to a 0° C. solution of LiAlH₄ (12.1 mL, 12. 1 mmol, 1.0 M in Et₂O) and Et₂O (18 mL). The ice bath was removed, and the solution was allowed to warm to rt. After 1 h the reaction was quenced by sequential addition of H₂O (600 μL), NaOH (600 μL, 3 N) and H₂O (1.8 mL). The resulting mixture was stirred for 1 h, then MgSO₄ was added. The mixture was filtered through celite and concentrated to afford a brown oil. Purification of the crude product by flash chromatography (10:1:0.1 CHCl₃-MeOH-conc. NH₄OH) afforded 2.05 g (88%) of amine 80 as a light yellow oil: ¹H NMR (500 MHz, CDCl₃) δ 5.39-5.29 (m, 2 H), 5.11 (br t, J=7.1 Hz, 1 H), 4.46 (app q, J=7.4 Hz, 1 H), 3.95-3.84 (m, 4 H), 3.15-3.11 (m, 1 H), 2.10-1.96 (m, 4 H), 1.83-1.69 (m, 4 H), 1.68 (s, 3 H), 1.63-1.31 (m, 6 H), 1.61 (s, 3 H), 1.05 (s, 21 H), 0.86 (t, J=7.5 Hz, 3 H); ¹³C NMR (125 MHz, CDCl₃) δ 134.3, 131.4, 127.9, 124.1, 100.4, 69.8, 59.4, 59.2, 46.7, 43.1, 38.8, 32.7, 31.6, 25.6, 25.3, 24.6, 22.1, 18.0, 18.0, 17.6, 12.3, 9.3 ppm; IR (film) 3387, 3310, 2942, 2866, 1464, 1381, 1366, 1246, 1109 cm⁻¹; MS: HRMS (FAB) (M+H⁺) m/z 482.4011, (482.4029 calcd for C₂₈H₅₆NO₃Si). Anal. Calcd for C₂₈H₅₅NO₃Si: C, 69.80; H, 11.51. Found: C, 69.85; H, 11.56; [α]²⁵_{D}+21. 2, [α]²⁵₅₇₇+22.7, [α]²⁵₅₄₆+26.1, [α]²⁵₄₃₅+47.2, [α]²⁵₄₀₅+58.1 (c 1.6, CHCl₃).

Synthesis of (6S, 11Z, 13S)-8-(1′,3′-Dioxan-2′-yl)-2-methyl-13-trisopropylsiloxy-6-uriedopentadeca-2,11-diene (81).

Trimethylsilyl isocyanate (0.55 mL, 4.1 mmol) was added to a rt solution of 80 (1.61 g, 3.35 mmol), CH₂Cl₂ (6.8 mL) and i-PrOH (0.31 mL). After 15 h, i-PrOH (3 mL) was added and the solution was maintained for 1 h, then concentrated. The resulting oil was purified on silica gel (100% EtOAc) to provide 1.57 g (89%) of 81 as a colorless oil: ¹HNMR (400 MHz, CDCl³) δ 5.24-5.36 (m, 2H), 5.03-5.15 (m,4H),4.41 (dd, J=13.2, 7.1 Hz, 1H),3.80-3.91 (m,4H),3.64(m, 1H), 1.71-2.03 (m, 8H), 1.63 (s, 3H), 1.55 (s, 3H), 1.36-1.63 (m, 6H), 1.00 (s, 21H), 0.82 (t, J=7.4 Hz, 3H); ¹³C NMR (100 MHz, CDCl₃) 159.3, 134.4, 131.8, 127.5, 123.7, 99.9, 69.7, 59.4, 59.2, 46.7, 36.9, 31.5, 31.1, 25.6, 25.0, 24.5, 22.1, 17.9, 17.8, 17.5, 12.2, 9.2 ppm; IR(film) 3354, 2960, 1660, 1600, 1556, 1463, 1381, 1109 cm⁻¹; [α]²⁵_{D}+7.0, [α]²⁵₅₇₇+12.0, [α]²⁵₅₄₆+17.3, [α]²⁵₄₃₅+20.7, [α]²⁵₄₀₅+25.4, (c 1.05, CHCl₃). Anal. Calcd for C₂₉H₅₆N₂O₄Si: C, 66.36; H, 10.75; N, 5.34. Found: C, 66.31; H, 10.70; N 5.41.

Synthesis of (4aR, 7S)-4-[15-(Allyloxycarbonyl)pentadecyloxycarbonyl]-1,2,4a,5,6,7-hexahydro-3-[(4S)-tert-butyldimethylsiloxypentyl)]-7-[α]-2-(1′,3′-dioxan-2′-yl)-7-triisopropylsiloxynon-5-enyl)]-1-oxo-pyrrolo[1,=2-c] pyrimidine (83).

Osmium tetroxide (0.75 mL, 0.1 M in t-BuOH) was added to a solution of 81 (524 mg, 1.00 mmol), NMO (406 mg, 3.46 mmol), and 10:1 ThF-H₂O (25 mL). After 1.5 h, florisil (3 g), NaHSO₃ (3 g), and EtOAc (50 mL) were added and the reaction mixture was stirred vigouously. After 30 min, the reaction mixture was filtered, and the filtrate concentrated to provide a colorless oil which was used without further purification.

A solution of this crude diol, Pb(OAc)₄. (532 mg, 1.20 mmol), and toluene (60 mL) was maintained at room temperature. The reaction mixture was filtered through a plug of Celite,® morpholinium acetate (300 mg, 2.0 mmol) was added, and the solution concentrated to provide the crude aminal 82 as a slightly yellow oil.

A solution of this crude aminal, 47 (1.95 g, 3.36 mmol) and 2,2,2-trifluoroethanol (1.0 mL) was maintained at 60° C. for 2 d. The reaction was quenched by adding Et₂O (20 mL) and 50% aqueous NH₄Cl (5 mL). The layers were separated, the organic layer was dried (MgSO₄), concentrated, and the resulting oil purified on silica gel (10:1 hexanes-EtOAc; 7:1 hexanes-EtOAc; 3:1 hexanes-EtOAc) to provide 1.5 g of 24 and 638 mg (61%) of a ˜6.5:1 mixture of 83 and 84, which was used without separation. For characterization purposes, a 50 mg sample of this mixure was purified by HPLC (7:1 hexanes-EtOAc; Altima 5_silica). 60: ¹H NMR (500 MHz, CDCl₃) δ 6.72 (s, 1H), 5.87-5.95 (m, 1H), 5.21-5.37 (m, 4H), 4.56 (d, J=5.7 Hz, 2H), 4.51 (dd, J=12.7, 7.1 Hz, 1H), 4.22 (dd, J=11, 4.6 Hz, 1H), 4.06-4.13 (m, 3H), 3.97-3.98 (m, 1H), 3.76-3.88 (m, 4H), 2.47-2.58 (m, 3H), 2.39 (d, J=13.6 Hz, 1H), 2.32 (t, J=7.5 Hz, 2H), 2.26-2.32 (m, 1H), 2.15 (dd, J=13.0, 6.0 Hz, 1H), 1.99-2.03 (m, 1H), 1.50-1.90(m, 13H), 1.41-1.48(m, 3H), 1.11-1.40(m, 23H), 1.10(d, J=6. 1 Hz, 3H), 0.91-1.07 (m, 21H), 0.82-0.91 (m, 12H), 0.03 (s, 3H), 0.02 (s, 3H); ¹³C NMR (125 MHz, CDCl₃) 173.5, 166.0, 151.9, 151.2, 134.3, 132.2, 128.2, 118.0, 102.1, 99.2, 69.9, 68.3, 64.9, 64.2, 59.3, 57.7, 52.7, 39.0, 37.4, 34.5, 34.2, 31.8, 31.3, 30.4, 29.6, 29.57, 29.5, 29.4, 29.3, 29.2, 29.1, 29.0, 26.1, 25.9, 25.3, 24.9, 24.4, 23.6, 21.8, 18.1, 12.3, 9.3, −4.4, −4.7 ppm; IR (film) 3211, 3095, 2927, 2856, 1741, 1682, 1627, 1463, 1435, 1107 cm⁻¹; [α]²⁵_{D}−4.5, [α]²⁵₅₇₇−4.9, [α9 ²⁵₅₄₆5.7, [α]²⁵₄₃₅15.5, [α]²⁵₄₀₅22.7, (c 0.75, CHCl₃). Anal. Calcd for C₅₉H₁₀₈N₂₀₉Si₂: C, 67.77; H, 10.41; N, 2.68. Found: C, 67.68; H, 10.27; N 2.65.

(3R,4R,4aR,6′R,7S)-4-[15-(Allyloxycarbonyl)pentadecyloxycarbonyl]-1,2,4a,5,6,7-hexahydro-1oxo-7-[(7S, 5Z)-7-hydroxy-2oxo-5-nonenyl]-pyrrolo[1,2-c]pyrimidine-3-spiro-6′-(2′-methyl)-3′,4′,5′,6′-tetrahydro-2H-pyran (85).

A solution of 83 (1.30 g, 1.24 mmol), TBAF (6.22 mL, 1.0 M solution in Et₂O), and DMF (31 mL) was maintained at rt for 5 h. The solution was diluted with Et₂O (150 mL) and washed with H₂O (50 mL) and brine (250 mL). The organic layer was dried (MgSO₄), filtered, and the filtrate concentrated. The resulting residue was used without further purification.

A solution of this crude diol, TsOH.H₂O (236 mg, 1.24 mmol), and CHCl₃ (180 mL) was maintained at 60° C. for 15 min. The reaction was quenched by adding saturated aqueous NaHCO₃ (20 mL). The layers were separated and the organic layer was washed with brine (20 mL), then the organic layer was dried (MgSO₄), concentrated, and the resulting oil purified on silica gel (1:3 hexanes-EtOAc; 100% EtOAc) to provide 630 mg (71%) of a ˜6.5:1 mixture isomers. 62: ¹H NMR (500 MHz, CDCl₃) δ 5.87-5.95 (m, 1H), 5.56 (s, 1H), 5.34-5.43 (m, 2H), 5.31 (dd, J=17.2, 1.5 Hz, 1H), 5.22 (dd, J=10.6, 1.3 Hz, 1H), 4.57(dd, J=4.3, 1.3 Hz, 2H), 4.38 (dd, J=14.5, 6.8 Hz, 1H), 4.29-4.31. (m, 1H), 4.08-4.18 (m, 2H), 4.02 (dt, J=11.1, 4.8 Hz, 1H), 3.77-3.80 (m, 1H), 3.37 (d, J=16.8 Hz, 1H), 2.52-2.60 (m, 2H), 2.43-2.50 (m, 1H), 2.32 (t, J=7.5 Hz, 2H), 2.22-2.27 (m, 2H), 2.04-2.20 (m, 4H), 1.69-1.76 (m, 4H), 1.56-1.65 (m, 7H), 1.42-1.48 (m, 3H), 1.24-1.28 (m, 21H), 1.06-1.09 (m, 1H), 1.05 (d, J=6.0 Hz, 3H), 0.89 (t, J=7.5 Hz, 3H); ¹³C NMR (125 MHz, CDCl₃) 209.0, 173.5, 168.9, 153.0, 134.1, 132.3, 129.8, 118.1, 82.2, 68.4, 66.2, 65.1, 64.9, 55.0, 54.0, 53.2, 46.2, 42.7, 34.3, 32.2, 32.1, 30.3, 30.0, 29.6, 29.57, 29.5, 29.4, 29.3, 29.2, 29.1, 28.7, 26.0, 24.9, 22.0, 21.7, 18.8ppm; IR (film)3450, 3231, 3081, 2927, 2855, 1732, 1715, 1659, 1651, 1470, 1373, 1262, 1013 cm⁻¹; [α]²⁵_{D}+42.2, [α]²⁵₅₇₇+42.7, [α]²⁵₅₄₆+49.8, [α]²⁵₄₃₅+91.0, [α]²⁵₄₀₅+114, (c 0.60, CHCl₃). Anal. Calcd for C₄₁H₆₈N₂O₈: C, 68.68; H, 9.56; N, 3.91. Found: C, 68.71; H, 9.51; N 3.84.

Synthesis of (3R,4R,4aR,6′R, 7S)-4-[15-Allyloxycarbonyl)pentadecyloxycarbonyl]-1,2,4a,5,6,7-hexahydro-1-oxo-7-[(7S, 5Z)- 7-chloroacetoxy-2oxo-5-nonenyl]-pyrrolo[1,2-c]pyrimidine-3-spiro-6′-(2′-methyl)-3′,4′,5′,6′-tetrahydro-2H-pyran (86).

Chloroacetyl chloride (0.34 mL, 0.46 mmol) was added dropwise to a 0° C. solution of 85 (0.63 g, 0.88 mmol), pyridine (1.42 mL, 17.6 mmol), and CH₂Cl₂ (50 mL). The solution was immediately allowed to warm to rt. After 1 h, the solution was quenched by adding Et₂O (200 mL) and washed with 1N NaOH (25 mL), CuSO₄ (225 mL), and brine (25 mL). The organic layer was dried (MgSO₄), filtered, and the filtrate concentrated. The resulting residue was purified on silica gel (2:1 hexanes-EtOAc; 1:1 hexanes-EtOAc; 1:2 hexanes-EtOAc) to yield 600 mg (86%) of the desired cis isomer 86 as a colorless oil, and ˜85 mg (˜12%) of an undesired trans isomer which was derived from 84. 86: ¹H NMR (500 MHz, CDCl₃) δ 6.34 (s, 1H), 5.87-5.94 (m, 1H), 5.48-5.56 (m, 2H), 5.27-5.32 (m, 2H), 5.22 (d, J=10.4 Hz, 1H), 4.56 (d, J=5.7 Hz, 2H), 4.31-4.33 (m, 1H), 4.09-4.19 (m, 2H), 4.03 (s, 2H), 4.00-4.06 (m, 1H), 3.77-3.81 (m, 1H), 3.34 (d, J=16.6 Hz, 1H), 2.40-2.48 (m, 3H), 2.25-2.38 (m, 5H), 2.05-2.17 (m, 3H), 1.69-1.74 (m, 4H), 1.55-1.62 (m, 7H), 1.42-1.50 (m, 1H), 1.24-1.31 (m, 22H), 1.06-1.15 (m, 1H), 1.05 (d, J=6.0 Hz, 3H), 0.89 (t, J=7.5 Hz, 3H); ¹³C NMR (125 MHz, CDCl₃) 207. 9, 173.4, 168.8, 166.6, 153.4, 133.0, 132.3, 127.8, 118.0, 82.1, 73.7, 66.1, 64.9, 64.8, 54.9, 53.9, 53.1, 46.3, 42.3, 41.1, 34.2, 32.2, 32.0, 29.5, 29.49, 29,4, 29.3, 29.2, 29.1, 29.07, 29.0, 28.6, 27.4, 25.9, 21.8, 21.6, 18.5, 9.3 ppm; IR (film) 3296, 2928, 2855, 1732, 1652, 1466, 1303, 1174, 1013 cm⁻¹; [α]²⁵_{D}+42.7, [α]²⁵₅₇₇+47.0, [α]²⁵₅₄₆+52.6, [α]²⁵₄₃₅+96.1, [α]²⁵₄₀₅+120, (c 1.00, CHCl₃). Anal. Calcd for C₄₃H69N₂O₉Cl: C, 65.09; H, 8.77; N, 3.53. Found: C, 65.16; H, 8.79; N 3.57.

Synthesis of Pentacyles 87 and 88.

A solution of 86 (327 mg, 0.412 mmol), MeOTf (1.29 mL, 8.21 mmol), 2,6-di-t-butylpyridine (0.46 mL, 2.1 mmol), and CH₂Cl₂ (20 mL) was maintained at room temperature for 8 h. The solution was then poured into Et₂O (100 mL) and washed with 1 N NaOH (2×10 mL) and brine (10 mL). The organic layer was dried (MgSO₄), filtered, and the filtrate concentrated. The resulting residue was used without further purification.

Ammonia was bubbled through a room temperature solution of the above crude pseudourea, N₄HCl (50 mg, 0.93 mmol), and allyl alcohol (5 mL) for 20 min (saturated solution). The reaction vessel was sealed and heated to 60° C. for 28 h. The reaction mixture was then cooled rt, concentrated, and the resulting oil purified by silica gel MPLC (100:0.6 CHCl₃-i-PrOH) to provide a 147 mg of 87 and 98 mg of 88.87 was twice recycled through the above guanylation reaction conditions to yield an additional 60 mg of 88 (52% combined yield). 87: ¹H NMR (500 MHz, CDCl₃) δ 8.68 (s, 1H), 8.56 (s, 1H), 5.88-5.95 (m, 1H), 5.64-5.67 (m, 1H), 5.48 (d, J=10.9 Hz, 1H), 5.33 (dd, J=17.2, 1.5 Hz, 1H), 5.25 (dd, J=10.4, 1.2 Hz, 1H), 4.57 (d, J=5.7 Hz, 2H), 4.48 (d, J=10.3 Hz, 1H), 4.32-4.38 (m, 1H), 4.10-4.24 (m, 3H), 3.78-3.81 (m, 1H), 2.56-2.61 (m, 2H), 2.45 (d, J=11.6 Hz, 1H), 2.32 (t, J=7.6 Hz, 2H), 2.26-2.36 (m, 3H), 2.15-2.18 (m, 2H), 2.00.(dt, J=13.8, 4.7 Hz, 1H), 1.87 (dd, J=14.6, 5.4 Hz, 1H), 1.61-1.78 (m, 10H), 1.53-1.58 (m, 1H), 1.42-1.49 (m, 1H), 1.23-1.35 (m, 22H), 1.05-1.15 (m, 1H), 1.05 (d, J=6.1 Hz, 3H), 0.81 (t, J=7.2 Hz, 3H); ¹³C NMR (125 MHz, CDCl₃) 173. 5, 167.6, 147.3, 133.4, 132.3, 129.7, 118.0, 83.9, 81.7, 70.9, 67.6, 65.7, 64.9, 53.4, 53.3, 36.8, 36.2, 34.2, 31.8, 30.6, 29.7, 29.6, 29.5, 29.46, 29.4, 29.2, 29.1, 29.08, 29.0, 28.5, 25.9, 24.9, 23.6, 21.3, 17.9, 10.1 ppm; IR (film) 3268, 3147, 3020, 2927, 2855, 1732, 1660, 1608, 1465, 1283, 1164, 1029 cm⁻¹; [α]²⁵_{D}+12.2, [α]²⁵₅₇₇+13.1, [α]²⁵₅₄₆+14.1, (c 2.00, CHCl₃). HRMS (FAB) m/z 698.5108 cald for C₄₁,H₆₈N₃O₆, found 698.5096 [M]. 88: ¹H NMR (500 MHz, CDCl₃) δ 8.54 (s, 1H), 8.43 (s, 1H), 5.88-5.95 (m, 1H), 5.64-5.67 (m, 1H), 5.48 (d, J=10.9 Hz, 1H), 5.31 (dd, J=17.2, 1.5 Hz, 1H), 5.22 (dd, J=10.4, 1.2 Hz, 1H), 4.57 (dd, J=5.7, 1.2 Hz, 2H), 4.48 (d, J=9.7 Hz, 1H), 4.29-4.33 (m, 1H), 4.08 (t, J=6.8 Hz, 2H), 3.99-4.05 (m, 1H), 3.84-3.87 (m, 1H), 2.93 (d, J=4.8 Hz, 1H), 2.55-2.63 (m, 2H), 2.32 (t, J=7.6 Hz, 2H), 2.26-2.36 (m, 2H), 2.13-2.24 (m, 3H), 1.98 (dd, J=14.7, 5.3 Hz, 1H), 1.78-1.84 (m, 1H), 1.51-1.76(m, 10H), 1.38-1.48(m, 2H), 1.21-1.30(m, 22H), 1.07-1.20(m, 1H), 1.05 (d, J=6.1 Hz, 3H), 0.81 (t, J=7.2 Hz, 3H); ¹³C NMR (125 MHz, CDCl₃) 173.5, 168.3, 148.4, 133.5, 132.3, 129.7, 118.0, 84.0, 80.8, 70.9, 67.2, 65.5, 64.9, 54.1, 52.2, 49.9, 36.7, 36.1, 34.4, 31.8, 31.7, 30.5, 29.6, 29.56, 29.5, 29.45, 29.4, 29.2, 29.1, 29.06, 28.4, 26.7, 25.8, 24.9, 23.6, 21.5, 17.7, 10.0 ppm; IR (film) 3263, 3154, 3025, 2928, 2854, 1732, 1652, 1614, 1516, 1464, 1298 cm⁻¹; [α]²⁵_{D}−9.6, [α]²⁵₅₇₇−10.5, [α]²⁵₅₄₆−9.5, [α]²⁵₄₃₅−16.5, [α]²⁵₄₀₅−17.2, (c 0.75, CHCl₃). HRMS (FAB) m/z 698.5108 cal'd for C₄₁H₆₈N₃O₆, Found 698.5106 [M].

Synthesis of Carboxylic Acid 89:

A solution of 88 (27 mg, 37 μmol), Pd(PPh₃)₄ (21 mg, 18 μmol), morpholine (13 μL, 0.15 mmol), and MeCN (1.0 mL) was maintained at rt for 5 h. The solution was diluted with Et₂O (30 mL), and washed with 0.1 N HCl (25 mL) and brine (5 mL). The organic layer was dried (MgSO₄), filtered, and the filtrate concentrated. The resulting residue was purified on silica gel (100:1 CHCl₃-MeOH; 33:1 CHCl₃-MeOH) to yield 24 mg (94%) of the desired product 89 as a colorless oil: ¹H NMR (500 MHz, CDCl₃) δ 5.63-5.66 (m, 1H), 5.46-5.49 (m, 1H), 4.48 (app d, J=10.2 Hz, 1H), 4.27-4.31 (m, 1H), 4.04-4.12 (m, 2H), 3.96-4.03 (m, 1H), 3.85-3.88 (m, 1H), 2.92 (d, J=4.9 Hz, 1H), 2.62 (t, J=13.8 Hz, 1H), 2.55 (dd, J=12.7, 4.7 Hz, 1H), 2.12-2.32 (m, 7H), 1.86 (dd, J=14.8, 5.3 Hz, 1H), 1.77-1.81 (m, 1H), 1.60-1.73 (m, 9H), 1.51-1.59 (m, 1H), 1.37-1.45 (m, 2H), 1.20-1.30 (m, 22H), 1.16-1.20 (m, 1H), 1.04 (d, J=6.1 Hz, 3H), 0.80 (t, J=7.2 Hz, 3H), the NH and OH signals are not observable; ¹³C NMR (125 MHz, CDCl₃) 179.1, 168.4, 148.7, 133.6, 129.8, 83.9, 80.8, 70.8, 67.0, 65.4, 54.0, 52.0, 50.0, 36.7, 36.0, 31.9, 31.8, 30.5, 29.4, 29.3, 29.29, 29.26, 29.1, 29.0, 28.4, 26.7, 25.8, 25.5, 23.7, 21.5, 17.8, 10.0 ppm; IR (film) 3261, 3138, 2919, 2849, 1728, 1658, 1606, 1465, 1284, 1154, 1031 cm⁻¹; [α]²⁵_{D}−17.7, [α]²⁵₅₇₇−17.0, [α]²⁵₅₄₆−18.7, [α]²⁵₄₃₅−28.5, (c 1.10, CHCl₃). HRMS (FAB) m/z 658.4795 cal'd for C₃₈H₆₄N₃O₆, found 658.4791 [M].

Synthesis of 41,45-bis-t-Butoxycarbony Crambescidin 800 (91).

Benzotriazol-1-yloxytris(dimethylamino)phosphonium hexafluorophosphate (22 mg, 50 μmol) was added to a rt solution of carboxylic acid 89 (23 mg, 33 μmol), amine 90 (18 mg, 50 μmol), Et₂N (0.15 mL, 1.1 mmol), and CH₂Cl₂ (5 mL). After 4 h, the reaction was diluted with Et₂O (20 mL), and washed with saturated aqueous NH₄Cl (5 mL) and brine (5mL). The organic layer was dried (MgSO₄), filtered, and the filtrate was concentrated. The resulting residue was purified on silica gel (50:1 CHCl₃-MeOH) to yield 28 mg (82%) of the desired product 91 as a colorless oil: ¹H NMR (500 MHz, d4-MeOD) δ 5.70-5.73 (m, 1H), 5.47-5.52 (m, 1H), 4.40 (br d, J=10.3 Hz, 1H), 4.33-4.37 (m, 1H), 4.10-4.16 (m, 2H), 4.02-4.09 (m, 1H), 3.75-3.85 (m, 2H), 3.34-3.59 (m, 2H), 3.23-3.29 (m, 2H), 3.12-3.20 (m, 2H), 3.07 (d, J=4.8 Hz, 1H), 2.94-3.06 (m, 2H), 2.64 (dd, J=13.0, 4.7 Hz, 1H), 2.26-2.46 (m, 6H), 2.10-2.20 (m, 1H), 2.00 (dd, J=13.9, 5.8 Hz, 1H), 1.79-1.85 (m, 3H), 1.50-1.77 (m, 1H), 1.36-1.47 (m, 20H), 1.22-1.35 (m, 25H), 1.09 (d, J=6.1 Hz, 3H), 0.85 (t, J=6.1 Hz, 3H); ¹³C NMR (125 MHz, d4-MeOD) 176.6/176.2, 170.2, 158.5, 150.2, 134.3, 131.3, 85.1, 82.2, 80.0, 79.96, 72.3, 69.1, 68.4, 68.37, 66.5, 55.6, 55.0, 54.2, 53.5, 50.7, 45.1, 38.9, 38.7, 38.3, 38.1, 37.9, 36.2, 34.3, 34.1, 33.0, 32.6, 31.5, 30.8, 30.7, 30.6, 30.5, 30.3, 30.2, 29.6, 28.8, 28.7, 27.6, 27.0, 26.7, 26.6, 24.4, 21.8, 19.5, 10.8 ppm; IR (film) 3356, 2934, 2858, 1732, 1706, 1657, 1613, 1509, 1459, 1251, 1170 cm⁻¹; [α]²⁵_{D}−3.0, [α]²⁵₅₇₇−2.2, [α]²⁵₅₄₆−2.8, [α]²⁵₄₃₅−3.5, [α]²⁵₄₀₅−3.6, (c 0.75, CHCl₃). HRMS (FAB) m/z 1001.7 cald for C₅₅H₉₇N₆O₁₀, found 1001.7 [M].

Synthesis of Crambescidin 800 Trihydrochloride (2).

A solution of 91 (13 mg, 13 μmol) and 1.3 mL of a 3.0 M solution of HCl in EtOAc was maintained at rt for 20 mins and then concentrated. Purification of the residue by reverse phase HPLC (4:1 MeOH-0. 1 M NaCl, Altima C18, 5 column) gave˜11.8 mg (75%) of crambescidin 800 as its trihydrochloride salt (a light yellow oil). Data for this sample were consistent with data published for natural 2. Data for synthetic 2: ¹H NMR (500 MHz, d4-MeOD) δ 5.71 (m, 1H), 5.50 (app d, J=10.9 Hz, 1H), 4.41 (m, 1H), 4.33 (m, 1H), 4.13 (m, 1H), 4.05 (m, 1H), 3.96 (m, 1H), 3.85 (m, 1H), 3.65 (m, 2H), 3.55 (m, 1H), 3.44 (m, 2H), 3.11 (m, 2H), 3.07 (d, J=4.8 Hz, 1H), 2.97 (m, 0.5H), 2.88 (m, 1.5H), 2.64 (dd, J=12.8, 4.7 Hz, 1H), 2.23-2.51 (m, 7H), 2.17 (m, 1H), 1.50-2.10(m, 15H), 1.42(t, J=12.2 Hz, 1H), 1.20-1.40 (m, 25H), 1.09(d, J=6. 1 Hz, 3H), 0.85 (t, J=7.2 Hz, 3H); ¹H NMR (500 MHz, CDCl₃) δ 9.74 (s, 1H), 9.50 (s, 1H), 8.00 (br s, 6H), 5.67 (app s, 1H), 5.47 (app d, J=10.4 Hz, 1H), 4.49 (m, 1H), 4.28 (m, 1H), 4.07 (m, 2H), 3.97 (m, 2H), 3.45-3.66 (m, 3H), 3.29 (m, 2H), 3.11 (m, 2H), 2.95 (m, 2H), 2.55 (m, 1H), 2.10-2.50 (m, 7H), 2.05 (m, 1H), 1.95 (m, 1H), 1.50-1.70 (m, 15H), 1.40-1.50 (m, 2H), 1.20-1.40 (m, 25H), 1.05 (d, J=5.4 Hz, 3H), 0.83 (t, J=6.6 Hz, 3H); ¹³C NMR (125 MHz, d4-MeOD) 177.5, 170.2, 150.2, 134.3, 131.3, 85.1, 82.1, 72.3, 69.4, 68.4, 66.5, 55.6, 54.8, 54.1, 50.7, 43.8, 38.5, 38.3, 38.2, 37.9, 33.0, 32.9, 32.6, 31.5, 30.8, .30.7, 30.67, 30.6, 30.3, 30.26, 29.6, 27.6, 27.0, 26.6, 26.55, 24.4, 21.8, 19.4, 10.8 ppm; ¹³C NMR (125 MHz, CDCl₃) 175.5/175.0, 168.3, 148.7, 133.6, 129.8, 83.5, 80.6, 70.9, 67.1, 65.4, 54.4, 53.9, 51.8, 49.5, 44.0, 37.9, 37.6, 37.0, 36.9, 33.5, 33.2, 32.0, 31.8, 30.8, 30.6, 29.7, 29.6, 29.59, 29.55, 29.5, 29.1, 29.0, 28.4, 26.9, 25.8, 25.5, 25.4, 23.4, 21.4, 18.3, 10.1 ppm; IR (film) 3382, 3231, 2923, 2852, 1732, 1659, 1614, 1469, 1167, 1086, 1015 cm^{-1; [α]}²⁵_{D}−4.4, [α]²⁵₅₇₇5.0, [α]²⁵₅₄₆−4.0, [α]²⁵₄₃₅−6.3, [α]²⁵₄₀₅−6.2, (c 0.70, CHCl₃). HRMS (FAB) m/z 801.6217 cald for C₄₅H₈₁N₆O₆, found 801.6222 [M].

Synthesis ofPeracetylcrambescidin 800 (92).

A solution of crambescidin 800 (2) (5.0 mg, 5.5 μg), Ac₂O (0.5 mL), and pyridine (1 mL) was maintained at rt for 23 then concentrated in vacuo (0.9 mm Hg, 23° C.), diluted with CHCl₃ (20 mL) and washed with 0.1 M HCl (5 mL), and brine (5 mL). The organic layer was dried (MgSO₄), filtered, and the filtrate was concentrated. The resulting residue was purified on silica gel (20:1 CHCl₃-MeOH; 10:1 CHCl₃-MeOH) to yield 2 mg (35%) of peracetylcrambescidin 800 (92) as a white wax. Data for this sample were consistent with data published for naturally derived peracetylcrambescidin 800. Data for synthetic 92: ¹H NMR (500 MHz, CDCl₃) δ 9.88 (s, 1H), 9.64 (s, 1H), 6.75 (br s, 0.7H), 6.38 (br s, 0.7H), 6.18 (br s, 0.7H), 5.67 (app t, J=10.5 Hz, 1H), 5.49 (app d, J=11.0Hz, 1H), 5.10 (m, 1H), 4.51 (m, 1H), 4.28 (app dt, J=9.8, 4.9Hz, 1H), 4.12 (m, 1H), 4.06 (m, 1H), 4.04 (m, 2H), 3.46-3.64 (m, 2H), 3.20-3.39 (m, 4H), 2.99-3.16 (m, 2H), 2.94 (d, J=4.6 Hz, 1H), 2.55 (dd, J=12.6, 4.6 Hz, 1H), 2.49 (m, 1H), 2.17-2.38 (m, 7H), 2.05 (s, 3H), 2.00 (s, 3H), 1.98 (s, 3H), 1.92-196 (m, 1H), 1.52-1.82 (m, 14H), 1.43 (t, J=12.2Hz, 1H), 1.20-1.40(m, 25H), 1.05 (d, J=6.1 Hz, 3H), 0.83 (t, J=7.2 Hz, 3H); ¹H NMR (500 MHz, d4-MeOH) 6 5.72 (app t, J=10.9 Hz, 1H), 5.51 (app d, J=11.0Hz, 1H), 5.15 (m, 1H), 4.32-4.39(m, 2H), 4.13 (dt, J=6.6, 1.8Hz, 2H), 4.07 (m, 1H), 3.83 (m, 1H), 3.39-3.63 (m, 4H), 3.14-3.25 (m, 4H), 3.08 (d, J=4.9 Hz, 1H), 2.64 (dd, J=13.0, 4.8 Hz, 1H), 2.29-2.47 (m, 7H), 2.17 (m, 1H), 2.02 (s, 3H), 2.01 (m, 1H),1.94 (s, 1.5H), 1.93 (s, 1.5H), 1.92 (s, 1.5H), 1.91 (s, 1.5H), 1.53-1.86 (m, 14H), 1.42 (t, J=12.6 Hz, 1H), 1.20-1.40 (m, 25H), 1.09 (d, J=6.2 Hz, 3H), 0.85 (t, J=7.2 Hz, 3H); ¹³C NMR (125 MHz, CDCl₃) 174.5, 171.0, 170.8, 170.5, 170.4, 168.3, 148.8, 133.7, 129.8, 83.6, 80.7, 71.0, 70.6, 67.3, 65.4, 53.9, 51.8, 50.5, 49.7, 42.6, 37.0, 36.96, 35.9, 35.3, 33.2, 32.4, 32.0, 30.6, 29.7, 29.6, 29.5, 29.4, 29.1, 28.5, 27.1, 26.8, 25.8, 25.5, 23.5, 21.4, 18.4, 10.1 ppm; IR (film) 3457, 3240, 2923, 1739, 1732, 1660, 1643, 1614, 1463, 1372, 1238, 1015 cm⁻¹; [α]²⁵_{D}−37 (c 0.2, CHCl₃). HRMS (FAB) m/z 927.6534 cald for C₅₁H₈₇N₆O₉, found 927.6547 [M].

These results demonstrate the first total synthesis of Crambescidin 800 (compound 2) in a convergent fashion with the longest linear sequence from commercially available starting material in 25 steps and in a 3.0% overall yield. These experiments confirm the stereochemical assigrnment of Crambescidin 800 (compound 2) and rigorously establish that the absolute configuration of it hydroxysperimidine side chain is S.

### EXAMPLE IV

Initial Method for Enantioselective Total Synthesis of 13,14,15-Isocrambescidin 800

Synthesis Plan.

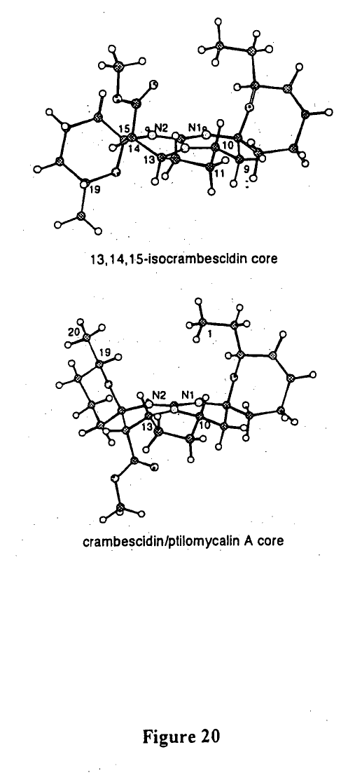

Molecular mechanics models of the methyl esters of the 13,14,15-Isocrambescidin 800 and Ptilomycalin A/Crambescidin are shown in FIG. 20. These models clearly illustrate the structural differences and similarities between the two structures. For instance, the C10 and C13 angular hydrogens are trans in the Isocrambescidin core and cis in the Ptilomycalin A/Crambescidin core, but the relationship between the substituents at C13, C14 and C15 is the same in both structures. Also, the C—O bonds in both structures are axial. Thus, as in the Ptilomycalin A synthesis, we surmised that the C8 and C15 spirocenters could be constructed with the desired stereochemistry if the required trans stereochemistry of the central triazacenaphthalene ring system was in place. This strategy would require setting the trans relationship of the C10 and C13 angular hydrogens and relating this chirality to the C3 and C19 stereocenters of the oxopene and hydropyran rings.

The initial synthetic challenge was constructing the triazacenaphthalene ring system with the desired trans stereochemistry. In the (−)-Ptilomycalin A synthesis the required cis stereochemistry was established via a “tethered Biginelli” condensation of a ureido aldehyde and a β-ketoester. (See Overman, L. E.; Rabinowitz, M. H. J. Org. Chem. 1993, 58, 3235-3237; Overman, L. E.; Rabinowitz, M. H.; Renhowe P. A. J Am. Chem. Soc. 1995, 117, 2657-2658 and Kappe, C. O. Tetrahedron 1993, 49, 6937−6963).

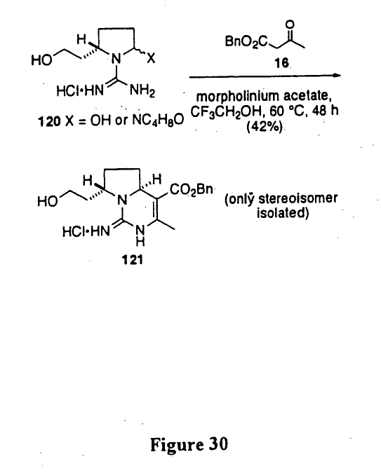

Further studies in our laboratories revealed that a Biginelli condensation between a tethered guanyl aldehyde and a β-ketoester afforded 1-iminohexahydropyrrolo[1,2-c]pyrimidine intermediates with the trans relationship about the pyrrolidine ring (FIG. 30) (McDonald, A. I.; Overman, L. E. J. Org. Chem. 1999, 64, 1520-1528) thus providing a strategy for constructing the Isocrambescidin core.

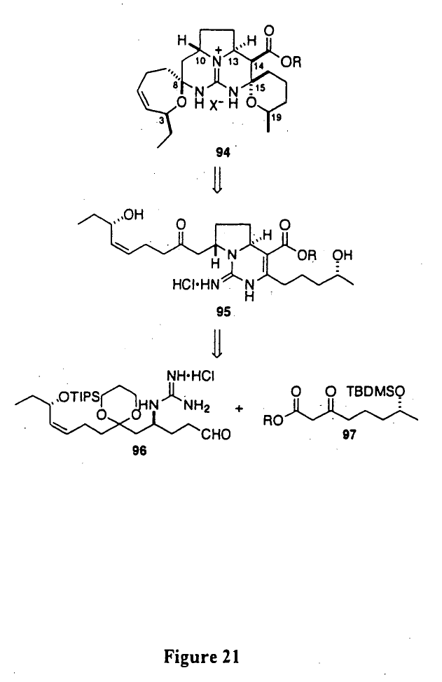

A retrosynthetic analysis of the Isocrambescidin core is shown in FIG. 21. Disconnection of the C8 and C15 aminals of 94 leads to a 1-iminohexahydropyrrolo[1,2-c]pyrimidine intermediate such as 95. With the guanidine unit in place, we envisaged formation of the final three rings of the pentacyclic core directly from an intermediate such as 95. We further envisaged 95 being formed via a Biginelli condensation of guanyl aldehyde 96 and a β-ketoester such as 97. As in the (−)-Ptilomycalin A synthesis, this strategy is very attractive since it is highly convergent. In order for this synthesis to be completed in this convergent fashion, however, the guanidine unit would have to be introduced very early in the synthesis. Early installation of the guanidine is attractive in that further manipulations to install the guanidine at a later stage can be avoided, but since we did not want to protect the guanidine, we were forced to deal with this highly polar functionality for several steps of the synthesis (FIG. 21).

Results and Discussion

Synthesis of13,14,15-Isocrambescidin 800.

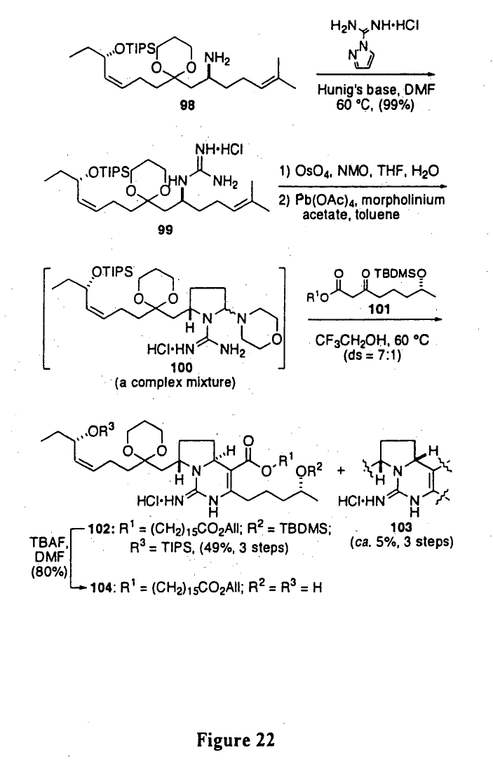

The synthesis began with amine 98, which had also been utilized in the second generation synthesis of (−)-Ptilomycalin A and the synthesis of Crambescidin 800 (See FIG. 22). Treatment of 98 with 1-H-pyrazole-1-carboxamidine hydrochloride (Bernatowicz et al., J. Org. Chem. 1992, 57:2497-2502) and Hunig's base at 60° C. afforded guanidine 99 (ca 99% yield) which was used without purification (FIG. 22). Before examining the key Biginelli condensation, the trisubstituted olefin had to be selectively oxidized. In the event, treatment of 99 with catalytic osmium tetroxide (OₛO₄) 10 resulted in selective dihydroxylation of the trisubstituted double bond. (Sharpless and Williams, Tetrahedron Lett. The corresponding diol was cleaved with Pb(OAc)₄ in the presence of morpholinium acetate affording aminal 100. Following cleavage of the diol, we found it was optimal if 100 was filtered, and used immediately in the Biginelli condensation, and not exposed to an aqueous workup. Furthermore, 100 is actually a mixture of several components as judged by ¹H and ¹³C NMR. Multiple signals are observed for many carbon atoms in the ¹³C NMR spectra, while broad peaks are seen in the ¹H NMR spectra and no aldehyde signal is apparent (FIG. 22).

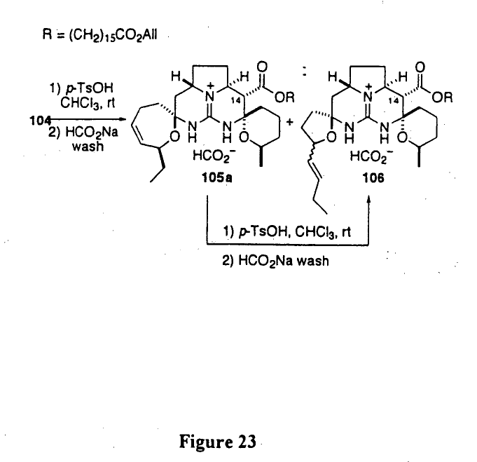

Biginelli condensation of 100 and β-ketoester 101 (Overman et al., J. Am. Chem. Soc., 1995, 117:2657-2658) in EtOH proceeded with modest selectivity (3:1 trans:cis). Fortunately, we found that heating 100 (1 equiv) and 101 (1.5 equiv) in 2,2,2-trifluoroethanol at60° C. for20 h improved the diastereoselectivity to 7:1 trans:cis. After purification on silica gel deactivated with pH 7.0 buffer. The deactivated silica was prepared by taking Merck silica gel (0.040-0.063) adding 10% (by weight) pH 7.0 buffer and mixing until homogeneous. The desired trans adduct 102 was isolated in 49% yield and cis adduct 103 in ca. 5% yield. The cis adduct 103 was slower moving on silica gel than trans adduct 102. Thus, it was somewhat difficult to isolate pure 103 since some of trans adduct 102 would trail off of the column. The stereochemistry of trans adduct 102 was initially assigned based on results from previous model studies (McDonald et al. J. Org. Chem 1999, 64, 1520-1528). This assignment was more rigorously established using pentacyclic intermediates (see 105a and 105b) produced later in the synthesis. Deprotection of 102 with TBAF in DMF for 36 h afforded diol 104 in 80% yield (FIG. 23). Often, this reaction did not go to completion, and partially deprotected intermediates (R²=TBS, R³=H) were isolated in 10-15% yield. Heating the reaction mixture at 60° C. consistently afforded fully deprotected 104, but other products were formed and the isolated yield of 104 was lower.

A. Initial Stereochemical Assignment of pentacycle 105a

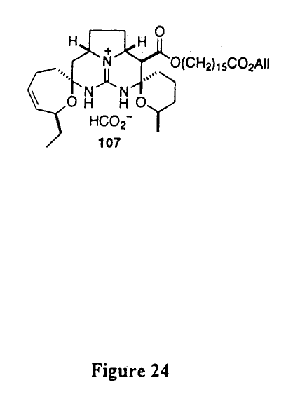

Diol 104 was now properly finctionalized for conversion to the isocrambescidin pentacyclic core. In the event, 104 was exposed to p-toluenesulfonic acid (p-TsOH) (3 equiv) in CHCl₃ for 24 h. The reaction mixture was then washed with aqueous HCO₂Na affording an approximate 1:1 mixture of the desired pentacycle 105a and isomerized pentacycle 106 (pentacycle 20 is an approximate 1:1 mixture of diastereomers, and the structure was assigned based on ¹H NMR COSY studies) in ca 50% yield (FIG. 23). Since the tosylate couterion interconverted slowly, several washings were required to obtain the formate salts exclusively, and we were concerned that the multiple washings may result in lower yields for this transformation. The formate salts were prepared to allow direct comparisons to pentacycle 107 (FIG. 24), an intermediate in the first generation synthesis of (−)-Ptilomycalin A (Overman et al. J. Am. Chem. Soc. 1995, 117, 2657-2658). ¹H NMR NOE studies confirm that 105a is epimeric at C13, C14 and C15 with 107. Thus, trans diaxial addition of the side chain alcohol to the double bond during the conversion of 104→105a orients the hydropyran in the Isocrambescidin core as in the Ptilomycalin A/Crambescidin core; Furthermore, that 105a is epimeric with 13,14,15-Isocrambescidin 800 at C14 was signaled by the 11.8 Hz coupling constant of the C14 methine hydrogen (McDonald et al. J. Org. Chem 1999, 64, 1520-1528) (See FIGS. 23 and 24 [Scheme 3, FIG. 3]).

Further studies revealed that the ratio of compounds 105a and 106 could be controlled by varying the reaction times and equivalents of p-TsOH. Larger amounts of p-TsOH and longer reaction times favored the formation of 106, and we found that treating pure 105a with p-TsOH also afforded 106. Conditions were found (2 equiv p-TsOH, CHCl₃, 7 h) that gave a 5:1 mixture of 105a and 106. Unfortunately, we could not find conditions using p-TsOH that gave 105a exclusively, and since 105a and 106 were somewhat difficult to separate, the isolated yields of 105a were never greater than 45-50%. Thus, a better method for the preparation of 105a was required.

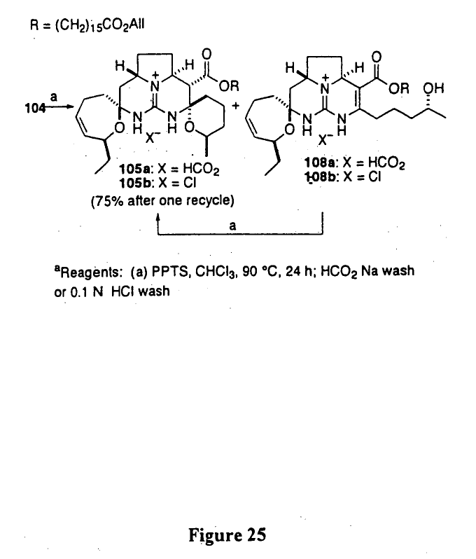

Treatment of 104 with pyridiniump-tolunesulfonate (PPTS) (2 equiv) at 60° C. in CHCl₃ for 5 h followed by a HCO₂Na wash afforded a 1:5 mixture of the desired 105a and tetracycle 108a (FIG. 25). Slight modifications of the reaction conditions (2 equiv PPTS, CHCl₃, 90° C. in a sealed tube, 24 h), however, afforded a 2:1 mixture of 105a and 108a (FIG. 25). Unfortunately, we could not find conditions using PPTS to convert 104 completely to 105a, but 105a and 108a could be separated by column chromatography. After separation, resubjection of 108a to the reaction conditions afforded, again, a 2:1 mixture of 105a and 108a. The desired 105a could be isolated in 75% combined yield after one recycle. Initially, for comparative purposes, 105a and 108a were converted to the formate salts before chromatography, and they were separated using 95:5:0.1EtOAc-isopropanol-formic acid. We later found, however, that the hydrochloride salts, 105b and 108b, were easier to separate than the corresponding formate salts (105a and 108a). The hydrochloride salts were prepared by washing the reaction mixture with 0.1 N HCl or saturated aqueous sodium chloride and separated on silica gel using 99:1 CHCl₃-MeOH→95:5 CHCl₃-MeOH. As in previous cases, several washings were required to completely convert the tosylate salt to the hydrochloride salt. Since the forinate and hydrochloride salts could be obtained in virtually identical yields, the ease of separation made use of the hydrochloride salts optimal (FIG. 25). Reagents used were PPTS, HCl₃, 90° C. 24 h; HCO₂ Na wash or 0.1 N HCl wash (“a”).

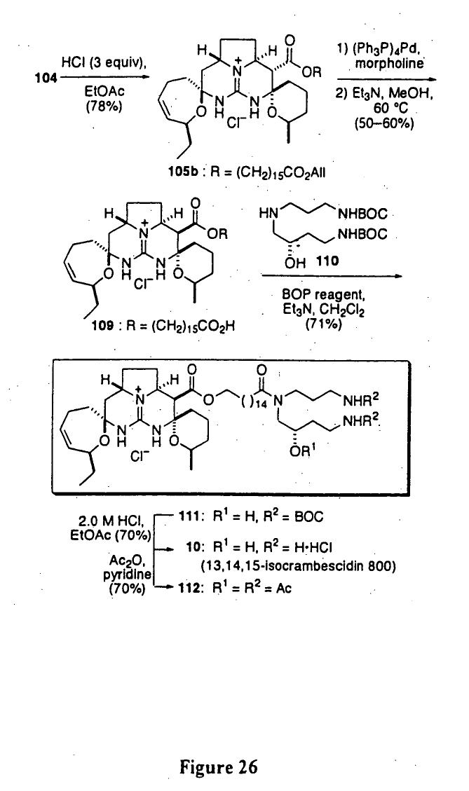

While pentacycles 105a and 105b could be prepared in synthetically useful yields, the sequence was somewhat cumbersome. Ideally, we needed to find conditions that did not promote the formation of isomerization products such as 106 but would afford complete conversion to 105a or 105b. Toward this end, we found that treatment of 104 with HCl (3 equiv) in EtOAc afforded 105b exclusively in 78% yield. Since the hydrochloride salt was formed during the reaction, the somewhat troublesome counterion exchange was avoided (FIG. 26).

Epimerization to the axial ester at C14 was best accomplished after removal of the allyl group of the terminal ester. To this end, the allyl group in 105b was removed with (Ph₃P)₄Pd and morpholine (FIG. 26). The C14 ester was then epimerized with Et₃N in MeOH at 60° C. affording a 2-3:1 mixture of the desired β-epimer, 109, and a mixture ofthe starting α-epimer and a product analogous to 108a resulting from elimination of the oxygen at C15. After purification by flash chromatography, 109 was isolated in 50-60% yield for the two steps. A diagnostic 3.0 Hz coupling constant is observed for the C14 methine hydrogen of 109. In contrast to precursors of (−)-Ptilomycalin A and Crambescidin 800, the axial ester is favored at equilibrium in the Isocrambescidin series.

The synthesis of 13,14,15-Isocrambescidin 800 was readily completed from 109 (FIG. 26). The (S)-7-hydroxyspermidine fragment 110 was prepared from (R)-epichlorohydrin and coupled with pentacycle 109 using benzotriazol-1-yloxytris(dimethylamino)phosphonium hexafluorophosphate (BOP) (Castro et al. Tetrahedron Lett. 1975, 1219-22) to provide 111 in 71% yield. Removal of the BOC groups with 2 M HCl in ethyl acetate (Stahl et al. J. Org. Chem. 1978, 43, 2285-2286) and purification of the crude product by reverse-phase HPLC provided the trihydrochloride salt of 13,14,15-Isocrambescidin 800(10), [α]_{D}²³−67.7 (c 0.7 MeOH), in 70% yield. The data for the trihydrochloride salt of synthetic 10 is in agreement with ¹H and ¹³C NMR data reported for natural 10. The ¹H NMR spectrum (500 MHz, CD3 OD) of synthetic d1 trihydrochloride is identical to the spectrum of natural 10 published in the Supplementary Materials of references 3b (Spectrum S-3); Spectrum S-3 is apparently also of the tricationic salt. There are errors in the tabulated data in Table I of reference 3b, since there are discrepancies between the tabulated data and Spectrum S-3. The ¹³C NMR spectrum of synthetic 10 trihydrochloride is also identical to the spectrum of natural 10 published in the Supplementary Material of reference 3b (Spectrum S-8a). Again, there are errors in the tabulated data in Table I of reference 3b, since there are discrepancies between the tabulated data and Spectrum S-8a. Synthetic 10 was also converted to the triacetate derivative, 112. Data for synthetic 112 is also in agreement with ¹H and ¹³C NMR data reported for 112 prepared from natural 10 (Jares-Erijman, et al J. Org. Chem. 1993 58:4805).

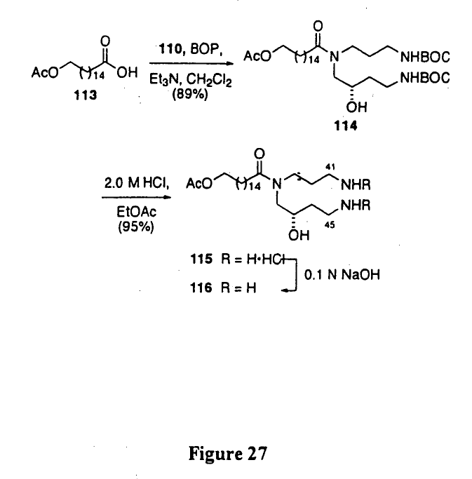

The trihydrochloride salt of 10 was believed to be obtained, since a basic workup was not performed after the removal of the BOC groups (111→10). Natural 10, however, was reported with the amines as the free bases, but the ¹H and ¹³C NMR spectra of synthetic 10 and natural 10 were indistinguishable. Treatment of synthetic 10 with 0.1 N NaOH saturated with NaCl resulted in downfield shifts of the C41 and C45 protons. To investigate this further, 114 was prepared from acid 113 to model the hydroxyspermidine region of 13,14,15-Isocrambescidin 800 (FIG. 27). Deprotection of 114 with 2.0 M HCl in EtOAc afforded 115 in 95% yield. The chemical shifts of the C37-C45 protons were identical in 115 and 10 [C41, 2.99-2.84 ppm (m), C45, 3.14-3.08 ppm (m)] (Table 1), but the absence of the guanidine unit made the spectral analysis of 115 somewhat easier. Treatment of 115 with 0.1 N NaOH afforded 116 as the free base. There were significant upfield shifts of the C41 and C45 protons in 116 [C41, 2.66-2.58 ppm (m), C45, 2.86-2.78 ppm (m)]. This upfield shift is consistent with conversion of a hydrochloride salt to a free base. Therefore, this supports the conclusion that natural 13,14,15-Isocrambescidin 800 was actually isolated as the trihydrochloride salt (FIG. 27).

TABLE 1
Comparison of the Chemical Shifts of the C41
and C45 Protons of Compounds 115 and 116

¹H NMR (CD₃OD, 500 MHz), (δ ppm), mult

| Position | 115 | 116 |
|---|---|---|
| 41 | 2.99-2.84, m | 2.6-2.58, m |
| 45 | 3.14-3.08, m | 2.86-2.78, m |

Assignment of the C43 Stereocenter of 13,14,15-Isocrambescidin 800.

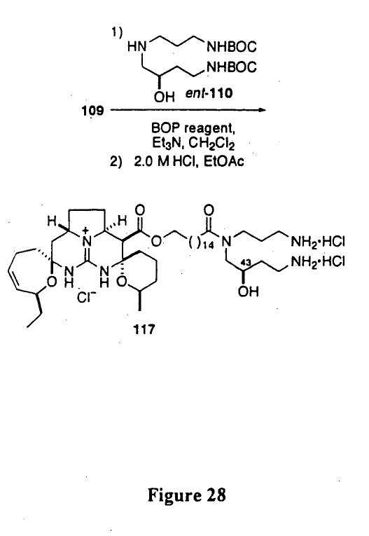

The C43 stereocenter in 13,14,15-Isocrambescidin 800 was assigned as S based on analogy to crambescidin 816 (compound 3). Our completed total synthesis of compound 10 seemed to confirm this assignment, but, since the C43 stereocenter is far removed the other stereocenters, we were not certain that (43S)-13,14,15-Isocrambescidin 800 could be spectroscopically distinguished from (43R)-13,14,15-Isocrambescidin 800. To this end (43R)-13,14,15-Isocrambescidin 800 (117) was prepared from 109 and ent-110. Ent 110 was prepared from (S)-epichlorohydrin (FIG. 28). As anticipated, 117 was indistinguishable from synthetic 10 and natural 10 by ¹H and ³C NMR and HPLC.

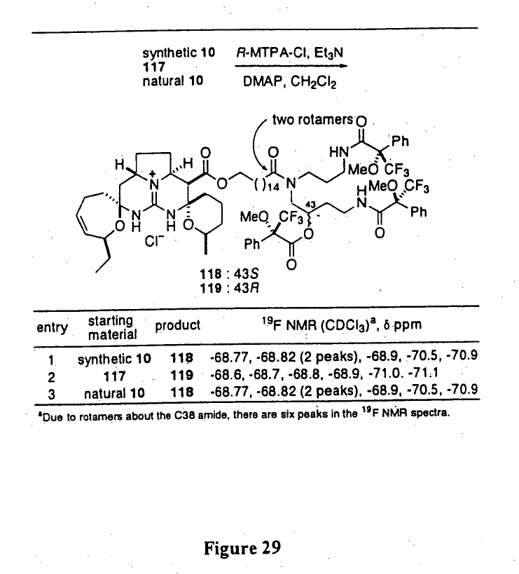

To unambiguously make this stereochemical assignment, we anticipated having to compare a derivative of natural 10 to derivatives of synthetic 10 and 117. The preparation of Mosher's derivatives of synthetic 10, 117 and natural 10 and subsequent analysis by 19F NMR was an appealing option. Mosher's derivatives 118 (43S) and 118 (43R) were prepared according to the method developed by Ward (FIG. 29). The corresponding Mosher's derivative of natural 10 was then prepared and compared to 118 and 119. The ¹⁹F NMR data is identical for the Mosher's derivatives prepared from natural 10 and synthetic 10. Due to rotamers about the C38 amide, there are 6 peaks in the ¹⁹F NMR spectra (See FIG. 27). This unambiguously establishes that the stereochemistry at C43 in 13,14,15-Isocrarnbescidin 800 is S.

The first total synthesis of 13,14,45-Isocrambescidin 800 (10) was accomplished in convergent fashion with the longest linear sequence from commercially available material being 21 steps. These investigations confirm the stereochemical assignment of 10 and rigorously establish that the absolute configuration of its hydroxyspermidine side chain is S. In its present form, this synthesis can provide substantial quantities of 10 and congeners for pharmacological evaluation. This enantioselective total synthesis demonstrates for the first time that: (a) the tethered Biginelli strategy can be extended to guandidine intermediates, (b) the key Biginelli condensation can be accomplished under sufficiently mild conditions that fragments containing the full functionality of the Crambescidin core can be employed, and (c) that the spiroaminal units in the Isocrambescidin series assemble with high stereochemical fidelity.

Experimental Section

General.

Dry THF, Et₂O, and CH₂Cl₂ from Aldrich were filtered through a column charged with Al₂O₃ (solvent purification system). Triethylamine (Et₃N), pyridine, diisopropylethylamine, diisopropylamine, and acetonitrile were distilled from CaH₂ at atmospheric pressure. Silica gel (0.040-0.063) by Merck was used for flash chromatography. Reverse phase HPLC separations were performed using an HPLC system composed of a Waters 590 pump and a Waters 486 UV detector. NMR spectra were recorded on Bruker instruments (500 MHz and 400 MHz). IR spectra were measured on Perkin-Elmer Series 1600 FTIR, and optical rotations were measured on Jasco DIP-360 polarimeter. Mass spectra were measured on a MicroMass Analytical 7070E (CI-isobutane) or a MicroMass AutoSpec E (FAB) spectrometer. Microanalyses were performed by Atlantic Microlabs, Atlanta, Ga. Other general experimental details have been described (Metais, E. et al. J. Org. Chem. 1997, 62, 9210-9216).

(6S, 11Z, 13S)-6-amino-N-carboxamidine-8-(1′,3′-dioxan-2′-yl)-2-methyl-13-trisopropylsiloxypentadeca-2,11-diene (99).

A solution of amine 98 (2.95 g, 6.12 mmol), 1-H-pyrazole-1-carboxamidine hydrochloride (2.70 g, 18.4 mmol), Hunig's base (4.37 mL, 24.5 mmol) and DMF (6.0 mL) was maintained at rt for 16 h then warmed to 60° C. and maintained for 4 h. The solution was cooled and partitioned between CHCl₃ (300 mL) and 0.1 N HCl (75 mL). The organic phase was washed with 0.1 N HCl (75 mL) and H₂O (75 mL), dried (Na₂SO₄), filtered and concentrated affording a 2:1 mixture of guanidine 99 and amine 98. This mixture was dissolved in DMF (6.0 mL) and treated with 1-H-pyrazole-1-carboxamidine hydrochloride (1.35 g, 9.2 mmol) and Hunig's base (2.19 mL, 12.3 mmol). The solution was maintained at room temperature (rt) for 16 h then warmed to 60° C. and maintained for 4 h. The reaction was worked up as previously described and placed on a vacuum pump (0.1 mm Hg) to remove the residual DMF. This afforded 3.20 g (99%) of guanidine 99 as a light yellow oil which was used without further purification: ¹H NMR (500 MHz, CDCl₃) δ 7.82 (app d, J=6.7 Hz, 1 H), 7.24 (br s, 1 H), 5.43-5.39 (m, 1 H), 5.29-5.24 (m, 1 H), 5.09 (br t, J=7.0 Hz, 1 H), 4.46 (app q, J=7.3 Hz, 1 H), 3.98-3.76 (m,4H), 3.60 (m, 1H), 2.20-2.13 (m, 2H), 2.02-1.74 (overlapping m, 2 H), 1.69 (s, 3 H), 1.64-1.58 (overlapping m, 2 H), 1.62 (s, 3 H), 1.51-1.38 (m, 2 H), 1.05 (m, 21 H), 0.87 (t, J=7.4 Hz, 3 H); ¹³C NMR (125 MHz, CDCl₃) δ 157.6, 135.0, 132.7, 126.9, 123.1, 100.5, 69.8, 59.8, 59.3, 46.6, 45.0, 36.5, 31.7, 30.5, 25.7, 25.0, 24.8, 22.2, 18.1, 18.0, 17.6, 12.3, 9.3 ppm; IR(film)2961, 2865, 1651, 1463, 1383, 1246, 1109 cm⁻¹; MS: HRMS (FAB) (M-Cl) m/z 524.4225, (524.4250 calcd for C₂₇H₅₈N₃O₃Si); [α]²⁵_{D}+1.7, [α]²⁵₅₇₇+2.7, [α]²⁵₅₄₆+3.2, [α]²⁵₄₃₅+7.3, [α]²⁵₄₀₅+9.3 (c 1.3, CHCl₃).

(4aS,7S)-4-[15-(Alloxycarbonyl)pentadecyloxycarbonyl]-3-[(4S)-4-t-butyldimethysiloxypentyl]-7-[(5Z,7S)-2-(1′,3′-dioxan-2′-yl)-7-triisopropyl-siloxy-5-nonenyl ]-1,2,4a,5,6,7-hexahydro-1-imino-pyrrolol[1,2,c]-pyrimidine hydrochloride (102).

4-Methylmorpholine-N-oxide (2.16 g, 18.4 mmol) and OₛO₄ (3.1 mL, 0.24 mmol, 2% in t-butanol) were added to a solution of guanidine 99 (3.2 g, ˜6.1 mmol), THF (105 mL) and H₂O (15 mL). The mixture was stirred for 8 h, Florisil (1.5 g) and NaHSO₃ (1.5 g) were added and the resulting mixture was stirred for an additional 10 h. Celite and MgSO₄ were added and the mixture was filtered and the eluent was concentrated to give a brown oil. This oil was dissolved in toluene (120 mL), then morpholinium acetate (3.6 g, 24.5 mmol) and Pb(OAc)₄ (3.3 g, 7.3 mmol) were added; The solution was maintained for 45 min. then Celite was added. The mixture was filtered through a plug of Celite, the eluent was diluted with toluene (200 mL) and this solution was concentrated to give a brown oil. The oil was azeotroped to dryness with toluene (200 mL) and the residue was combined with β-ketoester 15 (5.3 g, 9.2 mmol) and 2,2,2-trifluoroethanol (9.0 mL). This solution was maintained at 60° C. for 20 h and then partitioned between CHCl₃ (250 mL) and 0.1 N HCl (50 mL). The organic phase was washed with 0.1 N HCl (50 mL) and brine (50 mL), dried (Na₂SO₄), filtered and concentrated. ¹H NMR analysis revealed a 7:1 ratio of trans:cis Biginelli adducts. Purification of the crude mixture by flash chromatography (CHCl₃_{—}99:1 CHCl₃-MeOH 98:2 CHCl MeOH) using silica gel deactivated with pH 7.0 buffer afforded 3.22 g (49%) of the desired trans adduct, 102, as a light brown oil and 331 mg (5%) of cis adduct 103. Data for 102: ¹H NMR (500 MHz, CDCl₃) δ 9.06 (s, 1 H), 7.33 (s, 1 H), 5.95-5.88 (m, 1 H), 5.43 (app t, J=9.8 Hz, 1 H), 5.31 (app dq, J=17.2, 1.5 Hz, 1 H), 5.27-5.25 (m, 1 H), 5.23 (app dq, J=10.4, 1.3 Hz, 1 H), 4.57 (br d, J=5.7, 2 H), 4.46-4. 41 (m, 2 H), 4.27-4.24 (m, 1 H), 4.17-4.07 (m, 2 H), 4.01-3.95 (m, 2 H), 3.91-3.78 (m, 3 H), 2.77-2.71 (m, 2 H), 2.65-2.59 (m, 1 H), 2.45-2.40 (m, 1 H), 2.32 (t, J=7.6 Hz, 2 H), 2.07-1.88 (m, 6 H), 1.79-1.55 (m, 11 H), 1.53-1.43 (m , 4 H), 1.31-1.25 (m, 21 H), 1.13 (d, J=6.1 Hz, 3 H), 1.05 (s, 21 H),0.87 (t, J=7.4 Hz, 3 H), 0.86 (s, 9 H), 0.037 (s, 3 H), 0.032 (s, 3 H); ¹³C NMR (100 MHz, CDCl₃) δ 173.4, 165.0, 149.9, 147.3, 135.3, 132.2, 126.4, 117.9, 100.9, 100.3, 69.8, 68.3, 64.8, 64.7, 59.9, 59.4, 57.5, 54.1, 46.1, 39.0, 34.8, 34.2, 33.3, 31.6, 30.9, 30.3, 29.6, 29.52, 29.48, 29.42, 29.3, 29.1, 29.0, 28.5, 26.0, 25.8, 24.83, 24.76, 24.4, 23.6, 22.1, 18.01, 17.98, 12.3, 9.2, −4.5, −4.7 ppm; IR (film)2926, 2856, 1738, 1713, 1681, 1538, 1462, 1382, 1256, 1086cm⁻¹; HRMS (FAB) (M-Cl) m/z 1044.6, (1044.8 calcd for C₅₉H₁₁₀N₃O₈Si₂); [α]²⁵_{D}−21.2, [α]²⁵₅₇₇−21.3, [α]²⁵₅₄₆−23.3, [α]²⁵₄₃₅−28.8, [α]²⁵₄₀₅−25.1 (c 1.9, CHCl₃).

(4aS, 7S)-4-[15-(Allyloxycarbonyl)pentadecyloxycarbonyl]-7-[(5Z, 7S)-2-(1′,3′-dioxan-2′-yl)-7-hydroxy-5-nonenyl]-1,2,4a,5,6,7-hexahydro-3-[(4 S)-4-hydroxypentyl]-1-imino-pyrrolo[1,2,c]-pyrimidine hydrochloride (104).

A solution of 102 (2.80 g, 2.59 mmol), TBAF (13.0 mL, 13.0 mmol, 1.0 M) and DMF (26 mL) was maintained at rt for 24 h, then more TBAF (6.0 mL, 6.0 mmol, 1.0 M) was added. The solution was maintained for 6 h then partitioned between CHCl₃ (200 mL) and 0.1 N HCl (75 mL). The organic phase was washed with saturated aqueous NaHCO₂ (2×50 mL), dried (Na2SO₄), filtered and concentrated. The crude product was purified by flash chromatography (95:5:0.1 EtOAc-isopropanol:formic acid 90:10:0.1 EtOAc-isopropanormic acid 85:15:0.1 EtOAsopropanol:formic acid) using silica gel deactivated with pH 7.0 buffer to afford the formate salt of the diol 1.68g (80%) as a light brown oil. The formate salt was easier to purify, but the chloride salt was more stable. Therefore, after purification, the formate salt was converted quantitatively to chloride salt 104 by partitioning the formate salt between CHCl₃ (150 mL) and 0.1 N HCl (25 mL) and washing with 0.1 N HCl (25 mL) and brine (25 mL). The organic phase was dried (Na₂SO₄), filtered and concetrated to afford diol 104: ¹H NMR (500 MHz, CDCl₃) δ 8.63 (s, 1 H), 7.43 (s, 1 H), 5.95-5.87 (m, 1 H), 5.51-5.42 (m, 2 H), 5.31 (ddd, J=17.2, 3.0, 1.5 Hz, 1 H), 5.22 (ddd, J=9.2, 3.0, 1.3 Hz, 1 H), 4.57 (dt, J=5.7, 1.3 Hz, 2 H), 4.43 (dd, J=9.9, 4.3 Hz, 1 H), 4.32 (app q, J=7.1 Hz, 1 H), 4.28-4.25 (m, 1H), 4.17-4.08 (m, 2 H), 4.05-3.92 (m, 3 H), 3.89-3.82 (m, 2 H), 2.91-2.86 (m, 1 H), 2.62-2.58 (m, 1 H), 2.52 (td, J=11.8, 4.6 Hz, 1 H), 2.42-2.39 (m, 1 H), 2.32 (t, J=7.6 Hz, 2 H), 2.16-1.96 (m, 6 H), 1.86-1.44 (m, 14 H), 1.30-1.24 (m, 22 H), 1.19 (d, J=6.2Hz, 3 H), 0.91 (t, J=7.4Hz, 3 H); ¹³CNMR(125 MHz, CDCl₃) δ 173.5, 165.0, 149.7, 147.5, 133.5, 132.3, 130.4, 118.0, 101.0, 100.5, 68.7, 65.4, 64.85, 64.76, 60.1, 59.6, 57.6, 54.2, 45.8, 38.1, 34.7, 34.2, 33.1, 30.4, 30.2, 29.6, 29.51, 29.46, 29.37, 29.2, 29.1, 28.6, 26.0, 24.9, 24.7, 24.0, 23.5, 22.2, 9.7 ppm; IR (film) 3344, 2925, 2854, 1736, 1685, 1542, 1462, 1384, 1259, 1170, 1084, 1001 cm⁻¹; MS: HRMS (FAB) (M-Cl) m/z 774.5615, (774.5632 calcd for C₄₄H₇₆N₃O₈); [α]²⁵_{D}−39.4, [α]²⁵₅₇₇−40.2, [α]²⁵₅₄₆−44.8, [α]²⁵₄₃₅−66.0, [α]²⁵₄₀₅−70.0 (c 1.2, CHCl₃).

Pentacycle 105b.

Acetyl chloride (320 μL, 4.5 mmol) was added to a 0° C. solution of MeOH (200 μL, 5.0 mmol) and EtOAc (30 mL) to give a 0.15 M solution of HCl in EtOAc. Diol 104 (1.10 g, 1.36 mmol) was then dissolved in 27 mL of this solution (4.1 mmol of HCl). The solution was maintained at rt for 6 h, then partitioned between CHCl₃ (250 mL) and brine (50 mL). The organic phase was dried (Na₂SO₄), filtered and concentrated. Purification of the residue by flash chromatography (CHCl₃ 99:1 CH-MeOH 98:2 CHCl₃-MeOH) gave 780 mg (78%) of pentacycle 105b as a light yellow oil. In some instances, pentacycle 105b was contamintated with ca. 5% of an unidentified impurity. This impurity could be removed by further purification by reverse phase HPLC, but the recovery of the desired pentacycle, 105b, was low. Therefore, pentacycle 105b was not purified further, and the unknown impurity was removed after the next transformation.

Data for pure 105b: ¹H NMR (500 MHz, CDCl₃) δ 10.45 (br s, 1 H), 8.89 (br s, 1 H), 5.95-5.87 (m, 1 H), 5.68-5.64 (m, 1 H), 5.48 (broad d, J=11.0 Hz, 1 H), 5.31 (app dd, J=17.2, 1.5Hz, 1 H), 5.23 (app dd, J=10.4, 1.3 Hz, 1 H), 4.57 (brd, J=5.7 Hz, 2H), 4.51 (brd, J=7.7 Hz, 1 H), 4.25-4.21 (m, 2 H), 4.12-4.07 (m, 1 H), 3.98-3.95 (m, 1 H), 3.77-3.72 (m, 1 H), 2.91 (d, J=11.8 Hz, 1 H), 2.61-2.56 (m, 1H), 2.55 (dd, J=12.5, 2.9 Hz, 1 H), 2.32 (t, J=7.5 Hz, 2 H), 2.30-2.28 (m, 3H), 2.21-2.17 (m, 2H), 1.91 (dd, J=14.6, 5.3 Hz, 1 H), 1.85 (br d, J=12.9 Hz, 1 H), 1.78-1.36 (m, 13 H), 1.32-1.20 (m, 21 H), 1.12 (d, J=6.0 Hz, 3 H), 1.12-1.10 (m, 1 H), 0.86 (t, J=7.3 Hz, 3 H); ¹³C NMR(125 MHz, CDCl₃) δ 173.5, 169.0, 150.8, 133.3, 132.3, 129.8, 118.0, 85.5, 84.8, 70.8, 68.7, 65.5, 64.8, 58.7, 55.1, 52.1, 37.2, 37.1, 34.2, 32.9, 32.1, 30.9, 30.0, 29.6, 29.51, 29.45, 29.4, 29.2, 29.11, 29.07, 28.4, 25.9, 24.9, 23.8, 22.0, 17.9, 10.2 ppm; IR (film) 2926, 2853, 1732, 1659, 1615, 1462, 1349, 1202, 1022 cm⁻¹; HRMS (FAB) (M-Cl) m/z 698.5117, (698.5108 calcd for C₄₁H₆₈N₃O₆); [α]²⁵_{D}−54.6, [α]²⁵₅₇₇−55.6, [α]²⁵₅₄₆−64.2, [α]²⁵₄₃₅−114.8, [α]²⁵₄₀₅−141.3 (c 1.25, CHCl₃).

CarboxylicAcid 109.

A solution of pentacycle 105b (50 mg, 0.068 mmol), morpholine (24 μL, 0.27 mmol), (Ph₃P)₄Pd(16mg, 0.014 mmol)and CH₃CN(5 mL)was maintained for 2 h. More morpholine (12 μL, 0.13 mmol) and (Ph₃P)₄Pd (8 mg, 0.007 mmol) were added and the solution was maintained for 2 h. The solution was then partitioned between CHCl₃ (50 mL) and 0.1 N HCl (10 mL). The organic phase was washed with 0.1 N HCl (10 mL), dried (Na₂SO₄), filtered and concentrated to give a brown oil. The brown oil was filtered through a plug of silica gel (99:1 CHCl₃:MeOH 98:2 CHCMeOH), concentrated and dissolved in Et₃N (95 μL, 0.68 mmol) and MeOH (7 mL). The resulting solution was maintained at 60° C. for 36 h then partitioned between CHCl₃ (50 mL) and 0.1 N HCl (8 mL). The organic phase was washed with 0.1 N HCl (8 mL), dried (Na₂SO₄), filtered and concentrated. Purification of the crude product by flash chromatography (99:1 CHCl₃:MeOH 98:2 CHCl₃-MeOH 95:5 CHCl₃:MeOH) afforded 28 mg (60%) of 109 as a light yellow oil: ¹H NMR (500 MHz, CDCl₃) δ 10.07 (brs, 1 H), 9.28 (br s, 1 H), 5.64 (app t, J=8.1 Hz, 1 H), 5.50 (d, J=10.6 Hz, 1 H), 4.58 (br s, 1 H), 4.17-4.12 (m, 1 H), 4.02-3.97 (m, 2 H), 3.92-3.88 (m, 1 H), 3.71-3.68(m, 1 H), 3.46 (d, J=3.0 Hz, 1 H), 2.63-2.55 (m, 1 H), 2.52 (d, J=11.0 Hz, 1 H), 2.30 (t, J=7.4Hz, 2 H), 2.29-2.26 (m, 1 H), 2.22-2.16 (m, 3 H), 1.85-1.80 (m, 4 H), 1.73-1.42 (m, 11 H), 1.40-1.24 (m, 23 H), 1.18 (d, J=5.9 Hz, 3 H), 0.95 (t, J=7.2 Hz, 3 H); ¹³CNMR (125 MHz, CDCl₃) δ178.6, 167.8, 149.4, 133.7, 129.6, 85.0, 83.0, 70.7, 69.0, 65.2, 52.9, 52.1, 41.7, 37.9, 37.4, 35.1, 32.5, 31.5, 30.2, 29.7, 29.4, 29.33, 29.29, 29.19, 29.0, 28.4, 27.9, 25.7, 25.3, 24.1, 22.2, 19.7, 10.2 ppm; IR (film) 3200, 2924, 2852, 1732, 1660, 1621, 1189, 1167, 1027 cm⁻¹; HRMS (FAB) (M-Cl) m/z 658.4789, (658.4795 calcd for C₃₈H₆₄N₃O₆); α]²⁵_{D}−47.3, [α]²⁵₅₇₇−49.5, [α]²⁵₅₄₆−55.9, [α]²⁵₄₃₅−99.8, [α]²⁵₄₀₅−121.5 (c 1.2, CHCl₃).

41,45-bis-t-Butoxycarbonyl-13,14,15-Isocrambescidin 800 (111).

Benzotriazol-1-yloxytris(dimethylamino)phosphonium hexafluorophosphate (28 mg, 0.064 mmol) was added to a solution of carboxylic acid 109 (30 mg, 0.043 mmol), amine 110 (23 mg, 0.064 mmol), Et₃N (29 μL, 0.22 mmol) and CH₂Cl₂ (2.0 mL). The solution was maintained for 1 h and then was partitioned between Et₂O (40 mL) and 0.1 N HCl (10 mL). The organic phase was washed with brine (2×10 mL), dried (MgSO4), filtered and concentrated to afford a crude oil. Purification of this residue by flash chromatography (99:1 CHCl₃-MeOH 97:3 CHCl₃-MeOH) gave 32 mg (71%) of 111 as a colorless foam: ¹H NMR (500 MHz, CD₃OD) δ 5.70 (br t, J=8.8 Hz,1 H), 5.51 (d, J=11.1 Hz, 1 H), 4.45 (br s, 1 H), 4.19-4.06 (m,3 H), 3.92-3.78 (m, 3 H), 3.84 (d J=3.4 Hz, 1 H), 3.59-3.23 (m, 3 H), 3.19-3.12 (m, 3 H), 3.06-2.97 (m, 2 H), 2.58 (dd, J=12.8, 2.3 Hz, 1 H), 2.45-2.25 (m, 6 H), 2.18-2.12 (m, 1 H), 1.96 (dd, J=13.1, 6.1 Hz, 1 H), 1.81-1.44 (m, 18 H). 1.43 (s, 18 H), 1.38-1.17 (m, 23 H), 1.16(d, J=6.0 Hz, 3 H), 0.95 (t, J=7.3 Hz, 3 H); ¹³C NMR (125 MHz, CD₃OD)(the C38 amide exists as an approximate 1:1 mixture of rotamers). Some of the signals of carbons in close proximity to C38, including the carbons of the hydroxypermidine unit, are doubled. These signals are listed in parentheses: δ(176.6/176.2), 169.8, 158.6, 158.4, 150.2, 134.1, 131.3, 86.7, 84.6, 80.02, 79.96, 72.0, 70.1, (69.0/68.3), 66.2, (55.0/53.4), 54.8, 54.3, 45.0, 42.6, 39.1, (38.9/38.7), 38.1, 36.2, 34.3, 34.1, 33.7, 32.9, 31.0, 30.78, 30.75, 30.67, 30.64, 30.57, 30.54, 30.50, 30.24, 30.16, 29.7, 28.9, 28.8, 28.7, 27.0, (26.7/26.6), 25.0, 22.4, 21.0, 10.8 ppm; IR (film) 3385, 2927, 2854, 1731, 1668 (br), 1614, 1449, 1366, 1253, 1167, 1028 cm⁻¹; HRMS (FAB) m/z 1001.7 (M-Cl), (1001.7 calcd for C₅₅H₉₇N₆O₁₀). [α]²²_{D}−68.7, [α]²²₅₇₇−72.9, [α]²²₅₄₆−83.3, [α]²²₄₃₅−147.7 (c, 0.6, CHCl₃).

13,14,15-Isocrambescidin 800 Trihydrochloride (10). A solution of 111 (30 mg, 0.029 mmol) and 2.9 mL of a 2.0 M solution of HCl in EtOAc was maintained at rt for 30 min and then concentrated. Purification of the residue by reverse phase. H.PLC (3.5:1 MeOH-0.1 M NaCl, Altima C18, 5 μg column) gave 18 mg (70%) of 13,14,15-isocrambescidin 800 as its trihydrochloride salt (a light yellow oil). Data for this sample were consistent with data published for natural 10.

Data for synthetic 10: ¹H NMR (500 MHz, CD₃OD) δ 5.70 (br t, J=9.1 Hz, 1 H), 5.51 (br d, J=11.2, 1 H), 4.46 (br s, 1 H), 4.18-4.06 (m, 3 H), 3.97-3.86 (m, 3 H), 3.84 (d, J=3.3 Hz, 1 H), 3.70-3.38 (m, 3 H), 3.31-3.07 (m, 3 H), 2.99-2.84 (m, 2 H), 2.57 (dd, J=12.9, 2.4 Hz, 1 H),2.56-2.36 (m, 5 H), 2.31-2.24 (m, 3 H), 2.18-1.43 (m, 18 H), 1.28 (app s, 22H), 1.16-1.15 (overlapping m, 1 H), 1.16 (d, J=6.0 Hz, 3 H), 0.95 (t, J=7.4 Hz, 3 H); ¹³C NMR (125 MHz, CD₃OD) (the C38 amide exists as an approximate 3:1 mixture of rotamers. Only the carbons in close proximity to C38, including some carbons of the hydroxyspermidine unit, exhibit signals due to the minor rotamer. The carbon signals of the rotamers are listed in parentheses with the major rotamer listed first: δ(177.5/176.4), 169.8, 150.2, 134.1, 131.3, 86.8, 84.6, 72.0,70.2, (68.6/69.4), 66.2, (54.9/53.2),54.8, 54.3, (43.9/47.9), 42.7, 39.1, 38.6 (two peaks), (38.27,38.33),38.1, (34.2/34.0). 33.7, 32.9 (2 peaks), 31.0, 30.84, 30.80, 30.75, 30.67, 30.59, 30.54, 30.4, 30.3, 29.7, 28.9, 27.0, (26.63/26.57), 25.0, 22.5, 21.0, 10.8 ppm; MS: HRMS (FAB) m/z 801.6223 (M-Cl), (801.6217 calcd for C₄₅H₈₁N₆O₆). [α]²²_{D}−67.7, [α]²²₅₇₇−70.9, [α]²²₅₄₆−80.6, [α]²²₄₃₅−147.7 (c 0.73, MeOH).

Peracetyl-13,14,15-Isocrambescidin 800 Hydrochloride (112).

A solution of 13,14,15-isocrambescidin 800 (1), acetic anhydride (1.2 mL) and pyridine (2.4 mL) was maintained at rt for 20 then concentrated using a vacuum pump. The resulting residue was dissolved in CHCl₃ (40 ml) and washed sequentially with brine (10 mL), 0.1 N HCl (10 mL) and brine (10 mL). The solution was dried (Na₂SO₄), filtered and concentrated. Purification of the residue by flash chromatography (95:5 CHCl₃-MeOH) gave 8 mg (70%) of Peracetylisocrambescidin 800 (112). ¹H NMR and ¹³C NMR data for synthetic 112 was in agreement with data reported for Peracetylioscrambescidin 800 prepared from natural 13,14,15-Isocrambescidin 800.

Data for synthetic 112:

¹H NMR (500 MHz, CDCl₃) δ10.0 (s, 1 H), 9.97 (s, 1 H of minor rotamer), 9.23 (s, 1 H), 9.19 (s, 1 H of minor rotamer), 6.86 (s, 1 H), 6.70 (s, 1 H of minor rotamer), 6.57 (s, 1 H of minor rotamer), 6.40 (s, 1 H), 5.66 (br t, J=8.7 Hz, 1 H), 5.50 (br d, J=11.0 Hz, 1 H), 5.13-5. 07 (m, 1 H), 4.55 (br s, 1 H), 4.19-4.13 (m, 1 H), 4.02-3.97 (m, 2 H), 3.91-3.88 (m, 1 H), 3.72-3.69 (m, 1 H), 3.62-3.37 (m, 4 H), 3.46 (d, J=2.8 Hz, 1 H), 3.32-3.12 (m, 3 H), 3.05-2.98 (m, 1 H), 2.55-2.52 (m, 2 H), 2.37-2.18 (m, 7 H), [2.05, 2.04, 2.01, 2.00, 1.99 (singlets ofthe acetate methyl groups, 9 H)], 2.00-1.37 (m, 18 H), 1.28-1.18 (m, 23 H), 1.18 (t, J=6.0 Hz, 3 H), 0.96 (t, J=7.3 Hz, 3 H); ¹³C NMR(125MHz, CDCL₃) δ 174.4, 173.8 (minor rotamer), 170.9, 170.8, 170.7, 167.7, 149.3, 133.6, 129.7, 85.0, 82.9, 70.8, 70.5 (minor rotamer), 65.3, 52.9, 52.1, 50.5, 48.4 (minor rotamer), 46.4 (minor rotamer), 42.6, 41.73, 41.68 (minor rotamer), 38.2, 37.4, 37.0, 36.1, 35.6, 35.4, 33.2, 33.1, 32.9, 32.3, 31.4, 30.2, 29.6, 29.5, 29.4, 29.1, 29.0, 28.5, 27.9, 27.0, 25.8, 25.5, 25.4, 24.0, 23.2, 22.1, 21.2, 20.9, 19.6, 10.2 ppm; MS: LRMS (ES) m/z 927.70 (M-Cl), (927.6534 calcd for C₅₁H₈₇N₆O₉). [α]²⁵_{D}−56.1 (c 0.3, CHCl₃).

### EXAMPLE V

Synthesis Plan.

The structural differences and similarities between the two Crambescidin families are apparent in molecular mechanics models of the methyl esters of the 13,14,15-Isocrambescidin and Crambescidin/Ptilomycalin A pentacyclic guanidine moieties (FIG. 20). The lowest energy conformation found from Monte Carlo searches using Macromodel version 5.5 and the OPLS force field is depicted (Chang, G.; Guida, W. C., Still, W. C. J. Am. Chem. Soc. 1989, 111, 4379-4386). Ten thousand starting conformations were examined; in all cases, several conformations that differ only in the spatial orientation of the methyl ester fragment were within a few kcals of the global minimum. For instance, the C10 and C13 angular hydrogens are trans in the isocrambescidin core and cis in the corresponding Crambescidin/Ptilomycalin A unit, while the stereochemical relationship between the substituents at C13, C14 and C15 is the same in both structures. For both alkaloid families, the C—O bonds of the hydropyran and oxepene units are axial. Thus, as in the Crambescidin/Ptilomycalin A series (Snider, B. B.; Shi, Z. Tetrahedron Lett. 1993, 34, 2099-2102; Snider, B. B.; Shi, Z. J. Am. Chem. Soc. 1994, 116, 549-557; Overman, L. E.; Rabinowitz, M. H.; Renhowe P. A. J. Am. Chem. Soc. 1995, 117, 2657-2658;), it was anticipated that the C8 and C15 spirocenters of the Isocrambescidins would evolve with the desired stereochemistry if the central triazaacenaphthalene ring system was constructed with the proper trans stereochemistry.

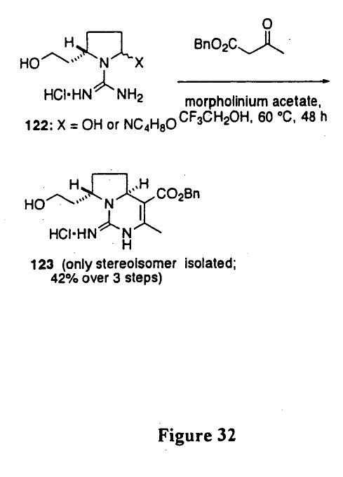

An intramolecular variant of the venerable Biginelli condensation that was introduced several years ago (Overman, L. E.; Rabinowitz, M. H. J. Org. Chem. 1993, 58, 3235-3237) has proven to be highly useful in the design of concise strategies for synthesizing complex guanidine alkaloids. As detailed in Example I, tethered Biginelli condensation of a ureido aldehyde and a β-ketoester can be employed to combine all the carbons of the Crambescidin/Ptilomycalin A pentacyclic core and set the pivotal cis relationship of the H10 and H13 hydrogens (Kappe, C. O. Tetrahedron 1993, 49, 6937-6963). Recent exploratory studies of stereoselection in tethered Biginelli condensations were critical in the planning on how to synthesize the Isocrambescidin alkaloids (McDonald, A. I.; Overman, L. E. J. Org. Chem. 1999, 64, 1520-1528). These investigations revealed that the stereochemical outcome of tethered Biginelli condensations could be reversed if the urea component was replaced with a basic guanidine. Thus, Biginelli condensation of guanidine aldehyde (or aminal) 122 with benzyl acetoacetate provided trans-1-iminohexahydropyrrolo[1,2-c]pyrimidine 123 with high selectivity (FIG. 32) (McDonald, A. I.; Overman, L. E. J. Org Chem. 1999, 64, 1520-1528).

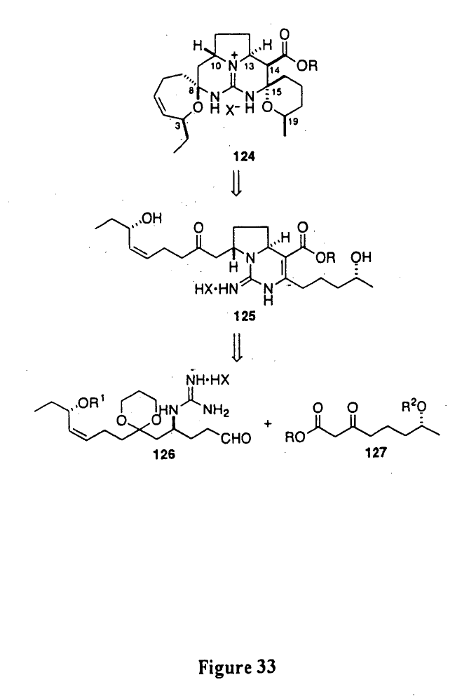

Based on these exploratory studies and the experience in the Crambescidin/Ptilomycalin A series, a convergent plan for preparing 13,14,1 5-Isocrambescidin 800 (10) readily emerged (FIG. 33). Tethered Biginelli condensation of guanidine aldehyde 126 and β-ketoester 127 would be employed to set the critical trans C10-C13 stereorelationship and unite all the heavy atoms of the pentacyclic guanidine moiety. It was hoped that acid promoted dehydration of 125 would then generate the remaining three heterocyclic rings of 124 in a single step. Mindful from the outset of one challenge posed by this strategy: the guanidine functional group would be introduced early in the synthesis. Unless protection and deprotection steps are added, this highly polar functionality would be forced to carry through several stages of the synthesis.

Results and Discussion

Synthesis of trans-1-Iminohexahydropyrrolo[1,2-c]pyrimidine 134.

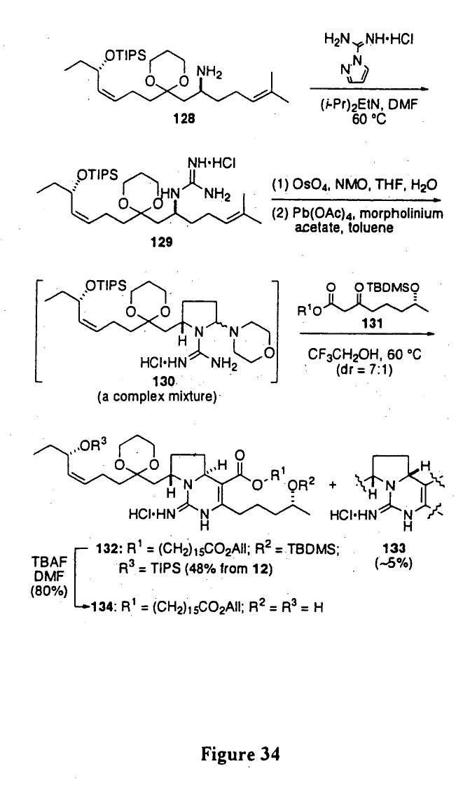

The total syntheses of 10 and 10a began with diene amine 128, which was also utilized in the synthesis of (−)-Crambescidin 800 (FIG. 34). Treatment of 128 with 1-H-pyrazole-1-carboxamidine hydrochloride (Bematowicz, M. S.; Wu, Y.; Matsueda, G. R. J. Org. Chem. 1992, 57, 2497-2502) and diisopropylethylamine at 60° C. generated guanidine 129, which was utilized directly without purification. The trisubstituted double bond of this intermediate next had to be cleaved to liberate the electrophilic component of the Biginelli condensation. Fortunately, the oxidation strategy that was employed to realize this degradation in the related urea series was compatible with the guanidine functionality. Thus, selective dihydroxylation of the trisubstituted double bond of 129 with catalytic osmium tetroxide (OSO₄) and N-methylmorpholine-N-oxide (NMO) (Sharpless, K. B.; Williams, D. R. Tetrahedron Lett. 1975, 3045-3046), followed by cleavage of the resulting diol with Pb(OAc)₄ in the presence of morpholinium acetate provided 130. This intermediate was purified only by filtration to remove PbO₂ and was a mixture of several components as judged by ¹H and ¹³C NMR analysis. Multiple signals were observed for many carbon atoms in ¹³C NMR spectra of 130 and ¹H NMR spectra showed several broad peaks; no aldehyde signal was apparent.

Biginelli condensation of crude 130-and β-ketoester 131 in EtOH at 60° C. proceeded with modest trans selectivity (3:1). Fortunately, it was found that heating 130 with 1.5 equiv of 131 in 2,2,2-trifluoroethanol at 60° C. for 20 h improved diastereoselection to 7:1. After purification of the crude products on silica gel deactivated with pH 7.0 buffer (deactivated silica was prepared by adding 10% (by weight). pH 7.0 phosphate buffer to Merck silica gel (0.040-0.063μ) and mixing until homogeneous), the desired trans adduct 132 was isolated in 48% yield and cis adduct 133 in ca. 5% yield (since the cis adduct 133 was slower moving on silica gel than 132, it was difficult to isolate pure 133). The stereochemistry of 132 was provisionally assigned based on the earlier exploratory studies (McDonald, A. I.; Overman, L. E. J. Org. Chem. 1999, 64, 1520-1528). As seen shortly, this assignment could be confirmed rigorously at a later stage. Deprotection of 132 with tetra-n-butylammonium fluoride (TBAF) in N,N-dimethylformamide (DMF) at room temperature for 36 h gave rise to diol 134 in 80% yield. In some runs, this reaction did not go to completion and intermediates in which only the TIPS group had been removed were isolated in 10-15% yield. Heating the reaction mixture at 60° C. avoided this complication, however, other unidentified products were formned and the isolated yield of 134 was not improved.

Cyclization to Form Pentacycle 135.

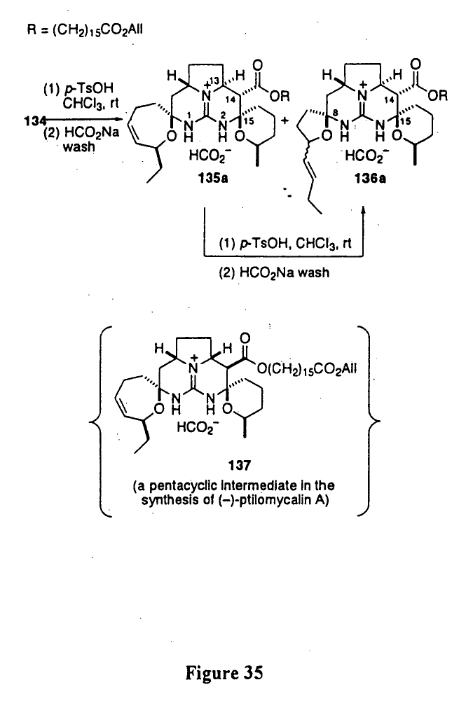

Initially guanidine diol 134 was exposed at room temperature to 3 equiv of p-toluenesulfonic acid monohydrate (p-TsOH.H₂O) in CHCl₃ for 24 h.(FIG. 35). After washing the reaction mixture with aqueous HCO₂Na, a 1:1 mixture of a pentacyclic product, subsequently shown to be 135a, and tetrahydrofuryl isomer 136a were isolated in ca. 50% yield (exchange of the tosylate couter ion for formate required several washings with aqueous sodium formate, which led to some erosion in yield).

The constitution of these pentacyclic products was ascertained as follows. The gross structure of pentacycle 136a, a ˜1:1 mixture of stereoisomers at the center carrying the 1-butenyl side chain, was secured by ¹H NMR COSY and ¹³C NMR studies. The stereochemistry of 136a at C15 (the crambescidin numbering system is employed in the discussion of synthetic intermediates; correct IUPAC names and numbering can be found in the Experimental Section) followed from the chemical shift of the C14 methine hydrogen (δ 2.88) (The C14 methine hydrogen of 135 is observed at δ 2.91, while this hydrogen of 139 is occurs at δ 2.30. The C15 stereochemistry of these products was rigorously determined (vide infra)), while the stereochemistry at C8 was not determined and is assigned on the basis of analogy only. Pentacyclic guanidines 135a and 136a were isolated as their formate salts to allow direct comparisons with pentacycle 137, an intermediate in the original synthesis of (−)-Ptilomycalin A (Example II and Overman, L. E.; Rabinowitz, M. H.; Renhowe P. A. J. Am. Chem. Soc. 1995, 117, 2657-2658). That 135a was epimeric to 137 at C13 was signaled by the absence of an ¹H N NOE between H10 and H13 in the former, while the 11.7 Hz coupling constant of the C14 methine hydrogen of 1353a showed that the ester side chain was equatorial.

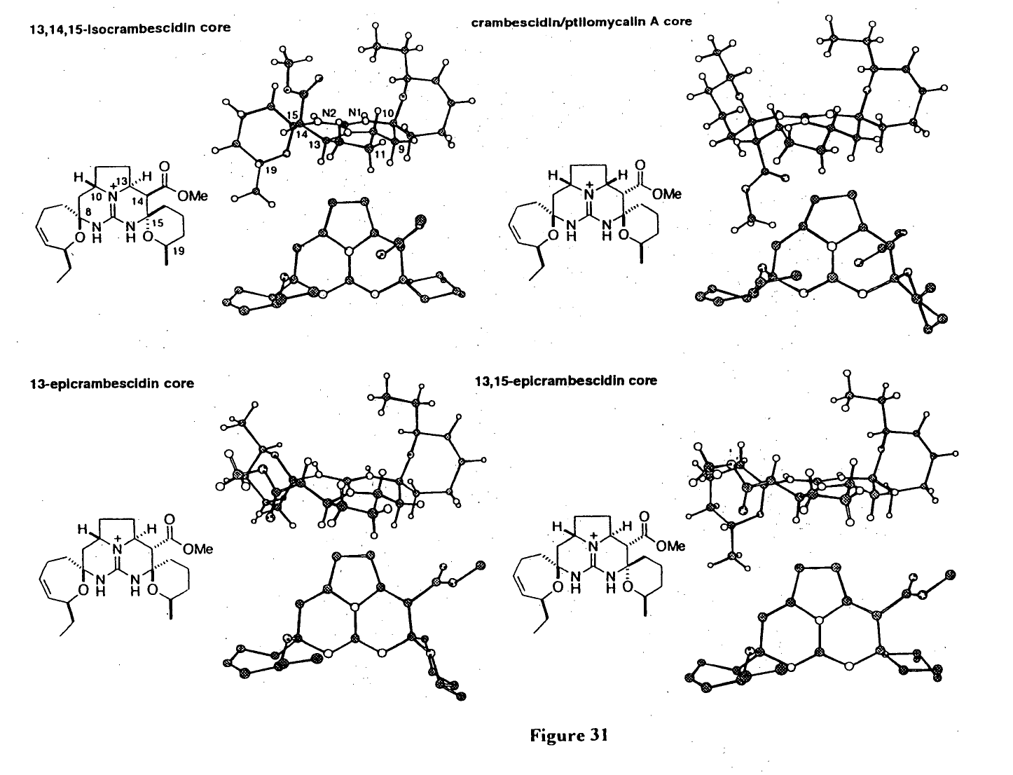

Since none of the pentacyclic guanidine intermediates or products prepared during the investigations were crystalline, ¹H NMR NOE studies proved indispensable in assigning stereochemistry. A molecular mechanics model of the guanidine moiety of 135a (an intermediate having the 13-Epicrambescidin core), which helped in analysis of critical NOE enhancements, is provided in FIG. 31 (the lowest energy conformation found from Monte Carlo searches using Macromodel version 5.5 and the OPLS force field is depicted. Ten thousand starting conformations were examined; in all cases, several conformations that differ only in the spatial orientation of the methyl ester fragment were within a few kcals of the global minimum. As discussed later in the text, the conformation of the 13,15-Isocrarnbescidin core depicted in FIG. 31 is undoubtedly not the lowest energy one). Also provided in FIG. 31 are models of the two additional guanidine pentacycles (13,14,15-isocrambescidin and 13,15-epicrambescidin ring systems) and, for reference, a model of the Crambescidin/Ptilomycalin A pentacyclic guanidine moiety.

Additional investigation revealed that formation of tetrahydrofuran isomer 136a from 134 could be controlled by varying reaction time and equivalents of p-TsOH.H₂O. Larger amounts of acid and longer reaction times favored the formation of 136a. Exposing 135a to p-TsOH.H₂O at room temperature for extended periods also led to 136a. The best conditions found for generating 135a involved exposing 134 to 2 equiv of p-TsOH.H₂O in CHCl₃ for 7 h at room temperature; a 5:1 mixture of 135a and 136a was produced. Since these isomers were difficult to separate, the isolated yield of 135a produced in this way was never greater than 50%.

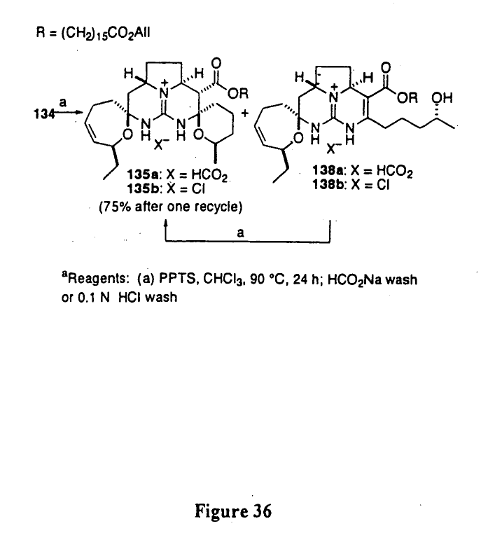

Pyridinium p-toluenesulfonate (PPTS) was examined to cleave the 1,3-dioxane protecting group of 134 and promote cyclization of the resulting keto guanidine diol. With this weaker acid, higher reaction temperatures were required and mixtures of 135a, tetracyclic vinylogous carbamate 138a and several unidentified minor byproducts were produced (FIG. 36). When 134 was heated with 2 equiv of PPTS at 60° C. in CHCl₃ for 5 h and the crude product was washed with aqueous HCO₂Na, 135a and 138a were generated in a 1:5 ratio. Increasing the reaction temperature to 90° C. (sealed tube) for 24 h provided 135a and 138a in a 2:1 ratio (a small amount, ˜10% relative to 138a, of the formate analog of 139 was also produced. When 138a was heated with PPTS at 90° C., 135a and 138a were formed also in a ˜2:1 ratio). Separation of these products on silica gel, followed by resubjection of 138a to PPTS at 90° C. gave 135a in 75% combined yield.

Initially, 135a and 138a were converted to their formate salts prior to chromatography and were eluted from deactivated silica gel using 95:5:0.1 EtOAc-isopropanol-formic acid. It was later found that the hydrochloride salts, 135b and 138b, were easier to separate on silica gel. These salts were prepared by washing the reaction mixture with 0.1 M HCl or saturated aqueous sodium chloride; several washings were required to completely exchange the tosylate counter ion.

Since both NH hydrogens were readily apparent in ¹H NMR spectra of 135b, extensive NMR studies (¹H COSY, HMQC, HMBC and NOESY) eventually revealed that 135b had the 13-Epicrambescidin stereochemistry (i.e., the spiro hydropyran and ester side chain are both epimeric to those of 10 and 10a). Key findings were diagnostic ¹H NMR NOEs observed between N2H and H19, N2H and H17(axial), and H13 and H16(axial); see the model of the 13-Epicrambescidin core in FIG. 31. It is shown in the following discussion, the stereochemistry of both the spiro hydropyran and ester side chain can be readily inverted, allowing 135b to be a viable intermediate for accessing Isocrambescidins 10 and 10a.

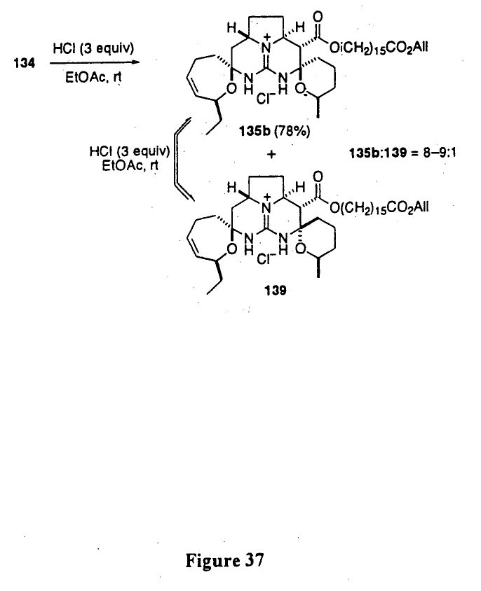

Although the procedures just described provided pentacyclic guanidine salts 135 in synthetically useful yields, these sequences were cumbersome. Ideally, it is needed to find acidic conditions for cyclizing 134 that would not promote allylic rearrangement of the C3 alcohol, yet would irreversibly transform tetracyclic vinylogous carbarnate intermediate 138 to a pentacyclic guanidine isomer. It was eventually discovered that treatnent of 134 with 3 equiv of HCl in EtOAc at room temperature delivered 135b in 78% yield (FIG. 37). Careful purification of the crude cyclization product by reverse phase HPLC (9:1 MeOH-0.1 M NaCl) afforded, in addition to 135b, 5-7% of pentacyclic guanidine 139.

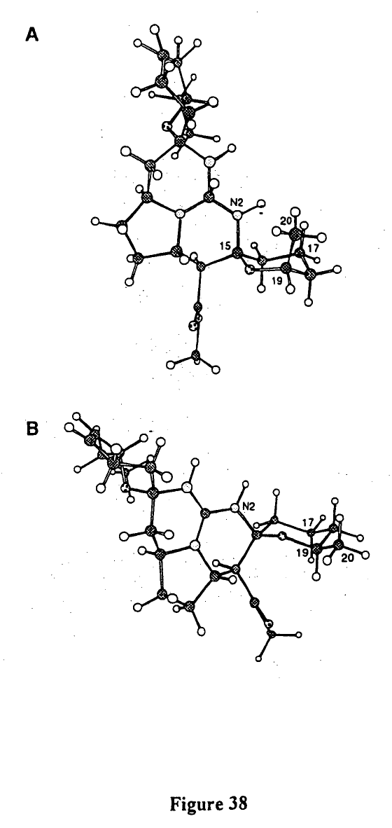

That 139 was epimeric to the Isocrambescidins only at C14 (ester side chain) was apparent from ¹H NMR COSY, HMQC, HMBC and NOESY experiments. The stereochemistry at C15 followed directly from diagnostic ¹H NMR NOEs observed between N2H and the H17(axial) and N2H and H20, and the lack of NOE between N2H and H19. This NOE data is consistent with the hydropyran ring of 139 preferentially adopting a chair conformation having the methyl substituent axial (FIG. 38, conformation A). This conformational preference undoubtedly derives from two factors: (1) In the alternate hydropyran chair conformer, destabilizing syn pentane interactions would exist between C17 and C19 of the hydropyran ring and the carbonyl carbon of the ester group; for two views of this conformation, see FIG. 38, conformation. B and the model of the 13,15-Epicrambescidin core in FIG. 31. (2) Conformer A would be stabilized by an anomeric interaction between the hydropyran oxygen and the C15-N2 bond (Kirby, A. J. Stereoelectronic Effects; Oxford University Press: Oxford, 1995; pp 3-24; Kirby, A. J. The Anomeric Effect and Related Stereoelectronic Effects at Oxygen; Springer: Berlin, 1983; Deslongchamps, P. Stereoelectronic Effects in Organic Chemistry; Pergamon: Oxford, 1983.).

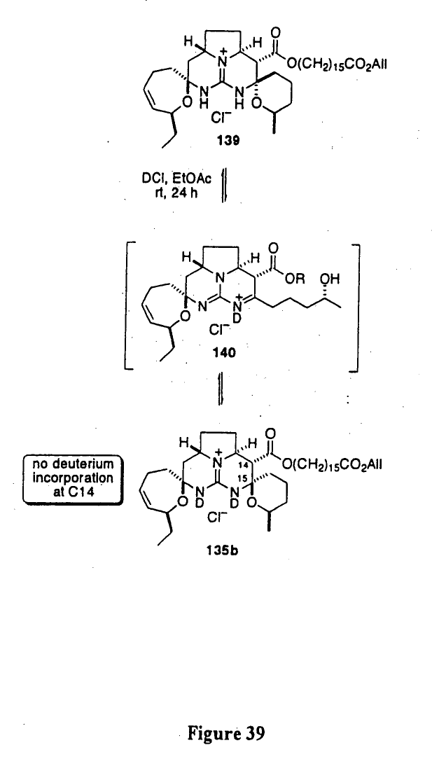

To gain more insight into the mechanism of hydropyran formation, pure 135b was resubjected to the cyclization conditions (3 equiv HCl in EtOAc at room temperature) to yield an approximate 8-9:1 mixture of 135b and 139 (FIG. 37). That this represents an equilibrium ratio of the C15 epimers under these conditions was established by: (a) demonstrating that the 8-9:1 mixture-of 135b and 139 was unchanged when resubjected to the reaction conditions for an additional 24 h, and (b) showing that pure 139 gives an identical ratio of epimers when exposed for 24 h to 3 equiv HCl in EtOAc. Since no intermediates or by-products having the ester side chain on the β face were detected in HCl-promoted cyclization of 134, or HCl-promoted equilibrations of the Spiro hydropyran epimers, it was surmised that the equilibration of 135b and 139 did not involve tetracyclic intermediates such as 138. Consistent with this hypothesis, exposure of 139 to DCl in EtOAc gave an approximate 8-9:1 mixture of 135b and 139 without incorporation of deuterium into 135b (FIG. 39). Iminium cation 140 is the likely intermediate in the equilibration of the spiro hydropyran epimers (although not rigorously precluded, the alternate possibility that epimerization at C15 occurs by cleavage of the N2-C15 bond to form a six-membered oxocarbenium ion intermediate to be less likely). It was concluded from these studies that formation of 135b as the major product from HCl-promoted cyclization of 134 arises from kinetically-controlled axial protonation of the vinylogous carbamate unit of 134 to generate the protio equivalent of 140, which undergoes thermodynamically-controlled spirocyclization to generate 135b preferentially (in our syntheses of Ptilomycalin A and Crambescidin 800, the only spirohydrofuran products formed from acid-promoted cyclization of related vinylogous carbamates have the oxygen axial. In those cases, equilibration of hydropyran epimers by a pathway related to that depicted in FIG. 39 would occur at slower rates since less stable N-acyliminium cations would be involved).

Epimerization of 135b at C14 and C15 to Give Pentacyclic Guanidine Acid 141 and Total Synthesis of 13,14,15-isocrambescidin 657(10a).

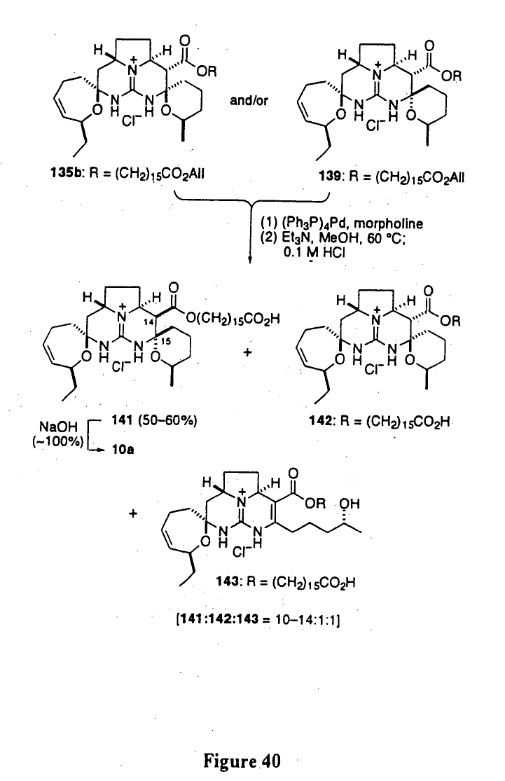

Not long after 135a was first prepared, it was established that exposure of this intermediate to Et₃N in hot methanol provided a pentacyclic guanidine whose stereochemistry was identical to that of 13,14,15-Isocrambescidin 800 (10). Although this fact was not initially appreciated, epimerization at C14 and C15 is a coupled event. This reorganization was best accomplished after removal of the allyl group of the hexadecanoate ester. To this end, the 8-9:1 mixture of 135b and 139 resulting from HCl-promoted cyclization of 134 was deprotected with (Ph₃P)₄Pd and morpholine (FIG. 40). The resulting mixture of acids was then epimerized by heating in MeOH at 60° C. in the presence of 10 equiv of Et₃N. Acidification of this product with 0.1 M HCl yielded a mixture of pentacyclic guanidine acids 141 and 142 and tetracyclic guanidine 143 in an approximate ratio of 10-1 4:1:1 (the ratio of 141 to (142 and 143) was determined from the crude product mixture by ¹H NMR analysis at 500 MHz. Due to the complexity of this spectrum and the presence of minor impurities, it was estimated that this ratio is only accurate to±20%. The ratio of 142:143 was more difficult to ascertain, although these products appeared to be formed in similar amounts. Attempts to resolve this mixture by HPLC were unsuccessful). The pentacyclic guanidine acid resulting from deallylation of 139 was not detected. After purification by flash chromatography on silica gel, 141, which exhibits a diagnostic 3.3 Hz coupling constant for the equatorial C14 methine hydrogen, was isolated in 50-60% yield for the two steps. A similar mixture of products was obtained when pure samples of 135b or 139 were individually deallylated and heated with Et₃N in MeOH. In contrast to precursors of (−)Ptilomycalin A.(1) and Crainbescidin 800 (2) (Overman, L. E.; Rabinowitz, M. H.; Renhowe P. A. J. Am. Chem. Soc. 1995, 117, 2657-2658), the axial ester is highly favored in the Isocrarnbescidin series.

The structure of 141 was secured by extensive ¹HNMR COSY, HMQC, HMBC and NOESY experiments. The stereochemistry of 141 at C15 followed from diagnostic ¹H NMR NOEs observed between H19 and H14 and between H19 and H13 (weaker), and the absence of NOEs between N2H and H 19 (see the 3-dimensional model of the 13,14,15-Isocrambescidin core in FIG. 2). Carboxylic acid 141 was quantitatively converted to the corresponding inner salt by washing with dilute NaOH. This product showed ¹H and ¹³C NMR data fully consistent with those reported (Kashman, Y.; Hirsh, S.; McConnell, O. J.; Ohtani, I.; Kusumi, T; Kakisawa, H. J. Am. Chem. Soc. 1989, 111, 8925-8926) for 13,14,1 5-Isocrambescidin 657 (10a). The specific rotation of synthetic 10a was [α]²³_{D}−35.4 .(c 0.8 MeOH), which agrees well with the specific rotation, [α]²³_{D}−32.7 (c 0.3 MeOH), reported (kashman, Y.; Hirsh, S.; McConnell, O. J.; Ohtani, I.; Kusumi, T; Kakisawa, H. J. Am. Chem. Soc. 1989, 111, 8925-8926) for natural 13,14,15-Isocrambescidin 657 (10a). Complete assignments of the ¹H and ¹³C chemical shifts of 10a and 141 are provided.

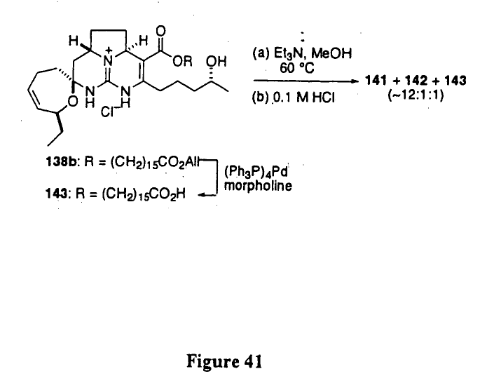

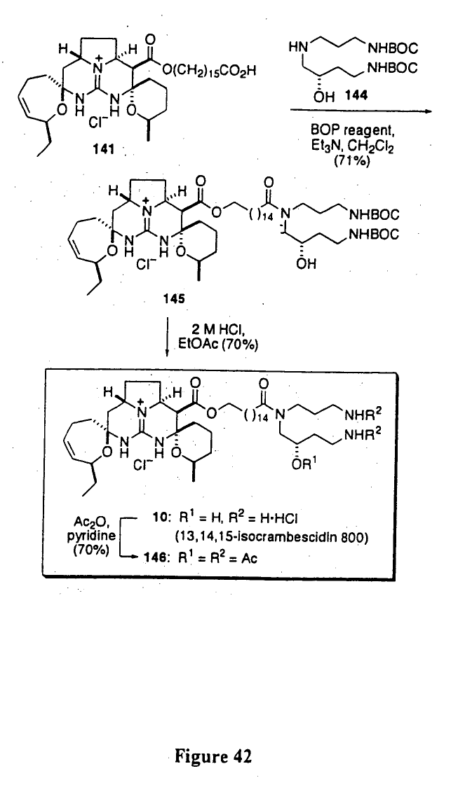

Since a pure sample of 138b was available from our earlier studies of the cyclization of 134 with PPTS, this tetracyclic guanidine was deallylated to form 143 (FIG. 41). Exposure of 143 to Et₃N and MeOH at 60° C. provided a product mixture containing 141, 142 and 143 in an approximate 12:1:1 ratio. As in the related conversions of 135b and 139, the acid congener of 139 was not detected. The experiment sununarized in FIG. 41 provides permissive evidence for the intermediacy of 143 in the epimerization of 135b at C14 and C15 to provide 141.

Total Synthesis of 13,14,15-isocrambescidin 800 (10).

The (S)-7-hydroxyspermidine fragment 144, which is available from (R)-epichlorohydrin (Coffey, D. S.; McDonald, A. I.; Overman, L. E. J. Org. Chem. 1999, 64, 8741-8742), was coupled to pentacyclic acid 141 using benzotriazol-1-yloxytris(dimethylamino)phosphonium hexafluorophosphate (BOP) (Castro, B.; Dormoy, J. R.; Evin, G.; Selve, C. Tetrahedron Lett. 1975, 1219-1222) to provide 145 in 71% yield. Removal of the BOC protecting groups with 2 M HCl in ethyl acetate (Stahl, G. L.; Walter, R.; Smith, C. W. J. Org. Chem. 1978, 43, 2285-2286) and purification of the crude product by reverse-phase HPLC gave the trihydrochloride salt of 13,14,15-isocrambescidin 800 (10), [α]_{D}²³−67.7 (c 0.7 MeOH), in 70% yield. A specific rotation of [α]²³_{D}−48 (c 0.5 MeOH) is reported for natural 13,14,15-Isocrambescidin 800 (10) (Jares-Erijman, E. A.; Ingrum, A. L.; Carney, J. R.; Rinehart, K. L.; Sakai, R. J. Org. Chem. 1993, 58, 4805-808). Since the counter ion of natural 10 was not described, the significance, if any, of this discrepancy in rotation magnitude is unknown. NMR data for the trihydrochloride salt of synthetic 10 were in good agreement with those reported for natural 10, (Jares-Erijman, E. A.; Ingrum, A. L.; Carney, J. R; Rinehart, K. L.; Sakai, R. J. Org. Chem. 1993, 58, 4805-4808). The trihydrochloride salt of 10 was obtained, since a basic workup was not performed after the removal of the BOC groups. However, natural 10 has been depicted with the spermidine nitrogens in the free base form (Berlinck, R. G. S.; Braekman, J. C.; Daloze, D.; Bruno, I.; Riccio, R.; Ferri, S.; Spampinato, S.; Speroni, E. J. Nat. Prod. 1993, 56, 1007-1015; Jares-Erijman, E. A.; Ingrum, A. L.; Carney, J. R; Rinehart, K. L.; Sakai, R. J. Org. Chem. 1993, 58, 4805-4808), yet the ¹H and ¹³C NMR spectra of synthetic 10 and natural 10 were indistinguishable. Treatment of synthetic 10 with 0.1 M NaOH saturated with NaCl resulted in downfield shifts of the C41 and C45 hydrogens. To investigate this issue further, i was prepared to model the hydroxyspermidine unit of 13,14,15-Isocrambescidin 800. Chemical shifts of the hydrogens of the hydroxyspermidine units of i and synthetic 10 were nearly identical; the absence of the guanidine unit made assignments for i straightforward. Treatment of i with 0.1 M NaOH gave ii as the free base. As summarized in the Table below, there were significant upfield shifts of the C41 and C45 hydrogens in ii upon deprotonation. This study and related experiments with synthetic 10, provide confidence that natural 13,14,15-Isocrambescidin 800 (10) was isolated as the trihydrochloride salt. Synthetic 10 was indistinguishable from a natural sample of 10 by HPLC comparisons using three eluents.

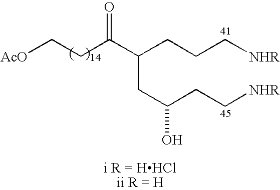

¹H NMR shifts of the C41 and C45 hydrogens.^{a}
δ (ppm), mult

| position | i | ii |
|---|---|---|
| 41 | 2.99-2.84, m | 2.66-2.60, m |
| 45 | 3.14-3.08, m | 2.86-2.78, m |

^{a}In CD₃OD at 500 MHz

To provide one additional point of comparison, synthetic 10 was converted to triacetylated derivative 146. Data for this product agreed perfectly with ¹H and ¹³C NMR data reported for this derivative of natural 10 (Berlinck, R. G. S.; Braekman, J. C.; Daloze, D.; Bruno, I.; Riccio, R.; Ferri, S.; Spampinato, S.; Speroni, E. J. Nat. Prod. 1993, 56, 1007-1015).

Proof that the C43 Stereocenter of ]13,14,15-Isocrambescidin 800 (10) is S.

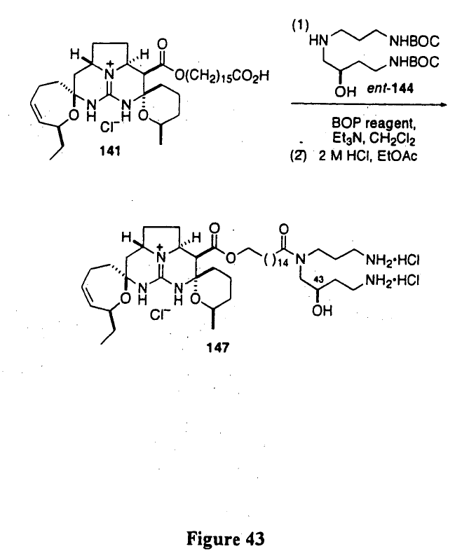

As noted earlier, the S configuration of the C43 stereocenter of 13,14,15-Isocrambescidin 800 (10) had been proposed solely by analogy with Crambescidin 816 (Berlinck, R. G. S.; Braekman, J. C.; Daloze, D.; Bruno, I.; Riccio, R.; Ferri, S.; Spampinato, S.; Speroni, E. J. Nat. Prod. 1993, 56, 1007-1015; Jares-Erijman, E. A.; Ingrum, A. L.; Carney, J. R.; Rinehart, K. L.; Sakai, R. J. Org. Chem. 1993, 58, 4805-4808). On the surface, our total synthesis of 10 appeared to confirm this assignment. However, since the C43 stereocenter is far removed from stereocenters of the pentacyclic guanidine moiety, it was not confident that epimers at this sterogenic center would be readily distinguished. To pursue this issue further, (43R)-13,14,15-Isocrambescidin 800 (147) was prepared from 141 and ent-144 (FIG. 43) (Hydroxyspermidine derivative ent-144 was prepared from (S)-epichlorohydrin (Coffey, D. S.; McDonald, A. I; Overman, L. E. J. Org. Chem. 1999, 64, 8741-8742)). 147 was indistinguishable from synthetic 10 and natural 10 by ¹H and ¹³C NMR comparisons as well as by HPLC analysis.

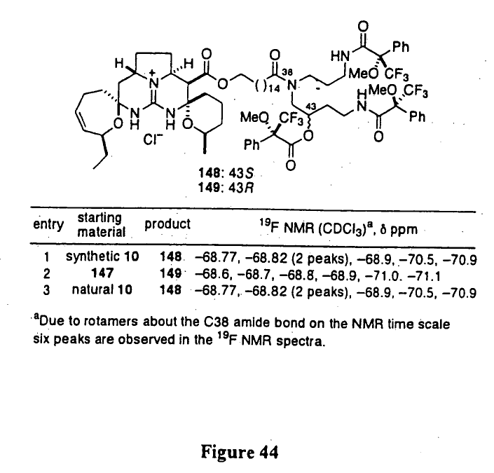

To unambiguously differentiate the C43 epimers of 13,14,15-Isocrambescidin 800, a common derivative of natural 10, synthetic 10 and 147 were prepared. Since only 200 μg of natural 10 was available, it was chosen to employ Mosher derivatives and do the analysis by ¹⁹F NMR spectroscopy (Dale, J. A.; Mosher, H. S. J. Am. Chem. Soc. 1973, 95, 512-519). The tris Mosher derivatives 148 (43S) and 149 (43R) were prepared from (S)-(−)-α-methoxy-α-(trifluoromethyl)phenylacetic acid (MTPA), synthetic 10 and 147 according to the method developed by Ward (Ward, D. E.; Rhee, C. K. Tetrahedron Lett. 1991, 32, 7165-7166) and their ¹⁹F NMR spectra were recorded. Since these products were mixtures of two rotamers on the NMR time scale, six ¹⁹F signals were observed. Several of the signals were substantially different in diastereomers 148 and 149 (FIG. 44). The (S)-WTA derivatives of natural and synthetic 10 were identical, thus unambiguously establishing that the stereochemistry of 13,14,15-isocrambescidin 800 (10) at C43 is S.

Relative Energies of Pentacyclic Guanidine Stereoisomers.

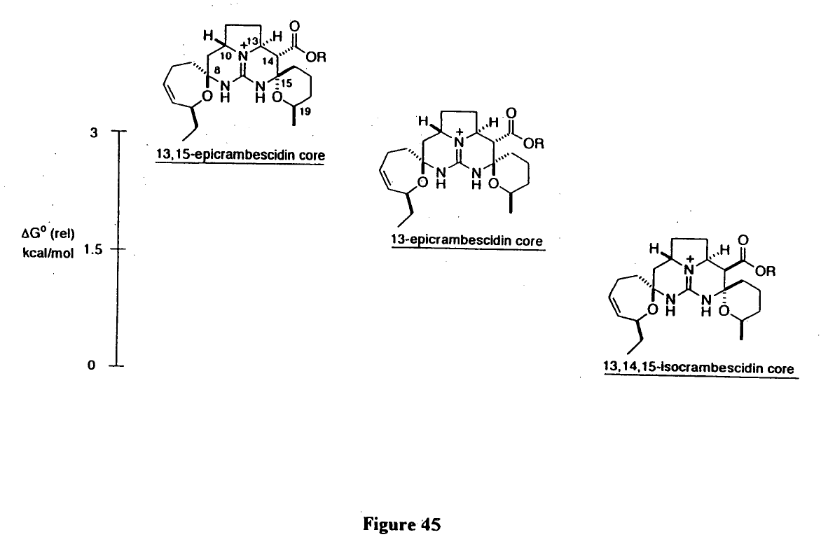

In contrast to the studies with Ptilomycalin A/Crambescidin compounds, the investigations with the Isocrambescidin compounds provided access to several pentacyclic guanidine stereoisomers. The relative energies of the 13,15-Epicrambescidin and 13-Epicrambescidin pentacyclic guanidine moieties are readily discerned, since 139 and 135 equilibrate at room temperature in the presence of HCl (FIG. 37). No similarly clean equilibration allows us to precisely specify the relative energy of the 13,14,15-Isocrambescidin ring system. Nonetheless, that the 13,14,15-Isocrambescidin ring system is considerably more stable than the 13-Epicrambescidin guanidine moiety was signaled early in the studies when it was observed that the 13-Epicrambescidin ester 135a was converted in good yield to the allyl ester analog of the 13,14,15-Isocrambescidin acid 141 upon treatment with Et₃N in hot methanol. Moreover, exposure of 142, 143, or the acid derived from 139 to methanolic Et₃N at 60° C. provided the 13,14,15-Isocrambescidin acid 141 and the 13-Epicrambescidin acid 142 in an approximate ratio of 12:1 (FIG. 40). Although the complexity of this reaction mixture, the inability to isolate 142 in pure form, and analytical difficulties prevents unambiguous specification that this ratio of 141 and 142 accurately represents thermodynamic equilibrium at 60° C., this ratio is a reasonable estimate. Using this estimate, the energetic ordering of the 13-Epicrambescidin, 13,15-Epicrambescidin and 13,14,15-Isocrambescidin pentacyclic guanidine ring systems depicted in FIG. 45 is obtained.

That epimerization of the 13,15-Epicrambescidin guanidine moiety at C14 would be highly favored is apparent in the molecular models shown in FIGS. 31 and 38. In one hydropyran chair conformer of the 13,15-Epicrambescidin ring system the ester substituent is thrust over the hydropyran ring (conformer B of FIG. 38 and alternate views shown in FIG. 31) and in the other chair conformer, which relieves this interaction, the methyl group is axial (conformer A of FIG. 38). No such destabilizing interactions exist in the 13,14,15-Isocrambescidin ring system.

Conclusion.

The first total syntheses of 13,14,15-Isocrambescidin 800 (10) and 13,14,15-Isocrambescidin 657 (10a) were accomplished in convergent fashion. The synthesis of 10 was achieved, in 11% overall yield from amine 128 by a sequence involving 5 isolated intermediates. As detailed previously, 128 can be accessed from commercially available 3-butyn-1-ol in 30% overall yield by way of nine isolated and purified intermediates. Thus, the approach to the Isocrambescidins recorded herein is capable of providing these guanidine alkaloids on meaningful scales.

The total syntheses detailed herein confirm the stereochemical assignments of 10 and 10a and rigorously establish that the absolute configuration of the hydroxyspermidine side chain of 10 is S. Moreover, this Example demonstrated for the first time that the tethered Biginelli strategy for preparing crambescidin alkaloids can be extended to guanidine intermediates and that the key Biginelli condensation can be accomplished under sufficiently mild conditions that fragments containing the full functionality of the Crambescidin core can be employed.

Experimental Section (Experimental Details are the Same as Those Described in the Preceding Example)

(6S,11Z,13S)-6-Amino-N-carboxamidine-8-(1′,3′-dioxan-2′-yl)-2-methyl-13-triisopropylsiloxypentadeca-2,11-diene (129).

A solution of amine 128 (2.95 g, 6.12 mmol), 1-H-pyrazole-1-carboxamidine hydrochloride (2.70 g, 18.4 mmol), i-Pr₂EtN (4.4 mL, 24 mmol) and DM (6 mL) was maintained at rt for 16 h and then at 60° C. for 4 h. The solution was cooled to rt and partitioned between CHCl₃ (300 mL) and 0.1 M HCl (75 mL). The organic phase was washed with 0.1 M HCl (75 mL) and H₂O (75 mL), dried (Na₂SO₄), filtered and concentrated to give a 2:1 mixture of guanidine 129 and amine 128. This mixture was dissolved in DMF (6 mL) and again allowed to react (rt for 16 h and 60° C. for 4 h) with 1-H-pyrazole-1-carboxamidine hydrochloride (1.35 g, 9.2 mmol) and i-Pr₂EtN base (2.2 mL, 12 mmol). The reaction was worked up as previously described, residual DMF was removed by evacuation for several hours at 0.1 Imm to provide 3.20 g (˜99%) of crude guanidine 129 as a light yellow oil. This intermediate was used without further purification: ¹H NMR (500 MHz, CDCl₃) δ 7.82 (app d, J=6.7 Hz, 1H), 7.24 (br s, 1H), 5.43-5.39 (m, 1H), 5.29-5.24 (m, 1H), 5.09 (br t, J=7.0 Hz, 1H), 4.45 (app q, J=7.3 Hz, 1H), 3.98-3.76 (m, 4H), 3.62-3.59 (m, 1 H), 2.20-2.1.3 (m, 2H), 2.02-1.74 (overlapping m, 6H), 1.74-1.67 (m, 2H), 1.69 (s, 3H), 1.64-1.58 (overlapping m, 2H), 1.62 (s, 3H), 1.51-1.38 (m, 2H), 1.05 (m, 21H), 0.87 (t, J=7.4 Hz, 3H), ¹³C NMR (125 MHz, CDCl₃) δ 157.6, 135.0, 132.7, 126.9, 123.1, 100.5, 69.8, 59.8, 59.3, 46.6, 45.0, 36.5, 31.7, 30.5, 25.7, 25.0, 24.8, 22.2, 18.1, 18.0, 17.6, 12.3, 9.3 ppm; IR (film) 2961, 2865, 1651, 1463, 1383, 1246, 1109 cm⁻¹; HRMS (FAB) m/z 524.4225 (524.4250 calcd for C₂₇H₅₈N₃O₃Si, M-Cl); [α]²⁵_{D}□+1.7, [α]²⁵₅₇₇+2.7, [α]²⁵₅₄₆+3.2, [α]²⁵₄₃₅+7.3, [α]²⁵₄₀₅+9.3 (c 1.3, CHCl₃).

(4aS,7S)-4-[15-(Allyloxycarbonyl)pentadecyloxycarbonyl]-3-[(4S)-4-t-butyldimethylsiloxypentyl]-7-[(5Z,7S)-2-(1′,3′-dioxan-2′-yl)-7-triisopropylsiloxy-5-nonenyl]-1,2,4a,5,6,7-hexahydro-1-imino-pyrrolo[1,2-c]-pyrimidine Hydrochloride (132).

N-Methylmorpholine-N-oxide (2.16 g, 18.4 mmol) and OₛO₄ (3.1 mL, 0.24 mmol, 2% in tert-butanol) were added to a solution of guanidine 129 (3.2 g, ˜6.1 mmol), THF (105 mL) and H₂O (15 mL). The mixture was stirred at rt for 8 h, Florisil (1.5 g) and NaHSO₃ (1.5 g) were added, and the resulting mixture was stirred for an additional 10 h. Celite and MgSO₄ then were added, the mixture was filtered and the eluent was concentrated to give the corresponding crude diol as a brown oil. This oil was dissolved in toluene (120 mL) and morpholinium acetate (3.6 g, 24 mmol) and Pb(OAc)₄ (3.3 g, 7.3 mmol) were added. The resulting mixture was maintained at rt for 45 min and Celite was added. This mixture was filtered through a plug of Celite, the eluent was diluted with toluene (200 mL) and the solution was concentrated to give a brown oil. This oil was azeotroped to dryness with toluene (200 mL) and the residue was combined with β-ketoester 131(5.3 g, 9.2 mmol) and 2,2,2-trifluoroethanol (9 mL). The resulting solution was maintained at 60° C. for 20 h and then partitioned between CHCl₃ (250 mL) and 0.1 M HCl (50 mL). The organic phase was washed with 0.1 M HCl (50 mL) and brine (50 mL), dried (Na₂SO₄), filtered and concentrated. Analysis by ¹H NMR revealed a 7:1 ratio of trans:cis Biginelli adducts. Purification of the crude mixture by flash chromatography (CHCl₃→99:1 CHCl₃-MeOH→98:2 CHCl₃-MeOH) on silica gel deactivated with pH 7.0 buffer (McDonald, A. I.; Overman, L. E. J. Org. Chem. 1999, 64, 1520-1528) provided 3.22 g (48% from 128) ofthe desired anti adduct 132 as a light brown oil and 331 mg (5% from 128) of syn adduct 133. Data for 132: ¹H NMR (500 MHz, CDCl₃) δ 9.06 (s, 1H), 7.33 (s, 1H), 5.95-5.88 (m, 1H), 5.43 (app t, J=9.8 Hz, 1H), 5.31 (app dq, J=17.2, 1.5 Hz, 1H), 5.27-5.25 (m, 1H), 5.23 (app dq, J=10.4, 1.3 Hz, 1H), 4.57 (br d, J=5.7, 2H), 4.46-4.41 (m, 2H), 4.27-4.24 (m, 1H), 4.17-4.07 (m, 2H), 4.01-3.95 (m, 2H), 3.91-3.78 (m, 3H), 2.77-2.71 (m, 2H), 2.65-2.59 (m, 1H), 2.45-2.40 (m, 1H), 2.32 (t, J=7.6 Hz, 2H), 2.07-1.88 (m, 6H), 1.79-1.55 (m, 11H), 1.53-1.43 (m, 4H), 1.31-1.25 (m, 21H), 1.13 (d, J=6.1 Hz, 3H), 1.05 (s, 21H), 0.87 (t, J=7.4 Hz, 3H), 0.86 (s, 9H), 0.037 (s, 3H), 0.032 (s, 3H); ¹³C NMR (100 MHz, CDCl₃) δ 173.4, 165.0, 149.9, 147.3, 135.3, 132.2, 126.4, 117.9, 100.9, 100.3, 69.8, 68.3, 64.8, 64.7, 59.9, 59.4, 57.5, 54.1, 46.1, 39.0, 34.8, 34.2, 33.3, 31.6, 30.9, 30.3, 29.6, 29.52, 29.48, 29.42, 29.3, 29.1, 29.0, 28.5, 26.0, 25.8, 24.83, 24.76, 24.4, 23.6, 22.1, 18.01, 17.98, 12.3, 9.2, −4.5, −4.7 ppm; IR (film) 2926, 2856, 1738, 1713, 1681, 1538, 1462, 1382, 1256, 1086 cm⁻¹; HRMS(FAB)nm/z 1044.6 (1044.8 calcd for C₅₉H₁₁₀N₃O₈Si₂ M-Cl); [α]²⁵_{D}−21.2, [α]²⁵₅₇₇−21.3, [α]²⁵₅₄₆−23.3, [α]²⁵₄₃₅ −28.8, [α]²⁵₄₀₅−25.1 (c 1.9, CHCl₃).

(4aS,7S)-4-[15-(Allyloxycarbonyl)pentadecyloxycarbonyl]-7-[(5Z,7S)-2-(1′,1,3′-dioxan-2′-yl)-7-hydroxy-5-nonenyl]-1,2,4a,5,6,7-hexahydro-3-[(4S)-4-hydroxypentyl]-1-iminopyrrolo[1,2-c]pyrimidine Hydrochloride (134).

A solution of 132 (2.80 g, 2.59 mmol), tetrabutylammonium fluoride (TBAF, 13 mL, 13 mmol, 1.0 M) and DMF (26 mL) was maintained at rt for 24 h, then more TBAF (6 mL, 6 mmol, 1.0 M) was added. The solution was maintained at for 6 h then partitioned between CHCl₃ (200 mL) and 0.1 M HCl (75 mL). The organic phase was washed with saturated aqueous HCO₂Na (2×50 mL), dried (Na₂SO₄), filtered and the filtrate was concentrated. The crude product was purified by flash chromatography (95:5:0.1 EtOAc-isopropanol-formic acid→90:10:0.1 EtOAc-isopropanol-formic acid→85:15:0.1 EtOAc-isopropanol-formic acid) on silica gel deactivated with pH 7.0 buffer to give the formate salt of the diol 1.68 g (80%) as a light brown oil.

The formate salt was easier to purify, but the chloride salt was more stable. Therefore, after purification, the formate salt was converted quantitatively to chloride salt 134 by partitioning the formate salt between CHCl₃ (150 mL) and 0.1 M HCl (25 mL) and washing the organic layer with 0.1 M HCl (25 mL) and brine (25 mL). The organic phase was dried (Na₂SO₄), filtered and concentrated to give diol 134: ¹H NMR (500 MHz, CDCl₃) δ 8.63 (s, 1H), 7.43 (s, 1H), 5.95-5.87 (m, 1H), 5.51-5.42 (m, 2H), 5.31 (ddd, J=17.2, 3.0, 1.5 Hz, 1H), 5.22 (ddd, J=9.2, 3.0, 1.3 Hz, 1H), 4.57 (dt, J=5.7, 1.3 Hz, 2H), 4.43 (dd, J=9.9, 4.3 Hz, 1H), 4.32 (app q, J=7.1 Hz, 1H), 4.28-4.25 (m, 1H), 4.17-4.08 (m, 2H), 4.05-3.92 (m, 3H), 3.89-3.82 (m, 2H), 2.91-2.86 (m, 1H), 2.62-2.58 (m, 1H), 2.52 (dt, J=11.8, 4.6 Hz, 1H), 2.42-2.39 (m, 1H), 2.32 (t, J=7.6 Hz, 2H), 2.16-1.96 (m, 6H), 1.86-1.72 (m, 3H), 1.70-1.44 (m, 11H), 1.30-1.24 (m, 22H), 1.19 (d, J=6.2 Hz, 3H), 0.91 (t, J=7.4 Hz, 3H); ¹³CNMR (125 MHz, CDCl₃) δ 173.5, 165.0, 149.7, 147.5, 133.5, 132.3, 130.4, 118.0, 101.0, 100.5, 68.7, 65.4, 64.85, 64.76, 60.1, 59.6, 57.6, 54.2, 45.8, 38.1, 34.7, 34.2, 33.1, 30.4, 30.2, 29.6, 29.51, 29.46, 29.37, 29.2, 29.1, 28.6, 26.0, 24.9, 24.7, 24.0, 23.5, 22.2, 9.7 ppm; JR (film) 3344, 2925, 2854, 1736, 1685, 1542, 1462, 1384, 1259, 1170, 1084, 1001 cm⁻¹; MS: HRMS (FAB) m/z 774.5615 (774.5632 calcd for C₄₄H₇₆N₃O₈, M-Cl); [α]²⁵_{D}−39.4, [α]²⁵₅₇₇−40.2, [α]²⁵₅₄₆−44.8, [α]²⁵₄₃₅−66.0, [α]²⁵₄₀₅−70.0 (c 1.2, CHCl₃).

Formation of Pentacycle 19b from 18 by Reaction with Methanolic HCl.

Acetyl chloride (320 μL, 4.5 mmol) was added to a 0° C. solution of MeOH (200 μL, 5.0 mmol) and EtOAc (30 mL) to give a 0.15 M solution of HCl in EtOAc. Diol 134 (1.10 g, 1.36 mmol) was then dissolved in 27 mL of this solution. This solution (containing 4.1 mmol of HCl) was maintained at rt for 6 h, then partitioned between CHCl₃ (250 mL) and brine (50 mL). The organic phase was dried (Na₂SO₄), filtered and concentrated. Purification of the residue by flash chromatography (CHCl₃→99:1 CHC₃→MeOH→98:2 CHCl₃→MeOH) gave 780 mg (78%) of an approximate 8-9:1 mixture of pentacycles 135b and 139 as a light yellow oil (it was difficult to measure accurately the ratio of 135b and 139, since many peaks in the ¹H NMR spectra overlapped). This mixture was used without further purification in the next step.

For characterization purposes, a sample of this mixture was purified by reverse phase HPLC (9:1 MeOH40.1 M NaCl). To insure that the counterions of 135b and 139 were uniquely chloride, pure samples of 135b and 139 were dissolved in CHCl₃ (50 mL), washed with 0.1 M HCl (10 mL), and the organic phases were dried (Na₂SO₄), filtered and concentrated (there are small differences in the ¹H NMR and ¹³C NMR spectra of 135b, 139 and 10a before and after washing with 0.1 M HCl. In Example IV, 135b and 10a were not washed with 0.1 M HCl after purification.

Data for 135b: ¹H NMR (500 Mz, CDCl₃) δ 10.37 (s, 1H), 9.81 (s, 1H), 5.95-5.87 (m, 1H), 5.69-5.65 (m, 1H), 5.48 (br d, J=10.9 Hz, 1H), 5.31 (dq, J=17.2, 1.5 Hz, 1H), 5.22 (dq, J=10.4, 1.3 Hz, 1H), 4.57 (dt, J=5.7, 1.4 Hz, 2H), 4.50 (br d, J=8.1 Hz, 1H), 4.31-4.27 (m, 1 H), 4.26-4.21 (m, 1H), 4.12-4.07 (m, 1H), 3.98-3.95 (m, 1H), 3.77-3.72 (m, 1H), 2.91 (d, J=11.7 Hz, 1H), 2.58-2.53 (m, 2H), 2.32 (t, J=7.6 Hz, 2H), 2.31-2.28 (m, 3H), 2.21-2.17 (m, 2H), 1.93 (dd, J=14.5, 5.3 Hz, 1H), 1.86-1.72 (m, 3H), 1.69-1.60 (m, 7H), 1.57-1.36 (m, 6H), 1.32-1.20(m, 19H), 1.17-1.12(m, 1H), 1.13(d, J=6.0Hz,3H),0.87(t, J=7.3 Hz, 3H); ¹³C NMR (125 MHz, CDCl₃) δ 173.4, 169.0, 150.9, 133.3, 132.3, 129.8, 118.0, 85.6, 84.7, 70.8, 68.8, 65.5, 64.8, 58.5, 55.1, 52.2, 37.5, 37.2, 34.2, 33.0, 32.1, 30.9, 30.0, 29.56, 29.53, 29.46, 29.38, 29.2, 29.11, 29.09, 28.5, 25.9, 24.9, 23.8, 22.0, 18.0, 10.2 ppm; IR (film) 2926, 2853, 1732, 1659, 1615, 1462, 1349, 1202, 1022 cm⁻¹; HRMS (FAB) m/z 698.5117 (698.5108 calcd for C₄₁H₆₈N₃O₆, M-Cl); [α]²⁵_{D}−54.6, [α]²⁵₅₇₇−55.6, [α]²⁵₅₄₆−64.2, [α]²⁵₄₃₅−115, [α9 ²⁵₄₀₅−141 (c 1.25, CHCl₃).

Data for minor pentacycle 139: ¹H NMR (500 MHz, CDCl₃) δ 10.23 (s, 1H), 9.59 (s, 1H), 5.96-5.88 (m, 1H), 5.68-5.64 (m, 1H), 5.48 (br d, J=11.0 Hz, 1H), 5.31 (dq, J=17.2, 1.5 Hz, 1H), 5.23 (dq, J=10.4, 1.3Hz, 1H),4.57(dt, J=5.7, 1.3 Hz, 2H), 4.56 (br s, 1H), 4.16 (t, J=6.7 Hz, 2H), 4.08 (dt, J=11.0, 5.4 Hz, 1 H), 3.97-3.92 (m, 1 H), 3.91-3.88 (m, 1H), 2.57-2.52 (m, 2H), 2.46-2.43 (m, 2H), 2.33 (t, J=7.5 Hz, 2H), 2.30 (d, J=11.1 Hz, 1H), 2.30-2.26 (m, 1H), 2.25-2.17 (m, 2H), 1.92 (dd, J=14.2, 5.8 Hz, 1H), 1.77-1.42 (m, 16H), 1.36 (t, J=12.3 Hz, 1H), 1.33 (d, J=6.7 Hz, 3H), 1.32-1.24 (m, 19H), 0.85 (t, J=7.3 Hz, 3H); ¹³C NMR (125 MHz, CDCl₃) δ 173.5, 167.9, 148.9, 133.3, 132.3, 129.8, 118.1, 84.7, 82.9, 70.7, 70.1, 65.6, 64.9, 54.6, 53.0, 52.5, 37.8, 36.8, 34.2, 31.1, 30.33, 30.31, 29.61, 26.56, 29.49, 29.42, 29.23, 29.15, 29.11, 28.6, 28.4, 25.9, 24.9, 23.9, 21.8, 14.1, 10.3 ppm; IR (film) 2926, 2853, 1732, 1662, 1620 cm⁻¹; LRMS (FAB) m/z 698.51 (698.5108 calcd for C₄₁H₆₈N₃O₆ M-Cl); [α]²⁵_{D}−73.2, [α]²⁵₅₇₇−67.3, [α]²⁵₅₄₆−81.5, [α]²⁵₄₃₅−149, [α]²⁵₄₀₅−184 (c 0.3, CHCl₃),

Carboxylic Acid 25 and 13,14,15-Isocrambescidin 657 (10a).

A solution of the 8-9:1 mixture of 135b and 139 (50 mg, 0.068 mmol), morpholine (24 μl, 0.27 mmol), (Ph₃P)₄Pd (16 mg, 0.014 mmol) and MeCN (5 mL) was maintained at rt for 2 h. Additional morpholine (12 μL, 0.13 mmol) and (Ph₃P)₄Pd (8 mg, 0.007 mmol) were added and the solution was maintained at rt for an additional 2 h. The solution was then partitioned between CHCl₃ (50 mL) and 0.1 M HCl (10 mL). The organic phase was washed with 0.1 M HCl (10 mL), dried (Na₂SO₄), filtered and concentrated to give a brown oil. The brown oil was filtered through a plug of silica gel (99:1 CHCl₃→MeOH→98:2 CHCl₃→MeOH), concentrated and the residue was dissolved in Et₃N (95 μL, 0.68 mmol) and MeOH (7 mL). The resulting solution was maintained at 60° C. for 36 h and then partitioned between CHCl₃ (50 mL) and 0.1 M HCl (8 mL). The organic phase was washed with 0.1 M HCl (8 mL), dried (Na₂SO₄), filtered and concentrated. Purification of the residue by flash chromatography (99:1 CHCl₃→MeOH→98:2 CHCl₃→MeOH→95:5 CHCl₃→MeOH) provided 28 mg (60%) of 141 as a light yellow oil. To insure that the counterion was uniquely chloride, 141 was dissolved in CHCl₃ (50 mL) and washed with 0.1 M HCl (10 mL). The organic phase was dried (Na₂SO₄), filtered and concentrated. Data for 141: ¹H NMR (500 MHz, CDCl₃) δ 10.00 (s, 1H), 9.23 (s, 1H), 5.64 (app t, J=8.1 Hz, 1H), 5.50 (brd, J=11.0Hz, 1H), 4.57 (br s, 1H), 4.16-4.11 (m, 1H), 4.03-3.99 (m, 1H), 4.00-3.97 (m, 1H), 3.92-3.88 (m, 1H), 3.72-3.68 (m, 1H), 3.45 (d, J=3.3 Hz, 1H), 2.59-2.51 (m, 2H), 2.33 (t, J=7.5 Hz, 2H), 2.29-2.24 (m, 1H), 2.24-2.17 (m, 3H), 1.89-1.80 (m, 4H), 1.75-1.45 (m, 10H), 1.39 (t, J=12.3 Hz, 1H), 1.30-1.24 (m, 23H), 1.18 (d, J=6.0 Hz, 3H),0.95 (t, J=7.3 Hz, 3H) ¹³C NMR (125 MHz, CDCl₃) δ 178.4, 167.7, 149.3, 133.6, 129.6, 85.0, 82.9, 70.8, 69.1, 65.3, 52.8, 52.0, 41.7, 38.1, 37.4, 33.9, 32.7, 31.4, 30.2, 29.5, 29.43, 29.37, 29.35, 29.2, 29.1, 29.0, 28.5, 27.9, 25.8, 24.7, 24.0, 22.1, 20.0, 10.2 ppm; IR (film) 3200, 2924, 2852, 1732, 1660, 1621, 1189, 1167, 1027 cm⁻¹; HRMS (FAB) m/z 658.4789, (658.4795 calcd for C₃₈H₆₄N₃O₆, M-Cl); [α]²⁵_{D}□−47.3, [α]²⁵₅₇₇−49.5, [α]²⁵₅₄₆−55.9, [α]²⁵₄₃₅−99.8, [α]²⁵₄₀₅−122 (c 1.2, CHCl₃).

Carboxylic acid 141 was quantitatively converted to the carboxylate inner salt by washing a CHCl₃ (5 mL) solution of the acid (5 mg) with 1 M NaOH (1 mL) and brine (1 mL). The organic layer was dried (Na₂SO₄) and then concentrated to provide 10a as a colorless oil: [α]²⁵_{D}−35.4 (c 0.8, MeOH). Spectroscopic and mass spectrometric data for this sample were consistent with data published for natural 10a.

41,45-Di-tert-butoxycarbonyl-13,14,15-isocrambescidin 800 (145).

A solution of carboxylic acid 141 (30 mg, 0.043 mmol), benzotriazol-1-yloxytris(dimethylamino)phosphonium hexafluorophosphate (28 mg, 0.064 mmol), (S)-hydroxyspermidine derivative 144 (23 mg, 0.064 mmol), Et₃N (29 μL, 0.22 mmol) and CH₂Cl₂ (2.0 mL) was maintained at rt for 1 h and then partitioned between Et₂O (40 mL) and 0.1 M HCl (10 mL). The organic phase was washed with brine (2×10 mL), dried (MgSO₄), filtered and concentrated. Purification ofthis residue by flash chromatography (99:1 CHCl₃→MeOH→97:3 CHCl₃→MeOH) gave 32 mg (71%) of 145 as a colorless foam: ¹H NMR (500 MHz, CD₃OD) δ 5.70 (br t, J=8.8 Hz, 1H), 5.51 (d, J=11.1 Hz, 1H), 4.45 (br s, 1H), 4.19-4.06 (m, 3H), 3.92-3.78 (m, 3H), 3.84 (d J=3.4 Hz, 1H), 3.59-3.23 (m, 3H), 3.19-3.12 (m, 3H), 3.06-2.97 (m, 2H), 2.58 (dd, J=12.8, 2.3 Hz, 1H), 2.45-2.32 (m, 4H), 2.31-2.24 (m, 2H), 2.18-2.12 (m, 1H), 1.96 (dd, J=13.1, 6.1 Hz, 1H), 1.81-1.44(m, 18H), 1.43 (s, 18H), 1.38-1.17(m,23H), 1.16(d, J=6.0Hz,3H), 0.95 (t, J=7.3 Hz, 3H); ¹³C NMR (125 MHz, CD₃OD) (The C38 amide exists on the NMR time scale as an approximate 1:1 mixture of rotamers. Some of the signals of carbons in close proximity to C38, including the carbons of the hydroxyspermidine unit, are doubled. In cases where the rotamers can be distinguished, these signals are listed in parentheses) δ (176.6/176.2),169.8, 158.6, 158.4, 150.2, 134.1, 131.3, 86.7, 84.6, 80.02, 79.95, 72.0, 70.1, (69.0/68.3),66.2, (55.0/53.4),54.8, 54.3, 45.0, 42.6, 39.1, (38.9/38.7),38.1, 36.2, 34.3, 34.1, 33.7, 32.9, 31.0, 30.78, 30.75, 30.67, 30.64, 30.57, 30.54, 30.50, 30.24, 30.16, 29.7, 28.9, 28.8, 28.7, 27.0, (26.7/26.6), 25.0, 22.4, 21.0, 10.8 ppm; IR (film) 3385, 2927, 2854, 1731, 1668(br), 1614, 1449, 1366, 1253, 1167, 1028 cm^{'1}; HRMS(FAB) m/z 1001.7 (1001.7calcd for C₅₅H₉₇N₆O₁₀, M-Cl); [α]²²_{D}□−68.7, [α]²²577−72.9, [α]²²₅₄₆−83.3, [α]²²₄₃₅−148 (c 0.6, CHCl₃).

13,14,15-Isocrambescidin 800 Trihydrochloride (10).

A solution of 145 (30 mg, 0.029 mmol) and 2.9 mL of a 2.0 M solution of HCl in EtOAc was maintained at rt for 30 min and then concentrated. Purification of the residue by reverse phase HPLC (3.5:1 MeOH-0. 1 M NaCl, 5μ Altima C18 column) gave 18 mg (70%) of 13,14,15-Isocrambescidin 800 (10), a light yellow oil, as its trihydrochloride salt: [α]²²_{D}□−67.7, [α]²²₅₇₇−70.9, [α]²²₅₄₆−80.6 (c 0.73, MeOH). NMR data for this sample were consistent with data published for natural 10 and synthetic 10 was indistinguishable from a natural sample of 10 by HPLC comparisons using three eluents.

Preparation of Peracetyl-13,14,15-isocrambescidin 800 Hydrochloride (146).

A solution of 13,14,15-isocrambescidin 800 (10), acetic anhydride (1.2 mL) and pyridine (2.4 mL) was maintained at rt for 20 h then concentrated using a vacuum pump. The residue was dissolved in CHCl₃ (40 mL) and washed sequentially with brine (10 mL), 0.1 M HCl (10 mL) and brine (10 mL). The solution was dried (Na2SO₄), filtered and concentrated. Purification of the residue by flash chromatography (95:5 CHCl₃-MeOH) gave 8 mg (70%) of peracetylisocrambescidin 800 (146). ¹H NMR and ¹³C NMR data for synthetic 146 were in perfect agreement with data reported for this derivative of natural 13,14,15-Isocrambescidin 800 (Berlinck, R. G. S.; Braekman, J. C.; Daloze, D.; Bruno, I.; Riccio, R.; Ferri, S.; Spampinato, S.; Speroni, E. J. Nat. Prod. 1993, 56, 1007-1015).

(4aR,7S)-4-[15-(Allyloxycarbonyl)pentadecyloxycarbonyl]-3-[(4S)-4-t-butyldimethylsiloxypentyl]-7-[(5Z,7S)-2-(1′,3′-dioxan-2′-yl)-7-triisopropyl-siloxy-5-nonenyl]-1,2,4a,5,6,7-hexahydro-1-iminopyrrolo[1,2c]-pyimidine Hydrochloride (133).

¹ H NMR (500 MHz, CDCl₃) δ 9.16 (s, 1H), 6.99 (s, 1H), 5.94-5.87 (m, 1H), 5.42 (br t, J=9.8 Hz, 1H), 5.30 (dq, J=17.2, 1.5 Hz, 1H), 5.27-5.24 (m, 1H), 5.22 (dq, J=10.4, 1.3 Hz, 1H), 4.56 (dt, J=5.6, 1.4Hz, 2H), 4.46-4.41 (m, 2H), 4.24-4.21 (m, 1H), 4.18-4.08 (m, 2H), 4.04-3.89 (m, 5H), 2.82-2.77 (m, 1H), 2.66-2.57 (m, 2H), 2.32 (t, J=7.6 Hz, 2H), 2.27-2.19 (m, 1H), 2.03-1.55 (m, 17H), 1.31-1.24 (m, 25H), 1.12 (d, J=6.0 Hz, 3H), 1.04 (s, 21H), 0.87 (t, J=7.6 Hz, 3H), 0.85 (s, 9H), 0.028 (s, 3H), 0.024 (s, 3H); ¹³C NMR (125 MHz, CDCl₃) δ 173.4, 164.8, 150.7, 149.6, 135.2, 132.2, 126.5, 117.9, 102.8, 100.0, 69.8, 69.7, 68.2, 64.8, 60.0, 59.4, 57.8, 52.2, 44.4, 39.0, 34.2, 33.5, 31.6, 30.8, 30.1, 30.0, 29.54, 29.52, 29.48, 29.41, 29.3, 29.1, 29.0, 28.5, 26.0, 25.8, 24.8, 24.4, 23.5, 22.1, 18.01, 17.99, 12.3, 9.2, −4.6, −4.7 ppm; MS (FAB) m/z 1044.3 (1044.8 calcd for C₅₉H₁₁₀N₃O₈Si₂, M-Cl).

Tetracyclic Guanidine 138b.

¹H NMR (500 MHz, CDCl₃) δ 10.46, (s, 1H), 5.94-5.87 (m, 1H), 5.67-5.64 (m, 1H), 5.48 (br d, J=10.9 Hz, 1H), 5.30 (dq, J=17.2, 1.5 Hz, 1H), 5.22 (dq, J=10.7, 1.3 Hz, 1H), 4.56 (dt, J=5.7, 1.3 Hz, 2H), 4.56 (br s, 1H), 4.20-4.08 (m, 3H), 4.05-3.99 (m, 1H), 3.94-3.91(m, 1H), 3.68 (br s, 1H), 2.99-2.94 (m, 1H), 2.70-2.58 (m, 3H), 2.52-2.45 (m, 1H), 2.39-2.30 (m, 3H), 2.32 (t, J=7.6 Hz, 2H), 2.24-2.19 (m, 1H), 2.04-1.99(m, 1H), 1.91 (dd, J=14.8, 5.2 Hz, 1H), 1.87-1.71 (m, 4H), 1.68-1.58 (m, 5H), 1.57-1.40 (m,4H), 1.38-1.23 (m, 20H), 1.21 (d, J=6.2Hz, 3H), 0.84 (t, J=7.2 Hz, 3H); ¹³C NMR (125 MHz, CDCl₃) δ 173.5, 164.4, 151.4, 149.4, 133.2, 132.3, 129.7, 118.0, 104.0, 85.1, 71.3, 65.9, 65.0, 64.9, 55.6, 51.8, 37.7, 37.0, 36.4, 34.2, 31.7, 31.3, 30.2, 29.6, 29.54, 29.48, 29.40, 29.2, 29.1, 28.6, 26.1, 24.9, 24.4, 24.2, 23.6, 10.4 ppm; IR (film) 3372, 2925, 1737, 1689, 1651, 1613, 1547, 1455, 1341 cm⁻¹; MS(FAB) m/z 698.5106(698.5108calcd for C₄₁H68N₃O₆, MCl).

N-Acylated Hydroxyspermidine Hydrochloride Salt i.

¹H NMR (500 MHz, CD₃OD) δ 4.04 (t, J=6.7 Hz,2H),3.97-3.95 (m, 1H),3.69-3.38 (m,3H),3.32-3.21 (m, 1H),3.14-3.08 (m, 2H), 2.99-2.84 (m, 2H), 2.54-2.39 (m, 2H), 2.01 (s, 3H), 2.00-1.80 (m, 3H), 1.74-1.71 (m, 1H), 1.63-1.58 (m, 4H), 1.33-1.29 (m 22H); ¹³C NMR (125 MHz, CD₃OD) (The amide exists as an approximate 3:1 mixture of rotamers on the NMR time scale. Carbons in close proximity to the amide, including some carbons of the hydroxyspermidine unit, exhibit two signals. In cases where two rotamers were observed, carbon signals of the rotamers are listed in parentheses with the major rotamer listed first) δ (177.5, 176.4), 173.0, (68.6, 69.4), 65.7, (54.8, 53.2), (43.9, 47.8), 38.5, (38.2, 38.3), 34.2, 34.0, (32.9, 33.0), 30.75, 30.72, 30.67, 30.61, 30.5, 30.3, 29.7, 27.0, (26.61, 27.8), (26.54, 26.59), 20.8 ppm.

N-Acylated Hydroxyspermidine Free Base ii.

¹H NMR (500 MHz, CD₃OD) δ 4.04 (t, J=6.7 Hz, 2H), 3.91-3.85 (m, 1H), 3.65-3.32 (m, 3H), 3.27-3.13 (m, 1H), 2.86-2.78 (m, 2H), 2.66-2.60 (m, 2H), 2.45-2.37 (m, 2H), 2.00 (s, 3H), 1.77-1.68 (m, 2H), 1.63-1.51 (m, 6H), 1.32-1.24 (m, 22H); ¹³C NMR (125 MHz, CD₃OD)(the amide exists as an approximate 1:1 mixture of rotamers) δ 176.6, 176.3, 173.1, 70.0, 68.9, 65.7, 55.0, 53.7, 44.5, 40.0, 39.5, 39.4, 38.1, 37.9, 34.3, 34.0, 32.8, 30.8, 30.73, 30.67, 30.63, 30.5, 30.4, 29.7, 27.0, 26.7, 20.8 ppm.

### EXAMPLE VI

This example describes methods for preparing novel pentacyclic intermediates for the preparation of the Crambescidin/Ptilomycalin family of guanidinium alkaloids and congeners. This example further relates to improved chemical synthesis of pentacyclic intermediates for the preparation of the Crambescidin/Ptilomycalin family of guanidinium alkaloids and congeners.

Synthesis

Tethered Biginelli Condensation.

The allyl ester was chosen to protect the C(22) carboxylic acid, since this protecting group can be removed in the presence of a guanidinium salt (Overman, L. E.; et al. J. Am. Chem. Soc. 1995, 117, 2657). Biginelli condensation between compounds 151 (Overman, L. E.; et al. J. Am. Chem. Soc. 1995, 117, 2657) and 152 (Overman, L. E.; et al. 1995, supra), using the previous conditions (Overman, L. E.; et al. 1995, supra), gave only 30-40% of product compound 153 with poor diastereoselectivity (2:1).

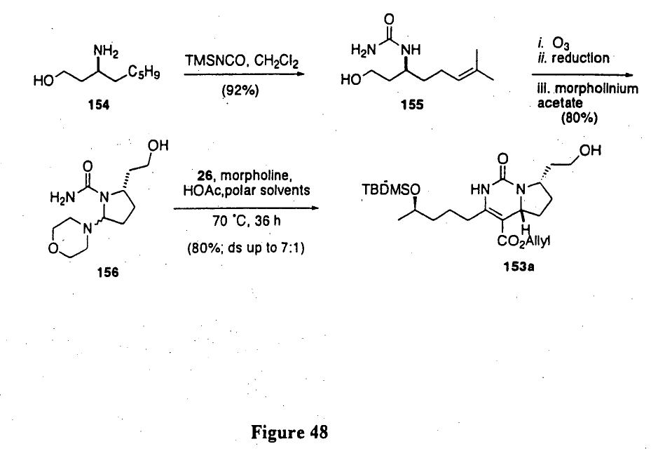

Attention was turned to the integrity of compound 152 (FIG. 48). Urea 155 (FIG. 48) was synthesized in an improved yield by reaction of precursor amine 154 (Overman, L. E.; et al. 1995, supra) with trimethylsilyl isothiocyanate (Vishnyakova, T. P.; et al. Russ. Chem. Rev. 1985, 54, 249) (FIG. 48). When the ozonolysis of compound 155 was quenched with H₂ and 10% Pd/C, followed by filtration and concentration, a solid product was obtained after 1 H under reduced pressure (0.1 mm) at 23° C. This material, gave superior yields in the Biginelli condensation (60%). Diastereoselectivity, however, was still poor (ds=2:1). Extensive optimization of reaction conditions showed that in the non-typical solvent trifluoroethanol, the Biginelli condensation proceeded with good diastereoselectivity (ds=6.5:1 (˜50%) 0.5 M, ds=4:1 (80%) 1.7 M). The use of this solvent to improve efficiency and stereoselectivity in Biginelli condensations was recently reported (McDonald and Overman, J. Org. Chemistry, 1999, 64:1520-1528).

Morpholinium acetate was selected as a catalyst for the Biginelli reaction (Renhowe, P. A. Ph.D. Thesis, University of California, Irvine. 1995). An important discovery regarding the use of morpholinium acetate was made during optimization of the Biginelli reaction. After reductive hydrogenation of the ozonolysis product of compound 152, but prior to filtration and concentration, morpholinium acetate was added to the methanolic solution of compound 152. The solution was then concentrated to give a viscous oil compound 156 that was characterized by HRMS analysis. Subjection of this oil to Biginelli condensation provided compound 153 in a much improved yield of 80%. Moreover, this modification resulted in halving the reaction time to 1.5 days.

Synthesis of Enantiopure Iodide Compound 166.

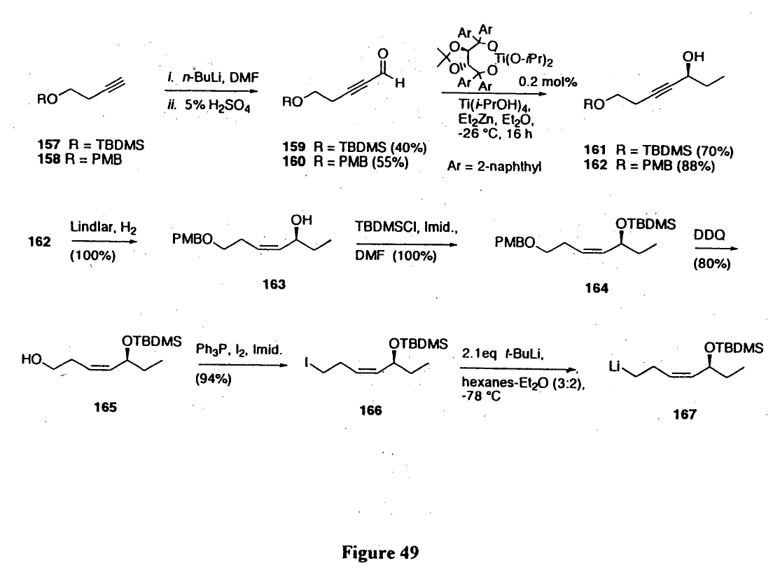

Previous synthesis formed iodide 166, the C(1)-C(7) fragment, in only moderate enantiomeric purity (86% ee) by enantioselective reduction of an ynone precursor (Overman, L. E.; et al. J. Am. Chem. Soc. 1995, 117, 2657; Renhowe, P. A. Ph.D. Thesis, University of California, Irvine. 1995). A shorter synthesis that provides this intermediate in enantiomeric purity is summarized in Scheme VIII (FIG. 34). Diethylzinc addition to aldehydes 159 or 160, which were synthesized from compounds 157 and 158 respectively, in the presence of a TADDOLate catalyst (Weber, B.; Seebach, D. Tetrahedron, 1994, 50, 7473-7484) gave chiral alcohols 161 and 162 in good yield and>99% ee as determined by GLC analysis of the derived Mosher esters (Seebach, D.; et al. Helv. Chim. Acta 1987, 70, 954; Seebach, D.; et al. Chimia 1991, 238; Seebach, D.; et al. Helv. Chim. Acta 1992, 75,438; Seebach, D.; et al. Helv. Chim.Acta 1992, 75, 2171; Seebach,D.; et al. Tetrahedron 1994, 50, 4363; Weber, B.; Seebach, D. Tetrahedron 1994, 50, 7473). Alcohol 161 was found identical, except for optical rotation, to the intermediate employed in our original synthesis (Overman, L. E.; et al. J. Am. Chem. Soc. 1995, 117, 2657). Enantiopure 163 was converted to (S)-(Z)-1-iodo-5-triisopropylsiloxy-3-heptene (Overman, L. E.; et al. J. Am. Chem. Soc. 1995, 117,2657). At this point, the TIPS protecting group was deemed unnecessarily robust and was replaced with TBDMS. PMB protection of the primary alcohol allowed for TBDMS protection of the secondary alcohol, thus delivering primary iodide 166 as summarized in FIG. 49. Organolithium 167 was generated from 166 by lithium-iodide exchange at −78° C. (Dale, J. A.; et al. J. Org. Chem. 1969, 34,2543; Dale, J. A.; Mosher, H. S. J. Am. Chem. Soc. 1973, 95, 512; Ward, D. E.; Rhee, C. K. Tettrahedron Lett. 1991, 32, 7165).

Coupling of the C(1)-C(7) Fragment with the Tricyclic Intermediate.

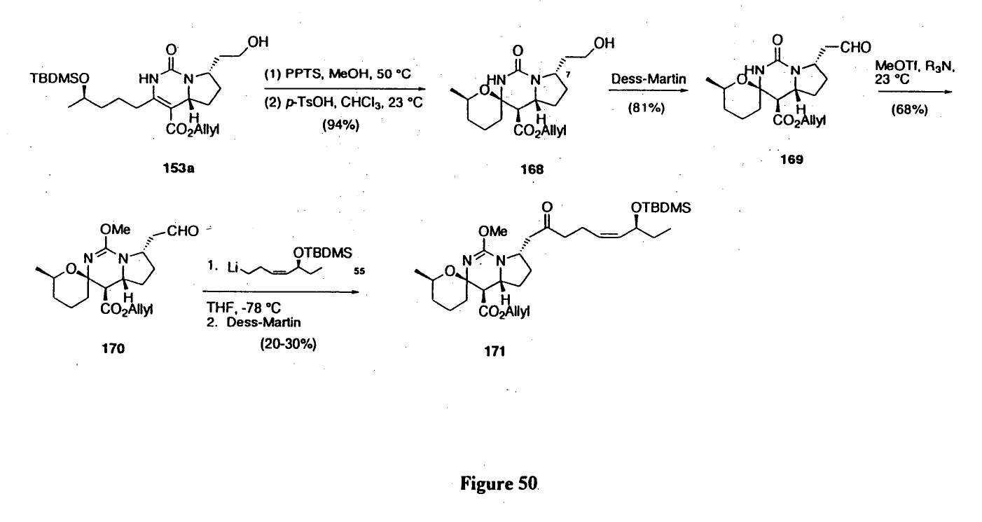

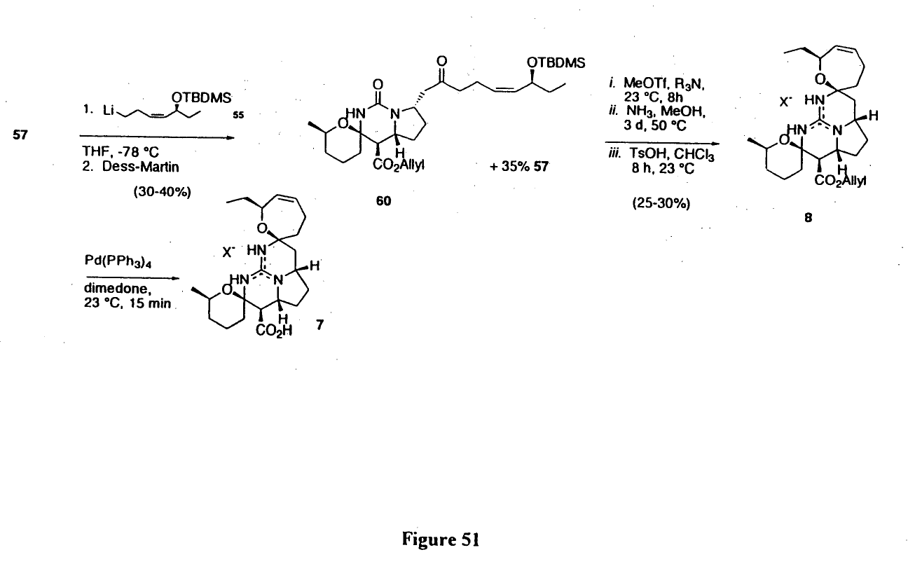

This stage of the synthesis was the least satisfactory of the earlier synthesis due to the lability of the aldehyde related to 169 (Overman, L. E.; et al. J. Am. Chem. Soc. 1995, 117, 2657; Renhowe, P. A. Ph.D. Thesis, University of California, Irvine. 1995). In the present allyl series, when compound 168 was oxidized with the Swern reagent substantial epimerization at C(8) occurred. No epimerization occurred with Dess-Martin periodinane oxidation (FIG. 50). The resulting aldehyde 169 was O-methylated according to established protocol (Overman, L. E.; et al. J. Am. Chem. Soc. 1995, 117, 2657; Renhowe, P. A. Ph.D. Thesis, University of California, Irvine. 1995). However, addition of compound 167 to aldehyde 170, followed by oxidation of the crude epimeric alcohols, provided ketone 171 in low yield (20-30%). When pure compound 170 or 171 was exposed to either commercial or deactivated silica gel for ˜1 h, significant lost of mass (˜30%) was observed. This observation partially explains of the low yields for these two steps. An alternative sequence of events was developed to overcome this difficulty (FIG. 51).

Addition of 2.2 equivalent of compound 167 to aldehyde 169, and subsequent oxidation, yielded ketone 172 in an unoptimized yield of 30-40% (46-51% based on consumed compound 169). Ketone 172 was O-methylated, guanylated, deprotected and cyclized to pentacyclic allyl ester 8 (without intermediate purification) in an unoptimized 25-30% overall yield. This sequence should be optimizable and minimizes the loss of material upon silica gel chromatography.

Synthesis of Pentacyclic Acid 7.

The allyl ester was successfully removed under standard conditions with Pd(0)/dimedone to furnsh pentacyclic acid 7 (FIG. 51).

The following examples are presented to illustrate the present invention and to assist one of ordinary skill in making and using the same. The examples are not intended in any way to otherwise limit the scope of the invention.

General Experimental Details:

All reactions were carried out under an atmosphere of Ar or N₂ and concentrations were performed under reduced pressure with a BU chi rotary evaporator. Tetrahydrofuran (THF), Et₂O and CH₂Cl₂. were degassed with Ar then passed through two 4×36 inch columns of anhydrous neutral A-2 alumina (8×14 mesh; LaRoche Chemicals; activated under a flow of Ar at 350° C. for 3h) to remove water. Toluene was degassed with Ar then passed through one 4×36 inch column of Q-5 reactant (Englehard; activated under a flow of 5% H₂/N₂ at 250° C. for 3 h) to remove O₂ then through one 4×36 inch column of anhydrous neutral A-2 alumina (8×14 mesh; LaRoche Chemicals; activated under a flow of Ar at 350° C. for 3h) to remove water. Triethylamine (Et₃N), pyridine, diisopropylethylamine (i-Pr₂NEt), diisopropylamine, and acetonitrile were distilled from CaH₂ at atmospheric pressure. Indicated molarities of organolithium reagents were established by titration with menthol/fluorene (Posner, G. H.; Lentz, C. M. J. Am. Chem. Soc. 1979, 101, 934). Instrumentation and Chromatography: 300 MHz ¹H and 75 MHz ¹³C spectra were obtained on a BrÚker QE 300 FT NMR; 500 MHz ¹H and 125 MHz ¹³C NMR spectra were obtained on a BrÚker GN 500 FT NMR or BrÚker Ω 500 FT NMR. ¹H NMR chemical shifts are reported as δ values in ppm. Coupling constants are reported in Hz and refer to apparent multiplicities.

Multiplicity is indicated as follows: s (singlet); d (doublet); t (triplet); m (multiplet); app t (apparent t); dd (doublet of doublets) etc. Mass spectra were measured on a MicroMass Analytical 7070E (CI-isobutane) or a MicroMass Auto Spec E (FAB) spectrometer. Infrared spectra were recorded using a Perkin Elmer 1600 FTIR spectrometer. Microanalyses were performed by Atlantic Microlabs, Atlanta, Ga. Optical rotations were measured using a JASCO DIP-360 digital polarimeter. TLC and column chromatography were performed using E. Merck silica gel (43-60 μm) with a loading of approximately 30:1 SiO₂:substrate. 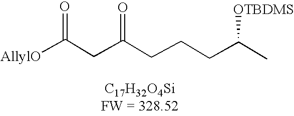
(R)-Allyl-7-(t-butyldimethylsiloxy)-3-oxoocianoate (Compound 151).

Freshly distilled allyl acetoacetate (5.0 mL, 37 mmol) was added dropwise to a 0° C. mixture of hexane-washed NaH (1.73 g, 43 nimol) and dry THF (50 mL). After 10 min, n-butyllithium (14.9 mL of a 2.7 M solution in hexanes) was then added dropwise and the resulting red solution was maintained for an additional 10 min at 0° C. A solution of compound 150 (4.53 g, 14.4 mmol) and dry THF (20 mL) was then added dropwise. After 20 min at 0° C. the reaction mixture was quenched with saturated aqueous NH₄Cl (20 mL). The layers were separated, and the H₂O layer was extracted with Et₂O (2×15mL), and the combined organic layers were washed with brine (15 mL), dried (MgSO₄) and concentrated. Purification of the residue on silica gel (20:1 hexanes-EtOAc) provided 2.84 g (60%) of compound 151 as a colorless oil (9:1 mixture of keto and enol forms by ¹H NMR analysis): ¹H NMR (500 MHz CDCl₃) δ 5.86-5.90 (m 1H) 5.31 (d J=17.0 Hz 1H) 5.23 (d J=10.5 Hz 1H) 4.60 (d J=5.7 Hz 2H) 3.76 (dd J=11.9 6.0 Hz 1H) 3.44 (s 2H) 2.52 (t J=7.3 Hz 2H) 1.63-1.66 (m 1H) 1.55-1.58(m 1H) 1.35-1.40(m 2H) 1.09(d J=6.0 Hz 3H) 0.85 (s 9H) 0.02 (s 6H); ¹³C NMR (125MHz CDCl₃) 202.5 166.9 131.5 118.8 68.2 65.9 49.1 43.1 38.8 25.9 23.7 19.7 18.1−4.4 −4.7ppm;IR(film) 2956 2857 1748 1716 1255 1149 836 775cm⁻¹; [α]²⁵_{D: -}12.8° [α]²⁵₅₇₇: −12.9° [α]²⁵₅₄₆: −14.1° [α]²⁵ ₄₃₅: −23.8° [α]²⁵₄₀₅: −28.10 (c=1.05 CHCl₃). Anal. Calcd for C₁₇H₃₂O₄Si: C 62.15: H 9.82. Found: C 62.42; H 9.93. 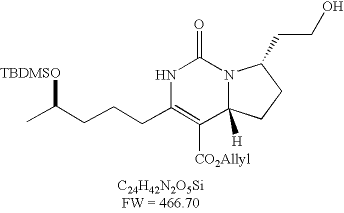
(4aR,7S)-4-(Allyloxycarbonyl)-1,2,4a,5,6,7-hexahydro-7-(2-hydroxyethyl)-3-[(4S)-4-(t-butyldimethylsiloxypentyl)l-1-oxopyrrolo[1,2-c]pyrimidine (153a) and (4aS,7S)-4-(Allyloxycarbonyl)-1,2,4a,5,6,7-hexahydro-7-(2-hydroxyethyl)-3-[(4S)-4-(t-butyldimethylsiloxypentyl)]-1oxopyrrolo[1,2-c]pyrimidine (Compound 153b).

A solution of crude (S)-156 (2.3 g 9 mmol) 151 (2.2 g 6.7 mmol) and trifluoroethanol (4 mL) was heated at 60° C. for 2 d. The reaction mixture was quenched by pouring into Et₂O (50 mL) washing with saturated aqueous NH₄Cl (2×10 mL) and brine (10 mL). The organic layer was dried (MgSO₄) concentrated and purified on silica gel (1:1 hexanes-EtOAc) to yield 2.01 g (64%) of the desired cis-Biginelli product 153a and 0.51 g (16%) of the trans-Biginelli product 153b.

Compound 153a:

¹H NMR (500 MHz CDCl₃) δ 8.26 (s, 1H) 5.89-5.97 (m 1H) 5.30 (dd J=16.7 1.2Hz 1H) 3.22(dd J=10.4 1.0Hz 1H) 4.60(ddd J=22.6 13.1 5.9Hz 2H) 4.26(dd J=11.3 4.9Hz 1H) 4.10-4.14(m 1H) 3.74-3.80(m 1H) 3.40-3.67(m 2H) 2.58 (t J=7.5 Hz 2H) 2.47-2.52 (m 1H) 2.02-2.11 (m 1H) 1.84 (m 1H) 1.60-1.77 (m 4H) 1.36-1.53 (m 3H) 1.10 (d J=6.0 Hz 3H) 0.85 (s 9H) 0.02 (s 3H) 0.01 (s 3H); ¹³C NMR(125 MHz CDCl₃) δ 165.2 155.0 152.9 132.3 118.5 102.2 68.6 64.8 59.0 58.3 52.2 39.4 38.9 31.1 30.6 29.8 25.8.25.0 23.7 18.1 −4.4−4.7ppm;IR(film)3450 3225 3095 2954 1682 1626 1431 1111 835 776cm¹; [α]²⁵_{D}: −28.6° [α]²⁵₅₇₇: −30.0° [α]¹⁵₅₄₆: −36.80 [α]²⁵₄₃₅: −97.7° [α]²⁵₄₀₅: −138° (c=2.20 CHCl₃). Anal. Calcd for C₂₄H₄₂N₂O₅Si: C, 61.77; H, 9.07; N, 6.00. Found: C, 61.66; H, 9.15; N, 5.92.

Compound 153b:

¹H NMR (500 MHz CDCl₃) δ 8.52 (s 1H) 5.87-5.94 (m 1H) 5.28 (d J=17.5 Hz 1H) 5.21 (d J=10.4 Hz 1H) 4.60 (ddd J=13.4 7.5 6.0 Hz 2H) 4.39-4.45 (m 1H) 4.34 (dd J=10.5 4.6 Hz 1H) 3.77 (dd J=11.5 5.7 Hz 1H) 3.56-3.66 (m 2H) 2.66-2.71 (m 1H) 2.49-2.54 (m 1H) 2.42-2.46 (m 1H) 2.08 (dd J=20.7 8.7 Hz 1H) 1.76-1.81 (m 1H) 1.62-1.67 (m 1H) 1.37-1.54 (m 6H) 1.10 (d J=6.04 Hz 3H) 0.8 (s 9H) 0.02 (s 3H)0.01 (s 3H); ¹³C NMR(125 Mz CDCl₃) 165.3 153.2 150.1 132.4 118.2 98.7 68.5 64.7 58.9 57.3 53.7 38.9 38.3 35.1 31.3 28.2 25.9 24.5 23.6 18.1−4.5-4.7ppm; IR (film) 3442 3256 3100 2930 2897 1708 1668 1634 1463 1236 1082 736cm⁻¹; [α]²⁵_{D}: −26.3° [α]²⁵₅₇₇: −26.8, [α]²⁵₅₄₆: −29.3° [α]²⁵₄₃₅: −54.7° [α]²⁵₄₀₅: −122° (c=2.30 CHCl₃). Anal. Calcd for C₂₄H₄₂N₂O₅Si: C, 61.77;H, 9.07;N, 6.00. Found: C, 61.75;H, 9.10; N, 5.96. 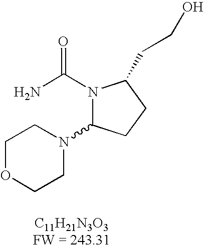
Compound (156).

Ozone was bubbled through a solution of compound 155 (1.74 g 9 mmol) and MeOH (50 mL) at −78° C. until the solution was saturated (blue color appeared and persisted for ˜10 min). Nitrogen was then bubbled through the solution to dissipate the excess ozone. 10% ,d/C (0.6 g) was added to the colorless solution and the reaction mixture was maintained at −78° C. under 1 at of H₂. After 30 min the cooling bath was removed morpholinium acetate (2.0 g 13 mmol) was added and the reaction mixture was allowed to warm to 23° C. After 4 h the reaction mixture was dried (MgSO₄) filtered and the filtrate was concentrated. The resulting residue was diluted with trifluoroethanol (30 mL) and concentrated to give a yellow oil which was used without further purification: MS (CI) m/e cald for C₁₁H₂₁N₃O₃ 243.1583 found 243.1588 (M). 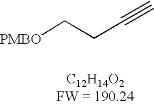
1-(4-Methyoxybenzyloxy)-3-butyne (Compound 158).`

According to established procedures (Takaku H. et al.; Tetrahedron Lett. 1983 24 5363; Nakajima N.; et al. Tetrahedron Lett. 1988 29 4139) TfOH (1.6 mL 18 mmol) was added dropwise to a 0° C. solution of MBOC(═NH)CCl₃ (169.3 g 0.6 mol) 3-butyn-1-ol (67 g 0.66 mol) dry EtO (600 mL). After 30 min the reaction mixture was quenched by the addition of saturated aqueous NaHCO₃ (100 mL) the layers were separated the aqueous layer was extracted with Et₂O (50 mL) and the combined organic layers are washed with brine (50 mL) dried (MgSO₄) and concentrated. The resulting residue was diluted with hexanes (300 mL) filtered through a plug of silica gel concentrated and stirred under vacuum (0.1 mm Hg) at 50° C. for 12 h yielding 158 (˜100%) which is used without further purification. ¹H NMR (500 MHz CDCl₃) δ 7.28 (d J=8.4 Hz 2H) 6.89 (d J=8.4 Hz 2H) 4.49 (s 2H) 3.80 (s 3H) 3.58 (t J=7.0Hz 2H) 2.49(dt J=7.0 2.7Hz 2H) 2.00(t J=2.6Hz 1H); ¹³C NMR(125MHz CDCl₃) δ 159.2 130.0 129.3 113.7 81.3 72.5 69.2 67.8 55.2 19.8ppm; IR(film)3292 3001 2936 2863 1614 1514 823 cm⁻¹; Anal. Calcd for C₁₂H14O₂: C, 75.76; H, 7.42; Found: C, 75.60; H, 7.49. 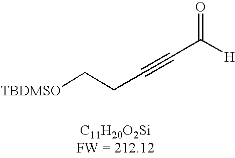
5-(t-Butyldimethylsiloxy)-2-pentynal (Compound 159).

A hexane solution of n-BuLi (2.5 M 4.8 mL) was added to a −78° C. solution of compound 157 (2.0 g 10.9 mmol) in dry THF (20 mL). After 10 min the reaction mixture was placed into an ice bath and dry DMF (5 mL) in THF (20 mL) was added. After 30 min at 0° C. the reaction mixture was quenched by pouring into a vigorously stirred solution of 5% H₂SO₄ (20 mL). After 1 h the layers were separated the H₂O layer was extracted with Et₂O (3×15 mL) and the combined organic layers were washed with saturated aqueous NaHCO₃ (1×15 mL) and brine (1×15 mL) dried (MgSO₄) and concentrated. ,urification of the residue on silica gel (4:1 hexanes-EtOAc) provided 0.921 g (55%) of compound 159 as a slightly yellow oil: ¹H NMR (500 MHz CDCl₃) δ 9.17 (s 1H) 3.79 (t J=6.7 Hz 2H) 2.62 (t J=6.7 Hz 2H) 0.9 (s 9H) 0.1 (s 6H); C NMR (125MHz CDCl₃) δ 177.0 96.2 82.3 60.6 25.8 23.5 18.3 −5.3 −5.4ppm; IR(film)2930 2205 1671 1111 cm⁻¹; HRMS (CI isobutane) m/e calcd for C₁₁H₂₀O₂Si 212.1232 found 197.0998 (M−CH₃). 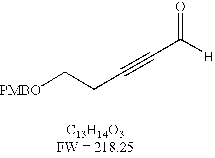
5-(4-Methoxybenzyloxy)-2-pentynal (compound 160).

A hexane solution of n-BuLi (2.5 M 9.34 mL) was added to a −78° C. solution of compound 158 (4.04 g 21.2 mmol) in dry THF (100 mL). After 10 min the reaction mixture was placed into an ice bath and dry DMF (10 mL) in THF (100 mL) was added. After 30 min at 0° C. the reaction mixture was quenched by pouring into a vigorously stirred solution of 5% aqueous H₂SO₄ (100 mL). After 1 h the layers were separated the H₂O layer was extracted with EtO (3×30 mL) and the combined organic layers were washed with saturated aqueous NaHCO₃ (1×30 mL) and brine (1×30 mL) dried (MgSO₄) and concentrated. ,urification of the residue on silica gel (4:1 hexanes-EtOAc) provided 2.55 g (55%) of compound 160 as a slightly yellow oil: ¹H NMR (500 MHz CDCl₃) δ 9.16 (s 1H) 7.26 (d J=8.5 Hz 2H) 6.88 (d J=8.6 Hz 2H) 4.48 (s 2H) 3.79 (s 3H) 3.61 (t J=6.7 Hz 2H) 2.69 (t J=6.7 Hz 2H); ¹³C NMR (125 MHz CDCl₃) δ 177.0 159.2 129.6 129.3 113.8 95.7 81.9 72.7 66.5 55.2 20.6ppm; IR (film) 3002 2865 2205 1668 1514 824 cm⁻¹; Anal. Calcd for C₁₃H₁₄O₃: C, 71.54; H, 6.47. Found: C, 71.42; H, 6.54. 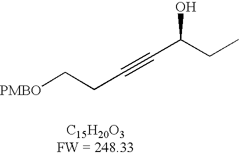
(5S)-Hydroxy-1-(4-methoxybenzyloxy)-3-heptyne (compound 162).

According to the general procedure of Seebach (Ti(Oi-,r)₄ (0.50 mL 1.68 mmol) was added to a 23° C. solution of (4R 5R)-2 2-Dimethyl-α,α,α [symbol] ,α [symbol] -τetra(naphth-2-yl)-1 3-dioxolan-4 5-dimethanol (1.12 g 1.67 rnnol) and dry toluene (15 mL). After 3 h solvent was removed under reduced pressure (0.1 mm). The resulting residue was dissolved in dry Et₂O (33 mL) and the reaction vessel was cooled to −26° C. whereupon Ti(Oi-,r)₄ (3.0 mL 10 mmol) compound 160 (1.83 g 8.37 mmol) and Et₂Zn (9.1 mL of a 1.1 M solution in toluene)were added. After 18 h at −26° C. the reaction mixture was quenched with saturated aqueous NH₄Cl (1 mL) dried (MgSO₄) filtered through Celite®, concentrated and the resulting residue was purified on silica gel (4:1 hexanes-EtOAc) to provide 1.833 g(88%)□ of compound 162 as a colorless oil: ¹H NMR (500 MHz CDCl₃) δ 7.25 (d J=8.4 Hz 2H) 6.86 (d J=8.4 Hz 2H) 4.46 (s 2H) 4.26 (t J=6.4 Hz 1H) 3.78 (s 3H) 3.53 (t J=7.0 Hz 2H) 2.58 (s 1H) 2.49(dt J=7.0 1.5Hz 2H) 1.66(m 2H) 0.97(t J=7.4Hz 3H); ¹³C NMR (125 MHz CDCl¹³) δ 159.1 129.9 129.2 113.7 82.3 81.7 72.4 67.9 63.5 55.1 30.9 19.9 9.4 ppm; IR (film) 3418 2965 1613 1514 1249 823 733 cm⁻¹; [α]²⁵ _{D}: −3.2° [α]²⁵₅₇₇: −3.6° [α]²⁵₅₄₅: −4.0° [α]²⁵₄₃₅: −6.5° [α]²⁵₄₀₅: −7.7° (c=2.35 CHCl₃). Anal. Calcd for C₁₅H₂₀O₃: C, 72.55; H, 8.12. Found: C, 72.26; H, 8.14.

Following the general procedure of Ward (Ward D. E.; Rhee C. K. Tetrahedron Lett. 1991 32 7165) compound 162 (23 mg) was treated with (R)-α-methoxy-α-(triflouromethyl)phenylacetic acid chloride [(R)-MT,ACl] to give the corresponding (R)-MT,A ester. Capillary GC analysis [150° C. to 200° C./2.0° C. min⁻¹² t_{R} 162-(R)-MT,A=21.13 min t_{R} ent-162-(R)-MT,A 20.69 min] showed a ratio for 99.7:0.3 of 162-(R)-MT,A and ent-162-(R)-MT,A. 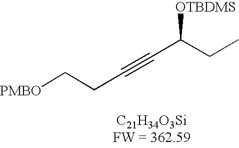
(5S)-(t-Butyldimethylsiloxyl)-1-(4-methoxybenzyloxy)-3-heptyne.

TBSCl (1.08 g 7.2 mmol) was added in portions over 15 min to a 23° C. solution of imidazole (0.53 g 7.8 mmol) compound 162 (1.48 g 6 mmol) and dry DMF (5 mL). After standing at 23° C. for 2 h the solution was poured into 20 mL H₂O and extracted with Et₂O (4×20 mL). The combined organic layers were washed with brine (20 mL) dried (MgSO₄) and concentrated. The crude oil was placed under vacuum (0.1 mm) overnight to provide 2.16 g (100%) of the desired product as a colorless oil which was used without further purification: ¹H NMR (500 MHz CDCl₃) δ 7.28 (d J=8.5 Hz 2H) 6.89 (d J=8.5 Hz 2H) 4.48 (s 2H) 4.28 (dt J=6.2 1.7 Hz 1H) 3.80 (d J=1.6 Hz 3H) 3.56 (dt J=7.2 1.52 Hz 2H) 2.51 (dt J=7.2 1.7 Hz 2H) 1.66 (appt J=7.0 Hz 2H) 0.96 (dt J=7.3 1.3 Hz 3H) 0.91 (d J=1.4 Hz 9H) 0.13 (d J=1.4Hz 3H) 0.11 (d J=1.4Hz 3H); ¹³C NMR(125 MHz CDCl₃) δ 159.2 130.2 129.2 113.73 82.8 80.9 72.6 68.3 64.4 55.2 31.9 25.8 20.1 18.3 9.2 −4.5 −5.0 ppm; IR (film)2930 1614 1514 1249 1099 837cm^{31 1};[α]²⁵_{D}: −34.5° [α]²⁵₅₇₇: −35.0° [α]²⁵₅₄₆: −40.9° [α]²⁵₄₃₅: −69.5° [α]²⁵₄₀₅: −83.5° (c=5.35 CHCl₃). Anal. Calcd for C₂₁H₃₄O₃Si: C, 69.56; H, 9.45. Found: C, 69.49; H, 9.50. 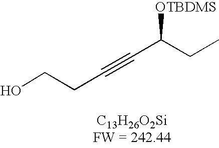
(S)-(5)-(t-Butyldimethylsiloxy)-3-heptynol.

A solution of (5S)-(t-butyldimethylsiloxyl)-1-(4-methoxybenzyloxy)-3-heptyne (0.17 g 0.46 mmol) DDQ (0.16 g 0.68 mmol) and 20:1 CH₂Cl₂−H₂O (3 mL) was maintained at 23° C. for 2 h. The reaction mixture was quenched by pouring into Et₂O (25 mL) and washing with saturated aqueous NaHCO₃ (2×5 mL) followed by brine (5 mL). The organic. layer was dried (MgSO₄) concentrated and the resulting residue was purified on silica gel (4:1 hexanes-EtOAc) to provide 88 mg (80%)□of the desired product as a colorless oil: ¹H NMR (500 MHz CDCl₃) δ 4.26 (t J=6.3 Hz 1H) 3.69 (t J=6.6 Hz 2H) 2.47 (dt J=6.3 1.6 Hz 2H) 1.98 (s 1H) 1.61-1.67 (m 2H) 0.94 (t J=7.4 Hz 3H) 0.9 (s 9H) 0.11 (s 3H) 0.10(s 3H); ¹³C NMR (75 MHz CDCl₃) δ 84.0 80.6 64.4 61.1 31.9 25.8 23.1 18.3 9.7 −4.6 −5.0 ppm; IR (film) 3388 2958 2858 1472 1256 1059 cm⁻¹; HRMS (EI-GCMS) m/e calcd for C₁₂H₂₆O₂Si 242.1701 found 242.1655 (M); [α]²⁵_{D}: −46.0° [α]²⁵₅₇₇: −48.1° [α]²⁵₅₄₆: −54.5° [α]²⁵₄₃₅: −93.2° [α]²⁵₄₀₅: −111.5° (c=1.4 CHCl₃). 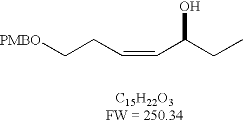
(3Z,5S)-1-(4-Methyloxybenzyloxy)-3-hepten-5ol (compound 163).

A mixture of compound 162 (13.8 g 55.6 mmol) freshly distilled quinoline (138 mL 1.20 mmol) Lindlar's catalyst (,d/CaCO₃ poisoned with ,bO 1.29 g) and dry 3:1 hexanes -EtOAc (138 mL) was maintained at 23° C. under 1 atm H₂ for 3 d. This mixture was then filtered through a plug of Celite the plug was washed with 3:1 hexanes-EtOAc (400 mL) and the elutent was concentrated to yield 13.9 g (100%) of compound 163 which was used without further purification: ¹H NMR (300 MHz CDCl₃) δ 7.24 (d J=8.4 2H) 6.86 (d J=8.5 2H) 5.51-5.55 (m 2H) 4.44 (s 2H) 4.30 (appq J=7.0 1H) 3.79 (s 3H) 3.34-3.53 (m 2H) 2.25-2.55 (m 2H) 2.21-2.24 (m 1H) 1.41-1.64 (m 2H) 0.86 (t J=7.4 3H); ¹³C NMR (75 MHz CDCl₃) δ 159.1 135.2 129.8 129.3 128.6 113.7 72.7 68.61 68.0 55.2 29.7 28.3 9.7 ppm; IR (film) 3421 3007 2961 2860 1613 1514 1249 1094 821cm⁻¹;[α]²⁵_{D}=−17.9° [α]²⁵₅₇₇=−17.8° [α]²⁵₅₄₆=−20.6° [α]²⁵₄₃₅ =−35.0° [α]²⁵₄₀₅=−42.6° (c=2.2 CDCl₃). Anal. Calcd for C₁₅H₂₂O₃: C, 71.97; H, 8.86. Found: C, 71.97; H, 8.90. 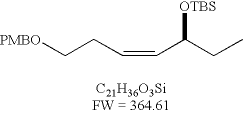
(3Z,5S)-4-(t-Butyldimethylsiloxy)-1-(4-methoxybenzyloxy)-3-heptene (compound 164).

TBSCl (0. 51 g 3.4 mmol) was added in portions over 15 min to a solution of imidazole (0.48 g 7.0 mmol) compound 163 (0.7 g 2.8 mmol) and dry DMF (1.4 mL) at 23° C. After standing at 23° C. for 2 h the solution was poured into 20 mL H2O and extracted with Et₂O (4×20 mL). The combined organic layers were washed with brine (20 mL) dried (MgSO₄) and concentrated. The crude oil was placed under vacuum (0.1 mm Hg) overnight to provide 1.02 g (100%) of 164 as a colorless oil: ¹H NMR (300 MHz CDCl₃) δ 7.26 (d J=8.3 Hz 2H) 6.88 (d J=8.5 Hz 2H) 5.34-5.46 (m 2H) 4.45 (s 2H) 4.38 (appq 1H) 3.80(s 3H) 3.45 (t J=7.0 Hz 2H) 2.35 (m 2H) 1.38-1.56 (m 2H) 0.84-0.88 (m 12H) 0.05 (s 3H) 0.02 (s 3H) ; ¹³C NMR (75 MHz CDCl₃) 159.1 135.8 130.4 129.2 124.6 113.7 72.6 70.2 69.4 55.2 31.3 28.6 25.8 18.2 9.8 −4.4 −4.8ppm; IR (film) 2967 2856 1616 1514 1464 1250 1098 836 cm⁻¹; [α]²⁵_{D}=14.4° [α]²⁵₅₇₇=15.7° [α]²⁵₅₄₆=18.40° [α]²⁵₄₃₅=33.5° [α]²⁵₄₀₅=42.4° (c=1.98 CHCl₃). Anal. Calcd for C₂₁H₃₆O₃Si: C, 69.18; H, 9.95. Found: C, 69.30; H, 10.03. 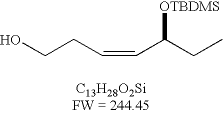
(S)-(Z)-5-(t-Butyldimethylsiloxy)-3-heptenol (compound 165).

A solution of compound 164 (0.17 g 0.47 mmol) DDQ (0.16 g 0.68 mmol) and 20:1 CH₂Cl₂—H₂O (3 mL) was maintained at 23° C. for 2 h. The reaction mixture was quenched by pouring into EtO (25 mL) and washing with saturated aqueous NaHCO₃ (2×5 mL) followed by brine (5 mL). The organic layer was dried (MgSO₄) concentrated and the resulting residue was purified on silica gel (4:1 hexanes-EtOAc) to provide 92 mg (80%)□of the desired product as a colorless oil: ¹HMR (500 MHz CDCl₃) δ 5.46-5.50 (m 1H) 5.30-5.36 (m 1H) 4.31 (dd J=14.6 6.7 Hz 1H) 3.63 (dt J=6.6 2.1 Hz 2H) 2.29-2.34(m 2H) 1.94 (s 1H) 1.50-1.60(m 1H) 1.37-1.24 (m 1H) 0.86 (app s 12H) 0.03 (s 3H) 0.01 (s 3H); ¹³C NMR (125 MHz C₆D₆) δ 136.6 125.2 70.4 62.1 31.8 31.7 26.1 18.4 10.0 −4.0 −4.1ppm; IR (film) 3354 3014 2958 1460 1253 1050 cm⁻¹; [α]²⁵_{D}: 20.8° [α]²⁵₅₇₇: 21.3° [α]²⁵₅₄₆: 25.2° [α]²⁵₄₃₅: 47.4° [α]²⁵₄₀₅: 59.7° (c=2.30 CDCl₃). Anal. Calcd for C₁₃H₂₈O₂Si: C, 63.88; H, 11.55. Found: C, 63.82;H, 11.53. 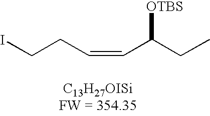
(S)-(Z)-1-Iodo-5-(tert-butyldimethylsiloxy)-3-heptene (compound 166).

Following the general procedure of Corey (Singh S. N.; et al. J. Am. Chem. Soc. 1987 109 6187; Garegg, .J.; Samuelsson B. J. Chem. Soc., Perkin Trans. 1 1980 2866) iodine (2.09 g 8.24 mmol) was added in portions over 15 min to a 0° C. solution of compound 165 (1.83 g 7.49 mmol), h₃ (2.03 g 9.0 mmol) imidazole (0.61 g 8.99 imnol) and Et₂O-MeCN (3:1 40 mL) and then allowed to warm to 23° C. After 1.5 h the solution was diluted with 1:1 hexanes-EtOAc (200 mL) then filtered through basic alumina (activity-IV) and concentrated. The resulting mixture was flushed though a plug of siiica gel (9:1 hexane-Et₂O) to yield 2.5 g (94%) of the desired product as a colorless oil which was used without any further purification: ¹HNMR (300 MHz CDCl₃) δ 5.46-5.52 (m 1H) 5.23-5.32 (m 1H) 4.25 (dd J=14.4 6.6 Hz 1H) 3.11-3.16(m 2H) 2.60-2.68(m 2H) 1.37-1.60(m 2H) 0.84-0.89(m 12H); ¹³C NMR (75 MHz CDCl₃) 136.2 127.0 70.2 32.0 31.3 25.8 18.2 9.8 4.6 −4.3 −4.7 ppm; IR (film) 3612 2957 2530 2857 1699 1650 1252cm⁻¹; [α]²⁵_{D}=21.9° [α]²⁵₅₇₇=22.6° [α]²⁵₅₄₆=26.2° [α]²⁵₄₃₅=49.5° [α]²⁵₄₀₅=62.2° (c=2.00 CHCl₃). Anal. Calcd. for C₁₃H₂₇OISi: C, 44.07; H, 7.68. Found: C, 44.24; H, 7.64. 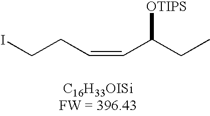
(S)-(Z)-1-Iodo-5-(triisopropylsiloxy)-3-hentene.

Following the general procedure of Corey (Singh S. N.; et al. J. Am. Chem. Soc. 1987 109 6187; Garegg J.; Samuelsson B. J. Chem. Soc., Perkin Trans. 1 1980 2866) iodine (0.80 g 3.5 mmol) was added in portions over 15 min to a 0° C. solution of (S)-(Z)-5-(triisopropylsiloxy)-3-heptenol (0.900 g 3.14 mmol) ,,h₃ (0.78 g 3.5 mmol) imidazole (0.24 g 3.5 mmol) and Et₂O-MeCN (3:1 5 mL) and then allowed to warm to 23° C. After 1.5 h the solution was diluted with 1:1 hexanes-EtOAc (50 mL) then filtered through basic alumina (activity-IV) and concentrated. The resulting mixture was flushed though a plug of silica gel (9:1 hexane-EtO) to yield 1.29 g (97%) of the desired product as a colorless oil which was used without any further purification: ¹H NMR (500 MHz CDCl₃) δ 5.49-5.53 (m 1H) 5.28-5.32 (m 1H) 4.41 (dd J=7.1 5.9 Hz 1H) 3.10-3.14 (m 2H) 2.59-2.66 (m 2H) 1.58-1.62 (m 1H) 1.48-1.52 (m 1H) 1.05 (s 21H) 0.86(t J=7.4Hz 3H); ¹³CNMR(125 MHz CDC₃) 136.2 126.9 70.0 32.2 31.6 18.1 12.3 9.3 4.4 ppm; IR (film) 3012 2942 1464 1105 883 cm⁻¹; [α]²⁵_{D}: 22.8° [α]²⁵₅₇₇: 24.4° [α]²⁵₅₄₆: 23.7° [α]²⁵₄₃₅: 53.1° [α]²⁵₄₀₅: 65.8° (c=1.2 CHCl₃). Anal. Calcd for C₁₆H₃₃OSiI: C, 48.48; H, 8.39. Found: C, 48.63;.H, 8.49. 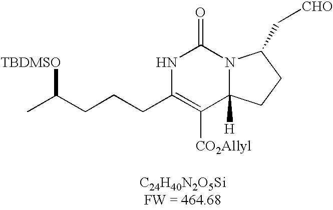
(4aR,7S)-4-(Allyloxycarbonyl)-1,2,4a,5,6,7-hexahydro-3-[(4S)-4-(t-butyldimethylsiloxypentyl)]-1-oxo-7-(2-oxyethyl)pyrrolo[1,2-c]pyrimidine.

Dess-Martin periodinane (Dess D. B.; Martin J. C. J. Org. Chem., 1983 48 415 5) (0.50 g 1.2 mmol) was added to a 23° C. solution of compound 153a (0.46 g 1 mmol) and CH₂Cl₂ (10 mL). After 1 h the reaction mixture was poured into Et₂O (50 mL) and washed with saturated aqueous Na₂S₂O₃ (2×10 mL) 1 N NaOH (2×10 mL) and brine (10 mL). The organic layer was dried (MgSO₄) concentrated and purified on silica gel (1:1 hexanes-EtOAc) to yield 0.404 g (81%) of desired product as a colorless oil: ¹HNMR (500 MHz CDCl₃) δ 9.73 (s 1H) 8.21 (s 1H) 5.88-5.95 (m 1H) 5.30 (dd J=16.7 1.2 Hz 1H) 5.22 (d J=10.4 Hz 1H) 4.60 (ddd J=22.6 13.1 5.9Hz 2H) 4.29-4.35(m 2H) 3.75-3.78(m 1H) 3.15(dd J=16.7 3.8Hz 1H) 2.57-2.62(m 1H) 2.52-2.56(m 2H) 2.44-2.51 (m 1H) 2.11-2.14(m 1H) 1.62-1.73 (m 2H) 1.57-1.61 (m 1H) 1.53-1.56 (m 1H) 1.39-1.45 (m 2H) 1.09 (d J=6.0 Hz 3H) 0.85 (s 9H) 0.02 (s 3H) 0.01 (s 3H); ¹³C NMR (75 MHz CDCl₃) δ 200.0 165.3 152.6 152.1 132.3 118.5 101.0 68.4 64.8 58.2 51.0 48.4 39.1 31.1 30.6 29.6 25.8 24.6 23.7 18.1 −4.5 −4.7ppm; IR (film) 3218 3096 2955 2856 2730. 1722.1679 1630 1439 1253 836 775 734 cm⁻¹; [α]²⁵_{D}: −35.4° [α]²⁵₅₇₇: −35.5° [α]²⁵₅₄₆: −44.6° [α]²⁵₄₂₅: −61.6° [α]²⁵₄₃₅: 19.8° (c=1.85 CHCl₃). Anal. Calcd for C₂₄H₄₀N₂O₅Si: C, 62.04; H, 8.68; N, 6.03. Found: C, 61.75; H, 8.68; N, 6.00. 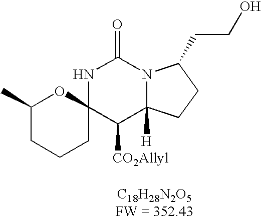
(3R,4R,4aR,6′R,7S)-4-(Allyloxycarbonyl)-1,2,4a,5,6,7-hexahydro-7-(2-hydroxyethyl)-1-oxopyrrolo[1,2-c]pyrimidine-3-spiro-6′-(2′-methyl)-3′,4′,5′,6′-tetrahydro-2H-pyran (compound 168). A solution of compound 153a (0.486 g, 1.04 mmol), PPTS (0.262 g, 1.04 mmol), and MeOH (20 mL) was heated at 50° C. for 5 h. The resulting solution was concentrated, flushed through a plug of silica gel (20:1 EtOAc-MeOH), and concentrated, The resulting residue was dissolved in a solution of CHCl₃ andp-TsOH (45 mg, 0.24 mmol), which was maintained at 23° C. for 1 h, then poured into Et₂O (60 mL). The solution was washed with saturated aqueous NaHCO₃ (2×10 mL), brine (10 mL), dried (MgSO₄), and concentrated to yield 0.345 g 168 (94%) as a slightly yellow oil, which was used without further purification: ¹H NMR (500 MHz, CDCl₃) δ 6.26 (s, 1 H), 5.84-5.92 (m, 1 H), 5.32 (d, J=17.4Hz, 1H), 5.22 (d, J=10.4 Hz, 1H), 4.62 (ddd, J=21.1, 12.5, 6.2 Hz, 2H), 4.33(s, 1H), 4.13-4.33 (m, 1H), 4.02 (dt, J=11.1, 5.0 Hz, 1H), 3.77-3.80 (m, 1H) 3.53-3.58 (m, 2H), 2.32 (d, J=11.1 Hz, 1 H), 2.13-2.23(m, 2H), 1.98-2.03 (m, 1 H), 1.52-1.74 (m, 8H), 1.05-1.09 (m, 1H), 1.02 (d, J=6.1 Hz, 3H); ¹³C NMR (125 MHz, CDCl₃) δ 6 168.4, 154.6, 131.7, 118.5, 82.5, 66.2, 65.4, 59.2, 55.3, 54.4, 53.6, 39.7, 32.4, 32.2, 30.3, 29.4, 21.7, 18.6 ppm; IR (film) 3297, 3084, 2934, 1731, 1659, 1633, 1480, 1012, 733 cm⁻¹; [α]²⁵_{D}: 139°, [α]²⁵₅₇₇: 145°, [α]²⁵₅₄₆: 166°, [α]²⁵₄₃₅: 285°, [α]²⁵₄₀₅: 345°, (c=2.25,CHCl₃). Anal. Calcd for C₁₈H₂₈N₂O₅: C, 61.34; H, 8.00; N, 7.95. Found: C, 61.08; H, 8.08; N, 7.78. 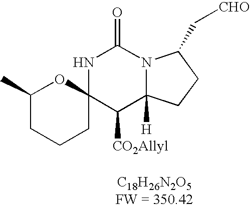
(3R,4R,4aR,6′R,7S)-4-(Allyloxycarbonyl)-1,2,4a,5,6,7-hexahydro-1-oxo-7-(2-oxyethyl)-pyrrolo[1,2-c]pyrimidine-3-spiro-6′-(2′-methyl)-3′,4′,5′,6′-tetrahydro-2H-pyran (compound 169).

Dess-Martin periodinane (Dess, D. b.; Martin, J. C. J. Org. Chem. 1983, 48, 4155) (0.72 g, 1.7 mmol) was added to a 23° C. solution of compound 168 (0.500 g, 1.42 mmol) and CH₂Cl₂ (35 mL). After 1 H the reaction mixture was poured into Et₂O (100 mL) and washed with saturated aqueous Na₂O₃ (2×10 mL), 1 N NaOH (2×20 mL), brine (20 mL). The organic layer was dried (MgSO₄), concentrated, and purified on silica gel (EtOAc; 20:1 EtOAc-MeOH) to yield 0.404 g (81%) of compound 169 as a colorless oil: ¹H NMR (500 1 MHz, CDCl₃) δ 9.66 (s, 1H), 6.84 (s, 1H),5.79-5.87 (m, 1H), 5.28 (d, J=17.1 Hz, 1H), 5.16 (d, J=10.5 Hz, 1 H), 4.59-4.63 (m, 1 H) 4.51-4.55 (m, 1 H) 4.32 (dd, J=12.5, 7.9 Hz, 1 H), 4.00 (dt, J=11.2, 4.7 Hz, 1H), 3.76 (dd, J=11.1, 5.9 Hz, 1H), 3.09 (dd, J=16.7, 4.1 Hz, 1H), 2.33 (dd, J=16.7, 7.9Hz, 1H), 2.28 (d, J=11.2 Hz, 1H), 2.09-2.13 (m, 1H), 1.96-2.07 (m, 2H), 1.82 (dd, J=25.8, 12.2 Hz, 1H), 1.39-1.64 (m, 5H), 1.00-1.04 (m, 1H), 0.97 (d, J=6.1 Hz, 3H); ¹³C NMR(125 MHz , CDCl₃) 200 .2, 168.3, 153.3, 131.6, 118.2, 82.1, 66.0, 65.2, 54.7, 53.8, 51.8, 48.6, 32.1, 31.9, 29.5, 29.3, 21.5, 18.2 ppm; IR (film) 3229, 3079, 2932, 2730, 1732, 1660, 1651, 1470, 1014, 733 cm⁻¹; [α]²⁵_{D}: 110°, [α]²⁵₅₇₇: 115°, [α]²⁵₅₄₆: 132°, [α]²⁵₄₃₅: 238°, [α]²⁵₄₀₅: 299°, (c=2.50, CHCl₃). Anal. Cacld for C₁₈H₂₆N₂O₅: C, 61.70; H, 7.48; N, 7.99. Found: C, 61.80; H, 7.53; N, 8.06. 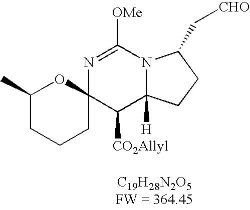
(3R,4R,4aR, 6′R,7S)-4-(Allyloxycarbonyl)-3,4,4a,5,6,7-hexahydro-1-methoxy-7-(2-oxyethyl)-pyrrolo[1,2-c]pyrimidine-3-spiro-6′-(2′-methyl)-3′,4′,5′,6′-tetrahydro-2H-pyran (compound 170).

A solution of compound 169 (0.285 g, 0.813 mmol), MeOTf (0.368 mL, 3.26 mmol), 2,6-di-t-butyl-4-methylpyridine (0.25 g, 1.22 mmol), and dry CH₂Cl₂ (5 mL) was maintained at 23° C. for 5 h. The solution was then poured into Et₂O (40 mL) and washed with 1 N NaOH (2×10 mL) and brine (10 mL), dried (Na₂SO₄), filtered, concentrated, and the resulting residue was purified on 10% pH 7 phosphate buffered silica gel (4:1 hexanes-EtOAc; 3:1 hexanes-EtOAc) to yield 200 mg (68%) of compound 170 as a colorless oil: ¹H NMR(500 Mz, CDCl₃) δ 9.68 (s, 1H) 5.87-5.95 (m, 1H) 5.35 (d, J=17.4 Hz, 1H) 5.21 (d, J=10.5, 1H) 4.64-4.68(m, 1H) 4.58-4.61 (m, 1H) 4.29-4.33(m, 1H) 4.03-4.07 (m, 1H) 3.87 (dt,J=11.1, 5.3 Hz, 1H) 3.66 (s, 3H) 2.76-2.80 (m, 1H) 2.34-2.39 (m, 1H) 2.14-2.24 (m, 2H) 2.02-2.10 (m, 2H) 1.96 (dt, J=12.8, 3.9 Hz, 1 H), 1.66 (dt, J=12.8, 6.5 Hz, 1 H) 1.51-1.55 (m, 2H) 1.39-1.46 (m, 1H) 1.34 (d, J=12.6 Hz, 1H) 1.05 (ddd, J=13.4, 11.6, 4.0 Hz, 1H) 0.97 (d, J=6.3 Hz, 3H); ¹³C NMR (125 MHz, CDCl₃) 200.5, 170.6, 150.3, 132.2, 117.8, 84.9, 65.6, 64.9, 56.6, 54.3, 52.5, 51.8, 50.0, 35.0, 33.6, 29.9, 29.2, 22.2, 19.4 ppm; IR (film) 2932, 2725, 1727, 1636, 1455, 1393, 1017, 754cm⁻¹; [α]²⁵_{D}: 177°, [α]²⁵₅₇₇: 185°, [α]²⁵₅₄₆: 213°, [α]²⁵₄₃₅: 387°, (c=2.00, CHCl₃). Anal. Calcd for C₁₉H₂₈O₅N₂: C, 62.62; H, 7.74; N, 7.69. Found: C, 62.36; H, 7.77; N, 7.52. 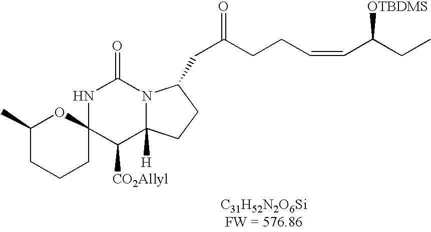
(3R,4R,4aR,6′R,7S)-4-(Allyloxycarbonyl)-1,2,4a,5,6,7-hexahydro-1-oxo-7-[(7S)-(Z)-2-oxo-(t-butyldimethylsiloxy)-5-nonenyl]-pyrrolo]1,2c]pyrimidine-3-spiro-6′-(2′-methyl)-3′,4′,5′,6′-tetrahydro-2H-pyran (compound 172).

t-BuLi (1.83 mL, 1.44 M in hexanes) was added to a −78° C. solution of compound 166 (439 mg, 1.24 mmol), Et₂O (5 mL), and hexanes (7.5 mL). After 20 min, the solution is cannulated into a −78° C. solution of compound 169 (0.20 g, 0.57 mmol) and THF (10 mL). After 5 min, the reaction mixture is quenched with saturated aqueous NH₄Cl (10 ml). The layers were separated, and the aqueous layer extracted with Et₂O (10 mL). The combined organic layers were washed with brine (5 mL), dried (MgSO₄), and concentrated to yield a slightly yellow oil, which was used without further purification.

The crude oil, Dess-Martin periodinane (48 mg, 0.11 mmol), and CH₂Cl₂ (10 mL) were maintained at 23° C. for 45 min. The reaction mixture was quenched with saturated aqueous Na₂S₂O₃ (10 mL), saturated aqueous NaHCO₃ (10 mL) and Et₂O (30 mL). The layers were separated and the organic layer was washed with brine (5 mL), dried (MgSO₄), and concentrated to yield a slightly yellow oil, which was purified on silica gel (1:1 hexanes-EtOAc) to obtain 99 mg.(30%) of the desired product as a colorless oil: ¹H NMR (500 MHz, CDCl₃) δ 6.31 (s, 1H), 5.84-5.90 (m, 1H), 5.29-5.34 (m, 2H), 5.17-5.22 (m, 2H), 4.64-4.68 (m, 1H),4.56-4.59(m, 1H),4.25-4.30(m, 2H), 4.02(dt, J=11.2, 5.0 Hz, 1H),3.77-3.81 (m, 1H), 3.38 (dd, J=16.6, 2.1 Hz, 1H), 2.33-2.45 (m, 2H), 2.20-2.27 (m, 4H), 2.00-2.26 (m, 3H), 1.22-1.77 (m, 8H), 1.06-1.09 (m, 1H), 1.02 (d, J=6.1 Hz, 3H), 0.79-0.83 (m, 12H), 0.00 (s, 3H), −0.03 (s, 3H); ¹³C NMR (75 MHz, CDCl₃) 208.2, 168.5, 153.1, 135.0, 131.6, 126.7, 118.4, 52.1, 69.8, 66.0, 65.3, 54.9, 53.8, 53.0, 46.4, 42.6, 32.2, 32.1, 29.4, 28.9, 25.7, 21.7, 21.6, 18.4, 18.1, 9.7, −4.5, −4.9 ppm; IR (film) 3226, 3079, 2931, 1736, 1717, 1652, 1472, 1375, 1255, 1084, 1015 cm⁻¹; [α]²⁵_{D}=64.6°, [α]²⁵₅₇₇=67.9°, [α]²⁵₅₄₆=78.1°, [α]²⁵₄₃₅=144°, [α]²⁵₄₀₅=177°, (c=2.25, CHCl₃). Anal. Calcd. for C₃₁H₅₂O₆N₂Si: C, 64.55; H, 9.06; N, 4.87. Found: C, 64.39; H, 8.98; N, 4.77. 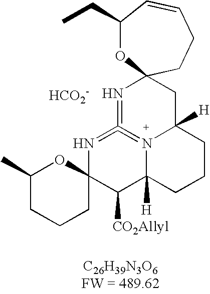
Allyl ester Compound 8.

A solution of compound 172 (110 mg, 0.19 mmol), MeOTf (0.37 μL, 3.3 mmol), 2,6-di-t-butyl-4-methylpyridine (10 mg, 0.05 mmol), and dry CH₂Cl₂ (8mL) was maintained at 23° C. for 12 h. The solution was then poured into Et₂O (30 mL) and washed with 1 N NaOH (2×5 mL) and brine (5 mL) dried (Na₂SO₄) filtered concentrated and the resulting residue was used without further purification.

Anhydrous NH₃ was bubbled through a 0° C. solution of the crude residue and MeOH (25 mL) in a resealable tube. After 15 min the tube was sealed and heated to 50° C. After 2 d the solution was concentrated and the crude residue was used without further purification.

The crude residue TsOH (95 mg 0.50 mmol) and CHCl₃ (10 mL) was maintained at 23° C. After 8 h the reaction mixture was quenched with saturated aqueous NaHCO₃ (2 mL). The layers were separated and the aqueous was extracted with Et₂O (2×5 mL). The combined organic layers were dried (MgSO₄) and concentrated to yield a slightly yellow oil which was purified on silica gel (10:1:0.1 CHCl₃:i-,rOH:HCO₂H) to obtain 23 mg (25%) of the desired product as a slightly yellow oil. 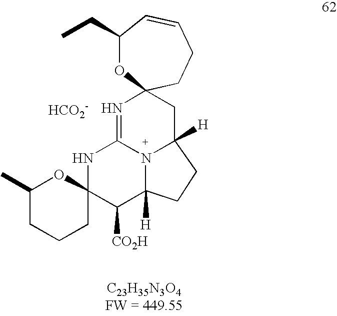
Pentacyclic Acid Compound 7.

A solution of compound 8 (23 mg 0.05 mmol), d(,,h₃) (4 mg 3 μmol) dimedone (35 mg 0.25 mmol) and THF (1 mL) was maintained at 23° C. After 10 min the reaction mixture was concentrated and purified on silica gel (10:1:0.1 CHCl₃:i-,rOH:HCO₂H−4:1 CHCl₃ :HCO₂H) to obtain 3 mg (13%) of the desired product as a slightly yellow oil: HRMS (FAB) m/z 404.2549 calcd for C₂₂H₃₄O₄N₃ found 404.2541. organic layers were dried (MgSO₄), and concentrated to yield a slightly yellow oil, which was purified on silica gel (10:1:0.1 CHCl_{3:}i-PrOH:HCO₂H) to obtain 23 mg (25%) of the desired product as a slightly yellow oil. 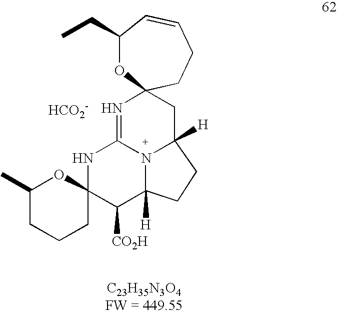
Pentacyclic Acid Compound 7.

A solution of compound 8 (23 mg, 0.05 mmol), Pd(PPh₃) (4 mg, 3 μmol), dimedone (35 mg, 0.25 mmol), and THF (1 mL) was maintained at 23° C. After 10 min, the reaction mixture was concentrated and purified on silica gel (10:1:0. I CHCl_{3:}i-PrOH:HCO₂H−4:1 CHCl_{3:}HCO₂H) to obtain 3mg (13%) of the desired product as a slightly, yellow oil: HRMS (FAB) m/z 404.2549 calcd for C₂₂H₃₄O₄N₃, found 404.2541.

### EXAMPLE VII

An Improved Synthesis of Pentacyclic Acid

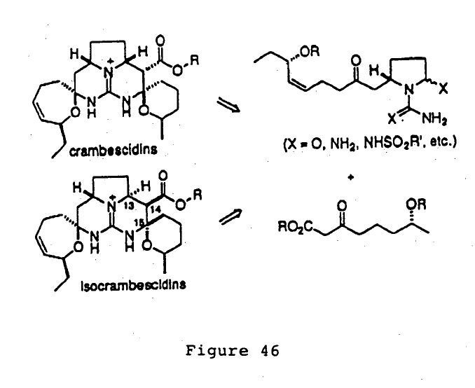

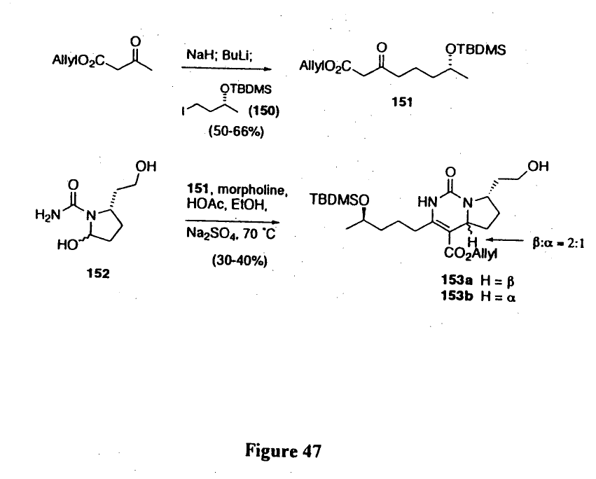

This Example provides an improved method for synthesizing pentacyclic acid comnpounds. Chemical synthesis procedures are as described above for Example VI. A convergent synthetic strategy for the guanidinium alkaloids is shown in FIG. 46.

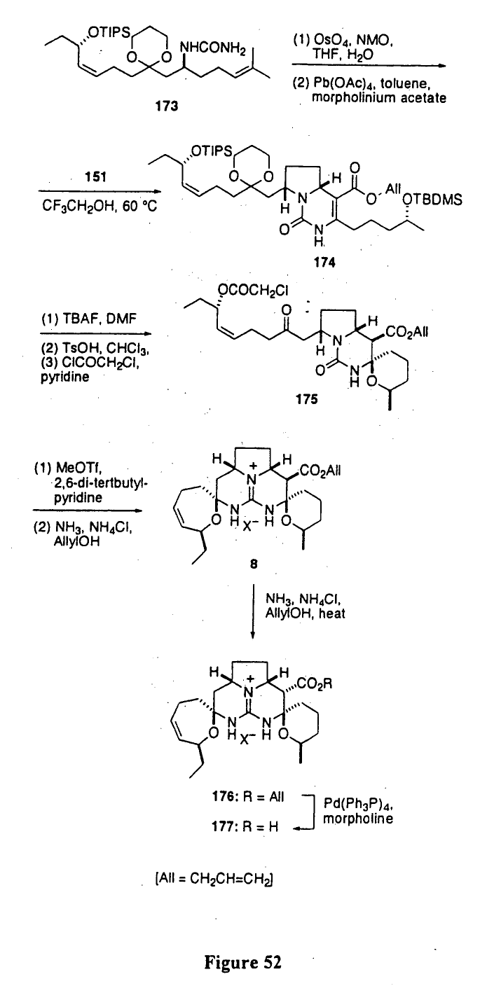

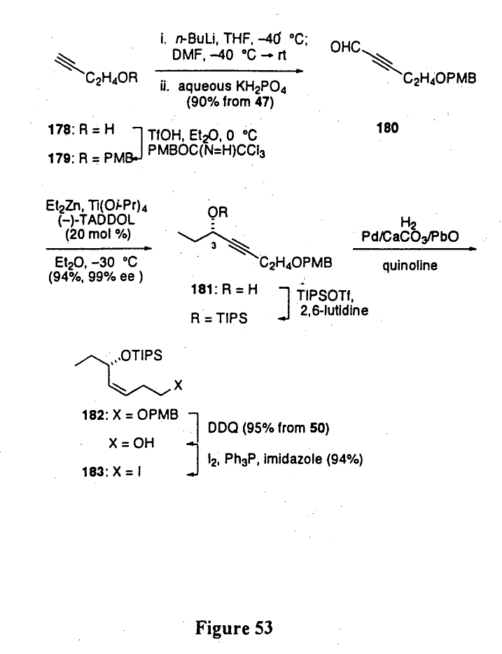

FIG. 52 depicts the synthesis strategy for a new method of preparing pentacyclic acid compounds and the compounds produced, e.g. compounds 176 and 177, using compound 173 as starting material. Compound 61 is an urea compound obtained as shown in FIGS. 53, 54 and 55 as follows: 3-butynol (compound 178) is converted to the p-methoxybenzyl (PMB) ether 179 (FIG. 53). The alkyne of compound 179 was deprotonated with n-buthyl lithium at −40° C. and the resulting acetylide treated with anhydrous DMF to provide ynal 180 in 90% yield, after quenching the intermediate -aminoalkoxide into aqueous phosphate buffer (Journet et al., Tetrahedron Lett., 1988, 39:6427). The C3 stereocenter was introduced bythe method of Weber and Seebach (Singh et al., J. Am. Chem. Soc., 1987, 109:6187) through condensation of ynal 180 with Et₂Zn in the presence of (−)-TADDOL (20 mol %) and Ti(Oi-Pr)₄ to give (S)-181 in 94% yield and >98% ee. This asymmetric transformation was reliably performed on a 45 g scale. Propargylic alcohol 181 was protected as the triisopropylsilyl (TIPS) ether and the alkyne partially hydrogenated with Lindlar's catalyst to provide cis alkene 182. The PMB-protecting group was oxidatively removed with DDZ and the resulting alcohol was converted to iodide 183 (Kitamura et al., Org. Synth., 1992, 71:1) in an overall yield of 89% from 181.

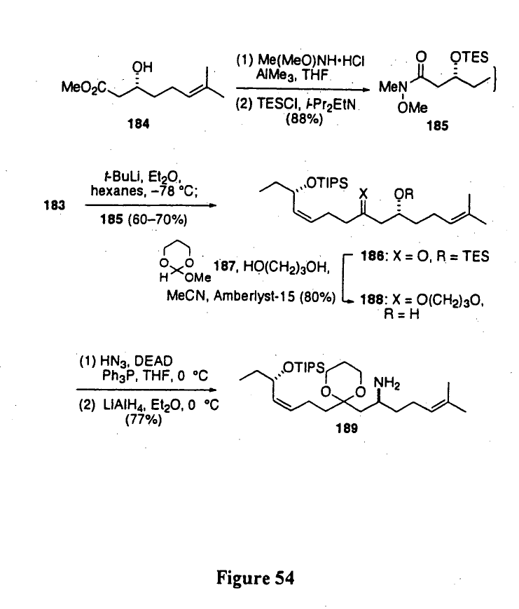

Enantiopure methyl R-3-hydroxy-7-methyloct-6-enoate (Kitamura et al., Org. Synth., 1992, 71:1) was converted to amide 185 in 88% yield by reaction with N,O-dimethylhydroxylaminde hydrochloride according to the procedure of Weinreb (Garigipati et al., J. Am. Chem. Soc., 1985, 107:7790) followed by protection of the secondary alcohol as the triethylsilyl (TES) ether (FIG. 54). Iodide 183 was converted to the corresponding lithium reagent and coupled with 185 to generate dienone 186 in 60-70% yield. Masking the C8 carbonyl of 186 as the ketal was necessary to prevent a β-hydroxy elimination, which occurred under the Mitsunobu conditions employed to install the β-amino functionality. Ketalization was sluggish, however, when the β-hydroxy group was protected, so optimized reaction conditions were found which cleaved the TES group, did not promote the β-hydroxy elimination of the intermediate β-hydroxy ketone and promoted ketalization. Optimized ketalization conditions involved treatment of 186 with orthoester 187 (Roush and Gillis, J. Org. Chem., 1980, 45:4283-4287; Baganz and Domascke, Chem. Ber., 1958, 91:650-653) and 1,3-propanediol in the presence of Amberlyst-15 to provide ketal 188 in 80% yield. Mitsunobu displacement of the secondary alcohol with azide followed by reduction to the amine provided compound 189 in 77% yield from compound 188.

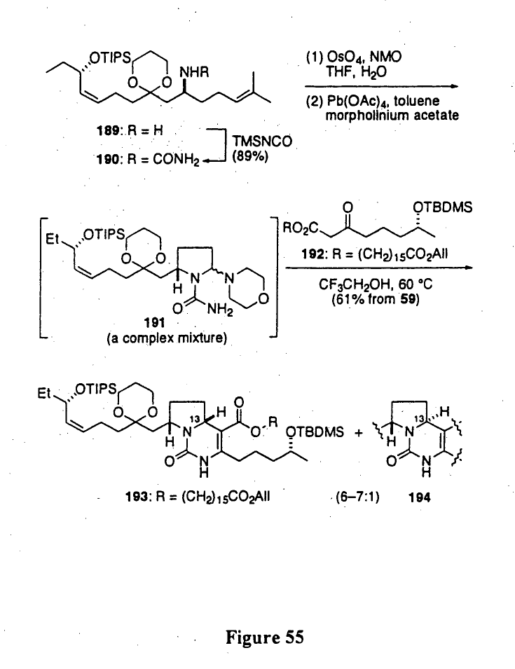

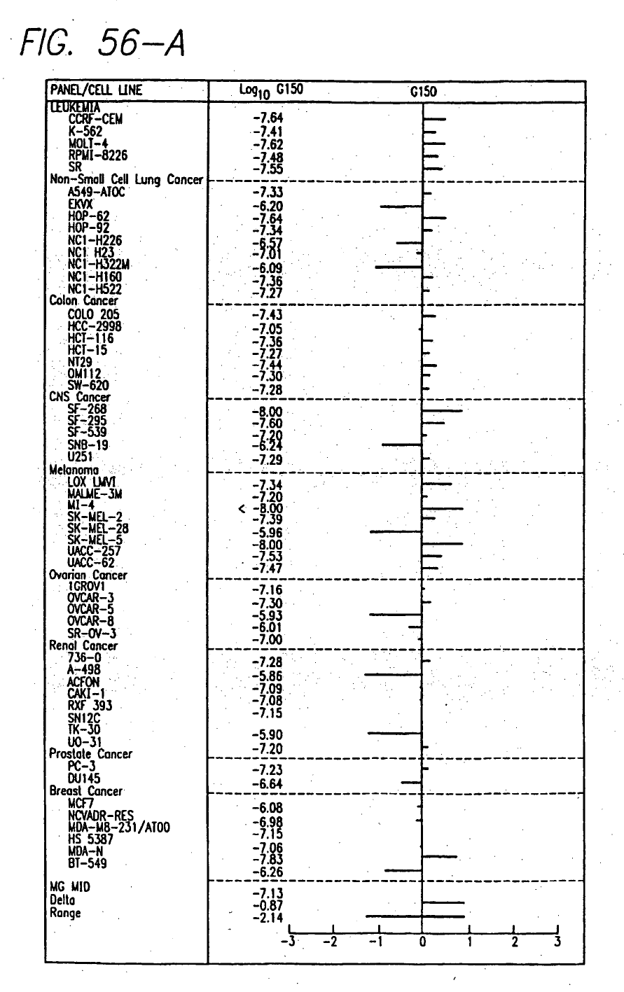

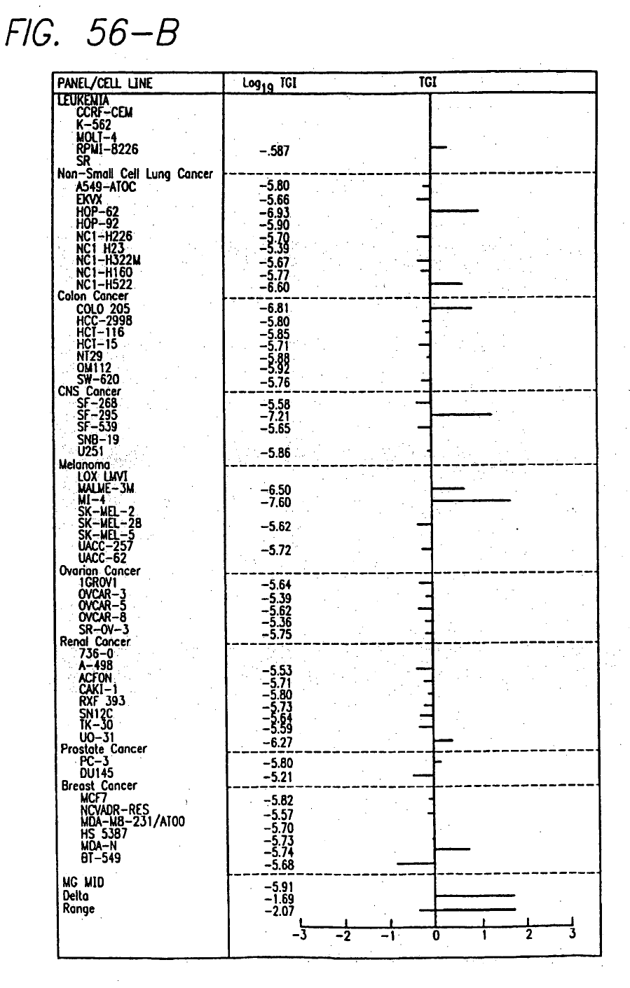

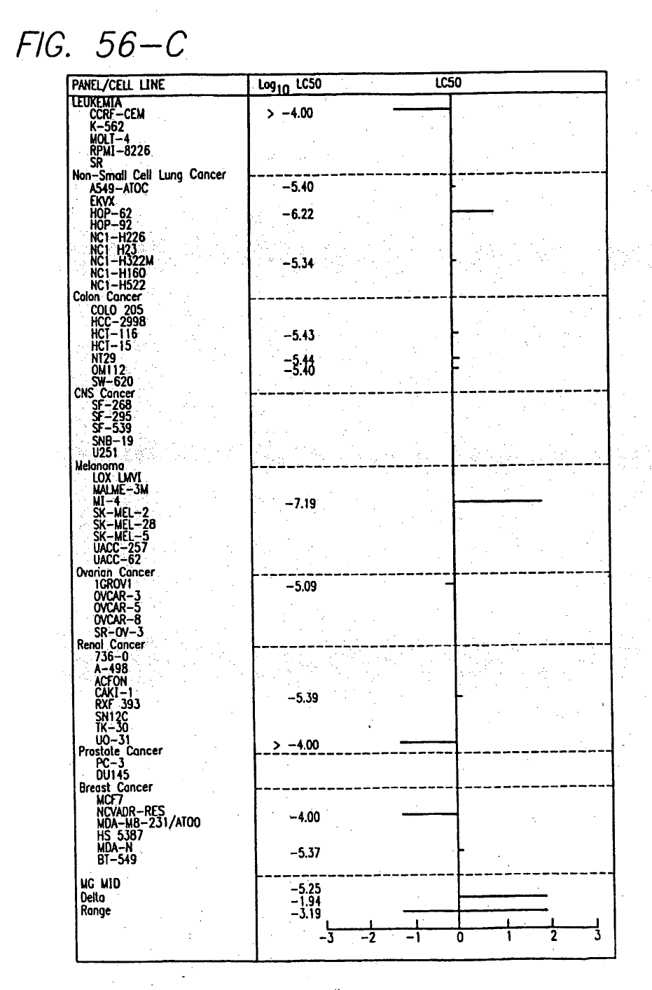

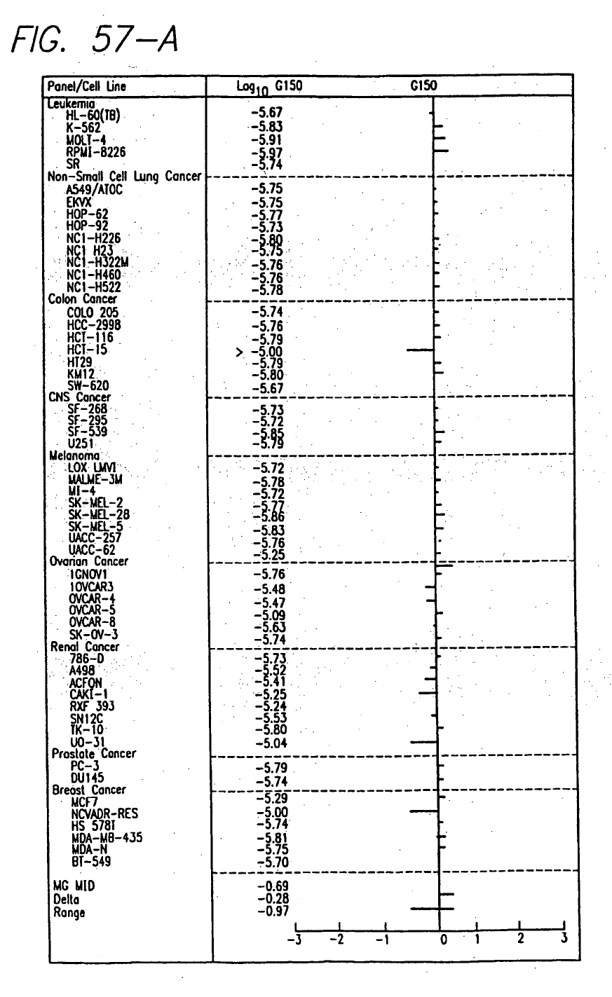

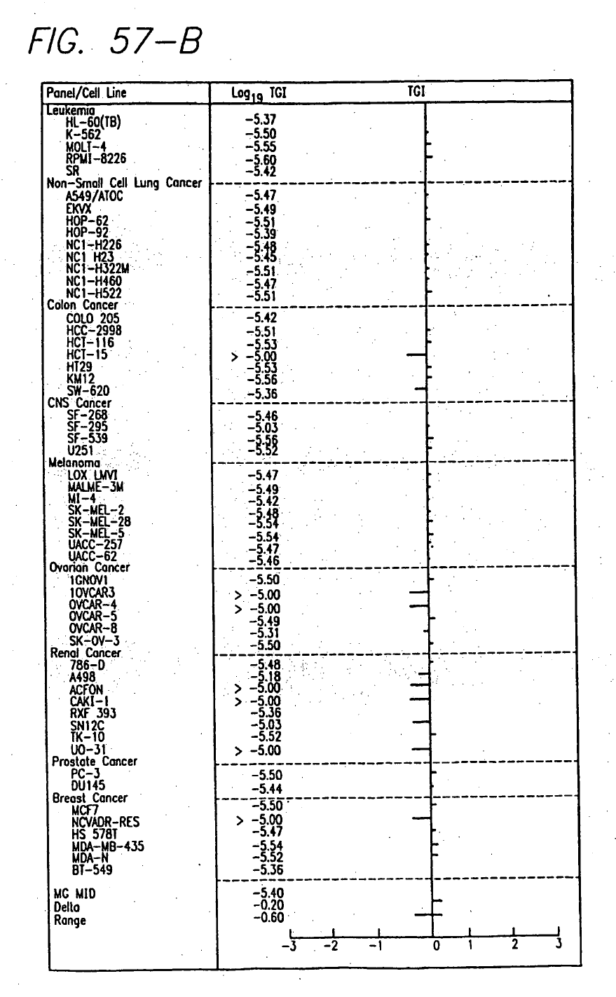

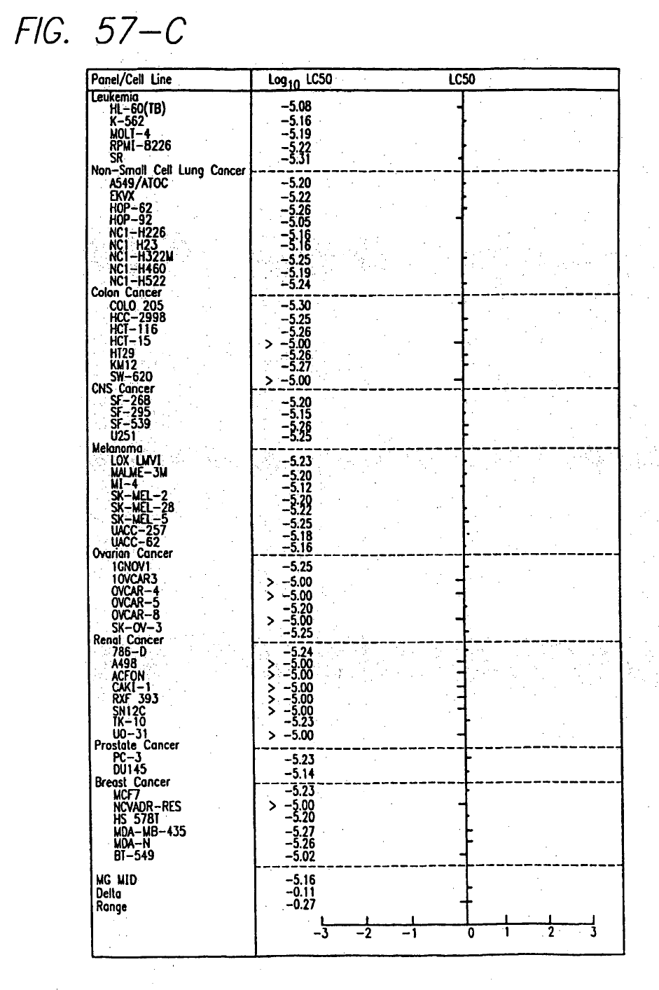

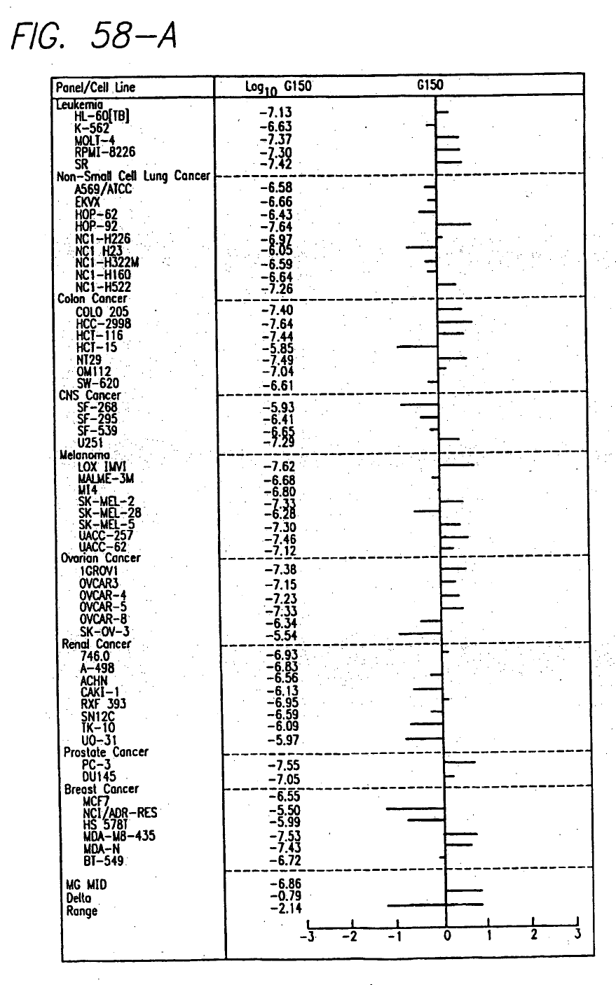

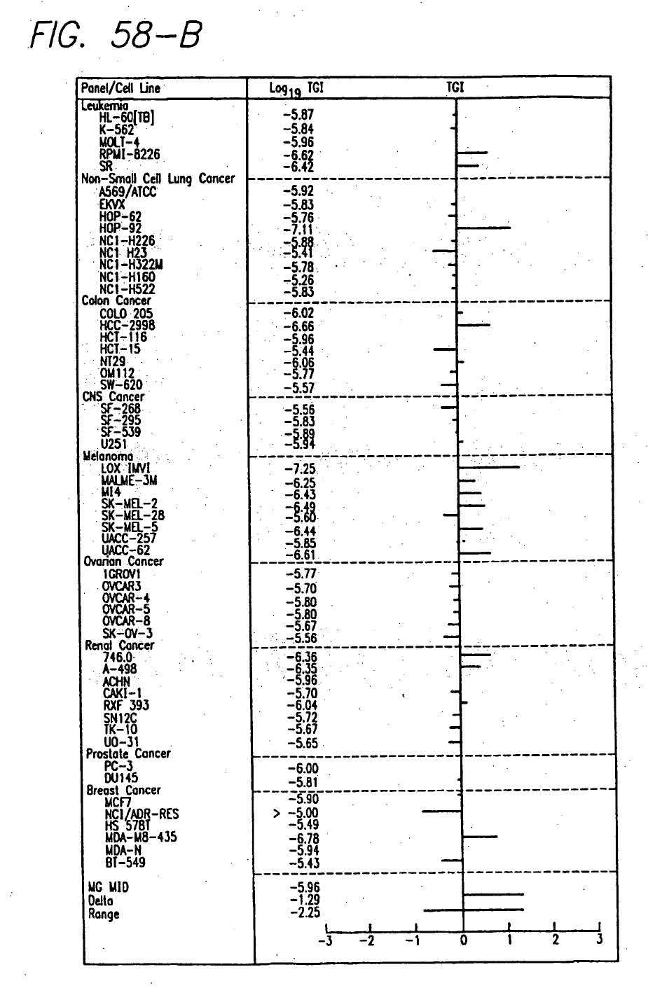

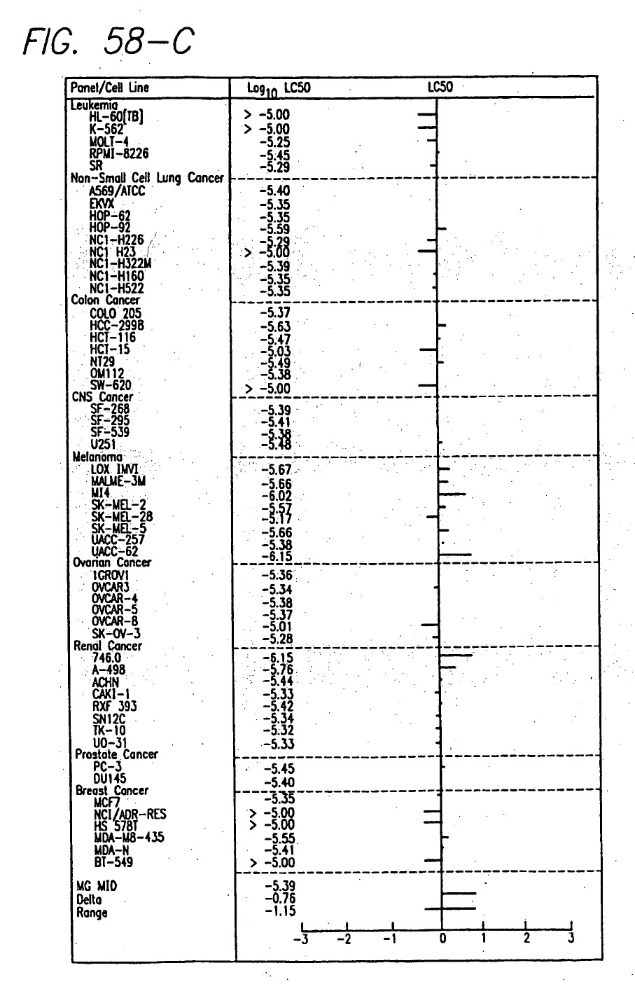

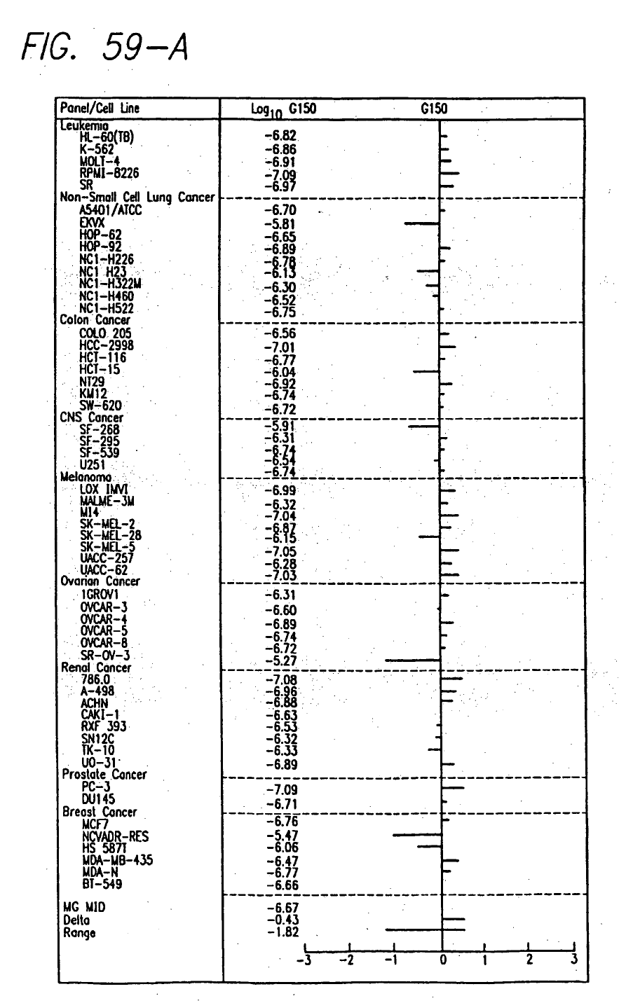

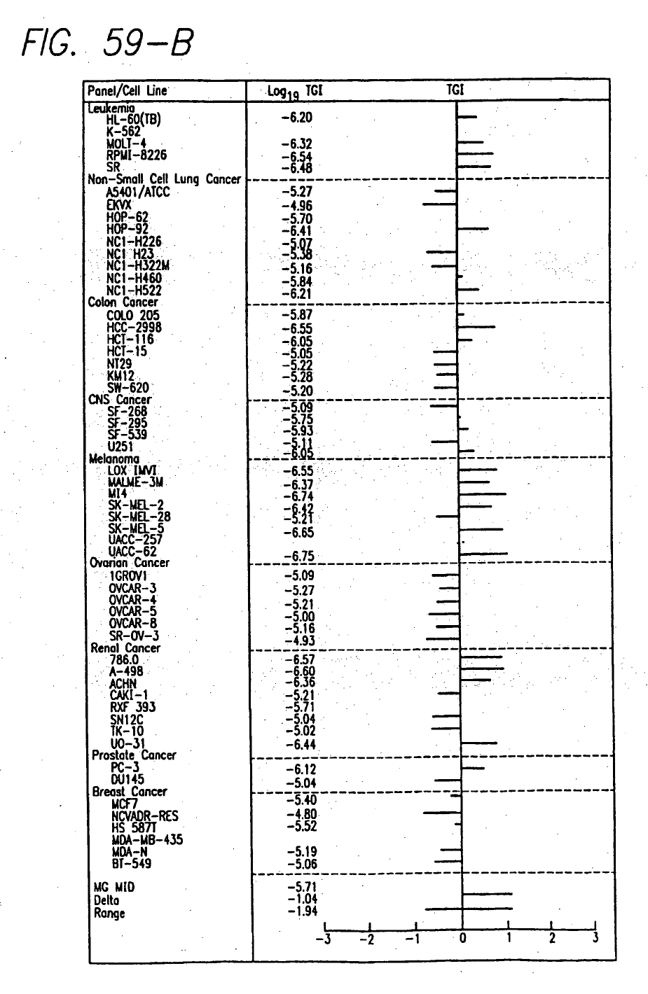

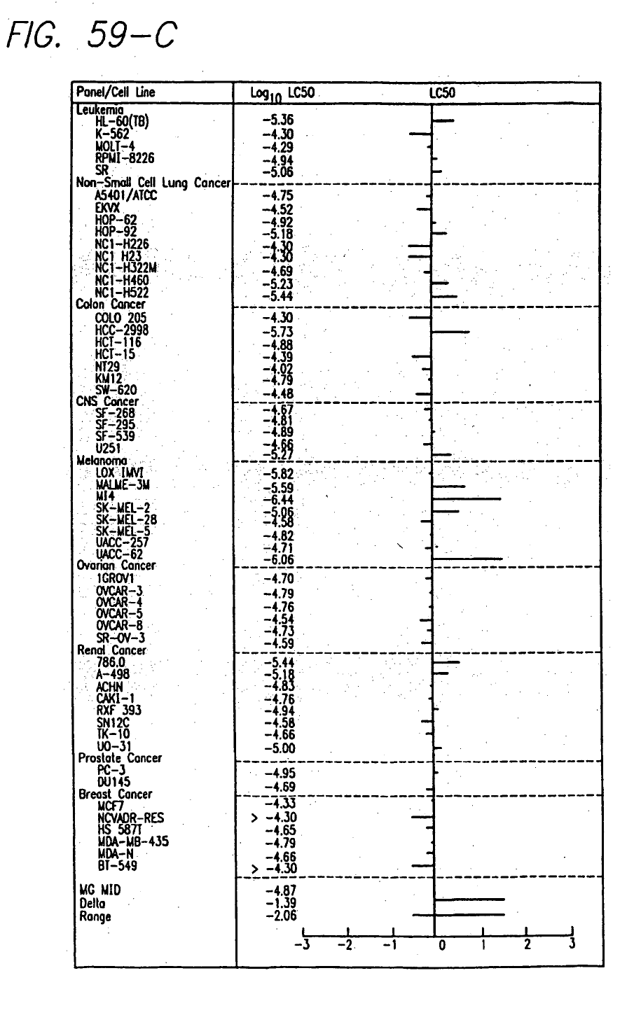

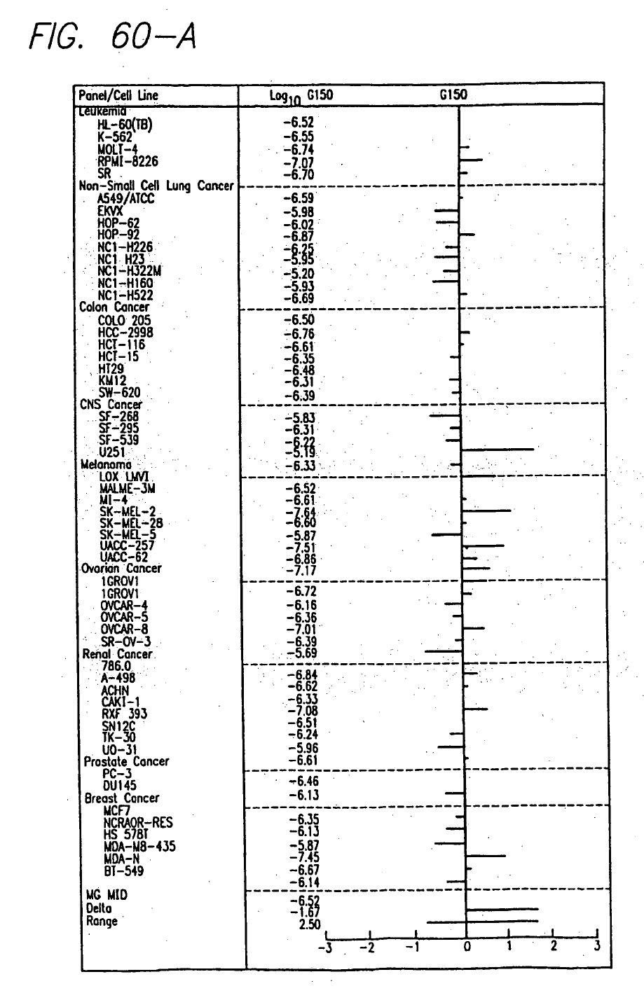

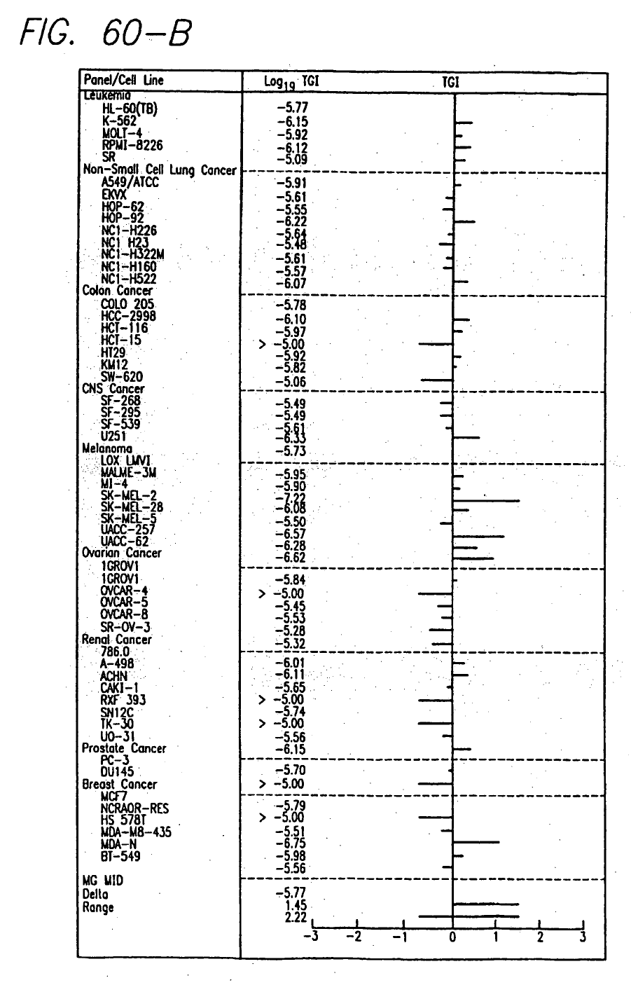

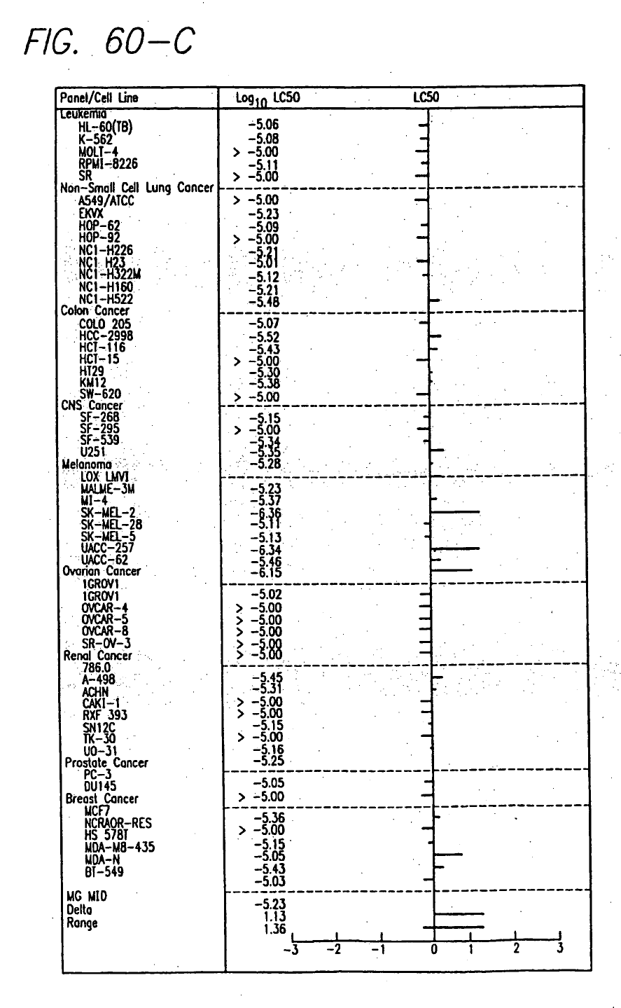

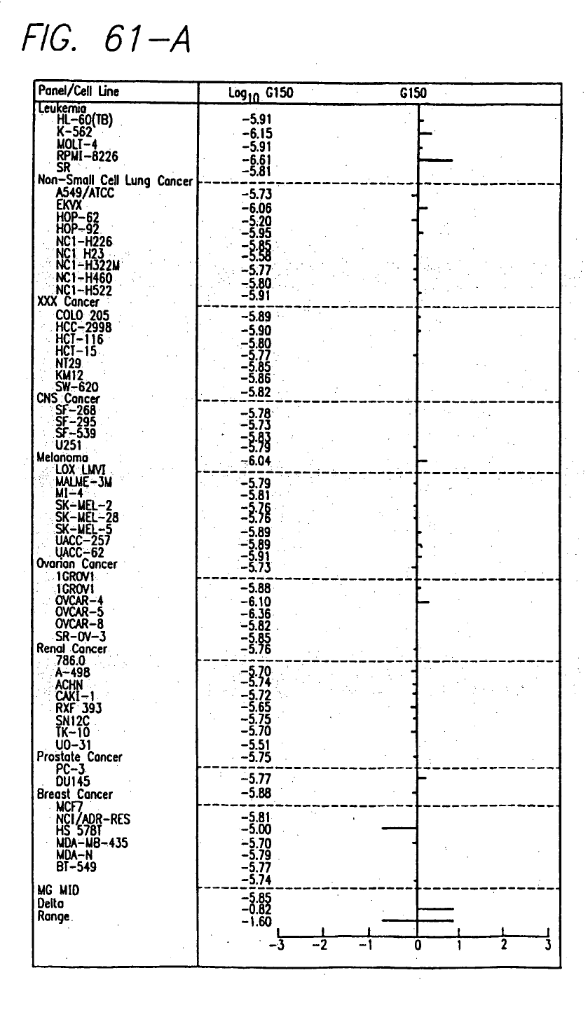

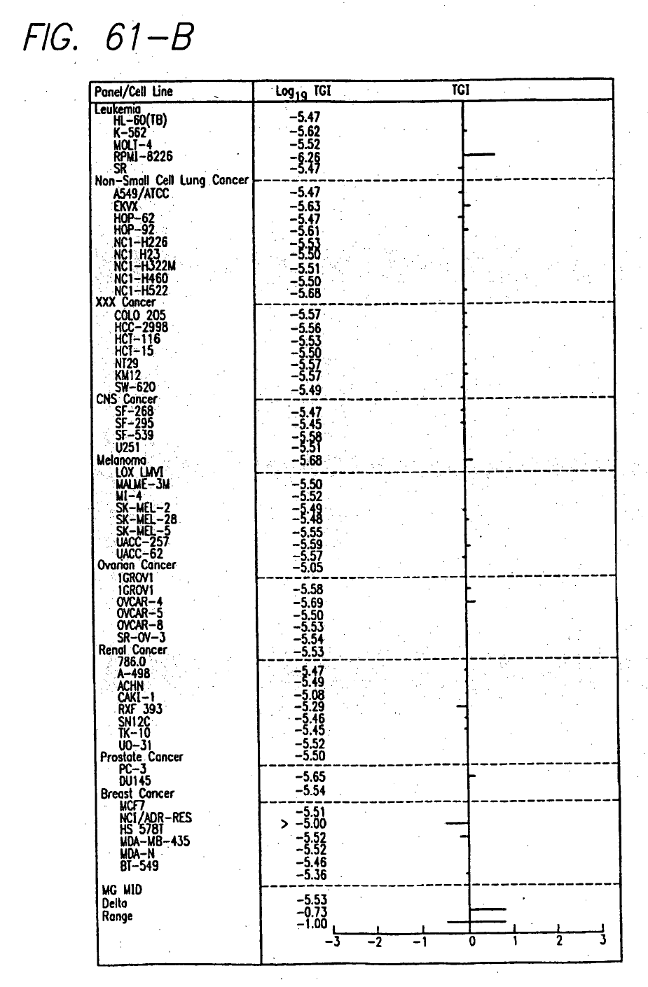

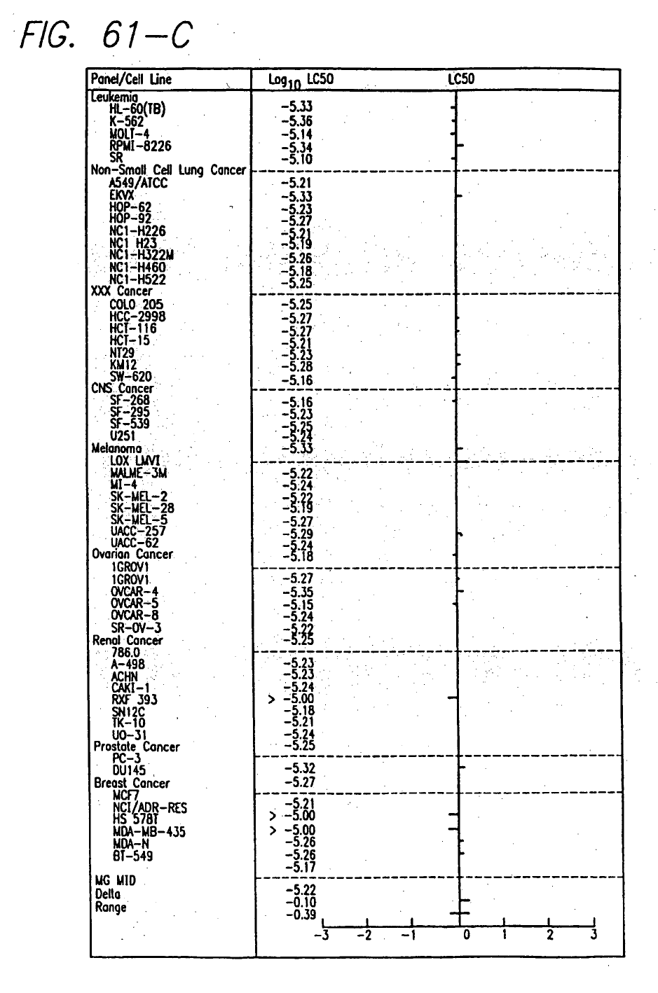

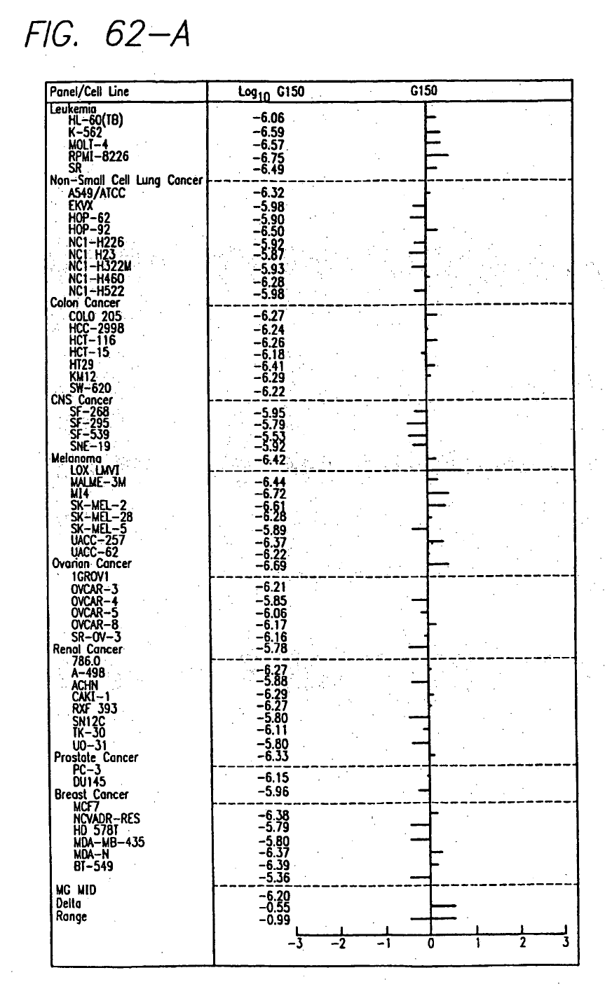

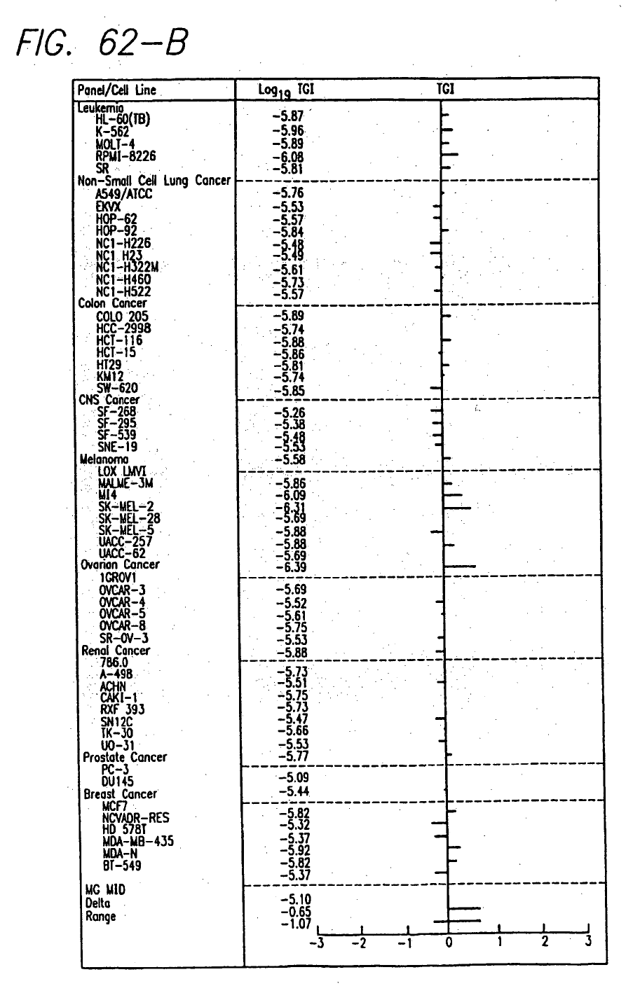

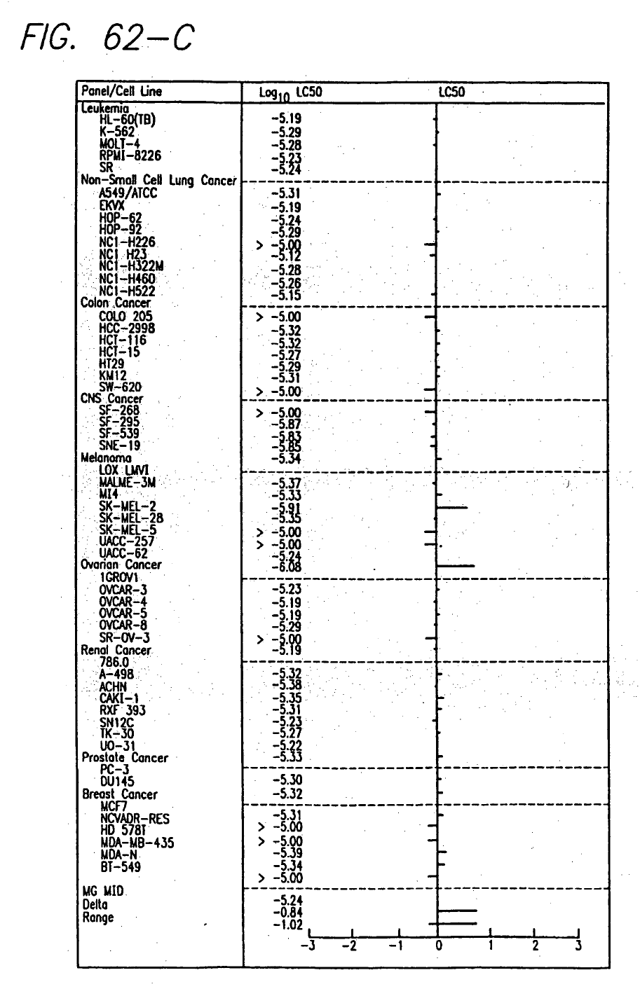

Condensation of amine 189 with TMSNCO yielded urea 190 in 89% yield (FIG. 55). Amine 189 is used to prepare pentacyclic compound 177 as shown in FIG. 52.

These results demonstrate a method that permits preparation of both pentacyclic compounds 7 and 177. Having access to both types of compounds, allows side chains to be attached either before or after epimerization of the ester of the pentacyclic compound.

### EXAMPLE VIII

This example describes the in vitro screening of 60 tumor cell lines against the compounds of the invention: ptilomycalin A, isocrambescidin 800 trihydrochloride, triacetylcrambescidin 800 chloride, crambescidin 657 hydrochloride, crambescidin 800 trihydrochloride, triacetylisocrambescidin 800 chloride, and 13-epiptilomycalin A to determine anti-tumor activity.

The screening methods utilized the National Cancer Institute (NCI) DTP Human Tumor Cell Line Screen protocol as described by Monks et al., J. Nat'l. Cancer Inst. 83:757-766 (1991); and Boyd In “Cancer Drug Discovery and Development, Vol; 2; Drug Development; Preclinical Screening, Clinical Trial and Approval, Humana Press, 1997, pp 23-43. The origins and processing of the cell lines used are described in Alley et al., Cancer Res., 1988, 48:589-601; Shoemaker et al., Prog. Clin. Biol. Res., 1988, 276:265-286; and Stinson et al., Proc. Am. Assoc. Cancer Res., 1989, 30:613.

In brief, in the screen protocol, cell suspensions were diluted depending on cell type and the expected target cell density (approximately 5000-40,000 cells per well) into a 96 well microtiter plate. Inoculates were preincubated for 24h at 37 □C for stabilization. Dilutions at twice the intended test concentrations were added at time zero in 100 μL aliquots to the microtiter plate wells. Test compounds were evaluated at five 10-fold dilutions. Routine test concentrations have the highest well concentration at 10E-4M, but for the standard agents, the highest well concentration used depended on the agent used. Incubations lasted 48 hours in 5% CO₂ atmosphere and 100% humidity. The cells were assayed by Sulforhodomine B assay as described by Rubenstein et al., JNCI, 1990, 82:1113-1118 and Skehan et al., JNCI, 1990, 82: 1107-1112. Optical densities were read with a plate reader and the data processed using a microcomputer into special concentration parameters.

NCI renamed the IC50 value, the concentration that causes 50% growth inhibition the “GI50” value to emphasize the correction for the cell count at time zero; thus GI50 is the concentration of test drug where 100×(T−T0)/(C−T0)=50 (Boyd et al., In Cytotoxic Anticancer Drugs: Models and Concepts for Drug Discovery and Development, Vieriote. et al., Eds., Kluwer Academic, Hingham, Mass., 1992, pp 11-34; and Monks et al., JNCI, 1991, 83, 757-766. The optical density of the test well after a 48 hr period of exposure to the test compound is “T”, the optical density at time zero is TO and the control optical density is “C.” The “50” is called the GI50PRCNT, a T/C-like parameter that can have values from +100 to −100. The GI50 also measures the growth inhibitory power of the test compound. The TGI is the concentration of test drug where 100×(T−T0)/(C−T0)=0. Thus, the TGI signifies a cytostatic effect. The LC50 which signifies a cytotoxic effect, is the concentration of the test compound where 100×(T−T0)/T0=−50. The control optical density is not used in the calculation of LC50.

These concentration parameters are interpolated values. The concentrations giving G150PRCNT values above and below the reference values (e.g. 50 for G150) are used to make interpolations on the concentration axis. Currently, about 45% of the G150 records in the database are “approximated.” In 42% of the records, the G150PRCNT for a given cell line does not go to 50 or below. For mean graph purposes, the value assumed for the G150 in such a case is the highest concentration tested (HICONC). Similar approximations are made when the G150 cannot be calculated because the G150PRCNT does not go as high as 50 or above (3% of total). In this case, the lowest concentration tested is used for the G150. Corresponding approximations are made for the TGI and for the LC50.

The results of the tumor cell screening are shown in the mean graphs of 56-62.

The mean graphs are a presentation of the in vitro tumor cell screen results developed by the NCI to emphasize differential effects of test compounds on various human tumor cell lines (Boyd et al., In Cancer:Principles and Practice of Oncology, DeVita et al., Eds., Lippincott, Philadelphia, Pa., 1989, Vol.3, pp. 1-12; Paull et al., JNCI, 1989, 81:1088-1092;and Paull et al., Proc. Am. Assoc. Cancer Res., 1988, 29:488. The mean graph bar graphs depict patterns created by plotting positive and negative values generated from a set of G150, TGI or LC50 values. The positive and negative values are plotted against a vertical line that represents the mean response of all the cell lines in the panel to the test compound. Positive values project to the right of the vertical line and represent cellular sensitivities to the test agent that exceed the mean. Negative values project to the left and represent cell sensitivities to the test compound that are less than the average value. The positive and negative values, called “deltas”, are generated from the G150 data (or TGI or LC40 data) by a three-step calculation. The G150 value for each cell line tested against a test compound is converted to its log10 G150 value. The log₁₀ G150 values are averaged. Each log₁ G150 value is subtracted from the average to create the delta. Thus, a bar projecting 3 units to the right denotes that the G150 (orTGI or LC50) for that cell line occurs at a concentration 1000 times less than the average concentration required for all the cell lines used in the experiment. Thus the cell line is usually sensitive to that compound. If for a particular compound and cell line it was not possible to determine the desired response parameter by interpolation, the bar length shown in either the highest concentration tested (and the listed log₁₀ of the response parameter will be preceded by a “>”) or the lowest concentration tested (and the listed logio will be preceded by a “<”). The, values at either limit (>or <) are also calculated in the mean used for the meangraph. Therefore, the mean used in the meangraph may not be the actual mean of the G150 for instance. For, this reason, this value is referred to as the MgMID (meangraph midpoint).

These results demonstrate that certain cancer cell lines are sensitive to ptilomycalin A, triacetylcrambescidin 800 chloride, crambescidin 657 hydrochloride, cranbescidin 800 trihydrochloride, and 13-epiptilomycalin A. Isocrambescidin 800 trihydrochloride and triacetylisocrambescidin 800 chloride display a lesser effect on the cell lines tested.

1. A compound of the formula: 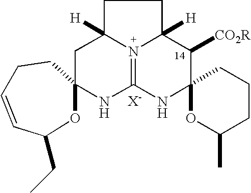
Wherein,
R=H, a carboxylic acid protecting group, an ω-alkoxycarboxylic acid or an ω-alkoxycarboxylic acid ester, and
X=any pharmaceutically acceptable counterion.

2. A compound of the formula: 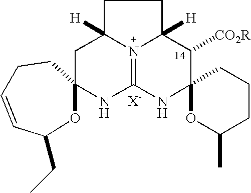
Wherein,
R=H, a carboxylic acid protecting group, an ω-alkoxycarboxylic acid or an ω-alkoxycarboxylic acid ester, and
X=any pharmaceutically acceptable counterion.

3. A compound of the formula: 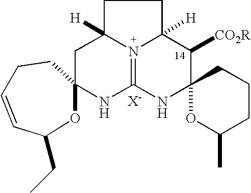
Wherein,
R=H, a carboxylic acid protecting group, an ω-alkoxycarboxylic acid or an ω-alkoxycarboxylic acid ester, and
X=any pharmaceutically acceptable counterion.

4. A compound of the formula: 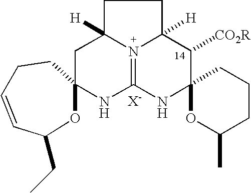
Wherein,
R=H, a carboxylic acid protecting group, an ω-alkoxycarboxylic acid or an ω-alkoxycarboxylic acid ester, and
X=any pharmaceutically acceptable counterion.

5. A compound of the formula: 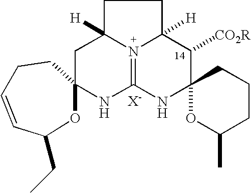
Wherein,
R=H, a carboxylic acid protecting group, an ω-alkoxycarboxylic acid or an ω-alkoxycarboxylic acid ester, and
X=any pharmaceutically acceptable counterion.

6. A compound of the formula:

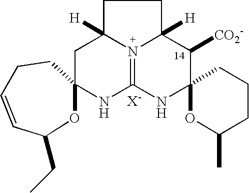

7. A compound of the formula:

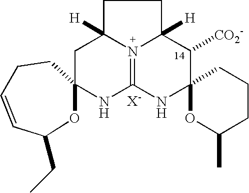

8. A compound of the formula:

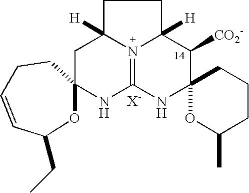

9. A compound of the formula:

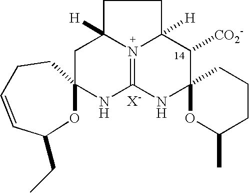

10. A compound of the formula:

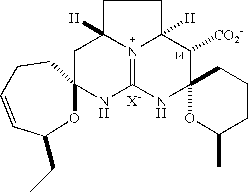

11. A method for synthesizing a pentacyclic compound of the formula: 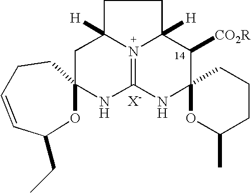
Wherein,
R=H, a carboxylic acid protecting group, an ω-alkoxycarboxylic acid or an ω-alkoxycarboxylic acid ester, and
X=any pharmaceutically acceptable counterion
which method comprises reacting a compound of the formula: 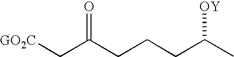
wherein G=a carboxylic acid protecting group, an ω-alkoxycarboxylic acid or and ω-alkoxycarboxylic acid ester, and
Y=alcohol protecting group
with a compound of the formula: 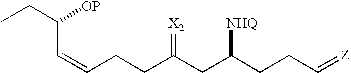
wherein X₂=O or ketone protecting group
Z=alkene or carbonyl protecting group
P=alcohol protecting group and
Q=amino carbonyl group
to produce a compound of the formula: 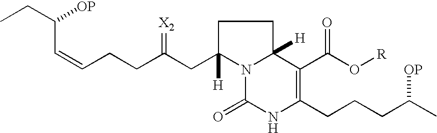
wherein X₂=O or ketone protecting group
P=alcohol protecting group, and
R=carboxylic acid protecting group, ω-alkoxycarboxylic acid or ω-alkoxycarboxylic acid ester
which compound is subsequently converted to the pentacyclic compound by deprotection, incorporation of ammonia, and cyclization.

12. The method of claim 11, wherein when R=a carboxylic acid protecting group, the method further comprises the step of deprotecting the pentacycle compound of claim 11.

13. A method for synthesizing a pentacyclic compound of the formula: 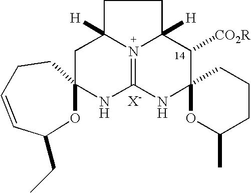
Wherein,
R=H, a carboxylic acid protecting group, an ω-alkoxycarboxylic acid or an ω-alkoxycarboxylic acid ester, and
X=any pharmaceutically acceptable counterion,
which comprises epimerizing the stereocenter at carbon-14 of the compound of the formula:

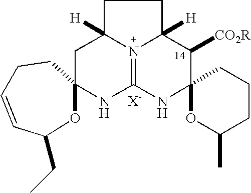

14. The method of claim 13, wherein when R=a carboxylic acid protecting group, the method further comprises the step of deprotecting the pentacycle compound of claim 13.

15. A method for synthesizing pentacyclic compounds B and C of the formulae: 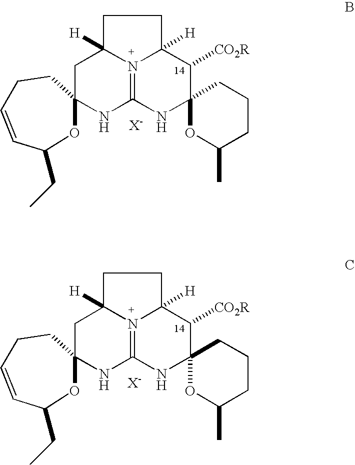
Wherein,
R=H, a carboxylic acid protecting group, an ω-alkoxycarboxylic acid or an ω-alkoxycarboxylic acid ester, and
X=any pharmaceutically acceptable counterion,
which comprises reacting a compound of the formula: 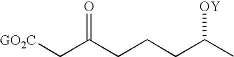
wherein G=a carboxylic acid protecting group, an ω-alkoxycarboxylic acid or
an ω-alkoxycarboxylic acid ester, and
Y=an alcohol protecting group
with a compound of the formula: 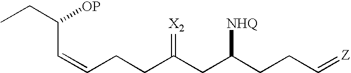
wherein X₂=O or a ketone protecting group
Z=an alkene or carbonyl protecting group
P=an alcohol protecting group, and
Q=an amidinyl group
To produce a compound of the formula: 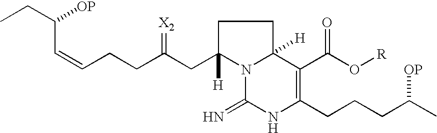
wherein X₂=O or a ketone protecting group
P=an alcohol protecting group and
R=a carboxylic acid protecting group, an ω-alkoxycarboxylic acid or an ω-alkoxycarboxylic acid ester
which is subsequently converted to the pentacyclic compound by deprotection and cyclization.

16. The method of claim 15, wherein when R=a carboxylic acid protecting group, the method further comprises the step of deprotecting the pentacycle compound B of claim 15.

17. The method of claim 15, wherein when R=a carboxylic acid protecting group, the method ftifther comprises the step of deprotecting the pentacycle compound C of claim 15.

18. A method for synthesizing a pentacyclic compound of the formula: 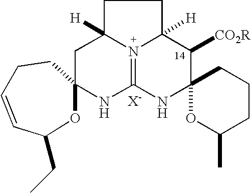
R=H, a carboxylic acid protecting group, an ω-alkoxycarboxylic acid or an ω-alkoxycarboxylic acid ester, and
X=any pharmaceutically acceptable counterion.
which comprises epimerizing the stereocenter at carbon-14 and carbon 15 of the compound of the formula:

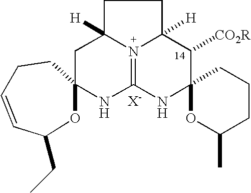

19. The method of claim 18, wherein when R=a carboxylic acid protecting group, the method further comprises the step of deprotecting the pentacycle compound of claim 18.

20. The compound of claim 1, 2, 3, 4, or 5 wherein R=allyl and X=Cl⁻

21. The compound of claim 1, 2, 3, 4, or 5 wherein R=H, and X=Cl⁻.

22. The compound of claim 1, 2, 3, 4, or 5 wherein R=(CH₂)₁₅CO₂G,
Wherein G=H, a counterion of a carboxyl ate salt, or a carboxylic acid protecting group, and X=Cl⁻

23. The compound of claim 1, wherein R=(CH₂)₁₅CO₂H and X=Cl⁻.

24. The compound of claim 2, wherein R=(CH₂)₁₅CO₂H and X=Cl⁻.

25. The compound of claim 3, wherein, R=(CH₂)₁₅CO₂H and X=Cl⁻.

26. The compound of claim 4, wherein R=(CH₂)₁₅CO₂H and X=Cl⁻.

27. The compound of claim 5, wherein R=(CH₂)₁₅CO₂H and X=Cl⁻.

28. A compound of the formula: 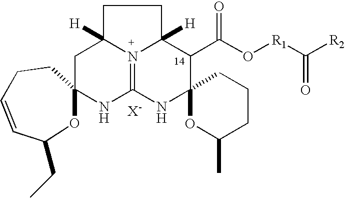
wherein R₁=any alkyl, aryl or substituted alkyl group
R₂=O⁻, OH, OG₁, a spermidine moiety or a substituted spermidine moiety
wherein G₁=a carboxylic acid protecting group and
X=any pharmaceutically acceptable counterion.

29. A compound of the formula: 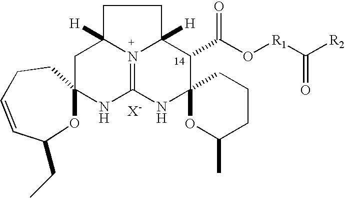
wherein R₁=any alkyl, aryl or substituted alkyl group
R₂=O⁻, OH, OG₁, a spermidine moiety or a substituted spermidine moiety
wherein G₁=a carboxylic acid protecting group and
X=any pharmaceutically acceptable counterion.

30. A compound of the formula: 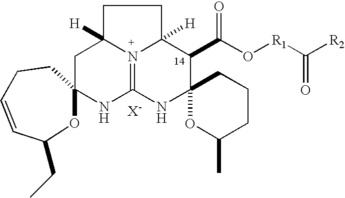
wherein R₁=any alkyl, aryl or substituted alkyl group
R₂=O^{-, OH, OG}₁, a spermidine moiety or a substituted spermidine moiety
wherein G₁=a carboxylic acid protecting group and
X=any pharmaceutically acceptable counterion.

31. A compound of the formula: 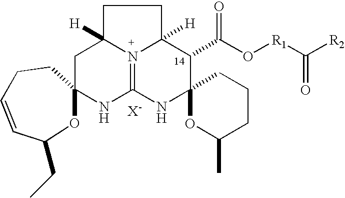
wherein R₁=any alkyl, aryl or substituted alkyl group
R₂=O⁻, OH, OG₁, a spermidine moiety or a substituted spermidine moiety
wherein G₁=carboxylic acid protecting group, and
X=any pharmaceutically acceptable countenon.

32. A compound of the formula: 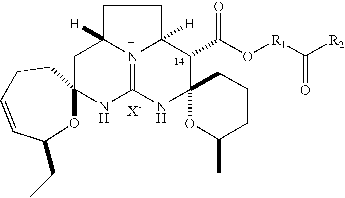
wherein R₁=any alkyl, aryl or substituted alkyl group
R₂=O⁻, OH, OG₁, a spermidine moiety or a substituted spermidine moiety
wherein G₁=carboxylic acid protecting group and
X=any pharmaceutically acceptable counterion.

33. The method of claim 11, wherein when R is an ω-alkoxycarboxylic acid, the method further comprises the step of reacting the pentacyclic compound of the formula: 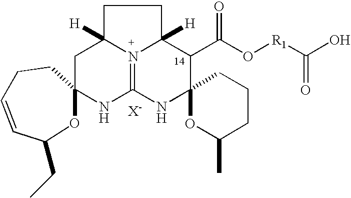
wherein, R₁=any alkyl, aryl or substituted alkyl group
with a protected spermidine or a protected substituted sperimidine and subsequently deprotecting to produce the compound of the formula: 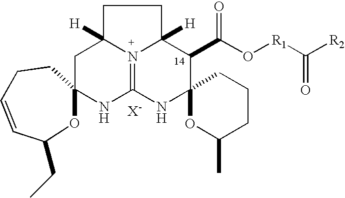
wherein R₁=any alkyl, aryl or substituted alkyl group
R₂=a spermidine moiety or a substituted spermidine moiety and
X=any pharmaceutically acceptable counterion.

34. The method of claim 13, wherein when R is an ω-alkoxycarboxylic acid the method further comprises the step of reacting the pentacyclic compound of the formula: 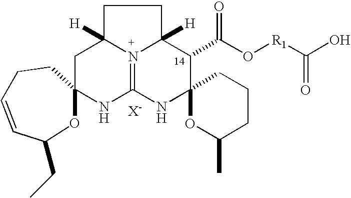
with a protected spermidine or a protected substituted sperimidine and subsequently deprotecting to produce the compound of the formula: 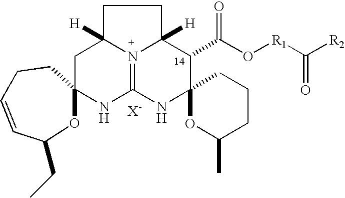
wherein R₁=any alkyl, aryl or substituted alkyl group
R₂=a spermidine moiety or a substituted spermidine moiety, and
X=any pharmaceutically acceptable counterion.

35. The method of claim 15, wherein when R is an ω-alkoxycarboxylic acid the method further comprises the step of reacting the pentacyclic compound of the formula: 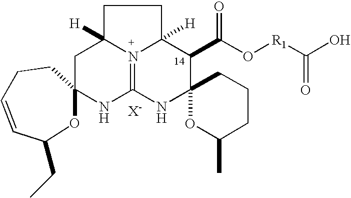
wherein, R₁=any alkyl, aryl or substituted alkyl group
with a protected spermidine or a protected substituted sperimidine and subsequently deprotecting to produce the compound of the formula: 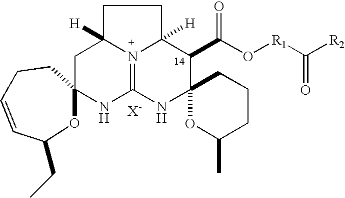
wherein R₁=any alkyl, aryl or substituted alkyl group
R₂=a spermidine moiety or a substituted spermidine moiety and
X=any pharmaceutically acceptable counterion.

36. The method of claim 15, wherein when R is an ω-alkoxycarboxylic acid the method futher comprises the step of reacting the pentacyclic compound of the formula: 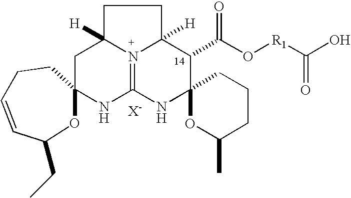
wherein, R₁=any alkyl, aryl or substituted alkyl group
with a protected spermidine or a protected substituted sperimidine and subsequently deprotecting to produce the compound of the formula: 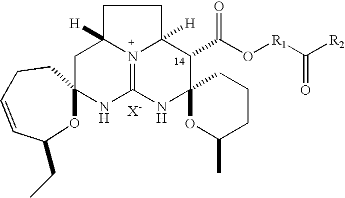
wherein R₁=any alkyl, aryl or substituted alkyl group
R₂=a spermidine moiety or a substituted spermidine moiety and
X=any pharmaceutically acceptable counterion.

37. The method of claim 18, wherein when R is an ω-alkoxycarboxylic acid the method further comprises the step of reacting the pentacyclic compound of the formula: 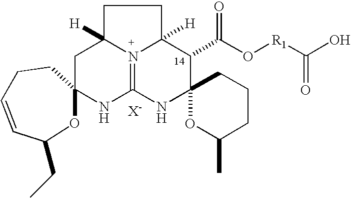
wherein, R₁=any alkyl, aryl or substituted alkyl group
with a protected spermidine or a protected substituted sperimidine and subsequently deprotecting to produce the compound of the formula: 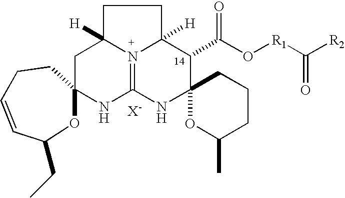
wherein R₁=any alkyl, aryl or substituted alkyl group
R₂=a spermidine moiety or a substituted spermidine moiety and
X=any pharmaceutically acceptable counterion.

38. A method for synthesizing Ptilomycalin of the formula: 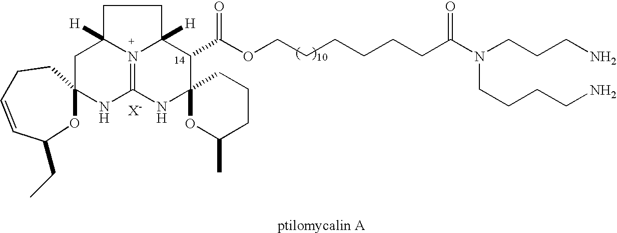
which comprises reacting the pentacyclic compound of claim 22 with the compound of the formula: 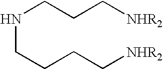
wherein R₂=an amine protecting group to produce a compound of the formula: 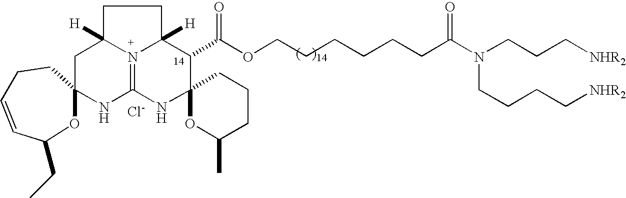
which is subsequently deprotected to produce Ptilomycalin A.

39. A method for synthesizing Crambescidin 800 of the formula: 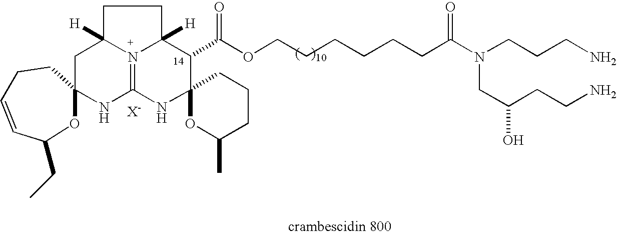
which comprises reacting the pentacyclic compound of claim 22 with the compound of the formula: 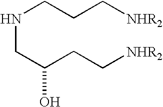
wherein R₂=an amine protecting group to produce a compound of the formula: 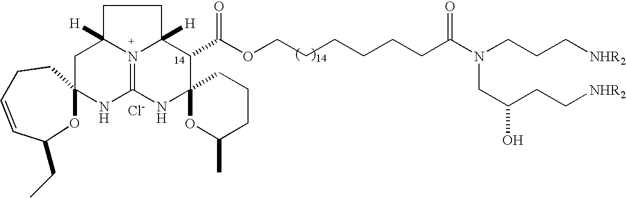
which is subsequently deprotected to produce Crambescidin 800.

40. A method for synthesizing 13,14,15-Isocrambescidin 800 of the formula: 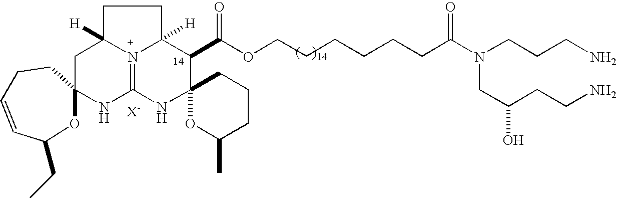
13,14,15isocrambescidin 800
which comprises reacting the pentacyclic compound of claim 24 with the compound of the formula: 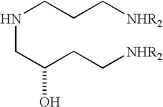
wherein R₂=an amine protecting group to produce a compound of the formula: 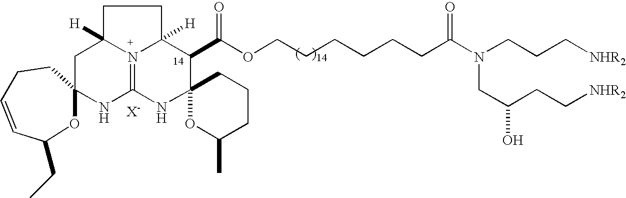
which is subsequently deprotected to produce 13,14,15-Isocrambescidin 800.

41. An antitumor composition comprising a compound of claim 1, 2, 3, 4, 5, 6, 7, 8, 9 or 10 in admixture with a pharmaceutically acceptable carrier.

42. An antiviral composition comprising a compound of claim 1, 2, 3, 4, 5, 6, 7, 8, 9, or 10 in admixture with a pharmaceutically acceptable carrier.

43. An antifungal composition comprising a compound of claim 1, 2, 3, 4, 5, 6, 7, 8, 9 or 10 in admixture with a pharmaceutically acceptable carrier.

44. A method for treating tumors comprising administering to a subject in need of said treatment, an effective amount of compound of claim 1, 2, 3, 4, 5, 6, 7, 8, 9 or 10.

45. A method for treating viral infections comprising administering to a subject in need of said treatment, an effective amount of compound of claim 1, 2, 3, 4, 5, 6, 7, 8, 9 or 10.

46. A method for treating fungal infections comprising administering to a subject in need of said treatment, an effective amount of compound of claim 1, 2, 3, 4, 5, 6, 7, 8, 9 or 10.
